(19) United States
(12) Patent Application Publication
(10) Pub. No.: US 2022/0340621 A1
(43) Pub. Date: Oct. 27, 2022

(54) TREATMENT OF CELIAC DISEASE

(71) Applicant: INTREXON ACTOBIOTICS NV d/b/a Precigen Actobio, Zwijnaarde (BE)

(72) Inventors: Lothar STEIDLER, Lokeren (BE); Karolien VAN HUYNEGEM, Asper (BE)

(73) Assignee: INTREXON ACTOBIOTICS NV d/b/a Precigen Actobio, Zwijnaarde (BE)

(21) Appl. No.: 17/763,593

(22) PCT Filed: Sep. 25, 2020
(86) PCT No.: PCT/IB2020/059013

§ 371 Date: Mar. 24, 2022

Related U.S. Application Data
(60) Provisional application No. 63/003,624, filed on Apr. 1, 2020.
(60) Provisional application No. 62/907,350, filed on Sep. 27, 2019.

(51) Int. Cl. C07K 14/415; C12N 1/20; C12N 15/62; C12N 15/74

(52) U.S. Cl. CPC C07K 14/415; C12N 1/205; C12N 15/625; C12N 15/746; A61K 39/00

(57) ABSTRACT

Microorganisms are provided, such as lactic acid bacteria (e.g., Lactococcus lactis) containing an exogenous nucleic acid encoding an IL-10 polypeptide and an exogenous nucleic acid encoding a CeD-specific antigen (e.g., a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide, wherein both exogenous nucleic acids are integrated into the bacterial chromosome. Such microbial strains are suitable for human therapy. Compositions (e.g., pharmaceutical compositions), methods of using the microorganisms and compositions are provided, e.g., for the treatment of celiac disease (CeD). The microorganism may be administered orally, delivering the microorganism into the gastrointestinal tract, where it is released and expresses the bioactive polypeptides.

### CROSS REFERENCE TO RELATED APPLICATIONS

This application claims benefit to U.S. Provisional Application No. 62/907,350, filed Sep. 27, 2019, and U.S. Provisional Application No. 63/003,624, filed Apr. 1, 2020, each of which is incorporated herein in its entirety.

### REFERENCE TO SEQUENCE LISTING

The instant application contains a Sequence Listing, which has been submitted electronically in ASCII format and is hereby incorporated by reference in its entirety. Said ASCII copy, created on September 23, 2020, is named 205350-0036-00-WO-605355_SL.txt and is 69,696 bytes in size.

### INCORPORATION BY REFERENCE

All publications, patents, and patent applications cited herein are incorporated by reference to the same extent as if each individual publication, patent, or patent application was specifically and individually indicates to be incorporated by reference. In the event of a conflict between a term herein and a term in an incorporated reference, the term herein controls.

### BACKGROUND

Genetically modified microorganisms (e.g., bacteria) have been used to deliver therapeutic molecules to mucosal tissues. See, e.g., Steidler, L., et aL, Nat. Biotechnol. 2003, 21(7): 785-789; and Robert S. and Steidler L., Microb. Cell Fact. 2014, 13 Suppl. 1: S11.

Gliadin peptides comprising a human leukocyte antigen (HLA)-DQ2-specific or HLA-DQ8-specific epitope-producing lactic acid bacteria have been previously described, and mucosally administered gliadin peptides comprising an HLA-DQ2-specific or HLA-DQ8-specific epitope have been described for the treatment of celiac disease (CeD). See, e.g., U.S. Patent No. 8,524,246; and Huibregtse et al., 2009, J. Immunol. 183:2390-2396. Interleukin-10 (IL-10) producing lactic acid bacteria have been previously described, and mucosally administered IL-10 in combination with a gliadin peptide comprising an HLA-DQ2-specific or HLA-DQ8-specific epitope have been described for the treatment of CeD. See, e.g., U.S. Pat. No. 8,748,126.

However, there is still a need in the art for genetically modified bacterial strains that are stable, and which constitutively or inducibly express more than one bioactive polypeptide and are suitable for clinical usage, e.g., for the treatment of CeD. The present disclosure addresses these needs.

### SUMMARY

The current disclosure provides genetically modified microorganisms containing chromosomally integrated nucleic acids encoding cytokine interleukin-10 (IL-10) and a gliadin peptide comprising at least one human leukocyte antigen (HLA)-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope, methods of preparing such microorganisms and nucleic acids useful in such methods of preparing, and methods of using such microorganisms. In alternative embodiments, interleukin-2 (IL-2) is used in place of IL-10. The genetically modified microorganisms can be suitable to human therapy, including but not limited to the treatment of celiac disease.

The present disclosure provides a lactic acid bacterium (LAB) comprising an exogenous nucleic acid encoding a secretion leader sequence fused in frame to a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope. The exogenous nucleic acid can be chromosomally integrated in the LAB. The secretion leader fused to the gliadin polypeptide can be selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36, and variants thereof having 1, 2, or 3 variant amino acid positions. In some examples of the LAB, the exogenous nucleic acid encoding a gliadin polypeptide encodes a gliadin polypeptide comprising or consisting of: LQLQPFPQPQLPYPQPQLPYPQPQLPYPQPQPF (SEQ ID NO: 3) (DQ2), LQLQPFPQPELPYPQPQLPYPQPELPYPQPQPF (SEQ ID NO: 7) (dDQ2), or LQLQPFPQPELPYPQPELPYPQPELPYPQPQPF (SEQ ID NO: 33). The exogenous nucleic acid encoding a gliadin polypeptide encodes a gliadin polypeptide comprising or consisting of: LQLQPFPQPELPYPQPQLPYPQPELPYPQPQPF (SEQ ID NO: 7) (dDQ2), and a secretion leader selected from the secretion leader group consisting of SL #17, SL #21, SL #22, and SL #23.

The present disclosure also provides a lactic acid bacterium (LAB) comprising: (i) an exogenous nucleic acid encoding human interleukin-10 (hIL-10) and (ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope. The exogenous nucleic acid encoding hIL-10 and the exogenous nucleic acid encoding a gliadin polypeptide can be chromosomally integrated in the LAB. The exogenous nucleic acid encoding the hIL-10 can further encode a secretion leader sequence fused to the hIL-10 coding sequence. The hIL-10 can be secreted as a mature hIL-10 without the secretion leader. Optionally, the hIL-10 comprises alanine (Ala) instead of proline (Pro) at position 2 of the mature sequence.

In certain examples, the exogenous nucleic acid encoding the gliadin polypeptide can further encodes a secretion leader sequence fused to the gliadin polypeptide coding sequence. In some examples, the secretion leader fused to the gliadin polypeptide is selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36, and variants thereof having 1, 2, or 3 variant amino acid positions. The secretion leader fused to the gliadin polypeptide can be selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36. In certain examples, the gliadin polypeptide comprises an HLA-DQ2 specific epitope and the secretion leader fused to the gliadin polypeptide is selected from the secretion leader group consisting of SL #1, SL #6, SL #8, SL #9, SL #13, SL #15, SL #17, SL #20, SL #21, SL #22, SL #23, SL #24, SL #25, and SL #36. In certain examples, the gliadin polypeptide comprises a deamidated HLA-DQ2 specific epitope, and the secretion leader fused to the gliadin polypeptide is selected from the secretion leader group consisting of SL #1, SL #6, SL #8, SL #9, SL #13, SL #15, SL #17, SL #20, SL #21, SL #22, SL #23, SL #25, and SL #36. In some examples, the gliadin polypeptide comprises an α1- and/or an α2-gliadin epitope. In some examples of the LAB, the exogenous nucleic acid encoding a gliadin polypeptide encodes a gliadin polypeptide comprising or consisting of: LQLQPFPQPQLPYPQPQLPYPQPQLPYPQPQPF (SEQ ID NO: 3) (DQ2), LQLQPFPQPELPYPQPQLPYPQPELPYPQPQPF (SEQ ID NO: 7) (dDQ2), or lqlqpfpqpelpypqpElpypqpelpypqpqpf (SEQ ID NO: 33). The exogenous nucleic acid encoding a gliadin polypeptide can encode a gliadin polypeptide comprising or consisting of: LQLQPFPQPELPYPQPQLPYPQPELPYPQPQPF (SEQ ID NO: 7) (dDQ2), and can further encode a secretion leader selected from the secretion leader group consisting of SL #17, SL #21, SL #22, and SL #23.

In certain examples of the LAB, the LAB comprises a polycistronic expression unit comprising the exogenous nucleic acid encoding hIL-10 and the exogenous nucleic acid encoding the gliadin polypeptide. In certain examples, the polycistronic expression unit comprises (i) an endogenous gene promoter of an endogenous gene, (ii) the endogenous gene positioned 3′ of the endogenous gene promoter, (iii) an intergenic region, and (iv) the exogenous nucleic acid encoding hIL-10. The exogenous nucleic acid encoding hIL-10 can further encode a secretion leader sequence fused in frame to the hIL-10 coding sequence, and the endogenous gene and the exogenous nucleic acid encoding hIL-10 can be transcriptionally and translationally coupled by the intergenic region. In some examples, the polycistronic expression unit can further comprises (i) a second intergenic region positioned 3′ of the exogenous nucleic acid encoding hIL-10, and (ii) the exogenous nucleic acid encoding the gliadin polypeptide. The exogenous nucleic acid encoding the gliadin polypeptide can further encodee a secretion leader sequence fused in frame to the gliadin polypeptide. The exogenous nucleic acid encoding the gliadin polypeptide and the exogenous nucleic acid encoding hIL-10 can be transcriptionally and translationally coupled by the second intergenic region.

In other examples, the polycistronic expression unit of the LAB comprises: (i) an endogenous gene promoter of an endogenous gene, (ii) the endogenous gene positioned 3′ of the endogenous gene promoter, (iii) an intergenic region, and (iv) the exogenous nucleic acid encoding the gliadin polypeptide. The exogenous nucleic acid encoding the gliadin polypeptide can further encode a secretion leader sequence fused to the gliadin polypeptide, and wherein the endogenous gene and the exogenous nucleic acid encoding the gliadin polypeptide can be transcriptionally and translationally coupled by thr intergenic region. In some examples, the polycistronic expression unit can further comprise (i) a second intergenic region positioned 3′ of the exogenous nucleic acid encoding the gliadin polypeptide, and (ii) the exogenous nucleic acid encoding hIL-10. The exogenous nucleic acid encoding hIL-10 further can encode a secretion leader sequence fused to the hIL-10 coding sequence, and wherein the exogenous nucleic acid encoding hIL-10 and the exogenous nucleic acid encoding the gliadin polypeptide can be transcriptionally and translationally coupled by the second intergenic region.

In some examples, the LAB constitutively expresses and secretes the hIL-10 and the gliadin polypeptide. In certain examples, the LAB comprises the following chromosomally integrated polycistronic expression cassettes:
a. a first polycistronic expression cassette comprising an eno promoter positioned 5′ of an eno gene, a first intergenic region, an hIL-10 secretion leader sequence, the exogenous nucleic acid encoding hIL-10; a second intergenic region, a gliadin polypeptide secretion leader sequence, and the exogenous nucleic acid encoding the gliadin polypeptide;
b. a second polycistronic expression cassette comprising a usp45 promoter, usp45, and an exogenous nucleic acid encoding a trehalose-6-phosphate phosphatase and optionally an intergenic region, such as rpmD, between the usp45 and the exogenous nucleic acid encoding the trehalose-6-phosphate phosphatase; and
c. a third polycistronic expression cassette comprising nucleic acid encoding one or more trehalose transporters positioned 3′ of an hllA promoter (PhllA).
   The LAB can be genetically modified to include:
d) inactivation or deletion of a trehalose-6-phosphate phosphorylase gene (trePP);
e) inactivation or deletion of a gene encoding a cellobiose-specific PTS system IIC component (ptcC); and
f) deletion of a thymidylate synthase gene (thyA).

In certain examples of the LAB, the trehalose-6-phosphate phosphatase is Escherichia coli otsB. In certain examples of the LAB, the third polycistronic expression cassette comprises trehalose transporters genes LLMG_RS02300 and LLMG_RS02305.

The disclosure further provides a composition comprising a LAB of any of the disclosed LAB. In an example, the composition comprises first LAB containing an exogenous nucleic acid encoding an interleukin-10 (IL-10) polypeptide and expresses the IL-10 polypeptide; and a second LAB containing an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope. In an example, the composition comprises (i) an exogenous nucleic acid encoding human interleukin-10 (hIL-10) and (ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope. The exogenous nucleic acid encoding hIL-10 and the exogenous nucleic acid encoding a gliadin polypeptide can be chromosomally integrated in the LAB. In an example, the composition comprises a first LAB containing an exogenous nucleic acid encoding an interleukin-10 (IL-10) polypeptide and expresses the IL-10 polypeptide; and a second LAB containing an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope. The exogenous nucleic acid encoding hIL-10 and the exogenous nucleic acid encoding a gliadin polypeptide can be chromosomally integrated in the LAB. In an example, the composition comprises a lactic acid bacterium (LAB) comprising an exogenous nucleic acid encoding a secretion leader sequence fused in frame to a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope. The exogenous nucleic acid can be chromosomally integrated in the LAB.

Also provided is the use of any of the above described LAB or a composition comprising an LAB in the treatment of celiac disease. Futher provided is the use of any of the above described LAB or a composition comprising an LAB for the preparation of a medicament for the treatment of celiac disease.

The disclosure provides a polynucleotide sequence comprising a polycistronic expression unit comprising (i) a nucleic acid encoding hIL-10, and (ii) a nucleic acid encoding a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope. In the polynucleotide sequence, the nucleic acid encoding hIL-10 can further encode a secretion leader sequence fused to the hIL-10, and/or the nucleic acid encoding the gliadin polypeptide can further encode a secretion leader sequence fused to the gliadin polypeptide. The nucleic acid encoding the gliadin polypeptide and the nucleic acid encoding hIL-10 can be transcriptionally and translationally coupled by an intergenic region. In an example, the polynucleotide sequence, further comprises an L. lactis promoter positioned 5′ to the exogenous nucleic acid encoding hIL-10, and the exogenous nucleic acid encoding hIL-10 is transcriptionally controlled by the L. lactis promoter. The L. lactis promoter can be selected from the group comprising eno promoter, P1 promoter, usp45 promoter, gapB promoter, thyA promoter, and hllA promoter.

Also provided is a polynucleotide sequence comprising a polycistronic integration vector comprising (i) a first intergenic region, (ii) a first open reading frame encoding a first therapeutic protein, (iii) a second intergenic region, and (iv) a second open reading frame encoding a second therapeutic protein. The first intergenic region is transcriptionally coupled at its 3′ end to the first open reading frame, the second intergenic region is transcriptionally coupled to the 3′ end of the first open reading frame, and the second intergenic region is transcriptionally coupled at its 3′ end to the second open reading frame. In an example, one of either the first open reading frame and second open reading frame encodes hIL-10, and the other of the first open reading frame and second open reading frame encodes a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope. In an example, the first open reading frame can further encode a secretion leader sequence fused to the first therapeutic protein and the second open reading frame further can further encode a secretion leader sequence fused to the second therapeutic protein. In some examples, the polynucleotide sequence can further comprise nucleic acid sequences flanking the 5′ and 3′ ends of the at least one intergenic region transcriptionally coupled to at least one open reading frame or coding region, and the 5′ flanking nucleic acid comprises a nucleic acid sequence that is identical to coding sequence at the 3′ end of an integration target gene.

Also provided is a polynucleotide sequence comprising (a) a polycistronic expression unit comprising: (i) a nucleic acid encoding hIL-10, and (ii) a nucleic acid encoding a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope. The nucleic acid encoding hIL-10 can further encode a secretion leader sequence fused to the hIL-10, and/or the nucleic acid encoding the gliadin polypeptide can further encode a secretion leader sequence fused to the gliadin polypeptide.

Also provided is a polynucleotide sequence comprising a polycistronic integration vector comprising (i) a first intergenic region, (ii) a first open reading frame encoding a first therapeutic protein, (iii) a second intergenic region, and (iv) a second open reading frame encoding a second therapeutic protein. The first intergenic region is transcriptionally coupled at its 3′ end to the first open reading frame, the second intergenic region is transcriptionally coupled to the 3′ end of the first open reading frame, and the second intergenic region is transcriptionally coupled at its 3′ end to the second open reading frame.

The present disclosure also provides therapeutic methods for celiac disease. In any of the therapeutic methods, the LAB administered can be one or more of the LAB described above and in the detailed disclosure. In some examples. the LAB is sAGX0868.

In an example, a method of inducing oral tolerance to gluten in a subject at risk of celiac disease is provided. The method comprises administering to a subject at risk of celiac disease a therapeutically effective amount of a lactic acid bacterium (LAB) engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope, thereby inducing oral tolerance. In an example, the exogenous nucleic acid encoding IL-10 and the exogenous nucleic acid encoding a gliadin polypeptide can be chromosomally integrated in the LAB. In some examples, the interleukin-10 is human interleukin-10 (hIL-10). In some examples, the subject at risk of celiac disease exhibits a risk factor, wherein the risk factor is a genetic predisposition. In some examples, administering the therapeutically effective amount of the LAB in the subject increases tolerance-inducing lymphocytes in a sample of lamina propria cells of the subject.

In some examples of the method of inducing oral tolerance to gluten in a subject, administering the therapeutically effective amount of the LAB in the subject increases CD4+ Foxp3+ regulatory T cells in a sample of lamina propria cells of the subject. In some examples of the method, administering the therapeutically effective amount of the LAB in the subject increases a ratio of CD4+ Foxp3+ regulatory T cells over TH1 cells expressing Tbet in a sample of lamina propria cell of the subject. In some examples of the method, the development of villous atrophy upon exposure to gluten is prevented, inhibited or minimized in the subject. In some examples of the method of inducing oral tolerance to gluten in a subject, more than one of the above described therapeutic effects is achieved.

In an example, a method of reducing villous atrophy in a subject diagnosed with celiac disease is provided. The method comprises comprising administering to the subject having villous atrophy a therapeutically effective amount of a LAB engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2 specific epitope and/or at least one deamidated HLA-DQ2 specific epitope, and (b) at least one HLA-DQ8 specific epitope and/or at least one deamidated HLA-DQ8 specific epitope, wherein the administration of the LAB produces at least a 55% reduction of the villous atrophy relative to a reference LAB that does not express IL-10 and the gliadin polypeptide in a mouse model of celiac disease. In some examples, the interleukin-10 is human interleukin-10 (hIL-10). In some examples, the villous atrophy is present in the subject due to intestinal gluten exposure. In some examples, adminstration of the LAB produces at least a 60%, 65%, 70%, 75%, 80%, 85%, 90%, 91%, 92%, 93%, 94%, 95%, 96%, 97% 98%, 99% or 100% reduction of the villous atrophy relative to the reference LAB that does not express IL-10 and the gliadin polypeptide in a mouse model of celiac disease. In some examples of the method of reducing villous atrophy in a subject, the adminstering step reduces intraepithelial lymphocytosis in the subject as compared to intraepithelial lymphocytosis prior to administration to the subject and/or reduces the level of CD3+ intraepithelial lymphocytes (IELs) in a sample obtained from the subject as compared to CD3+ IELs present in a sample obtained from the subject prior to the administering step. In some examples of the method, the administering step reduces the number of cytotoxic CD8+ IELs in the subject as compared to the cytotoxic CD8+ IELs present in a sample of the subject prior to administration. In some examples of the method, the administering step reduces the level of Foxp3−Tbet+CD4+ T cells of the subject as compared to the Foxp3−Tbet+CD4+ T cells present in a sample of the subject prior to administration and/or increases the level of Foxp3+Tbet-CD4+ T cells in a sample of lamina propria lymphocytes of the subject compared to the Foxp3-Tbet+CD4+ T cells present in a sample of the subject prior to administration. In some examples of the method, the administering step prevents, inhibits or minimizes villous atrophy recurrence in the subject upon exposure to gluten. In some examples of the method, the administering step improves villous height (Vh) -to-crypt depth (Cd) ratio in the subject and/or restores the Vh/Cd ratio to a normal range in the subject. In some examples of the method of reducing villous atrophy in a subject , more than one of the above described therapeutic effects is achieved.

The current disclosure further provides kits containing (1) a microorganism (e.g., LAB such as sAGX0868) according to any of the embodiments disclosed herein, a composition containing a microorganism (e.g., LAB) according to any of the embodiments described herein, a pharmaceutical composition containing a microorganism (e.g., LAB) according to any of the embodiments described herein, or a unit dosage form containing a microorganism (e.g., LAB) according to any of the embodiments described herein; and (2) instructions for administering the microorganism (e.g., LAB), the composition, the pharmaceutical composition, or the unit dosage form to a mammal, e.g., a human (e g , human patient).

In each of the above-described above methods, products, and compositions, and as further disclosed herein, interleukin-10 is the primary cytokine of choice. In each of the above-described above methods, products, and compositions, and as further disclosed herein, interleukin-2 is an alternative to interleukin-10.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1, comprised of FIGS. 1A and 1B, depicts graphs of villous atrophy (VA) (FIG. 1A) and villous to crypt ratio (FIG. 1B). Villous atrophy (VA) was assessed on H&E stained sections. The villous height to crypt depth ratio (Vh/Cd; labeled V/Cr in the figure) was determined by measuring up to 6 villous (V) and crypts (Cr) from the most damaged areas. Atrophy was confirmed when the Vh/Cd ratio ≤2.0. Kruskal-Wallis with Dunn's multiple comparison test (FIG. 1A), or ANOVA with Tukey's multiple comparison test (FIG. 1B) was used to test for statistical differences.

FIG. 2 is a graph of number of CD3+ intraepithelial lymphocytes (IELs) per 100 intestinal epithelial cells in mice treated with different strains of L. lactis (LL) as compared to the control group. IELs counts were evaluated by an independent and blinded investigator. Kruskal-Wallis with Dunn's test for multiple comparisons was used as a statistical test and did not show significant differences between the groups.

FIG. 3, comprised of FIGS. 3A, 3B, 3C, and 3D, are graphs depicting flow cytometry analysis of intraepithelial lymphocytes (IELs) from L. lactis (LL)-treated mice. FIGS. 3A and 3B depict data for expression of NKG2D in CD8αβ+ T cells. FIGS. 3C and 3D depict data for expression of NKG2D in CD4+ T cells. FIGS. 3A and 3C show the percentage of the indicated population. FIGS. 3B and 3D show the absolute number of CD3+ cells among 100 epithelial cells (IECs). Kruskal-Wallis with Dunn's multiple comparison test was used as statistical test and did not reveal any significant differences between the groups. FIG. 3A, LL-empty vector versus (vs.) LL-[dDQ8] P=0.2302, and vs. LL-[dDQ8]+IL10 P=0.8, other comparisons P>0.99. FIG. 3B, LL-empty vector vs. LL-[dDQ8] P=0.3898, and vs. LL-[dDQ8]+IL10 P=0.351, other comparisons P>0.99. FIG. 3C, P=0.634 LL-Empty vector vs. LL-[dDQ8]; P=0.3521 LL-Empty vector vs. LL-[dDQ8]+IL-10. FIG. 3D, P=0.2823 LL-Empty vector vs. LL-[dDQ8]; P=0.2229 LL-Empty vector vs. LL-[dDQ8]+IL-10.

FIG. 4, comprised of FIGS. 4A, 4B, and 4C, are graphs depicting flow cytometry analysis of lamina propria cells. FIG. 4A depicts the data for CD4⁺ Foxp3⁺ regulatory T cells (Tregs). FIG. 4B depicts the data for CD4⁺Tbet⁺ T_{H}1 population. FIG. 4C depicts the ratio of Tregs over T_{H}1 is shown. Kruskal-Wallis with Dunn's multiple comparison test were used as statistical tests, and no significant differences between groups was found.

FIG. 5, comprised of FIGS. 5A, 5B, 5C, and 5D, are graphs depicting levels of gene expression in epithelial cells. The expression of Qa-1 (FIG. 5A), Rae1 (FIG. 5B), Mult1 (FIG. 5C) and Prf1 (FIG. 5D) was assessed. mRNA was isolated from the IEL fraction and transcribed to cDNA to perform qPCR for the indicated genes. Kruskal-Wallis with Dunn's multiple comparison test (FIG. 5A) or ANOVA with Tukey's multiple comparison test (FIGS. 5B-5D) were used to test for statistical differences. The mean with standard error of the mean is displayed.

FIG. 6, comprising FIGS. 6A and 6B, are graphs depicting ELISA assay data. Anti-deamidated gluten peptides (anti-DGP) IgG and anti-gliadin IgG2c antibody serum levels were determined by ELISA assays. Data for anti-DGP IgG is shown in FIG. 6A, and data for anti-gliadin IgG2c is shown in FIG. 6B. Data are expressed as the OD450 nm. Kruskal-Wallis with Dunn's multiple comparison test was used to determine statistical differences between the groups.

FIG. 7, comprised of FIGS. 7A and 7B, depicts graphs of villous atrophy (VA) (FIG. 7A) and villous height to crypt depth ratio (FIG. 7B). Villous atrophy (VA) was assessed on H&E stained sections. The villous height to crypt depth ratio (Vh/Cd; labeled Villous/crypt ratio in the figure) was determined by measuring up to 6 villous (V) and crypts (Cr) from the most damaged areas. Atrophy was confirmed when the Vh/Cd ratio ≤2.0. ANOVA with Tukey's multiple comparison test was used to test for statistical differences. *P<0.05.

FIG. 8 is a graph of number of CD3⁺ intraepithelial lymphocytes (IEL) per 100 intestinal epithelial cells in mice treated with different strains of L. lactis (LL) as compared to the control group. IEL counts were evaluated by an independent and blinded investigator. ANOVA with Tukey's multiple comparison test was used to test for statistical differences and did not show significant differences between the groups.

FIG. 9, comprised of FIGS. 9A, 9B, and 9C, are graphs depicting flow cytometry analysis of intraepithelial lymphocytes (IELs) from L. lactis (LL)-treated mice. FIGS. 9A and 9B depict data for expression of NKG2D on CD8αβ⁺ T cells and on CD4+ T cells, respectively. FIGS. 9A and 9B show the absolute number of CD3+ cells among 100 epithelial cells (IECs). The expression of Granzyme B was also determined on CD8αβ⁺ T cells (FIG. 9C). ANOVA with Tukey's multiple comparison test was used as statistical test.

FIG. 10, comprised of FIGS. 10A, 10B, and 10C, are graphs depicting flow cytometry analysis of lamina propria cells. FIG. 10A depicts the data for CD4+^{F}oxp3⁺ regulatory T cells (Tregs). FIG. 10B depicts the data for CD4⁺ Tbet⁺T_{H}1 population. FIG. 10C depicts the ratio of Tregs over T_{H}1 is shown. ANOVA with Tukey's multiple comparison test was used as statistical test, and no significant differences between groups was found.

FIG. 11, comprised of FIGS. 11A, 11B, 11C, and 11D, are graphs depicting levels of gene expression in epithelial cells. The expression of Qa-1 (FIG. 11A), Rae1 (FIG. 11B), Mult1 (FIG. 11C) and Prf1 (FIG. 11D) was assessed. mRNA was isolated from the IEL fraction before Percoll separation (FIGS. 11A-11C) and after (FIG. 11D), and transcribed to cDNA to perform qPCR for the indicated genes. Mean with standard error of the mean is displayed; ANOVA with Tukey's multiple comparison test were used to test for statistical differences. *P<0.05. **P<0.01.

FIG. 12 includes images of Western blots of candidate secretion leaders sequences for DQ2. The secretion leader tested is indicated by the corresponding Uniprot number of its parent protein. The plate number and well for each clone is indicated, followed by the secretion leader number (SL #; see Table 14). Expected sizes are indicated in the left column. The mass of the secretion leaders ranges from 2.3 to 3 kDa. MG1363[pAGX0043] was used as reference material to check for antibody reactivity. MG1363[pT1NX] was used as empty vector control. SeeBlue™ Plus2 (Thermo Fisher Scientific #LC5925) was used as Molecular Weight Marker (MWM). Clones with mutations in promoter, SL or DQ2 are marked in red.

FIG. 13 includes images of Western blots of candidate secretion leaders sequences for dDQ2. The secretion leader is indicated by the corresponding Uniprot number of its parent protein. The plate number and well for each clone is indicated, followed by the secretion leader number (SL #; see Table Ex. I). Expected sizes are indicated in the left column. The mass of the secretion leaders ranges from 2.3 to 3 kDa. MG1363[pAGX0043] was used as reference material to check for antibody reactivity. MG1363[pT1NX] was used as empty vector control. SeeBlue™ Plus2 (Thermo Fisher Scientific #LC5925) was used as Molecular Weight Marker (MWM). Clones with mutations in promoter, SL or DQ2 are marked in red.

FIG. 14 includes images of Western blots of select secretion leaders sequences for DQ2. The secretion leader is indicated by the secretion leader number (SL #; see Table Ex. I) and the corresponding Uniprot number of its parent protein. Expected sizes are indicated in the left column. The mass of the secretion leaders ranges from 2.3 to 3 kDa. MG1363[pAGX0043] was used as reference material to check for antibody reactivity. MG1363[pT1NX] was used as empty vector control. SeeBlue™ Plus2 (Thermo Fisher Scientific #LC5925) was used as Molecular Weight Marker (MWM).

FIG. 15 includes images of Western blots of select secretion leaders sequences for dDQ2. The secretion leader is indicated by the secretion leader number (SL #; see Table Ex. I) and the corresponding Uniprot number of its parent protein. Expected sizes are indicated in the left column. The mass of the secretion leaders ranges from 2.3 to 3 kDa. MG1363[pAGX0043] was used as reference material to check for antibody reactivity. MG1363[pT1NX] was used as empty vector control. SeeBlue™ Plus2 (Thermo Fisher Scientific #LC5925) was used as Molecular Weight Marker (MWM).

FIG. 16 depicts a schematic overview of relevant genetic loci of sAGX0868 as described: eno>>hil-10>>ddq2, ΔthyA, otsB, trePTS, ΔtrePP, ΔptcC, (/truncated/) genetic characters, intergenic regions (IR), PCR amplification product sizes (base pairs or bp).

FIG. 17, comprised of FIGS. 17A, 17B, 17C, and 17D (SEQ ID NO: 26) are collectively a representation of a deletion of the trehalose-6-phosphate phosphorylase gene (trePP; Gene ID: 4797140); Insertion of the constitutive promoter of the HU-like DNA-binding protein gene (PhllA; Gene ID: 4797353) to precede the putative phosphotransferase genes in the trehalose operon (trePTS; llmg_0453 (LLMG_RS02300) and llmg_0454 (LLMG_RS02305); ptsI and ptsII; Gene ID: 4797778 and Gene ID: 4797093 respectively), insertion of the intergenic region preceding the highly expressed L. lactis MG1363 50S ribosomal protein L30 gene (rpmD; Gene ID: 4797873) in between ptsI and ptsII. In FIG. 17D, pgmB refers to beta-phosphoglucomutase gene (Locus tag LLMG_RS02315).

FIG. 18, comprised of FIGS. 18A, 18B, and 18C (SEQ ID NO: 27) are collectively a representation of insertion of trehalose-6-phosphate phosphatase gene (otsB; Gene ID: 1036914) downstream of unidentified secreted 45-kDa protein gene (usp45; Gene ID: 4797218). Insertion of the intergenic region preceding the highly expressed L. lactis MG1363 505 ribosomal protein L30 gene (rpmD; Gene ID: 4797873) between usp45 and otsB. In FIG. 18C, asnH refers to asparagine synthase gene (Locus tag LLMG_RS12590).

FIG. 19, comprised of FIGS. 19A, 19B, 19C, and 19D (SEQ ID NO: 28) are collectively a representation of deletion of the gene encoding cellobiose-specific PTS system IIC component (ptcC; Gene ID: 4796893). In FIGS. 19B-19D, bglA refers to 6-phospho-beta-glucosidasegene (Gene ID 4798119; Locus tag LLMG_RS0224).

FIG. 20, comprised of FIGS. 20A and 20B (SEQ ID NO: 29) are collectively a representation of deletion of thymidylate synthase gene (thyA; Gene ID: 4798358). In FIGS. 20A and B, PTS refers to Locus tag LLMG_RS04900 (GeneID 4796722; llmg_0963).

FIG. 21, comprised of FIGS. 21A, 21B, 21C, and 21D (SEQ ID NO: 30), are collectively a representation insertion of a gene (SEQ ID NO: 22 including TAA stop codon) encoding a fusion of usp45 secretion leader (SSusp45) with the hil-10 gene, encoding human interleukin-10 (hIL-10; UniProt: P22301, aa 19-178, variant Pro2Ala (P2A); Steidler et al., Nat. Biotechnol. 2003, 21(7): 785-789) downstream of the phosphopyruvate hydratase gene (eno; Gene ID: 4797432), to allow expression and secretion of hIL-10. The hil-10 expression unit is transcriptionally and translationally coupled to eno by use of IRrpmD. A gene (SEQ ID NO: 25 including TAA stop codon) encoding a fusion of ps356 secretion leader (SSps356) with a fragment encoding deamidated DQ2 (ddq2), a protease-resistant 33-mer based on 6 overlapping α1- and α2-gliadin epitopes (UniProt: Q9M4L6_wheat, amino acids 57-89, glutamine deamidation at positions 66 and 80), is positioned downstream of this hil-10 gene, to allow expression and secretion of dDQ2. The ddq2 expression unit is transcriptionally and translationally coupled to hil-10 by use of IR preceding the highly expressed L. lactis MG1363 505 ribosomal protein L14 gene (rplN; Gene ID: 4799034). In FIGS. 21C-21D, xerD refers to integrase-recombinase gene (GeneID 4796855; Locus tag LLMG_RS03220).

### DETAILED DESCRIPTION

Provided are compositions and methods for the treatment of CeD, and/or for restoring tolerance to a CeD-specific antigen polypeptide, such as human leukocyte antigen (HLA)-specific gliadin antigens, e.g., an HLA-DQ2-specific epitope and/or an HLA-DQ8 -specific epitope, in a subject.

### A. Detailed Description

### Microorganisms and Compositions

The present disclosure provides microorganisms, e.g., Gram-positive bacteria, such as a lactic acid bacterium (LAB) containing an exogenous nucleic acid encoding an IL-10 polypeptide, and an exogenous nucleic acid encoding a CeD-specific antigen, such as a gliadin peptide comprising at least one human leukocyte antigen (HLA)-DQ2-specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope, wherein the exogenous nucleic acid encoding the IL-10 polypeptide and the exogenous nucleic acid encoding the CeD-specific antigen (e.g., a gliadin peptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) are both chromosomally integrated, i.e., are integrated into (or located on) the bacterial chromosome.

The microorganism can be a Gram-positive bacterium, such as an LAB. The LAB can be a Lactococcus species bacterium. An exemplary LAB species includes a Lactobacillus species, or a Bifidobacterium species. The LAB can be Lactococcus lactis. The LAB can be Lactococcus lactis subspecies cremoris. Another exemplary LAB is a Lactococcus lactis strain MG1363. See, e.g., Gasson, M. J., J. Bacteriol. 1983, 154: 1-9.

In some examples according to any of the above embodiments, the CeD-specific antigen comprises at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope from a gluten associated with CeD. In some examples, the CeD-specific antigen comprises at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope from gliadin of a wheat gluten, a rye gluten or a barley gluten. In some examples, the gliadin is wheat gliadin (UniProtKB Q9M4L6):

(SEQ ID NO: 1)
MVRVPVPQLQPQNPSQQQPQEQVPLVQQQQFPGQQQPFPPQQPYPQPQPF
PSQQPYLQLQPFPQPQLPYPQPQLPYPQPQLPYPQPQPFRPQQPYPQSQP
QYSQPQQPISQQQQQQQQQQQQKQQQQQQQQILQQILQQQLIPCRDVVLQ
QHSIAYGSSQVLQQSTYQLVQQLCCQQLWQIPEQSRCQAIHNVVHAIILH
QQQQQQQQQQQQPLSQVSFQQPQQQYPSGQGSFQPSQQNPQAQGSVQPQQ
LPQFEEIRNLALETLPAMCNVYIPPYCTIAPVGIFGTNYR.

The underlined sequence (amino acid residues 57 to 89) is an exemplary polypeptide comprising at least one HLA-DQ2-specific epitope. An exemplary nucleic acid encoding wheat gliadin (UniProtKB Q9M4L6) is given in GenBank Accession no. AJ133611.1:

| (SEQ ID NO: 2) | |
|---|---|
| ATGGTTAGAG TTCCAGTGCC ACAATTGCAG CCACAAAATC CATCTCAGCA | 50 |
| ACAGCCACAA GAGCAAGTTC CATTGGTACA ACAACAACAA TTTCTAGGGC | 100 |
| AGCAACAACC ATTTCCACCA CAACAACCAT ATCCACAGCC GCAACCATTT | 150 |
| CCATCACAAC TACCATATCT GCAGCTGCAA CCATTTCCGC AGCCGCAACT | 200 |
| ACCATATTCA CAGCCACAAC CATTTCGACC ACAACAACCA TATCCACAAC | 250 |
| CGCAACCACA GTATTCGCAA CCACAACAAC CAATTTCACA GCAGCAGCAG | 300 |
| CAGCAGCAGC AGCAGCAACA ACAACAACAA CAACAACAAC AAATCCTTCA | 350 |
| ACAAATTTTG CAACAACAAC TGATTCCATG CATGGATGTT GTATTGCAGC | 400 |
| AACACAACAT AGCGCATGGA AGATCACAAG TTTTGCAACA AAGTACTTAC | 450 |
| CAGCTGTTGC AAGAATTGTG TTGTCAACAC CTATGGCAGA TCCCTGAGCA | 500 |
| GTCGCAGTGC CAGGCCATCC ACAATGTTGT TCATGCTATT ATTCTGCATC | 550 |
| AACAACAAAA ACAACAACAA CAACCATCGA GCCAGGTCTC CTTCCAACAG | 600 |
| CCTCTGCAAC AATATCCATT AGGCCAGGGC TCCTTCCGGC CATCTCAGCA | 650 |
| AAACCCACAG GCCCAGGGCT CTGTCCAGCC TCAACAACTG CCCCAGTTCG | 700 |
| AGGAAATAAG GAACCTAGCG CTACAGACGC TACCTGCAAT GTGCAATGTC | 750 |
| TACATCCCTC CATATTGCAC CATCGCGCCA TTTGGCATCT TCGGTACTAA | 800 |
| CTATCGATGA | |

In some examples of any of the above embodiments, the CeD-specific antigen is a CeD-specific antigen variant that is a truncated version of gliadin, e.g., a truncated wheat gliadin polypeptide. The CeD-specific antigen can comprise or consist of an HLA-DQ2-specific epitope that is a 33 amino acid fragment of wheat gliadin comprising 6 overlapping α1- and α2-gliadin epitopes LQLQPFPQPQLPYPQPQLPYPQPQLPYPQPQPF (SEQ ID NO: 3). An exemplary coding sequence for this epitope is:

(SEQ ID NO: 4)
CTTCAACTTCAACCATTTCCACAACCACAACTTCCATACCCACAACCACA
ACTTCCATACCCACAACCACAACTTCCATACCCACAACCACAACCATTT.

In some examples according to any of the above embodiments, the CeD-specific antigen can comprise or consist of a an HLA-DQ8-specific epitope having the amino acid sequence QYPSGQGSFQPSQQNPQA (SEQ ID NO: 5 ; amino acid residues 225-242 of wheat gliadin (UniProtKB Q9M4L6)). An exemplary coding sequence for this epitope is:

(SEQ ID NO: 6)
CAATACCCATCAGGTCAAGGTTCATTTCAACCATCACAACAAAACCCACA
AGCT.

In other examples, the CeD-specific antigen is a CeD-specific antigen variant that is a mutated version of a gliadin, such as a mutated version of a wheat gliadin. A CeD-specific antigen can comprise or consist of a HLA-DQ2-specific epitope that is a 33 amino acid fragment of wheat gliadin comprising 6 overlapping α1- and α2-gliadin epitopes that is modified to replace two specific glutamine residues with glutamate residues: LQLQPFPQPELPYPQPQLPYPQPELPYPQPQPF (SEQ ID NO: 7). An exemplary coding sequence for this epitope is CTTCAACTTCAACCATTTCCACAACCAGAACTTCCATACCCACAACCACAACTTCC ATACCCACAACCAGAACTTCCATACCCACAACCACAACCATTT (SEQ ID NO: 8). In some examples according to any of the above embodiments, the CeD-specific antigen can comprise or consist of an HLA-DQ8-specific epitope having the amino acid sequence QYPSGEGSFQPSQENPQA (SEQ ID NO: 9). An exemplary coding sequence for this epitope is:

(SEQ ID NO: 10)
CAATACCCATCAGGTGAAGGTTCATTCCAACCATCACAAGAAAACCCACA
AGCT.

In other examples of any of the above embodiments, the CeD-specific antigen can be a CeD-specific antigen variant that is a mutated version of a gliadin, such as a mutated version of a wheat gliadin, wherein the antigen retains HLA-DQ8-specific or HLA-DQ2 specific antigenic properties. Alternatively, the CeD-specific antigen variant polypeptide can have an amino acid sequence at least 90%, at least 92%, at least 95%, at least 96%, at least 97%, at least 98%, or at least 99% identical to wheat gliadin (UniProtKB Q9M4L6), or to a fragment thereof, such as LQLQPFPQPQLPYPQPQLPYPQPQLPYPQPQPF (SEQ ID NO: 3) or QYPSGQGSFQPSQQNPQA (SEQ ID NO: 5). The CeD-specific antigen variant polypeptide can have an amino acid sequence at least 90%, at least 92%, at least 95%, at least 96%, at least 97%, at least 98%, or at least 99% identical to LQLQPFPQPELPYPQPQLPYPQPELPYPQPQPF (SEQ ID NO: 7) or QYPSGEGSFQPSQENPQA (SEQ ID NO: 9). The wild-type CeD-specific antigen, such as wheat gliadin, may be encoded by a nucleotide sequence that is at least 90%, at least 92%, at least 95%, at least 96%, at least 97%, at least 98%, or at least 99% identical to a DNA sequence from GenBank Accession no. AJ133611.1: CTTCAACTTCAACCATTTCCACAACCACAACTTCCATACCCACAACCACAACTTCC ATACCCACAACCACAACTTCCATACCCACAACCACAACCATTT (HLA-DQ2; SEQ ID

NO: 4) or CAATACCCATCAGGTCAAGGTTCATTTCAACCATCACAACAAAACCCACAAGCT (HLA-DQ8; SEQ ID NO: 6), or to a codon-optimized sequence thereof, wherein the sequence of SEQ ID NO:-- or of SEQ ID NO:-- is altered to reflect codon usage of L. lactis.

In some examples according to any of the above embodiments, the IL-10 polypeptide is human IL-10 (hIL-10; UniProtKB P22301), having the sequence:

(SEQ ID NO: 11)
MHSSALLCCLVLLTGVRASPGQGTQSENSCTHFPGNLPNMLRDLRDAFSR
VKTFFQMKDQLDNLLLKESLLEDFKGYLGCQALSEMIQFYLEEVMPQAEN
QDPDIKAHVNSLGENLKTLRLRLRRCHRFLPCENKSKAVEQVKNAFNKLQ
EKGIYKAMSEFDIFINYIEAYMTMKIRN

(wherein underlined residues 1-18 are a signal peptide and residues 19-178 are the mature polypeptide). In other examples, the IL-10 can be an IL-10 variant polypeptide, e.g., including at least one point mutation, e.g., to increase expression of the IL-10 polypeptide by the bacterium. In some examples according to these embodiments, the IL-10 expressed is “mature” human IL-10 (hIL-10), i.e. without its signal peptide. An exemplary sequence is residues 19-178 of hIL-10 (UniProtKB P22301). In some embodiments, the hIL-10 comprises a proline (Pro) to alanine (Ala) substitution, at position 2, when counting the amino acids in the mature peptide. An exemplary mature human IL-10 sequence including the P2A substitution is: SAGQGTQSEN SCTHFPGNLP NMLRDLRDAF SRVKTFFQMK DQLDNLLLKE SLLEDFKGYL GCQALSEMIQ FYLEEVMPQA ENQDPDIKAH VNSLGENLKT LRLRLRRCHR FLPCENKSKA VEQVKNAFNK LQEKGIYKAM SEFDIFINYI EAYMTMKIRN (SEQ ID NO:12). Such polypeptides are described, e.g., in Steidler et al., Nat. Biotechnol. 2003, 21(7): 785-789. In some examples, the IL-10 polypeptide can be the wild-type human IL-10. In other examples, the IL-10 polypeptide is human IL-10 without its own signal peptide and has an amino acid sequence at least 90%, at least 92%, at least 95%, at least 96%, at least 97%, at least 98%, or at least 99% identical to SAGQGTQSEN SCTHFPGNLP NMLRDLRDAF SRVKTFFQMK DQLDNLLLKE SLLEDFKGYL GCQALSEMIQ FYLEEVMPQA ENQDPDIKAH VNSLGENLKT LRLRLRRCHR FLPCENKSKA VEQVKNAFNK LQEKGIYKAM SEFDIFINYI EAYMTMKIRN (SEQ ID NO: 12). In other examples, the exogenous nucleic acid encoding the IL-10 polypeptide has a nucleotide sequence at least 90%, at least 92%, at least 95%, at least 96%, at least 97%, at least 98%, or at least 99% identical to:

(SEQ ID NO: 13)
tcagctggtc aaggtactca atcagaaaac tcatgtactc
actttccagg taacttgcca aacatgcttc gtgatttgcg
tgatgctttt tcacgtgtta aaactttttt tcaaatgaaa
gatcaacttg ataacttgct tttgaaagaa tcacttttgg
aagattttaa aggttacctt ggttgtcaag ctttgtcaga
aatgatccaa ttttaccttg aagaagttat gccacaagct
gaaaaccaag atccagatat caaagctcac gttaactcat
tgggtgaaaa ccttaaaact ttgcgtcttc gtttgcgtcg
ttgtcaccgt tttcttccat gtgaaaacaa atcaaaagct
gttgaacaag ttaaaaacgc ttttaacaaa ttgcaagaaa
aaggtatcta caaagctatg tcagaatttg atatctttat
caactacatc gaagcttaca tgactatgaa aatccgtaac.

In some examples according to any of the above embodiments, an IL-2 polypeptide is used in place of the IL-10 polypeptide. In some embodiments, the IL-2 polypeptide is human IL-2 (hIL-2). An amino acid sequence of wild-type human IL-2 is represented by: MYRMQLLSCIALSLALVTNSAPTSSSTKKTQLQLEHLLLDLQMILNGINNYKNPKLTR MLTFKFYMPKKATELKHLQCLEEELKPLEEVLNLAQSKNFHLRPRDLISNINVIVLELK GSETTFMCEYADETATIVEFLNRWITFCQSIISTLT(Uniprot P60568; SEQ ID NO: 14). An exemplary IL-2 coding nucleic acid sequence is represented by: gctccaacttcatcatcaactaaaaaaactcaattgcaacttgaacacttgcttaggatatcaaatgatcttgaacggtatcaacaactaca aaaacccaaaacttactcgtatgagactataaatatacatgccaaaaaaagctactgaacttaaacacttgcaatgtcttgaagaagaattg aaaccacttgaagaagattgaaccagctcaatcaaaaaactacacttgcgtccacgtgatcttatctcaaacatcaacgttatcgattgga acttaaaggttcagaaactactatatgtgtgaatacgctgatgaaactgctactatcgagaatattgaaccgaggatcactattgtcaatca atcatctcaactttgacttaa (SEQ ID NO: 15). An exemplary amino acid sequence is the mature wild-type human IL-2 represented by amino acids 21-153 of Uniprot P605658: APTSSSTKKTQLQLEHLLLDLQMILNGINNYKNPKLTRMLTFKFYMPKKATELKHLQC LEEELKPLEEVLNLAQSKNFHLRPRDLISNINVIVLELKGSE (SEQ ID NO: 16). An exemplary coding sequence for mature wild-type hIL-2 is: gctccaacttcatcatcaactaaaaaaactcaattgcaacttgaacacttgcttaggatatcaaatgatcttgaacggtatcaacaactaca aaaacccaaaacttactcgtatgagactataaatatacatgccaaaaaaagctactgaacttaaacacttgcaatgtcttgaagaagaattg aaaccacttgaagaagattgaaccagctcaatcaaaaaactacacttgcgtccacgtgatcttatctcaaacatcaacgttatcgattgga acttaaaggttcagaaactactatatgtgtgaatacgctgatgaaactgctactatcgagaatattgaaccgaggatcactattgtcaatca atcatctcaactttgacttaa (SEQ ID NO: 15). In other examples, the IL-2 polypeptide is human IL-2 without its own signal peptide and has an amino acid sequence at least 90%, at least 92%, at least 95%, at least 96%, at least 97%, at least 98%, or at least 99% identical to amino acids 21-153 of Uniprot P605658, provided that the IL-2 variant polypeptide retains some IL-2 activity (functional polypeptide). In some examples, the IL-2 is a variant as described in U.S. Pat. No. 4,518,584 or in U.S. Pat. No. 4,752,585. Other forms of IL-2 that may be used include IL-2 variant sequences such as those found in aldesleukin, or proleukin (Prometheus Laboratories), teceleukin (Roche), bioleukin (Glaxo), as well as variants as described in Taniguchi et al., Nature 1983, 302(5906): 305-10 and Devos et al., Nucleic Acids Res. 1983, 11(13): 4307-23; European Patent Application Nos. 91,539 and 88,195; U.S. Pat. Nos. 4,518,584. 9,266,938; 7,569,215; 5,229,109; U.S. Patent Publication No. 2006/0269515; EP Patent Publication No. EP 1730184A2; and PCT Publication WO 2005/086751.

In some examples according to any of the above embodiments, the microorganism, (e.g., LAB) expresses (e.g., constitutively expresses) the IL-10 polypeptide. In other examples, the microorganism (e.g., LAB) constitutively expresses and secretes the IL-10 polypeptide (e.g., hIL-10). The LAB can constitutively express the CeD-specific antigen polypeptide (e.g., a gliadin peptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope). The microorganism (e.g., LAB) can constitutively express and secrete the CeD-specific antigen (e.g., a gliadin peptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide. In yet other examples, the microorganism (e.g., LAB) can constitutively express and secrete the IL-10 polypeptide (e.g., hIL-10) and the CeD-specific antigen (e.g., a gliadin peptide comprising at least one HLA-DQ2-specific and/or HLA-DQ8-specific epitope) polypeptide (e.g., wheat gliadin).

In some examples according to any of the above embodiments, the microorganism, (e.g., LAB) expresses (e.g., constitutively expresses) the IL-10 polypeptide, and preferably human IL-10 polypeptide for administration to a human. In other examples, the microorganism (e.g., LAB) constitutively expresses and secretes the IL-10 polypeptide (e.g., hIL-10). The LAB can inducibly express the CeD-specific antigen polypeptide (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope). In other examples, the microorganism (e.g., LAB) inducibly expresses and secretes a CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide. In yet other examples, the microorganism (e.g., LAB) inducible expresses and secretes the IL-10 polypeptide (e.g., hIL-10) and the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope (e.g., wheat gliadin). Inducible expression can be directly inducible or can be indirectly inducible.

In some examples according to the above methods, products, and compositions, the exogenous nucleic acid encoding the IL-10 polypeptide is positioned 3′ of another gene, and expression and secretion of IL-10 is coupled to the other gene, e.g., a polycistronic expression cassette. The IL-10 expression cassette can be chromosomally integrated downstream of the phosphopyruvate hydratase gene (eno; Gene ID: 4797432) and the eno promoter Peno. In the microorganism (i.e., LAB), preferably, the eno gene of the expression cassette is located in its native chromosomal locus. In some examples, the IL-10 expression unit can be transcriptionally and translationally coupled to eno by using an intergenic region. Preferably, the intergenic region is positioned immediately 3′ of the stop codon of the eno gene. An exemplary intergenic region in the polycistronic expression cassette is rpmD gene 5′ intergenic region (i.e. the region preceding rpmD; referred to herein as IRrpmD). An exemplary IRrpmD has a nucleotide sequence of TAAGGAGGAAAAAATG (SEQ ID NO: 17), which includes the stop codon TAA of the first gene, and the start codon ATG of a second gene). Without the start and stop codons, the intergenic region rpmD has a nucleic acid sequence of GGAGGAAAAA (SEQ ID NO: 18). Preferably, the intergenic region is positioned immediately 5′ of the start codon of the secretion sequence. An exemplary IL-10 secretion sequence is a nucleotide sequence encoding a secretion leader of unidentified secreted 45-kDa protein (usp45) MKKKIISAILMSTVILSAAAPLSGVYA (SEQ ID NO: 19), encoded by, for instance, atgaaaaaaaagattatctcagctattttaatgtctacag tgatactttctgctgcagccccgttgtcaggtgtttacgcc (SEQ ID NO: 20) or atgaagaagaaaatcattagtgccatcttaatgtctacag tgattctttcagctgcagctcctttatcaggcgtttatgca (SEQ ID NO: 21). Such secretion sequence is referred to herein as SSusp45. An exemplary gene encoding a fusion of usp45 secretion leader (SSusp45) with the hil-10 gene is

(SEQ ID NO: 22)
ATGAAAAAAAAGATTATCTCAGCTATTTTAATGTCTACAGTGATACTTTC
TGCTGCAGCCCCGTTGTCAGGTGTTTACGCCTCAGCTGGTCAAGGTACTC
AATCAGAAAACTCATGTACTCACTTTCCAGGTAACTTGCCAAACATGCTT
CGTGATTTGCGTGATGCTTTTTCACGTGTTAAAACTTTTTTTCAAATGAA
AGATCAACTTGATAACTTGCTTTTGAAAGAATCACTTTTGGAAGATTTTA
AAGGTTACCTTGGTTGTCAAGCTTTGTCAGAAATGATCCAATTTTACCTT
GAAGAAGTTATGCCACAAGCTGAAAACCAAGATCCAGATATCAAAGCTCA
CGTTAACTCATTGGGTGAAAACCTTAAAACTTTGCGTCTTCGTTTGCGTC
GTTGTCACCGTTTTCTTCCATGTGAAAACAAATCAAAAGCTGTTGAACAA
GTTAAAAACGCTTTTAACAAATTGCAAGAAAAAGGTATCTACAAAGCTAT
GTCAGAATTTGATATCTTTATCAACTACATCGAAGCTTACATGACTATGA
AAATCCGTAACTAA.

In some examples, SSusp45 has an amino acid sequence that is at least 90%, at least 92%, at least 95%, at least 96%, at least 97%, at least 98%, or at least 99% identical to MKKKIISAILMSTVILSAAAPLSGVYA (SEQ ID NO: 19). In other examples, SSusp45 can be encoded by a nucleic acid sequence that is at least 90%, at least 92%, at least 95%, at least 96%, at least 97%, at least 98%, or at least 99% identical to atgaaaaaaaagattatctcagctatataatgtctacagtgatactactgctgcagccccgagtcaggtgatacgcc (SEQ ID NO: 20) or atgaagaagaaaatcattagtgccatcttaatgtctacagtgattattcagctgcagctcattatcaggcgatatgca (SEQ ID NO: 21). In some examples, a SSusp45 in the IL-10 expression cassette can be encoded by a nucleic acid sequence that is at least 90%, at least 92%, at least 95%, at least 96%, at least 97%, at least 98%, or at least 99% identical to atgaaaaaaaagattatctcagctattttaatgtctacag tgatactttctgctgcagccccgttgtcaggtgtttacgcc (SEQ ID NO: 20). In some examples, the IL-10 expression cassette is illustrated by: Peno>>eno>>IRrpmD>>SSu sp45-hIL-10.

In other examples using the compositions and methods described herein, the exogenous nucleic acid encoding the IL-10 polypeptide is positioned 3′ of an hllA promoter (PhllA), such as a Lactococcus lactis PhllA. An exemplary PhllA sequence is: aaaacgccttaaaatggcattttgacttgcaaactgggctaagatttgctaaaatgaaaaatgcctatgtttaaggtaaaaaacaaatggag gacatttctaaaatg (SEQ ID NO: 23) which is_constitutive promoter of the HU-like DNA-binding protein gene (Gene ID: 4797353; Locus tag LLMG_RS02525). An exogenous nucleic acid encoding the IL-10 polypeptide can be transcriptionally regulated by the PhllA. In other examples, the LAB includes an IL-10 expression cassette containing a PhllA promoter (e.g., a Lactococcus lactis PhllA), an IL-10 secretion sequence (e.g., positioned 3′ of the PhllA), and the exogenous nucleic acid encoding the IL-10 polypeptide (e.g., positioned 3′ of the IL-10 secretion sequence). In some examples, the IL-10 expression cassette is chromosomally integrated. In some examples, the IL-10 expression cassette is chromosomally integrated thereby replacing or partially replacing another gene.

In some examples according to any of the above embodiments, the exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide can be positioned 3′ of the IL-10 expression cassette, and expression and secretion of the CeD-specific antigen is coupled to the IL-10 expression cassette, e.g., a polycistronic expression cassette. Preferably, the intergenic region is positioned immediately 3′ of the stop codon of the IL-10 expression cassette and is positioned immediately 5′ of the start codon of the CeD-specific antigen or the start codon of a secretion leader fused to the CeD-specific antigen. In some examples, the CeD-specific antigen expression unit is transcriptionally and translationally coupled to IL-10 by use of IRrplN (GCAAAACTAGGAGGAATATAGC; (SEQ ID NO: 24), the IR preceding the highly expressed L. lactis MG1363 505 ribosomal protein L14 gene (rplN; Gene ID: 4799034). In some examples, the expression cassette is illustrated by: hIL-10>>IRrplN>>CeD-specific antigen In some examples according to any of the above embodiments, the exogenous nucleic acid encoding the IL-10 polypeptide is positioned 3′ of another gene, and expression and secretion of IL-10 is couple to the other gene, such eno. In some examples, the expression cassette is illustrated by: Peno>>eno>>IRrpmD>>hil-10>>IRrplN>>CeD-specific antigen, or by Peno>>eno>>IRrpmD>>SSusp45-hil-10>>IRrplN>>CeD-specific antigen. In the microorganism (i.e., LAB), preferably, the eno gene of the expression cassette is located in its native chromosomal locus. In other examples according to any of the above embodiments, the CeD-specific antigen secretion sequence is a nucleotide sequence encoding a secretion leader (SL) selected from the group consisting of: SL #1, SL #6, SL #8, SL #9, SL #13, SL #15, SL #17, SL #20, SL #21, SL #22, SL #23, SL #24, SL #25, SL #32, SL #34, SL #35, and SL #36 (see Table 1). For example, the CeD-specific antigen can be an HLA-DQ2 specific epitope and the secretion sequence is a nucleotide sequence encoding a secretion leader selected from the group consisting of: (SL #1, SL #6, SL #8, SL #9, SL #13, SL #15, SL #17, SL #20, SL #21, SL #22, SL #23, SL #24, SL #25, SL #32, SL #34, and SL #36) or from the group consisting of SL #8, SL #17, SL #20, SL #21, SL #22, SL #23, and SL #34). Alternatively, the CeD-specific antigen is a deamidated HLA-DQ2 specific epitope, e.g., ddq2, and the secretion sequence is a nucleotide sequence encoding a secretion leader selected from the group consisting of: (SL #15, SL #17, SL #21, SL #22, SL #23, SL #32, SL #34, SL #35, and SL #36) or from the group consisting of SL #17, SL #21, SL #22, SL #23, and SL #34. Each and all embodiments are operable without SL #34 as the secretion sequence. The CeD-specific antigen secretion sequence also can be a nucleotide sequence encoding the secretion leader of ps356 endolysin (ps356). Such secretion sequence is referred to herein as SSps356 (SL #21). In some examples, the expression cassette is illustrated by: Peno>>eno>>IRrpmD>>SSusp45-hil-10>>1RrplN>>SSps356-CeD-specific antigen. In the microorganism (i.e., LAB), preferably, the eno gene of the expression cassette is located in its native chromosomal locus. The nucleotide sequence of an exemplary Peno>>eno>>IRrpmD>>SSusp45-hil-10>>IRrplN>>SSps356-CeD-specific antigen expression cassette is depicted in FIG. 23, wherein the CeD-sepcific antigen is the deamidated HLA-DQ2-specific epitope of wheat gliadin LQLQPFPQPELPYPQPQLPYPQPELPYPQPQPF (SEQ ID NO: 7) (referred to herein as ddq2).

In other examples according to the above methods, products, and compositions, the exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide is positioned 3′ of another gene, and expression and secretion of the CeD-specific antigen polypeptide is coupled to the other gene, e.g., a polycistronic expression cassette. The CeD-specific antigen polypeptide expression cassette can be chromosomally integrated downstream of the phosphopyruvate hydratase gene (eno; Gene ID: 4797432) and the eno promoter Peno. In some examples, the CeD-specific antigen polypeptide expression unit can be transcriptionally and translationally coupled to eno by using an intergenic region. An exemplary intergenic region in the polycistronic expression cassette is rpmD gene 5′ intergenic region (i.e. the region preceding rpmD; referred to herein as IRrpmD). An exemplary IRrpmD has a nucleotide sequence of taaggaggaaaaaatg (SEQ ID NO: 17), which includes the stop codon TAA of the first gene, and the start codon ATG of a second gene). Without the start and stop codons, the intergenic region rpmD has a nucleic acid sequence of ggaggaaaaa (SEQ ID NO: 18). In other aspects according to any of the above embodiments, the CeD-specific antigen secretion sequence is a nucleotide sequence encoding a secretion leader (SL) selected from the group consisting of: SL #1, SL #6, SL #8, SL #9, SL #13, SL #15, SL #17, SL #20, SL #21, SL #22, SL #23, SL #24, SL #25, SL #32, SL #34, SL #35, and SL #36 (see Table 1). For example, the CeD-specific antigen can be an HLA-DQ2 specific epitope and the secretion sequence is a nucleotide sequence encoding a secretion leader selected from the group consisting of: SL #1, SL #6, SL #8, SL #9, SL #13, SL #15, SL #17, SL #20, SL #21, SL #22, SL #23, SL #24, SL #25, SL #32, SL #34, and SL #36, or from the group consisting of SL #8, SL #17, SL #20, SL #21, SL #22, SL #23, and SL #34 Alternatively, the CeD-specific antigen is a deamidated HLA-DQ2 specific epitope, e.g., ddq2, and the secretion sequence is a nucleotide sequence encoding a secretion leader selected from the group consisting of: SL #15, SL #17, SL #21, SL #22, SL #23, SL #32, SL #34, SL #35 and SL #36, or from the group consisting of SL #17, SL #21, SL #22, SL #23, and SL #34. Each and all embodiments are operable without SL #34 as the secretion sequence. The CeD-specific antigen secretion sequence also can be a nucleotide sequence encoding the secretion leader of ps356 endolysin (ps356). Such secretion sequence is referred to herein as SSps356 (SL #21). In some examples, the expression cassette is illustrated by: Peno>>eno>>IRrpmD>>SSps356-CeD-specific antigen. An exemplary gene encoding a fusion of ps356 secretion leader (SSps356) with a fragment encoding deamidated DQ2 (ddq2), a protease-resistant 33-mer based on 6 overlapping α1- and α2-gliadin epitopes (UniProt: Q9M4L6_wheat) is:

(SEQ ID NO: 25)
atgaaaaaagtgattaaaaaagcggcgattggcatggtggcgttttttgt
ggtggcggcgagcggcccggtgtttgcgcttcaacttcaaccatttccac
aaccagaacttccatacccacaaccacaacttccatacccacaaccagaa
cttccatacccacaaccacaaccattttaa.

In other examples using the compositions and methods described herein, the exogenous nucleic acid encoding the CeD-specific antigen polypeptide is positioned 3′ of an hllA promoter (PhllA), such as a Lactococcus lactis PhllA. An exogenous nucleic acid encoding the CeD-specific antigen polypeptide can be transcriptionally regulated by the PhllA. In other examples, the LAB includes an CeD-specific antigen polypeptide expression cassette containing a Ph11A promoter (e.g., a Lactococcus lactis PhllA), an CeD-specific antigen secretion sequence (e.g., positioned 3′ of the PhllA), and the exogenous nucleic acid encoding the CeD-specific antigen polypeptide (e.g., positioned 3′ of the CeD-specific antigen secretion sequence). In some examples, the CeD-specific antigen expression cassette is chromosomally integrated. In some examples, the CeD-specific antigen expression cassette is chromosomally integrated thereby replacing or partially replacing another gene.

In some examples according to any of the above embodiments, the exogenous nucleic acid encoding hIL-10 polypeptide can be positioned 3′ of the CeD-specific antigen expression cassette, and expression and secretion of the hIL-10 is coupled to the the CeD-specific antigen expression cassette, e.g., a polycistronic expression cassette.

In some examples, the hIL-10 expression unit is transcriptionally and translationally coupled to CeD-specific antigen by use of IRrplN (gcaaaactaggaggaatatagc (SEQ ID NO: 24), the IR preceding the highly expressed L. lactis MG1363 50S ribosomal protein L14 gene (rplN; Gene ID: 4799034; Locus tag LLMG_RS11895). In some examples, the expression cassette is illustrated by: CeD-specific antigen>>IRrplN>>hil-10. In some examples according to any of the above embodiments, the exogenous nucleic acid encoding the CeD-specific antigen polypeptide is positioned 3′ of another gene, and expression and secretion of CeD-specific antigen is coupled to the other gene, such eno. In some examples, the expression cassette is illustrated by: Peno>>eno>>IRrpmD>>CeD-specific antigen>>IRrplN >>hil-1 0, or by Peno>>eno>>IRrpmD>>SSusp45-CeD-specific antigen>>IRrplN>>hil-10. In some examples according to any of the above embodiments, the hIL-10 secretion sequence is a nucleotide sequence encoding SSusp45, MKKKIISAILMSTVILSAAAPLSGVYA (SEQ ID NO: 19), encoded by, for instance, atgaaaaaaaagattatctcagctattttaatgtctacag tgatactttctgctgcagccccgttgtcaggtgtttacgcc (SEQ ID NO: 20) or atgaagaagaaaatcattagtgccatcttaatgtctacag tgattctttcagctgcagctcctttatcaggcgtttatgca (SEQ ID NO: 21). In some examples, SSusp45 has an amino acid sequence that is at least 90%, at least 92%, at least 95%, at least 96%, at least 97%, at least 98%, or at least 99% identical to MKKKIISAILMSTVILSAAAPLSGVYA (SEQ ID NO: 19). In other examples, SSusp45 can be encoded by a nucleic acid sequence that is at least 90%, at least 92%, at least 95%, at least 96%, at least 97%, at least 98%, or at least 99% identical to atgaaaaaaaagattatctcagctattttaatgtctacag tgatactttctgctgcagccccgttgtcaggtgtttacgcc (SEQ ID NO: 20) or atgaagaagaaaatcattagtgccatcttaatgtctacag tgattctttcagctgcagctcctttatcaggcgtttatgca (SEQ ID NO: 21). In some examples, SSusp45 in the hil-10 expression cassette can be encoded by a nucleic acid sequence that is at least 90%, at least 92%, at least 95%, at least 96%, at least 97%, at least 98%, or at least 99% identical to atgaaaaaaaagattatctcagctattttaatgtctacag tgatactttctgctgcagccccgttgtcaggtgtttacgcc (SEQ ID NO: 20). In some examples, the expression cassette is illustrated by: Peno>>eno>>IRrpmD>>SSps356-CeD-specific antigen>>IRrplN>>SSusp45-hil-10.

Further embodiments are contemplated for L. lactis secreting a CeD-specific antigen, such as dDQ2, and interleukin-10, such as human IL-10. These further embodiments encompass expression units integrated downstream of one or more highly expressed endogenous genes. Contemplated embodiments are disclosed in Example 5 and Tables XI-X4. The cassettes disclosed in Tables X1-X4 optionally further comprise components described herein. For instance, the cassettes can further comprise at least one intergenic region transcriptionally coupling, e.g., the CeD-specific antigen to the endogenous gene. The cassettes can further comprise a sequence encoding a secretion leader fused 5′ to the coding sequence of the CeD-specific antigen and a sequence encoding a secretion leader fused 5′ to the coding sequence of IL-10, thereby encoding a first fusion polypeptide of a secretion leader and CeD-specific antigen and a second fusion polypeptide of a secretion leader and IL-10.

In some examples according to any of the above embodiments, the microorganism (e.g., LAB) further comprises an exogenous nucleic acid encoding a trehalose-6-phosphate phosphatase, e.g., otsB, such as Escherichia coli otsB. In some examples according to these embodiments, the exogenous nucleic acid encoding the trehalose-6-phosphate phosphatase is chromosomally integrated. In some examples, the exogenous nucleic acid encoding the trehalose-6-phosphate phosphatase is chromosomally integrated 3′ of unidentified secreted 45-kDa protein gene (usp45). In some examples according to this embodiment, the LAB comprises a second polycistronic expression cassette comprising a usp45 promoter, the usp45 gene (e.g., 3′ of the promoter), and the exogenous nucleic acid encoding a trehalose-6-phosphate phosphatase (e.g., 3′ of the usp45 gene). In some examples, the second polycistronic expression cassette further comprises an intergenic region between the usp45 gene and the exogenous nucleic acid encoding a trehalose-6-phosphate phosphatase. In some examples, the second polycistronic expression cassette is illustrated by: Pusp45>>usp45>>intergenic region>>otsB. In some examples according to these embodiments, the intergenic region is IRrpmD as described herein above (e.g., having taaggaggaaaaaatg (SEQ ID NO: 17) or ggaggaaaaa (SEQ ID NO: 18).

The second polycistronic expression cassette may then be illustrated by: Pusp45>>usp45>>IRrpmD>>otsB.

In some examples according any of the above compositions, a trehalose-6-phosphate phosphorylase gene (trePP) is disrupted or inactivated in the microorganism (e.g., LAB). For example, the trePP has been inactivated by removing the trePP gene or a fragment thereof, or the trePP has been disrupted by inserting a stop codon. Thus, in some examples, the microorganism (e.g., LAB) lacks trePP activity.

In other examples, a cellobiose-specific PTS system IIC component gene (ptcC) has been disrupted or inactivated in the microorganism (e.g., LAB). For example, the ptcC can be been disrupted by inserting a stop codon, such as a TGA at codon 30, or ptcC has been inactivated by removing the ptcC or a fragment thereof. Thus, in some examples, the microorganism (e.g., LAB) lacks ptcC activity.

In other examples according to the compositions and methods, the LAB further comprises one or more genes encoding one or more trehalose transporter(s). In some examples, the one or more genes encoding the one or more trehalose transporter(s) are endogenous to the LAB. In some examples, the LAB overexpresses the one or more genes encoding the one or more trehalose transporter(s). In some examples according to these embodiments, the one or more genes encoding the one or more trehalose transporter(s) is positioned 3′ of an exogenous promoter, e.g., an hllA promoter (PhllA). For example, the one or more genes encoding the one or more trehalose transporter(s) are transcriptionally regulated by the PhllA. In some examples, the one or more genes encoding the one or more trehalose transporter(s) is selected from LLMG_RS02300 (Gene ID: 4797778; formerly llmg_0453), LLMG_RS02305 (Gene ID: 4797093; formerly llmg_0454), and any combination thereof. In some examples, LLMG_RS02300 and LLMG_RS02305 are transcriptionally regulated by PhllA.

In some examples, the one or more genes encoding one or more trehalose transporter(s) comprises two genes encoding two trehalose transporters, wherein an intergenic region is located between the two genes. In some examples, the intergenic region is IRrpmD, e.g., having taaggaggaaaaaatg (SEQ ID NO: 17) or ggaggaaaaa (SEQ ID NO: 18). In some examples, the microorganism (e.g., LAB) comprises a polycistronic expression cassette comprising two nucleic acid sequences (e.g., genes) encoding two different trehalose transporters (transporter 1 and transporter 2 sequences) and an intergenic region between the two nucleic acids encoding the two different trehalose transporters. Such expression cassette may be illustrated by: PhllA>>transporter 1>>intergenic region>>transporter 2. In some examples according to these embodiments, the intergenic region is rpmD as described herein above (e.g., having taaggaggaaaaaatg (SEQ ID NO: 17) or ggaggaaaaa (SEQ ID NO: 18)). The polycistronic expression cassette may then be illustrated by: PhllA>>transporterl>>IRrpmD >>transporter2.

Thus, in some embodiments, the LAB comprises, in a single strain, several useful features. In one embodiment, the LAB is Lactococcus lactis, comprising:
(A) a chromosomally integrated promoter>>secretion signal>>>therapeutic protein, such as an interleukin;
(B) a chromosomally-integrated promoter>>secretion signal>>second therapeutic protein, such as an antigen; and
(C) a combination of mutations and insertions to promote trehalose accumulation, which enhances LAB survivability against bile salts and drying. The mutations are selected from:
   (i) chromosomally-integrated trehalose transporter(s), such as PhllA>>transporter 1>>intergenic region>>transporter 2, such as LLMG_RS02300 and/or LLMG_RS02305, for uptake of trehalose;
   (ii) chromosomally-integrated Trehalose-6-phosphate phosphatase gene (otsB; Gene ID: 1036914; Locus tag c2311) positioned downstream of usp45 (Gene ID: 4797218; Locus tag LLMG_RS12595) to facilitate conversion of trehalose-6-phosphate to trehalose;
   (iii) inactivated (e.g., through gene deletion) Trehalose-6-phosphate phosphorylase gene (trePP; Gene ID: 4797140; Locus tag LLMG_RS02310; formerly llmg_0455); and
   (iv) inactivated cellobiose-specific PTS system IIC component (Gene ID: 4796893; Locus tag LLMG_RS02240; formerly llmg_0440), ptcC, (e.g. tga at codon position 30 of 446; tga30) or deleted cellobiose-specific PTS system IIC component (Gene ID: 4796893), ΔptcC.
      The LAB may also contain an auxotrophic mutation for biological containment, such as thyA.

In one embodiment, the LAB is Lactococcus lactis, comprising:
(A) a chromosomally integrated promoter>>secretion signal>>hIL-10 to secrete mature hIL-10 from LAB, such as Peno>>eno>>IRrpmD>>SSusp45-h1L-10 or PhllA>>SSusp45-hil-10;
(B) a chromosomally-integrated intergenic region>>secretion signal>>CeD-specific antigen, to secrete a CeD-specific antigen, e.g., ddq2, from LAB; such as intergenic region>>IRrplN>>CeD-specific antigen. The intergenic region could be, for example, IRrplN; and
(C) a combination of mutations and insertions to promote trehalose accumulation, which enhances LAB survivability against bile salts and drying. The mutations are selected from
   (i) chromosomally-integrated trehalose transporter(s), such as PhllA>>transporter 1>>intergenic region>>transporter 2, such as LLMG_RS02300 and/or LLMG_RS02305 , for uptake of trehalose;
   (ii) chromosomally-integrated Trehalose-6-phosphate phosphatase gene (otsB; Gene ID: 1036914) positioned downstream of usp45 (Gene ID: 4797218) to facilitate conversion of trehalose-6-phosphate to trehalose;
   (iii) inactivated (e.g. through gene deletion) Trehalose-6-phosphate phosphorylase gene (trePP; Gene ID: 4797140); and
   (iv) inactivated cellobiose-specific PTS system IIC component (Gene ID: 4796893), ptcC, (e.g., tga at codon position 30 of 446; tga30) or deleted cellobiose-specific PTS system TIC component (Gene ID: 4796893), ΔptcC.
      The LAB may also contain an auxotrophic mutation for biological containment, such as thyA.

In one embodiment, the LAB is Lactococcus lactis, comprising:
(A) a chromosomally integrated polycistronic cassette to secrete both IL-10 and CeD-specific antigen from LAB, such as Peno>>eno>>IRrpmD>>SSusp45-hil-10>>1RrplN>>SSps356-CeD-specific antigen, e.g., ddq2; and
(B) a combination of mutations and insertions to promote trehalose accumulation, which enhances LAB survivability against bile salts and drying. The mutations are selected from:
   (i) chromosomally-integrated trehalose transporter(s), such as PhllA>>transporter 1>>intergenic region>>transporter 2, such as LLMG_RS02300 and/or LLMG_RS02305, for uptake of trehalose;
   (ii) chromosomally-integrated Trehalose-6-phosphate phosphatase gene (otsB; Gene ID: 1036914) positioned downstream of usp45 (Gene ID: 4797218) to facilitate conversion of trehalose-6-phosphate to trehalose;
   (iii) inactivated (e.g. through gene deletion) Trehalose-6-phosphate phosphorylase gene (trePP; Gene ID: 4797140); and
   (iv) inactivated cellobiose-specific PTS system IIC component (Gene ID: 4796893), ptcC, (e.g. tga at codon position 30 of 446; tga30) or deleted cellobiose-specific PTS system TIC component (Gene ID: 4796893), ΔptcC.
      The LAB may also contain an auxotrophic mutation for biological containment, such as thyA.

The LAB is Lactococcus lactis and may contain
(A) thyA mutation, for biological containment;
(B) a chromosomally-integrated polycistronic cassette of Peno>>eno>>IRrpmD>>SSusp45-hil-10>>IRrplN>>SSps356-CeD-specific antigen, e.g., ddq2;
(C) chromosomally-integrated trehalose transporter(s), such as PhllA>>transporter 1>>intergenic region>>transporter 2, such as LLMG_RS02300 and/or LLMG_RS02305, for uptake of trehalose;
(D) inactivated (e.g., through gene deletion) trehalose-6-phosphate phosphorylase gene (trePP; Gene ID: 4797140);
(E) chromosomally integrated Trehalose-6-phosphate phosphatase gene (otsB; Gene ID: 1036914) (positioned downstream of usp45 (Gene ID: 4797218) to facilitate conversion of trehalose-6-phosphate to trehalose; and
(F) deleted cellobiose-specific PTS system IIC component (Gene ID: 4796893), ΔptcC.

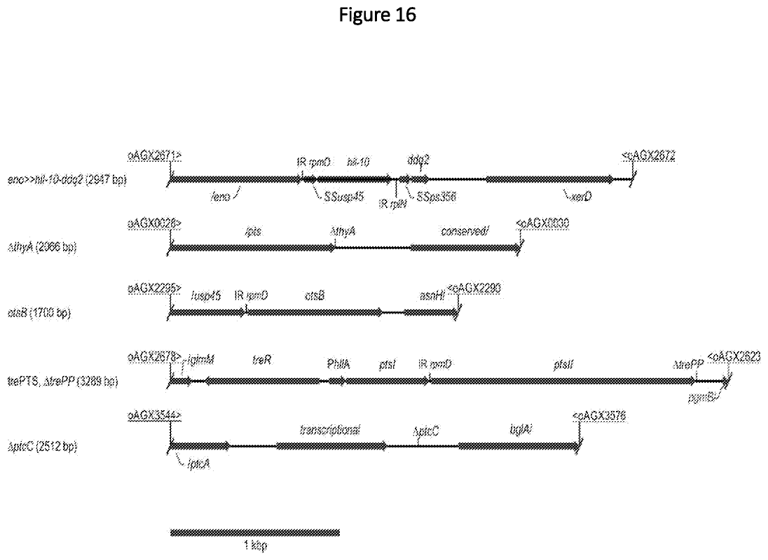

In one embodiment, the LAB is Lactococcus lactis strain sAGX0868. sAGX0868 is a derivative of Lactococcus lactis (L. lactis) MG1363. In sAGX0868:
Thymidylate synthase gene (thyA; Gene ID: 4798358) is absent, to warrant environmental containment (Steidler, L., et al., Nat. Biotechnol. 2003,21(7): 785-789).
Trehalose-6-phosphate phosphorylase gene (trePP; Gene ID: 4797140) is absent, to allow accumulation of exogenously added trehalose.
Trehalose-6-phosphate phosphatase gene (otsB; Gene ID: 1036914) is positioned downstream of usp45 (Gene ID: 4797218) to facilitate conversion of trehalose-6-phosphate to trehalose. The otsB expression unit was transcriptionally and translationally coupled to usp45 by use of the intergenic region (IR) preceding the highly expressed L. lactis MG1363 50S ribosomal protein L30 gene (rpmD; Gene ID: 4797873).
The constitutive promoter of the HU-like DNA-binding protein gene (PhllA; Gene ID: 4797353) is preceding the putative phosphotransferase genes in the trehalose operon (trePTS; LLMG_RS02300 and LLMG_RS02305, Gene ID: 4797778 and Gene ID: 4797093 respectively) to potentiate trehalose uptake.
The gene encoding cellobiose-specific PTS system IIC component (Gene ID: 4796893), ptcC, is deleted (AptcC). This mutation ascertains trehalose retention after accumulation.
Insertion of a fragment encoding a fusion usp45 secretion leader (SSusp45) with the hil-10 gene, encoding human interleukin-10 (hIL-10; UniProt: P22301, aa 19-178, variant P2A [1]), downstream of the phosphopyruvate hydratase gene (eno; Gene ID: 4797432). To allow expression and secretion of hIL-10, the hil-10 expression unit was transcriptionally and translationally coupled to eno by use of IRrpmD.
Insertion, downstream of the hil-10 gene, of a fragment encoding a fusion of ps356 endolysin gene (ps356; Gene ID: 4798697) secretion leader (SSps356) with a fragment encoding deamidated DQ2 (ddq2), a protease-resistant 33-mer based on 6 overlapping α1- and α2-gliadin epitopes (UniProt: Q9M4L6_wheat, amino acids 57-89, glutamine deamidation at positions 66 and 80). To allow expression and secretion of dDQ2, the ddq2 expression unit was transcriptionally and translationally coupled to hil-10 by use of IR preceding the highly expressed L. lactis MG1363 50S ribosomal protein L14 gene (rplN; Gene ID: 4799034).
   FIG. 16 shows a schematic overview of relevant genetic loci of sAGX0868. All genetic traits of sAGX0868 reside on the bacterial chromosome. The genetic background of sAGX0868 warrants:
Constitutive secretion of hIL-10.
Constitutive secretion of dDQ2.
Strict dependence on exogenously added thymidine for growth and survival.
The capacity to accumulate and retain trehalose and so acquire the capacity to resist bile acid toxicity.

The present disclosure further provides compositions containing a microorganism (e.g., an LAB) as described herein, e.g., a microorganism (e.g., LAB) in accordance with any of the above embodiments.

The present disclosure further provides compositions comprising a first LAB containing an exogenous nucleic acid encoding an IL-10 polypeptide and expresses the IL-10 polypeptide and a second LAB containing an exogenous nucleic acid encoding a CeD-specific antigen polypeptide, such as a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope and that expresses the CeD-specific antigen polypeptide. For instance, provided is a composition comprising: a first LAB containing an exogenous nucleic acid encoding an IL-10 polypeptide and expresses the IL-10 polypeptide; and a second LAB containing an exogenous nucleic acid encoding a gliadin polypeptide comprising at least one HLA-DQ2-specific epitope, at least one deamidated HLA-DQ2 specific epitope, at least one HLA-DQ8-specific epitope, at least one deamidated HLA-DQ8 specific epitope, or a combination of (i) at least one HLA-DQ2-specific epitope and/or at least one HLA-DQ2 specific epitope, and (ii) at least one HLA-DQ8-specific epitope and/or at least one HLA-DQ8 specific epitope. In embodiments of such compositions, the exogenous nucleic acid is chromosomally integrated in at least one of the two LAB. The above described embodiments regarding exogenous nucleic acid structure and sequence are applicable to the first and second LAB of these compositions. For instance, in an embodiment, the exogenous nucleic acid encoding a gliadin polypeptide further encodes a secretion leader sequence fused to a gliadin polypeptide, wherein the secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36.

The present disclosure further provides pharmaceutical compositions containing a microorganism (e.g., LAB) as described herein, e.g., a microorganism (e.g., LAB) in accordance with any of the above described modifications, and further containing a pharmaceutically acceptable carrier.

The present disclosure further provides a microbial suspension (e.g., bacterial suspension) containing a microorganism (e.g., LAB) in accordance with any of the modifications, and further containing a solvent, and a stabilizing agent. In some examples, the solvent can be selected from water, oil, and any combination thereof. For example, the present disclosure provides a bacterial suspension containing an LAB of the present disclosure, an aqueous mixture (e.g., a drink), and a stabilizing agent. Exemplary stabilizing agents are selected from a protein or polypeptide (e.g., glycoprotein), a peptide, a mono-, di- or polysaccharide, an amino acid, a gel, a fatty acid, a polyol (e.g., sorbitol, mannitol, or inositol), a salt (e.g., an amino acid salt), or any combination thereof.

The present disclosure further provides a microorganism as described herein (e.g., an LAB in accordance with any of the above embodiments), a composition as described herein, or a pharmaceutical composition as described herein, for use in the treatment of celiac disease (CeD).

The present disclosure further provides a microorganism as described herein (e.g., an LAB in accordance with any of the above embodiments), a composition as described herein, or a pharmaceutical composition as described herein, for use in the preparation of a medicament, e.g., for the treatment of a disease, e.g., an autoimmune disease, such as celiac disease (CeD).

### Method 1: Methods of Treating Disease

The present disclosure further provides methods for the treatment of CeD in a subject in need thereof. Exemplary methods include administering to the subject a therapeutically effective amount of a microorganism (e.g., LAB) as disclosed herein (e.g., an LAB in accordance with any of the above embodiments), a composition as disclosed herein, or a pharmaceutical composition as disclosed herein. In some examples according to any of these embodiments, the subject is a human, e.g., a human patient. In some examples, the method further comprises administering an additional immunomodulatory agent (e.g., an anti-CD3 antibody) to the subject. In some examples, the method excludes administering an additional immunomodulatory agent, e.g., excludes administration of an anti-CD3 antibody) to the subject. Thus, in some examples, a method is provided for the treatment of CeD in a human subject in need thereof. Exemplary methods include administering to the human subject a therapeutically effective amount of an LAB as disclosed herein (e.g., an LAB in accordance with any of the above embodiments), a composition as disclosed herein, or a pharmaceutical composition as disclosed herein.

The subject treated by the disclosed methods can be diagnosed with genetic susceptibility to CeD, e.g., having HLA-DQ2 and/or HLA-DQ8. In some embodiments, the mammalian subject in the above methods, has been diagnosed with CeD. Standard means to diagnosis of CeD are known. See, e.g., Rubio-Tapia et al., 2013, “ACG Clinical Guidelines: Diagnosis and Management of Celiac Disease,” Am. J. Gastroenterol. 108: 656-676. Diagnosis of CeD can be based on a combination of findings from medical history, physical examination, serological testing and upper endoscopy with histological analysis of multiple biopsies of the duodenum biopsy, followed by a favorable clinical and serological response to the gluten free diet to confirm the diagnosis. Serological testing can include testing for IgA anti-tissue transglutaminase (TGA) and/or IgG anti-deamidated gluten peptide (DGP). Histological analysis can include assessing villous height/crypt depth ratio (Villous atrophy and/or crypt hyperplasia) and/or intraepithelial lymphocyte count (IEL proliferation). The diagnosed subject can have had recent previous exposure to gluten, e.g., within the previous week, 2 weeks, 3 weeks, 1 month, 2 month, 3 month, 4 or 5 months, prior to administering the microorganism (e.g., LAB). Alternatively, the diagnosed subject can have had less recent previous exposure to gluten, e.g., 6 months previous, 9 months previous, 12 months previous, 24 months previous, 36 months previous, or greater than 36 months previous, prior to administering the microorganism (e.g., LAB).

In some examples according to any of the variations to Method 1, the method further includes measuring a clinical marker (e.g., an immune biomarker and/or a histopathological marker) in the subject, e.g., the subject's organ or blood. Exemplary clinical markers include serological testing IgA anti-tissue transglutaminase (TGA) and/or IgG anti-deamidated gluten peptide (DGP), and histological analysis assessing villous height/crypt depth ratio (villous atrophy and/or crypt hyperplasia) and/or Intraepithelial lymphocyte count (IEL proliferation). See also Hindryckx et al., 2016, “Disease activity indices in coeliac disease: systematic review and recommendations for clinical trials,” Gut 67: 61-69.

In related embodiments, the invention is a method of increasing oral tolerance to gluten. In other embodiments, the invention is a method of prevention of or substantially reducing, preferably eliminating, villous atrophy in a subject exposed to intestinal gluten. In other embodiments, the invention is a method of substantially increasing villous height to crypt depth ratio to greater than 2.0, or equal or greater than 2.1, 2.2, 2.3, 2.4, or 2.5 in a subject exposed to gluten. In other embodiments, disclosed is a method of substantially decreasing the amount of CD4⁺ and CD8αβ⁺ intraepithelial cells (IELs) expressing the activating natural killer (NK) receptor NKG2D and/or increasing the amount of CD4⁺ and CD8αβ⁺ intraepithelial cells (IELs) expressing the inhibitory natural killer (NK) receptor NKG2A. In other embodiments, a method is also disclosed of substantially decreasing the number of intraepithelial lymphocytes, e.g. CD3⁺ IELs per 100 intestinal epithelial cells, of a subject who has been exposed to gluten. Another method is also disclosed of substantially increasing the ratio of CD4⁺ Foxp3⁺ regulatory T cells over T_{H}1 cells expressing Tbet in lamina propria cells of a subject exposed to gluten. Another method is also disclosed of substantially increasing the ratio of CD4⁺ Foxp3⁺ regulatory T cells over T_{H}1 cells expressing Tbet in lamina propria cells of a subject exposed to gluten. Another method is also disclosed of increasing tolerance-inducing lymphocytes in lamina propria cells of a subject exposed to gluten. In other embodiments, the invention is a method of reducing the amount of one or more of IgA anti-tissue transglutaminase (TGA), IgG anti-deamidated gluten peptide (DGP), and IgG anti-gliadin peptide.

In any of the above methods, the microorganism (e.g., LAB) can be administered to the subject orally. For example, the microorganism (e.g., LAB) is administered to the subject in the form of a pharmaceutical composition for oral administration (e.g., a capsule, tablet, granule, or liquid) comprising the microorganism (e.g., LAB) and a pharmaceutically acceptable carrier. In other examples, the microorganism (e.g., LAB) can be administered to the subject in the form of a food product, or is added to a food (e.g., a drink). In other examples, the microorganism (e.g., LAB) is administered to the subject in the form of a dietary supplement. In yet other examples, the microorganism (e.g., LAB) is administered to the subject in the form of a suppository product. In some examples, the compositions of the present disclosure are adapted for mucosal delivery of the polypeptides, which are expressed by the microorganism (e.g., LAB). For example, compositions may be formulated for efficient release in the gastro-intestinal tract (e.g., gut) of the subject.

The various described methods also contemplates establishing tolerance to a CeD-specific antigen (e.g., a gliadin peptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide in a subject in need thereof. Exemplary methods include administering to the subject a therapeutically effective amount of a microorganism (e.g., LAB) as disclosed herein (e.g., an LAB in accordance with any of the above embodiments), a composition as disclosed herein, or a pharmaceutical composition as disclosed herein. In some examples according to any of these embodiments, the subject is a human, e.g., a human patient.

In a further embodiment, the therapeutic method can be used to prevent CeD, such as by administration prior to any clinical symptoms. Preferably, the subject is identified as having one or more CeD risk factors, as discussed herein and including a genetic predisposition, i.e., a genotype including HLA-DQ2 and/or HLA-DQ8. Other CeD risk factors include first-degree family members with confirmed diagnosis of CeD (especially siblings), T1D diabetes, Down and Turner's syndrome, dermatitis herpetiformis, autoimmune endocrinopathy especially thyroid disease, autoimmune hepatitis and primary biliary cirrhosis. See, e.g., Gujral et al., 2012, “Celiac disease: prevalence, diagnosis, pathogenesis and treatment,” World J. Gastroenterol. 18(42): 6036-59.

### Method 2: Method of Preparing a Genetically-Modified Organism for Treatment of CeD

The current disclosure further provides methods for preparing a genetically modified microorganism (e.g., an LAB) as disclosed herein. Varied methods of site-directed integration (including site-directed chromosomal integration, which is also known as site-specific recombination) are well known and may be applied to generate the recombinant LABs disclosed herein. Exemplary methods include (i) contacting a microorganism (e.g., LAB) with an exogenous nucleic acid encoding an IL-10 polypeptide; and (ii) contacting the microorganism (e.g., LAB) with an exogenous nucleic acid encoding a CeD-specific antigen (e.g., a gliadin peptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide, wherein the exogenous nucleic acid encoding the IL-10 polypeptide and the exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide are chromosomally integrated (i.e., integrated into the chromosome of the microorganism, e.g., LAB). When the nucleic acids are integrated into the microbial (e.g., bacterial) genome, e.g. in the chromosome, the genetically modified microorganism (e.g., LAB) is formed. The microorganism (e.g., LAB) subjected to the genetic modification of the current method can be any microbial strain, e.g., can be a wild-type bacterial strain, or can be genetically modified prior to contacting it with the exogenous nucleic acid encoding the IL-10 polypeptide and the exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide.

In some examples, the above methods employ homologous recombination to integrate the nucleic acids into the microbial (e.g., bacterial) chromosome. Thus, in some examples, the exogenous nucleic acid encoding the IL-10 polypeptide and the exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide are chromosomally integrated using homologous recombination (e.g., employing one or more integration plasmid containing the respective nucleic acids). In some examples, contacting the microorganism (e.g., LAB) with an exogenous nucleic acid encoding the IL-10 polypeptide (e.g., an integration plasmid containing the exogenous nucleic acid encoding the IL-10 polypeptide) occurs prior to contacting the LAB with an exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide (e.g., an integration plasmid containing the exogenous nucleic acid encoding the a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope polypeptide). In other examples, contacting the microorganism (e.g., LAB) with an exogenous nucleic acid encoding the IL-10 polypeptide (e.g., an integration plasmid containing the exogenous nucleic acid encoding the IL-10 polypeptide) occurs subsequent to contacting the LAB with an exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide (e.g., an integration plasmid containing the exogenous nucleic acid encoding the a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope polypeptide). In yet other examples according to any of these embodiments, the microorganism (e.g., LAB) is contacted concurrently with an exogenous nucleic acid encoding the IL-10 polypeptide (e.g., an integration plasmid containing the exogenous nucleic acid encoding the IL-10 polypeptide) and an exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide (e.g., an integration plasmid containing an exogenous nucleic acid encoding a gliadin peptide comprising at least one HLA-DQ2-specific and/or HLA-DQ8-specific epitope polypeptide), or a exogenous nucleic acid encoding both hIL-10 and a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope.

In some examples, the method can further include combining a culture of the genetically modified microorganism (e.g., LAB) with at least one stabilizing agent (e.g., a cryopreserving agent) to form a microbial (e.g., bacterial) mixture. In some examples, the method further includes removing water from the microbial (e.g., bacterial) mixture forming a dried composition. For example, the method can further include freeze-drying the microbial (e.g., bacterial) mixture to form a freeze-dried composition. In other examples, the method may further include combining the genetically modified microorganism (e.g., LAB) or the dried composition (e.g., the freeze-dried composition) with a pharmaceutically acceptable carrier to form a pharmaceutical composition. The method may also include formulating the dried composition (e.g., the freeze-dried composition) or the pharmaceutical composition into a pharmaceutical dosage form.

The current disclosure further provides a genetically modified microorganism (e.g., a genetically modified LAB) prepared by a method described herein (e.g., a method in accordance with any of the above embodiments of Method 2).

### Method 3: Method of Preparing a Pharmaceutical Composition

The disclosure further provides methods for preparing a pharmaceutical composition. Exemplary methods include contacting a culture of a microorganism (e.g., LAB) as disclosed herein (e.g., an LAB in accordance with any of the above embodiments) with at least one stabilizing agent (e.g., a cryopreserving agent), thereby forming a microbial (e.g., bacterial) mixture. In some examples, the at least one stabilizing agent comprises at least one cryopreserving agent. In some examples, the microorganism (e.g., LAB) may contain an exogenous nucleic acid encoding an IL-10 polypeptide, and further contains an exogenous nucleic acid encoding a CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide, wherein the exogenous nucleic acid encoding the IL-10 polypeptide and the exogenous nucleic acid encoding the CeD-specific antigen (e.g., a gliadin peptide comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope) polypeptide are both chromosomally integrated, i.e., are integrated into (or located on) the microbial (e.g., bacterial) chromosome.

Such methods may further include removing water from the microbial (e.g., bacterial) mixture, thereby forming a dried composition. For example, the methods may include freeze-drying the microbial (e.g., bacterial) mixture thereby forming a freeze-dried composition.

In some examples according to Method 3, the method can further include contacting the dried composition (e.g., the freeze-dried composition) with a pharmaceutically acceptable carrier forming a pharmaceutical composition. The methods may further include formulating the dried composition (e.g., freeze-dried composition) into a pharmaceutical dosage form, such as a tablet, a capsule, or a sachet.

### Unit Dosage Forms

Accordingly, the present disclosure further provides a unit dosage form comprising a microorganism (e.g., LAB such as sAGC0868) of the present disclosure, a dried composition of the present disclosure (e.g., a freeze-dried composition of the present disclosure), or a pharmaceutical composition of the present disclosure. In some examples, the unit dosage form is an oral dosage form, such as a tablet, a capsule (e.g., a capsule containing a powder or containing micro-pellets or micro-granules), a granule, or a sachet (e.g., containing dried bacteria for suspension in a liquid for oral administration). In some embodiments, the non-pathogenic microorganism (e.g., LAB) contained in the dosage form is in a dry-powder form or compacted version thereof.

In some examples according to these embodiments, the unit dosage form contains from about 1×10⁴ to about 1×10¹² colony-forming units (cfu) of the microorganism (e.g., LAB). In other examples, the unit dosage form contains from about 1×10⁶ to about 1×10¹² colony forming units (cfu) of the microorganism (e.g., LAB). In other examples, the unit dosage form contains from about 1×10⁸ to about 1×10¹¹ cfu. In yet other examples, the unit dosage form contains about 1×10⁹ to about 1×10¹² cfu. In some examples, the unit dosage contains about 1×10⁴ to about 1×10¹² colony-forming units (cfu) of sAGX0868. In some examples, the unit dosage form contains from about 1×10⁸ to about 1×10¹¹ cfu, or about 1×10¹⁰ to about 1×10¹¹ cfu, or about 1×10¹¹ cfu sAGX0868

### Kits

The current disclosure further provides kits containing (1) a microorganism (e.g., LAB such as sAGX0868) according to any of the embodiments disclosed herein, a composition containing a microorganism (e.g., LAB) according to any of the embodiments described herein, a pharmaceutical composition containing a microorganism (e.g., LAB) according to any of the embodiments described herein, or a unit dosage form containing a microorganism (e.g., LAB) according to any of the embodiments described herein; and (2) instructions for administering the microorganism (e.g., LAB), the composition, the pharmaceutical composition, or the unit dosage form to a mammal, e.g., a human (e g , human patient).

In each of the above-described above methods, products, and compositions, and as further disclosed herein, interleukin-10 is the primary cytokine of choice. In each of the above-described above methods, products, and compositions, and as further disclosed herein, interleukin-2 is an alternative to interleukin-10.

### B. Definitions and Further Detailed Description

As used in the specification, embodiments, and embodiments, the singular forms “a,” “an” and “the” include plural references unless the context clearly dictates otherwise. For example, the term “a cell” includes a plurality of cells, including mixtures thereof. Similarly, use of “a compound” for treatment or preparation of medicaments as described herein contemplates using one or more compounds of this invention for such treatment or preparation unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the term “expressing” a gene or polypeptide or “producing” a polypeptide (e.g., an IL-10 polypeptide or CeD-specific antigen polypeptide), or “secreting” a polypeptide is meant to include “capable of expressing” and “capable of producing,” or “capable of secreting,” respectively. For example, a microorganism, which contains an exogenous nucleic acid can under sufficient conditions (e.g., sufficient hydration and/or in the presence of nutrients) express and secrete a polypeptide encoded by an exogenous nucleic acid. However, the microorganism may not always actively express the encoded polypeptide. The microorganism (e.g., bacterium) may be dried (e.g., freeze-dried), and in that state can be considered dormant (i.e., is not actively producing polypeptide). However, once the microorganism is subjected to sufficient conditions, e.g., is administered to a subject and is released (e.g., in the gastro-intestinal tract of the subject) it may begin expressing and secreting polypeptide. Thus, a microorganism “expressing” a gene or polypeptide, “producing” a polypeptide, or “secreting” a polypeptide of the current disclosure includes the microorganism in its “dormant” state. As used herein, “secrete” means that the protein is exported outside the cell and into the culture medium/supernatant or other extracellular milieu.

As used herein, the term “constitutive” in the context of a promoter (or by extension relating to gene expression or secretion of a polypeptide) refers to a promoter that allows for continual transcription of its associated gene. A constitutive promoter compares to an “inducible” promoter.

As used herein, the term “inducible” in the context of a promoter (or by extension relating to gene expression or secretion of a polypeptide) refers to a promoter that allows for increased transcription of the gene it is operably linked to when in the presence of an inducer of said promoter.

The term “about” in relation to a reference numerical value, and its grammatical equivalents as used herein, can include the reference numerical value itself and a range of values plus or minus 10% from that reference numerical value. For example, the term “about 10” includes 10 and any amount from and including 9 to 11. In some cases, the term “about” in relation to a reference numerical value can also include a range of values plus or minus 10%, 9%, 8%, 7%, 6%, 5%, 4%, 3%, 2%, or 1% from that reference numerical value. In some embodiments, “about” in connection with a number or range measured by a particular method indicates that the given numerical value includes values determined by the variability of that method.

Ranges: throughout this disclosure, various aspects of the invention can be presented in a range format. It should be understood that the description in range format is merely for convenience and brevity and should not be construed as an inflexible limitation on the scope of the invention. It is understood that any and all whole or partial integers between the ranges set forth are included herein. The description of a range should also be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As envisioned in the present disclosure with respect to the disclosed compositions of matter and methods, in one aspect, the embodiments of the disclosure comprise the components and/or steps disclosed therein. In another aspect, the embodiments of the disclosure consist essentially of the components and/or steps disclosed therein. In yet another aspect, the embodiments of the disclosure consist of the components and/or steps disclosed therein.

The term “chromosomally integrated” or “integrated into a chromosome” or any variation thereof means that a nucleic acid sequence (e.g., gene; open reading frame; exogenous nucleic acid encoding a polypeptide; promoter; expression cassette; and the like) is located on (integrated into) a microbial (e.g., bacterial) chromosome, i.e., is not located on an episomal vector, such as a plasmid. In some embodiments, in which the nucleic acid sequence is chromosomally integrated, the polypeptide encoded by such chromosomally integrated nucleic acid is constitutively expressed. For example, an exemplary nucleic acid sequence that is chromosomally integrated may inducibly express the polypeptide the integrated nucleic acid encodes.

An “IL-10 gene” refers to an interleukin 10 gene encoding an “IL-10 polypeptide.” The term “IL-10 gene” includes “IL-10 variant genes” encoding “IL-10 variant polypeptides.” The IL-10 gene can be a mammalian gene (e.g., bovine, equine, ovine, caprine, murine, primate, etc.). The IL-10 gene preferably encodes a human IL-10 polypeptide to a variant of a human IL-10 polypeptide. The DNA sequence encoding IL-10 in an LAB may be codon optimized to facilitate expression in LAB, and as such, may differ from that in the native organism (e.g., humans).

The term “IL-10” or “IL-10 polypeptide” refers to a functional, IL-10 polypeptide (e.g., human IL-10 polypeptide) that has at least the amino acid sequence of the mature form (i.e. without its secretion signal), but also includes membrane-bound forms and soluble forms, as well as “IL-10 variant polypeptides.”

An “IL-10 variant” or “IL-10 variant polypeptide” refers to a modified (e.g., truncated or mutated), but functional IL-10 polypeptide, e.g., a truncated or mutated version of human IL-10. The term “IL-10 variant polypeptide” includes IL-10 polypeptides with enhanced activity or diminished activity when compared to a corresponding wild-type IL-10 polypeptide. An “IL-10 variant polypeptide” retains at least some IL-10 activity (functional polypeptide).

An “IL-2 gene” refers to an interleukin 2 gene encoding an “IL-2 polypeptide.” The term “IL-2 gene” includes “IL-2 variant genes” encoding “IL-2 variant polypeptides.” The DNA sequence encoding IL-2 in an LAB may be codon optimized to facilitate expression in LAB, and as such may differ from that in the native organism (e.g., humans).

The term “IL-2” or “IL-2 polypeptide” refers to a functional, IL-2 polypeptide (e.g., human IL-2 polypeptide) that has at least the amino acid sequence of the mature form (i.e. without its secretion signal), but also includes membrane-bound forms and soluble forms, as well as “IL-2 variant polypeptides.”

An “IL-2 variant” or “IL-2 variant polypeptide” refers to a modified (e.g., truncated or mutated), but functional IL-2 polypeptide, e.g., a truncated or mutated version of human IL-2. The term “IL-2 variant polypeptide” includes IL-2 polypeptides with enhanced activity or diminished activity when compared to a corresponding wild-type IL-2 polypeptide. An “IL-2 variant polypeptide” retains at least some IL-2 activity (functional polypeptide).

Celiac disease, also known as celiac sprue or gluten-sensitive enteropathy, is a chronic inflammatory disease that develops from an immune response to specific dietary grains that contain gluten. Upon ingestion of gluten, the immune system responds by attacking the small intestine and inhibiting the absorption of important nutrients. Celiac is a complex multigenic disorder that is strongly associated with the genes that encode the human leukocyte antigen (HLA) variants HLA-DQ2 or HLA-DQ8. There are two HLA-DQ2 isoforms, HLA-DQ2.2 and HLA-DQ2.5, of which HLA-DQ2.5 is the haplotype associated with the highest risk of CeD (Fallang et al., 2009, “Differences in the risk of celiac disease associated with HLA-DQ2.5 or HLA-DQ2.2 are related to sustained gluten antigen presentation,” Nat. Immunol. 10(10): 1096-1101). Approximately 90% of CeD patients carry the HLA-DQ2 haplotype whereas HLA-DQ8 is found in 5-10% of patients (Sollid, 2000, “Molecular basis of celiac disease,” Annu. Rev. Immunol. 18: 53-81). One of the most important aspects in the pathogenesis of celiac disease is the activation of a T-helper 1 immune response. This arises when antigen-presenting cells (APCs) that express HLA-DQ2/DQ8 molecules present the toxic gluten peptides to CD4+ T-cells. Certain components of gluten, namely gliadins, glutenins, hordein, and secalins, contain a high content of proline and glutamine residues making them resistant to degradation by gastro-intestinal enzymes (Gujral et al., 2012, “Celiac disease: prevalence, diagnosis, pathogenesis and treatment,” World J. Gastroenterol. 18(42): 6036-6059). As a result, following a gluten-containing meal an elevated intestinal concentration of potentially immunoactive peptides is maintained. These undigested peptide fragments are subject to deamidation by tissue transglutaminase 2 (tTG2) which converts glutamine to glutamate. This introduces negative charges that have stronger binding affinity for HLA-DQ2 and HLA-DQ8 on antigen presenting cells (APCs) (Kupfer et al., 2012, “Pathophysiology of celiac disease,” Gastrointest. Endosc. Clin. N. Am. 22(4): 639-660), which leads to a more rigorous gluten-specific CD4+ T helper type 1 cell (Th1) activation (Schuppan et al., 2009, “Celiac disease: from pathogenesis to novel therapies,” Gastroenterology 137(6): 1912-1933). Thus, deamidations enhance immunogenicity of the epitopes. The gluten components contain peptides that specifically bind HLA-DQ2 and HLA-DQ8, i.e., celiac-specific T cell epitopes. More than a dozen celiac-specific T cell epitopes have been identified thus far, mostly from gliadins (Arentz-Hansen et al., 2002, “Celiac lesion T cells recognize epitopes that cluster in regions of gliadins rich in proline residues,” Gastroenterology 123(3): 803-809), the majority of which are HLA-DQ2-restricted (Tollefsen et al., 2006, “HLA-DQ2 and -DQ8 signatures of gluten T cell epitopes in celiac disease,” J. Clin. Invest. 116(8): 2226-2236). Both classes of gluten proteins, gliadins and glutenins, contain peptide sequences that specifically bind HLA-DQ2 and HLA-DQ8.

An “CeD-specific antigen polypeptide” refers to a gluten protein that comprises at least one peptide sequence that specifically binds HLA-DQ2 and/or HLA-DQ8. Exemplary CeD-specific antigen polypeptides are gliadin and glutenin. A peptide sequence that specifically binds HLA-DQ2 and/or HLA-DQ8 is a CeD-specific T cell epitope. As used herein, a HLA-DQ2 specific epitope is a CeD-specific T cell epitope that binds HLA-DQ2, and a HLA-DQ8 specific epitope is a CeD-specific T cell epitope that binds HLA-DQ8.

An “CeD-specific antigen polypeptide gene” refers to a gene encoding a “CeD-specific antigen polypeptide.” The term “CeD-specific antigen polypeptide gene” includes nucleic acids encoding variants of a “CeD-specific antigen” or a “CeD-specific antigen variant polypeptide.” The DNA sequence encoding CeD-specific antigen in an LAB may be codon optimized to facilitate expression in LAB, and as such may differ from that in the native organism (e.g. humans).

The term “CeD-specific antigen polypeptide” refers to a functional, e.g., full-length, polypeptide, as well as “CeD-specific antigen variant polypeptides,” which may have enhanced activity or diminished activity when compared to a corresponding wild-type polypeptide.

The term “CeD-specific antigen variant” or “CeD-specific antigen variant polypeptide” refers to a modified (e.g., truncated and/or mutated), but functional polypeptide, e.g., a truncated and/or mutated version of gliadin or glutenin. In particular, the term “CeD-specific antigen variant polypeptide” refers to a polypeptide fragment of gliadin comprising at least one HLA-DQ2-specific or HLA-DQ8-specific epitope. The gliadin can be selected from any gluten associated with CeD, and in particular, wheat (e.g., Triticum aestivum and Triticum spelta), rye (e.g., Secale cereale), or barley (e.g., Hordeum vulgare) gluten. HLA-DQ2-specific epitopes and HLA-DQ8-specific epitopes are known in the art. See, e.g., U.S. Pat. Nos. 8,748,126, 9,017,690, 10,105,437, and 10,053,497, and Vader et al., 2003, “Characterization of cereal toxicity for celiac disease patients based on protein homology in grains,” Gastroenterology 1225: 1105-1113. Alpha-gliadins comprise the main T-cell epitopes, e.g., DQ2.5-glia-al, DQ2.5-glia-a2, and DQ2.5-glia-a3, and are the most immunogenic fraction of gluten proteins (see, e.g., Ruiz-Carnicer et al., 2019, Nutrients, 11, 220; doi:10.3390/nu11020220). Wheat a-gliadin proteins contain three major immunogenic peptides in a 33 amino acid peptide six overlapping copies of three highly stimulatory epitopes (see, e.g., Ozuna et al., 2015, The Plant Journal, 82: 794-805. Exemplary HLA-DQ2-specific epitopes in the present disclosure include the 33 amino acid fragment comprising 6 overlapping α1- and α2-gliadin epitopes LQLQPFPQPQLPYPQPQLPYPQPQLPYPQPQPF (amino acids 57-89 of UniProt: Q9M4L6; SEQ ID NO: 3) and a corresponding deamidated version thereof LQLQPFPQPELPYPQPQLPYPQPELPYPQPQPF (amino acids corresponding to positions 66 and 80 of UniProt: Q9M4L6 are deamidated; SEQ ID NO: 7). Exemplary HLA-DQ8-specific epitopes include QYPSGQGSFQPSQQNPQA (amino acids 225-242 of UniProt Q9M4L6; SEQ ID NO: 5) and a corresponding deamidated version thereof QYPSGEGSFQPSQENPQA (SEQ ID NO: 9). Sequence variants of known epitopes retaining antigenic properties (e.g., HLA-DQ8-specific or HLA-DQ2 specific) are also useful in the compositions and methods of the current disclosure. Generally, truncated versions of a CeD-specific antigen are efficiently expressed and secreted by the microorganism (e.g., Lactococcus lactis).

The “percentage identity” between polypeptide sequences can be calculated using commercially available algorithms which compare a reference sequence with a query sequence. In some embodiments, polypeptides are 70%, at least 70%, 75%, at least 75%, 80%, at least 80%, 85%, at least 85%, 90%, at least 90%, 92%, at least 92%, 95%, at least 95%, 97%, at least 97%, 98%, at least 98%, 99%, or at least 99% or 100% identical to a reference polypeptide, or a fragment thereof (e.g., as measured by BLASTP or CLUSTAL, or other alignment software) using default parameters. Similarly, nucleic acids can also be described with reference to a starting nucleic acid, e.g., they can be 50%, at least 50%, 60%, at least 60%, 70%, at least 70%, 75%, at least 75%, 80%, at least 80%, 85%, at least 85%, 90%, at least 90%, 95%, at least 95%, 97%, at least 97%, 98%, at least 98%, 99%, at least 99%, or 100% identical to a reference nucleic acid or a fragment thereof (e.g., as measured by BLASTN or CLUSTAL, or other alignment software using default parameters). When one molecule is said to have a certain percentage of sequence identity with a larger molecule, it means that when the two molecules are optimally aligned, the percentage of residues in the smaller molecule finds a match residue in the larger molecule in accordance with the order by which the two molecules are optimally aligned, and the “%” (percent) identity is calculated in accord with the length of the smaller molecule.

### Celiac Disease

The term “celiac disease” encompasses a spectrum of conditions in a subject caused by varying degrees of gluten sensitivity, including a severe form characterized by flat small intestinal mucosa (hyperplastic villous atrophy) and other forms characterized by milder symptoms. See, e.g., Rubio-Tapia et al., 2013, Am. J. Gastroenterol. 108:656-676 and Ludvigsson et al., 2014, “BSG Coeliac Disease Guidelines Development Group; British Society of Gastroenterology. Diagnosis and management of adult coeliac disease: guidelines from the British Society of Gastroenterology,” Gut 63(8): 1210-28; Epub 2014 Jun. 10.

### Subject

A “subject” is an organism, which may benefit from being administered a composition of the present disclosure, e.g., according to methods of the present disclosure. The subject may be a mammal (“mammalian subject”). Exemplary mammalian subjects include humans, farm animals (such as cows, pigs, horses, sheep, goats), pets or domesticated animals (such as a dogs, cats, and rabbits), and other animals, such as mice, rats, and primates. In some examples, the mammalian subject is a human patient

### Promoter

By “promoter” is meant generally a region on a nucleic acid molecule, for example DNA molecule, to which an RNA polymerase binds and initiates transcription. A promoter is for example, positioned upstream, i.e., 5′, of the sequence the transcription of which it controls. The skilled person will appreciate that the promoter may be associated with additional native regulatory sequences or regions, e.g. operators. The precise nature of the regulatory regions needed for expression may vary from organism to organism, but shall in general include a promoter region which, in prokaryotes, contains both the promoter (which directs the initiation of RNA transcription) as well as the DNA sequences which, when transcribed into RNA, will signal the initiation of protein synthesis. Such regions will normally include those 5′-non-coding sequences involved with initiation of transcription and translation, such as the Pribnow-box (cf. TATA-box), Shine-Dalgarno sequence, and the like.

The terms “secretion leader sequence,” “secretion leader,” and “secretion signal sequence” are used interchangeably herein. The terms are used in accordance with their art recognized meaning, and generally refer to a nucleic acid sequence, which encodes a “signal peptide” or “secretion signal peptide.” As used herein, “secretion leader” can also refer to the polypeptide encoded by the nucleic acid sequence. A signal peptide or secretion signal peptide or secretion leader causes a polypeptide being expressed by a microorganism and comprising the signal peptide or secretion leader to be secreted by the microorganism, i.e., causes the polypeptide to leave the intracellular space, e.g., be secreted into the surrounding medium, or be anchored in the cell wall with at least a portion of the polypeptide be exposed to the surrounding medium, e.g. on the surface of the microorganism.

The term “operably linked” refers to a juxtaposition wherein the components described are in a relationship permitting them to function in their intended manner A control sequence “operably linked” to a coding sequence is ligated in such a way that expression of the coding sequence is achieved under condition compatible with the control sequences. For example, a promoter is said to be operably linked to a gene, open reading frame or coding sequence, if the linkage or connection allows or effects transcription of said gene. In a further example, a 5′ and a 3′ gene, cistron, open reading frame or coding sequence are said to be operably linked in a polycistronic expression unit, if the linkage or connection allows or effects translation of at least the 3′ gene. For example, DNA sequences, such as, e.g., a promoter and an open reading frame, are said to be operably linked if the nature of the linkage between the sequences does not (1) result in the introduction of a frame-shift mutation, (2) interfere with the ability of the promoter to direct the transcription of the open reading frame, or (3) interfere with the ability of the open reading frame to be transcribed by the promoter region sequence.

As used herein, a fusion polypeptide refers as a polypeptide derived from a single nucleotide sequence that may contain 2 or more coding sequences of different origin or portions of coding sequences of different origin with or without intervening amino acid linker sequences. With respect to fusion polypeptides, and as used herein, the term “fused” refers to the fact that each of the components performs the same function in the fusion to the other component as it would if it were not so fused. “Fused” as used in this context encompasses both direct covalent linkage between a first and a second polypeptide sequence, and indirect covalent linkage, e.g., there is an intervening amino acid linker sequence between the first and second polypeptide sequences. With respect to nucleic acid sequence encoding fusion polypeptides, the phrase “operabably linked” refers to the fact that the sequences of the two or more coding sequences of different origin or portions of coding sequences of different origin with or without sequence encoding an intervening amino acid linker are such that the coding sequences are in the same frame to yield, when translated, the correct amino acid seequence for the polypeptide encoded by the two or more two or more coding sequences of different origin or portions of coding sequences of different origin.

### Expression Cassette

The term “expression cassette” or “expression unit” is used in accordance with its generally accepted meaning in the art, and refers to a nucleic acid containing one or more genes and sequences controlling the expression of the one or more genes. Exemplary expression cassettes contain at least one promoter sequence and at least one open reading frame

### Polycistronic Expression Cassette

The terms “polycistronic expression cassette” “polycistronic expression unit” or “polycistronic expression system” are used herein interchangeably and in accordance with their generally accepted meaning in the art. They refer to a nucleic acid sequence wherein the expression of two or more genes is regulated by common regulatory mechanisms, such as promoters, operators, and the like. The term polycistronic expression unit as used herein is synonymous with multicistronic expression unit. Examples of polycistronic expression units are without limitation bicistronic, tricistronic, and tetracistronic expression units. Any mRNA comprising two or more, such as 3, 4, 5, 6, 7, 8, 9, 10, or more, open reading frames or coding regions encoding individual expression products such as proteins, polypeptides and/or peptides is encompassed within the term polycistronic. A polycistronic expression cassette includes at least one promoter, and at least two open reading frames controlled by the promoter, wherein an intergenic region is optionally placed between the two open reading frames.

In some examples, the “polycistronic expression cassette” includes one or more endogenous genes and one or more exogenous genes that are transcriptionally controlled by a promoter which is endogenous to the microorganism (e.g., LAB). The polycistronic expression unit or system as described herein can be transcriptionally controlled by a promoter that is exogenous to the microorganism (e.g., LAB). In a further embodiment, the translationally or transcriptionally coupled one or more endogenous genes and one or more exogenous genes as described herein are transcriptionally controlled by the native promoter of (one of) said one or more endogenous genes. Preferably, in the microorganism (e.g., LAB), the polycistronic expression cassette is integrated into the chromosome such that the endogenous gene is located in its native chromosomal locus in the microorganism. In another embodiment, the polycistronic expression unit is transcriptionally controlled by the native promoter of (one of) said one or more endogenous genes comprised in said polycistronic expression unit. In another embodiment, the polycistronic expression unit is operably linked to a gram-positive endogenous promoter. In an exemplary embodiment, the promoter may be positioned upstream of, i.e., 5′ of the open reading frame(s) to which it is operably linked. In a further embodiment, the promoter is the native promoter of the 5′ most, i.e., most upstream, endogenous gene in the polycistronic expression unit. Accordingly, in some examples, the polycistronic expression unit contains an endogenous gene and one or more exogenous genes transcriptionally coupled to the 3′ end of said one or more endogenous gene, for example wherein said one or more exogenous gene(s) is (are) the most 3′ gene(s) of the polycistronic expression unit. Exemplary polycistronic expression systems are disclosed in WO 2012/164083 and U.S. Pat. No. 9,920,324 each of which is incorporated herein by reference.

In an embodiment, the polycistronic expression unit comprises: (i) an endogenous gene promoter; (ii) the endogenous gene positioned 3′ of the endogenous gene promoter; (iii) an intergenic region; and (iv) the exogenous nucleic acid encoding hIL-10, wherein the exogenous nucleic acid encoding hIL-10 further encodes a secretion leader sequence fused in frame to the hIL-10 coding sequence, and wherein the endogenous gene and said exogenous nucleic acid encoding hIL-10 are transcriptionally and translationally coupled by the intergenic region. In an embodiment, the polycistronic expression unit further comprises (i) a second intergenic region positioned 3′ of said exogenous nucleic acid encoding hIL-10; and (ii) said exogenous nucleic acid encoding the gliadin polypeptide, wherein the exogenous nucleic acid encoding said gliadin polypeptide further encodes a secretion leader sequence fused in frame to the gliadin polypeptide, and wherein the exogenous nucleic acid encoding the gliadin polypeptide and the exogenous nucleic acid encoding hIL-10 are transcriptionally and translationally coupled by the second intergenic region.

As used herein, a “polycistronic integration vector” is a vector for integrating a polycistronic expression unit into a target nucleic acid. A polycistronic integration vector is a nucleic acid construct and refers to a polynucleic acid sequence comprising at least one intergenic region transcriptionally coupled to at least one open reading frame or coding region. In some examples, a polycistronic integration vector includes two or more open reading frames or coding regions. The at least one intergenic region transcriptionally couples two open reading frames or coding regions. In some examples, a polycistronic integration vector includes at least two intergenic regions and at least two open reading frames or coding regions. In some examples, a polycistronic integration vector further comprises a sequence encoding a secretion leader fused in frame to a coding region. In some embodiments, the polycistronic integration vector includes a first intergenic region transcriptionally coupled at its 3′ end to a first open reading frame or coding region, a second intergenic region that is transcriptionally coupled to the 3′ end of the first open reading frame or coding region and the second intergenic region is transcriptionally coupled at its 3′ end to a second open reading frame or coding region. The structure of this polycistronic integration vector can be represented as intergenic region>>open reading frame>>intergenic region>>open reading frame.

In further embodiments, the polycistronic integration vector includes a first intergenic region transcriptionally coupled at its 3′ end to a sequence encoding a secretion leader fused in frame to a coding region, a second intergenic region that is transcriptionally coupled to the 3′ end of the coding region, and the second intergenic region is transcriptionally coupled at its 3′ end to a sequence encoding a secretion leader fused in framed to a second coding region.

The 5′ to 3′ structure of this polycistronic integration vector can be represented as intergenic region>>secretion leader fused to coding region>>intergenic region>>secretion leader fused to a coding region. In some embodiments, the polycistronic integration vector structure can be represented as intergenic region>>secretion secretion leader fused to hil-10>>intergenic region>>secretion leader fused to CeD-specific antigen. In some embodiments, the polycistronic integration vector structure can be represented as intergenic region>>SSusp45 fused to hil-10>>intergenic region>>SSps356 fused to deamidated HLA-DQ2-specific epitope. In an embodiment, the polycistronic integration vector can be represented as IRrpmD>>SSusp45-hil-10>>IRrplN>>SSps356-CeD-specific antigen. In some embodiments, the polycistronic integration vector further comprises regulatory sequences, such as stop codons and start codons.

The polycistronic integration vector is suitable for cloning an open reading frame or coding sequence at the 3′ end of an intergenic region into another nucleic acid sequence. In some examples, the polycistronic integration vector is suitable for being replicated in a microorganism, such as a gram-positive bacterium. In some examples, the polycistronic integration vector is suitable for effecting homologous recombination in a microorganism, such as a gram-positive bacterium. In particular, the polycistronic integration vector is suitable for chromosomal integration of the intergenic region and open reading frame or coding region. In some examples, the polycistronic integration vector further comprising nucleic acid sequences flanking the 5′ and 3′ ends of the at least one intergenic region transcriptionally coupled to at least one open reading frame or coding region. The 5′ flanking nucleic acid comprises a nucleic acid sequence that is identical to coding sequence at the 3′ end of an integration target gene. The 5′ flanking nucleic acid sequence comprises at least about 50 nucleotides, at least about 100 nucleotides, or at least about 150 nucleotides identical to the 3′ end of the integration target gene. The 5′ flanking nucleic acid sequence may comprise up to about 1000 nucleotides, about 1500 nucleotides, or about 2000 nucleotides of a sequence identical to the 3′ end of the integration target gene, or more as needed for integration. In an embodiment, the 5′ flanking sequence comprises the stop codon of the target gene immediately 5′ to the first at least one intergenic region. The 3′ flanking nucleic acid comprises a nucleic sequence that is identical to a DNA sequence that is 3′ to the integration target gene. The 3′ flanking nucleic acid sequence comprises at least about 50 nucleotides, at least about 100 nucleotides, or at least about 150 nucleotides identical to the DNA sequence that is 3′ to the integration target gene. The 3′ flanking nucleic acid sequence may comprise up to about 1000 nucleotides, about 1500 nucleotides, or about 2000 nucleotides of a sequence identical to the DNA sequence that is 3′ to the integration target gene, or more as needed for integration. In an embodiment, the 3′ flanking region sequence is identical to the sequence that is immediately 3′ to the integration target gene. In yet another embodiment, the polycistronic integration vector further comprises one or more selection markers, such as antibiotic resistance genes, that are positioned 5′ of the 5′ flanking nucleic acid sequence targeting the integration target gene, and/or 3′ to the 3′ flanking nucleic acid sequence.

As used herein, the term “transcriptionally coupled” is synonymous with “transcriptionally linked” or “transcriptionally connected”. These terms generally refer to polynucleic acid sequences comprising two or more open reading frames or coding sequences which are commonly transcribed as one mRNA, and which can be translated into two or more individual polypeptides.

As used herein, the term “translationally coupled” is synonymous with “translationally linked” or “translationally connected”. These terms in essence relate to polycistronic expression cassettes or units. Two or more genes, open reading frames or coding sequences are said to be translationally coupled when common regulatory element(s) such as in particular a common promoter effects the transcription of said two or more genes as one mRNA encoding said two or more genes, open reading frames or coding sequences, which can be subsequently translated into two or more individual polypeptide sequences. The skilled person will appreciate that bacterial operons are naturally occurring polycistronic expression systems or units in which two or more genes are translationally or transcriptionally coupled.

### Intergenic Region

As used herein, the term “intergenic region” is synonymous with “intergenic linker” or “intergenic spacer.” An intergenic region is defined as a polynucleic acid sequence between adjacent (i.e., located on the same polynucleic acid sequence) genes, open reading frames, cistrons or coding sequences. By extension, the intergenic region can include the stop codon of the 5′ gene and/or the start codon of the 3′ gene, which are linked by said intergenic region. As defined herein, the term intergenic region specifically relates to intergenic regions between adjacent genes in a polycistronic expression unit. For example, an intergenic region as defined herein can be found between adjacent genes in an operon. Accordingly, in an embodiment, the intergenic region as defined herein is an operon intergenic region. Exemplary intergenic region disclosure is found in WO 2012/164083 and U.S. Pat. No. 9,920,324, the disclosure of each of which is incorporated herein by reference in its entirety.

In some examples, the intergenic region, linker or spacer is selected from intergenic regions preceding, i.e., 5′ to, more particularly immediately 5′ to, rplW, rplP, rpmD, rplB, rpsG, rpsE or rplN of a gram-positive bacterium. In an embodiment, said gram positive bacterium is a lactic acid bacterium, for example a Lactococcus species, e.g., Lactococcus lactis, and any subspecies or strain thereof. In an embodiment, said intergenic region encompasses the start codon of rplW, rplP, rpmD, rplB, rpsG, rpsE or rplN and/or the stop codon of the preceding, i.e. 5′, gene. In a preferred embodiment, the invention relates to a gram-positive bacterium or a recombinant nucleic acid as described herein, wherein the endogenous gene and the one or more exogenous genes are transcriptionally coupled by intergenic region or regions active in the gram-positive bacterium, for example wherein the intergenic region or regions is endogenous to said gram-positive bacterium, for example, wherein the endogenous intergenic region is selected from intergenic regions preceding rplW, rplP, rpmD, rplB, rpsG, rpsE or rplN.

The skilled person will appreciate that if the intergenic region encompasses a 5′ stop codon and/or a 3′ start codon, these respective codons in some cases are not present in the genes which are linked by said intergenic regions, in order to avoid double start and/or stop codons, which may affect correct translation initiation and/or termination. Methods for identifying intergenic regions are known in the art. By means of further guidance, intergenic regions can for instance be identified based on prediction of operons, and associated promoters and open reading frames, for which software is known and available in the art. Exemplary intergenic regions (IRs) are described in for example international patent publication WO2012/164083 and U.S. Pat. No. 9,920,324, the disclosure of each of which is incorporated herein by reference in its entirety.

The term “international unit” (IU) is used herein in accordance with its art-recognized meaning and represents an amount of a substance (e.g., polypeptide). The mass or volume that constitutes one international unit varies based on which substance is being measured. The World Health Organization (WHO) provides unit characterizations for bioactive polypeptides.

### CeD Specific Antigen

The at least one microorganism of the present disclosure contains an exogenous nucleic acid encoding at least one disease-specific (i.e., CeD-specific) antigen gene, and can express such gene under conditions sufficient for expression. In particular, the term “CeD-specific antigen variant polypeptide” refers to a polypeptide fragment of gliadin comprising at least one HLA-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope. The gliadin can be selected from any gluten associated with CeD, and in particular, wheat (e.g., Triticum aestivum and Triticum spelta), rye (e.g., Secale cereale), or barley (e.g., Hordeum vulgare) gluten. An exemplary wheat gliadin sequence is UniProtKB Q9M4L6:

(SEQ ID NO: 1)
MVRVPVPQLQPQNPSQQQPQEQVPLVQQQQFPGQQQPFPPQQPYPQPQPF
PSQQPYLQLQPFPQPQLPYPQPQLPYPQPQLPYPQPQPFRPQQPYPQSQP
QYSQPQQPISQQQQQQQQQQQQKQQQQQQQQILQQILQQQLIPCRDVVLQ
QHSIAYGSSQVLQQSTYQLVQQLCCQQLWQIPEQSRCQAIHNVVHAIILH
QQQQQQQQQQQQPLSQVSFQQPQQQYPSGQGSFQPSQQNPQAQGSVQPQQ
LPQFEEIRNLALETLPAMCNVYIPPYCTIAPVGIFGTNYR.

An exemplary nucleic acid sequence encoding the wheat gliadin is GenBank Accession no. AJ133611.1. Additional exemplary gliadin sequences include rye gliadin: MKTFLILSLLAIVATTTTIAVRVPVPQLQPQNPSQQQPQEQVPLVQQQQFPGQQQPFPP RQPYPQPQPFPSQQPYLQLQPFPQPQQPYPQPQLLYPQPQPFRPQQPYPQPQPQYSQPQ QPISQQQQQQQQQQQQQILQQILQQQLIPCRDVVLQQHSIAHGSSQVLQQSTYQLVQQ LCCQQLWQIPEQSRCQAIHNVVHAIILHQQQQQQQQQQQQQQQPLSQVSFQQPQQQY PSGQGSFQPSQQNPQAQGSVQPQQLPQFEEIRNLALETLPAMCNVYIPPYCTIAPVGIFG TN (SEQ ID NO: 31) (UniProtKB I3RXX8 and GenBank Accession no. JQ728948) and barley gliadin (also called B1-hordein): MKTFLIFALLAIAATSTIAQQQPFPQQP IPQQPQPYPQQPQPYPQQPFPPQQPFPQQPVP QQPQPYPQQPFPPQQPFPQQPPFWQQKPFPQQPPFGLQQP ILSQQQPCTPQQTPLPQGQL YQTLLQLQ IQYVHP SI LQQLNPCKVFLQQQCSPVPVPQRIARSQMLQQS SCHVLQQQCCQ QLPQIPEQFRHEAIRAIVYS IFLQEQPQQLVEGVSQPQQQLWPQQVGQCSFQQPQPQQVG QQQQVPQSAFLQPHQIAQLEATTSIALRTLPMMCSVNVPLYRILRGVGPSVGV (SEQ ID NO: 32) (where residues 1-18 are a signal peptide; UniProtKB P06470 and GenBank Accession no. X03103).

HLA-DQ2-specific epitopes and HLA-DQ8-specific epitopes are known in the art. See, for instance, U.S. Pat. Nos. 8,748,126, 9,017,690, 10,105,437, and 10,053,497, each of which is incorporated by reference herein. See also Vader et al., 2003, “Characterization of cereal toxicity for celiac disease patients based on protein homology in grains,” Gastroenterology 1225: 1105-1113; and Tye-Din et al., 2010, “Comprehensive, quantitative mapping of T cell epitopes in gluten in celiac disease, Sci. Transl. Med. 2(41):41ra51. Exemplary HLA-DQ2-specific epitopes include a 33 amino acid fragment comprising 6 overlapping α1- and α2-gliadin epitopes LQLQPFPQPQLPYPQPQLPYPQPQLPYPQPQPF (amino acids 57-89 of UniProt: Q9M4L6; SEQ ID NO: 3) and corresponding deamidated forms including LQLQPFPQPELPYPQPQLPYPQPELPYPQPQPF (amino acids corresponding to positions 66 and 80 of UniProt: Q9M4L6 are deamidated; SEQ ID NO: 7) and LQLQPFPQPELPYPQPELPYPQPELPYPQPQPF (amino acids corresponding to positions 66, 73, and 80 of UniProt: Q9M4L6 are deamidated; SEQ ID NO: 33). Exemplary HLA-DQ8-specific epitopes include QYPSGQGSFQPSQQNPQA (SEQ ID NO: 5) (amino acids 225-242 of UniProtKB Q9M4L6) and a corresponding deamidated form QYPSGEGSFQPSQENPQA (SEQ ID NO: 9). Sequence variants of known epitopes retaining antigenic properties (e.g., HLA-DQ8-specific or HLA-DQ2 specific) are also useful in the compositions and methods of the current disclosure. Examples are epitopes having 1, 2 or 3 amino acid differences from any known HLA-DQ2-specific epitope or HLA-DQ8-specific epitope. Generally, truncated versions of a CeD-specific antigen are efficiently expressed and secreted by the microorganism (e.g., Lactococcus lactis).

Any nucleotide sequence encoding the amino acid sequence of wheat gliadin (UniProtKB Q9M4L6), or any nucleotide sequence encoding at least about 20, at least about 30, at least about 40, at least about 50, at least about 60, at least about 70, or at least about 80 consecutive amino acids thereof, or any nucleotide sequence encoding a polypeptide having at least about 60%, at least about 70%, at least about 80%, at least about 90%, at least about 95%, at least about 96%, at least about 97%, at least about 98%, or at least about 99% sequence identity with SEQ ID NO: 3 may be used.

A person of ordinary skill in the art will appreciate that the optimal amount of CeD-specific antigen to be delivered to the subject using the methods of the present disclosure varies, e.g., with the type of antigen, the microorganism expressing the antigen, and the genetic construct, e.g., the strength of the promoter used in the genetic construct. Typically, the microorganism will be administered in an amount equivalent to a particular amount of expressed antigen, or in an amount, which generates a desired PK profile for the respective antigen polypeptide in the respective subject. Exemplary daily antigen doses are from about 10 fg (femptogram) to about 100 μg (microgram) of active polypeptide per day. Other exemplary dose ranges are from about 1 pg (picogram) to about 100 μg per day; or from about 1 ng to about 100 μg per day.

The above antigen doses may be realized by administering to the subject effective amounts of the microorganism per day, wherein the microorganism is adapted to express a sufficient amount of bioactive polypeptide to realize the desired dose, such as those above. The microorganism secreting the antigen polypeptide may be delivered in a dose of from about 10⁴ colony forming units (cfu) to about 10¹² cfu per day, e.g., from about 10⁶ cfu to about 10¹² cfu per day, or from about 10⁹ cfu to about 10¹² cfu per day. In some examples, the unit dosage contains about 1×10⁴ to about 1×10¹² colony-forming units (cfu) of sAGX0868. In some examples, the unit dosage form contains from about 1×10⁸ to about 1×10¹¹ cfu, or about 1×10¹⁰ to about 1×10¹¹ cfu, or about 1×10¹¹ cfu sAGX0868.

The amount of secreted antigen polypeptide can be determined based on cfu, for example in accordance with the methods described in Steidler et al., Science 2000; 289(5483): 1352-1355, or by using ELISA. For example, a particular microorganism may secrete at least about 1 ng (nanogram) to about 1 μg of active polypeptide per 10⁹ cfu. Based thereon, the skilled person can calculate the range of antigen polypeptide secreted at other cfu doses.

Each of the above doses/dose ranges may be administered in connection with any dosing regimen as described herein. The daily dose of active polypeptide may be administered in 1, 2, 3, 4, 5, or 6 portions throughout the day. Further, the daily doses may be administered for any number of days, with any number of rest periods between administration periods. For example, a dose of from about 0.01 to about 3 0 million international units (MIU) of IL-10/day/subject may be administered every other day for a total of 6 weeks.

### Treating

The terms “treatment”, “treating”, and the like, as used herein means ameliorating or alleviating characteristic symptoms or manifestations of a disease or condition, e.g., CeD. For example, treatment of CeD as described herein can result in the restoration or induction of antigen-specific immune tolerance in the subject. In other examples, treatment means reducing or eliminating villous atrophy and/or increasing the villous height/crypt depth ratio in small intestine of the subject, for instance, increasing the villous height/crypt depth ratio (Vh/Cd) to a normal range. As used herein, “normal range” for Vh/Cd may be the Vh/Cd in a reference subject who does not have CeD at all, or may refer to the Vh/Cd of the subject being treated, when that subject has not be exposed to intestinal gluten. As used herein these terms also encompass, preventing or delaying the onset of a disease or condition or of symptoms associated with a disease or condition, including reducing the severity of a disease or condition or symptoms associated therewith prior to affliction with said disease or condition. Such prevention or reduction prior to affliction refers to administration of the compound or composition of the invention to a patient that is not at the time of administration afflicted with the disease or condition. “Preventing” also encompasses preventing the recurrence or relapse-prevention of a disease or condition or of symptoms associated therewith, for instance after a period of improvement. Treatment of a subject “in need thereof” conveys that the subject has a diseases or condition, and the therapeutic method of the invention is performed with the intentional purpose of treating the specific disease or condition.

### Patient Populations and Sub-Populations

The subject can have celiac disease (symptomatic or asymptomatic) or can be suspected of having it. The subject may be on a gluten-free diet (GFD). The subject can be on a GFD subsequent to a period of gluten ingestion of, for instance, from 1 day up to 21 days. The subject may be in an acute phase response (for example they are diagnosed with celiac disease, but have only ingested gluten in the last 24 hours before which they had been on a gluten-free diet for 14 to 30 days). In some examples according to these embodiments, the subject has villous atrophy as determined, for instance, by histopathology assessment of small intestinal biopsy. In some examples according to these embodiments, the subject has intraepithelial lymphocytosis and/or elevated level CD3+ intraepithelial lymphocytes (IELs). In some examples according to these embodiments, the subject has an elevated number of cytotoxic CD8+ IELs. In some examples according to these embodiments, the subject has an elevated level of Foxp3-Tbet+CD4+ T cells and/or reduced level of Foxp3+Tbet-CD4+ T cells in the lamina propria lymphocytes.

The subject may be susceptible to celiac disease, such as a genetic susceptibility. Genetic susceptibility can be determined by identifying the presence of genes, such as HLA-DQ2 and HLA-DQ8, which cause predisposition to celiac disease, having relatives with celiac disease, and/or other autoimmune disease as discussed elsewhere herein.

The treatments described herein can reverse, ameliorate, or reduce the villous atrophy present in a subject having exposure to gluten. The treatments described herein can prevent or reduce villous atrophy recurrence upon exposure to a gluten. The treatments described herein can improve villous height-to-crypt depth ratio (Vh/Cd) and/or restore the villous-to-crypt ratio to a normal range. The treatments described herein can reduce intraepithelial lymphocytosis and/or reduce an elevated level of CD3+ intraepithelial lymphocytes (IELs). The treatments described herein can reducing the amount of one or more of IgA anti-tissue transglutaminase (TGA), IgG anti-deamidated gluten peptide (DGP), and IgG anti-gliadin peptide. The treatments described herein can alleviate symptoms of malabsorption, such as diarrhea, abdominal distension and pain, reduce acid reflux, abdominal bloating and distention, and/or flatulence.

As demonstrated herein, mice with celiac disease induced by intestinal exposure to gluten, treated with LL-[dDQ8]+IL10, and then subjected to a gluten challenge had no villous atrophy. In contrast, mice with celiac disease induced by intestinal exposure to gluten, treated with LL-IL10, and the subjected to a gluten challenge had 20% villous atrophy. Two controls were examined. Mice with celiac disease induced by intestinal exposure to gluten, treated with vehicle or empty LL vector, and then subjected to a gluten challenge had 55% villous atrophy, and 40% villous atrophy, respectively. These data indicate that treatment with LL-IL10 provided 64% reduction in the incidence of villous atrophy, relative to mice treated with vehicle, and 50% reduction in the incidence of villous atrophy, relative to mice treated with empty LL vector. In contrast, these data indicate that treatment with LL-[dDQ8]+IL10 provided 100% reduction in the incidence of villous atrophy, relative to mice treated with vehicle, empty LL vector or LL-IL10. Administration of an L. lactis strain engineered to express IL-10 and a gliadin peptide comprising an HLA-DQ2-specific or HLA-DQ8-specific epitope to a subject with celiac disease can provide a reduction of greater than 50% and up to 100% of villous atrophy, relative to a reference L. lactis strain that does not express IL-10 and a gliadin peptide comprising an HLA-DQ2-specific or HLA-DQ8-specific epitope in a mouse model of celiac disease. As used herein, “mouse model of celiac disease” refers to the mouse model described in Example 1. A reduction of at least about 55% to 100%, at least about 60% to 100%, at least about 65% to 100%, at least about 70% to 100%, at least about 75% to 100%, at least about 80% to 100%, at least about 85% to 100%, at least about 90% to 100%, at least about 91%, 92%, 93%, 94%, 95%, 96%, 97%, 98% or 99% to 100% can be provided by administration of an L. lactis strain engineered to express IL-10 and a gliadin peptide comprising at least one human leukocyte antigen (HLA)-DQ2-specific at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope, relative to a reference L. lactis strain, in a mouse model of celiac disease. A “reference L. lactis strain” refers to an L. lactis strain having identical genetic traits as the administered engineered therapeutic L. lactis strain, except not expressing either of (i) functional IL-10 or (ii) a gliadin peptide comprising the same at least one human leukocyte antigen (HLA)-DQ2-specific at least one deamidated HLA-DQ2 specific epitope, at least one HLA-DQ8 specific epitope, at least one deamidated HLA-DQ8 specific epitope, or the same combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope as the therapeutic L. lactis strain. A suitable reference L. lactis strain may be the parent L. lactis strain of the engineered therapeutic L. lactis strain that does not comprise IL-10 and gliadin peptide expression units. Alternatively, a suitable reference L. lactis strain may comprise IL-10 and gliadin peptide expression units that are not expressed, or express non-functional IL-10 and/or non-antigenic gliadin peptide. Administration of a reference L. lactis strain is an example of a mock treatment.

### Therapeutically Effective Amount

As used herein, the term “therapeutically effective amount” refers to an amount of a non-pathogenic microorganism or a composition of the present disclosure that will elicit a desired therapeutic effect or response when administered according to the desired treatment regimen. In some cases, the compounds or compositions are provided in a unit dosage form, for example a tablet or capsule, which contains an amount of the active component equivalent with the therapeutically effective amount when administered once, or multiple times per day.

A person of ordinary skill in the art will appreciate that a therapeutically effective amount of a recombinant microorganism, which is required to achieve a desired therapeutic effect (e.g., for the effective treatment of CeD), will vary, e.g., depending on the nature of the IL-10 polypeptide expressed by the microorganism, the nature of the CeD-specific antigen polypeptide expressed by the microorganism, the route of administration, and the age, weight, and other characteristics of the recipient.

The amount of secreted polypeptide can be determined based on cfu, determined by state-of-the-art methods such as quantitative polymerase chain reaction (Q-PCR), or by using ELISA. For example, a particular microorganism may secrete at least about 1 ng to about 1 μg of active polypeptide per 10⁹ cfu. Based thereon, the skilled person can calculate the range of antigen polypeptide secreted at other cfu doses.

Therapeutically effective amounts may be administered in connection with any dosing regimen as described herein. The daily dose of active polypeptide may be administered in 1, 2, 3, 4, 5, or 6 portions throughout the day. Further, the daily doses may be administered for any number of days, with any number of rest periods between administration periods. For example, a dose of the active agent (e.g. CeD-specific antigen and/or IL-10) of from about 0.01 to about 3.0 MIU/day/subject may be administered every other day for a total of 6 weeks. In other examples, the CeD-specific antigen and/or IL-10 is administered at doses ranging from 0.1 to 1000 mg per day, such as doses of 1-100 mg at each meal

### Mucosa

The term “mucosa” or “mucous membrane” is used herein in accordance with its art recognized meaning. The “mucosa” can be any mucosa found in the body, such as oral mucosa, rectal mucosa, gastric mucosa, intestinal mucosa, urethral mucosa, vaginal mucosa, ocular mucosa, buccal mucosa, bronchial or pulmonary mucosa, and nasal or olfactory mucosa. Mucosa may also refer to surface mucosa, e.g., those found in fish and amphibians.

The term “mucosal delivery” as used herein is used in accordance with its art recognized meaning, i.e., delivery to the mucosa, e.g., via contacting a composition of the present disclosure with a mucosa. Oral mucosal delivery includes buccal, sublingual and gingival routes of delivery. Accordingly, in some embodiments, “mucosal delivery” includes gastric delivery, intestinal delivery, rectal delivery, buccal delivery, pulmonary delivery, ocular delivery, nasal delivery, vaginal delivery and oral delivery. The person of ordinary skill will understand that oral delivery can affect delivery to distal portions of the gastrointestinal tract.

The term “mucosal tolerance” refers to the inhibition of specific immune responsiveness to an antigen in a mammalian subject (e.g., a human patient), after the subject has been exposed to the antigen via the mucosal route. In some cases, the mucosal tolerance is systemic tolerance. Low dose oral tolerance is oral tolerance induced by low doses of antigens, and is characterized by active immune suppression, mediated by cyclophosphamide sensitive regulatory T-cells that can transfer tolerance to naive hosts. High dose oral tolerance is oral tolerance induced by high doses of antigens, is insensitive to cyclophosphamide treatment, and proceeds to induction of T cell hyporesponsiveness via anergy and/or deletion of antigen specific T-cells. The difference in sensitivity to cyclophosphamide can be used to make a distinction between low dose and high dose tolerance (Strobel et al., 1983). In some cases, the oral tolerance is low dose oral tolerance as described by Mayer and Shao (2004)

### Immuno-Modulating Compound

The terms “immuno-modulating compound” or immuno-modulator” are used herein in accordance with their art-recognized meaning. The immuno-modulating compound can be any immuno-modulating compound known to a person skilled in the art.

In some embodiments, the immuno-modulating compound is a tolerance inducing compound. Tolerance induction can be obtained, e.g., by inducing regulatory T-cells, or in an indirect way, e.g., by activation of immature dendritic cells to tolerizing dendritic cells and/or inhibiting Th2 immune response inducing expression of “co-stimulation” factors on mature dendritic cells Immuno-modulating and immuno-suppressing compounds are known to the person skilled in the art and include, but are not limited to, bacterial metabolites such as spergualin, fungal and streptomycal metabolites such as tacrolimus or cyclosporine, immuno-suppressing cytokines such as IL-4, IFNα, TGFI3 (as selective adjuvant for regulatory T-cells) Flt3L, TSLP and Rank-L (as selective tolerogenic DC inducers), antibodies and/or antagonist such as anti-CD40L, anti-CD25, anti-CD20, anti-IgE, anti-CD3, and proteins, peptides or fusion proteins such as the CTL-41 g or CTLA-4 agonist fusion protein. The immuno-modulating compound can be an immuno-suppressing compound. The immuno-suppressing compound can also an immuno-suppressing cytokine or antibody. In other embodiments, the immuno-suppressing cytokine is a tolerance-enhancing cytokine or antibody. It will be appreciated by the person skilled in the art that the term “immuno-modulating compound” also includes functional homologues thereof. A functional homologue is a molecule having essentially the same or similar function for the intended purposes, but can differ structurally. In some examples, the immuno-modulating compound is anti-CD3, or a functional homologue thereof. In other examples, anti-CD3 antibodies is excluded from the treatment

### Microorganisms

The invention relates to the use of at least one microorganism. In the compositions and methods using the composition, the microorganism is a non-pathogenic and non-invasive bacterium. The microorganism also can be a non-pathogenic and non-invasive yeast.

The microorganism can also be a yeast strain selected from the group consisting of Saccharomyces sp., Hansenula sp., Kluyveromyces sp. Schizzosaccharomyces sp. Zygosaccharomyces sp., Pichia sp., Monascus sp., Geothchum sp and Yarrowia sp. In some embodiments, the yeast is Saccharomyces cerevisiae. In other embodiments, the S. cerevisiae is of the subspecies boulardii. In one embodiment of the present invention, the recombinant yeast host-vector system is a biologically contained system. Biological containment is known to the person skilled in the art and can be realized by the introduction of an auxotrophic mutation, for example a suicidal auxotrophic mutation such as the thyA mutation, or its equivalents.

In other embodiments of the present invention, the microorganism is a bacterium, such as a non-pathogenic bacterium, e.g., a food grade bacterial strain. In some examples, the non-pathogenic bacterium is a Gram-positive bacterium, e.g., a Gram-positive food-grade bacterial strain. Exemplary Gram-positive food grade bacterial strains include a lactic acid fermenting bacterial strain (i.e., a lactic acid bacterium (LAB) or a Bifidobacterium).

In some embodiments, the lactic acid fermenting bacterial strain is a Lactococcus, Lactobacillus or Bifidobacterium species. As used herein, Lactococcus or Lactobacillus is not limited to a particular species or subspecies, but meant to include any of the Lactococcus or Lactobacillus species or subspecies. Exemplary Lactococcus species include Lactococcus garvieae, Lactococcus lactis, Lactococcus piscium, Lactococcus plantarum, and Lactococcus raffinolactis. In some examples, the L. lactis is Lactococcus lactis subsp. cremoris, Lactococcus lactis subsp. hordniae, or Lactococcus lactis subsp. Lactis.

Exemplary Lactobacillus species include Lactobacillus acetotolerans, Lactobacillus acidophilus, Lactobacillus agilis, Lactobacillus algidus, Lactobacillus alimentarius, Lactobacillus amylolyticus, Lactobacillus amylophilus, Lactobacillus amylovorus, Lactobacillus animalis, Lactobacillus aviarius, Lactobacillus aviarius subsp. araffinosus, Lactobacillus aviarius subsp. aviarius, Lactobacillus bavaricus, Lactobacillus bifermentans, Lactobacillus brevis, Lactobacillus buchneri, Lactobacillus bulgaricus, Lactobacillus camis, Lactobacillus casei, Lactobacillus casei subsp. alactosus, Lactobacillus casei subsp. casei, Lactobacillus casei subsp. pseudoplantarum, Lactobacillus casei subsp. rhamnosus, Lactobacillus casei subsp. tolerans, Lactobacillus catenaformis, Lactobacillus cellobiosus, Lactobacillus collinoides, Lactobacillus confiisus, Lactobacillus coryniformis, Lactobacillus coryniformis subsp. coryniformis, Lactobacillus coryniformis subsp. torquens, Lactobacillus crispatus, Lactobacillus curvatus, Lactobacillus curvatus subsp. curvatus, Lactobacillus curvatus subsp. melibiosus, Lactobacillus delbrueckii, Lactobacillus delbrueckii subsp. bulgaricus, Lactobacillus delbrueckii subsp. delbrueckii, Lactobacillus delbrueckii subsp. lactis, Lactobacillus divergens, Lactobacillus farciminis, Lactobacillus fermentum, Lactobacillus fornicalis, Lactobacillus fructivorans, Lactobacillus fructosus, Lactobacillus gallinarum, Lactobacillus gasseri, Lactobacillus graminis, Lactobacillus halotolerans, Lactobacillus hamsteri, Lactobacillus helveticus, Lactobacillus heterohiochii, Lactobacillus hilgardii, Lactobacillus homohiochii, Lactobacillus iners, Lactobacillus intestinalis, Lactobacillus jensenii, Lactobacillus johnsonii, Lactobacillus kandleri, Lactobacillus kefiri, Lactobacillus kefiranofaciens, Lactobacillus kefirgranum, Lactobacillus kunkeei, Lactobacillus lactis, Lactobacillus leichmannii, Lactobacillus lindneri, Lactobacillus malefennentans, Lactobacillus mali, Lactobacillus maltaromicus, Lactobacillus manihotivorans, Lactobacillus minor, Lactobacillus minutus, Lactobacillus mucosae, Lactobacillus murinus, Lactobacillus nagelii, Lactobacillus oris, Lactobacillus panis, Lactobacillus parabuchneri, Lactobacillus paracasei, Lactobacillus paracasei subsp. paracasei, Lactobacillus paracasei subsp. tolerans, Lactobacillus parakefiri, Lactobacillus paralimentarius, Lactobacillus paraplantarum, Lactobacillus pentosus, Lactobacillus perolens, Lactobacillus piscicola, Lactobacillus plantarum, Lactobacillus pontis, Lactobacillus reuteri, Lactobacillus rhamnosus, Lactobacillus rimae, Lactobacillus rogosae, Lactobacillus ruminis, Lactobacillus sakei, Lactobacillus sakei subsp. camosus, Lactobacillus sakei subsp. sakei, Lactobacillus salivarius, Lactobacillus salivarius subsp. salicinius, Lactobacillus salivarius subsp. salivarius, Lactobacillus sanfranciscensis, Lactobacillus sharpeae, Lactobacillus suebicus, Lactobacillus trichodes, Lactobacillus uli, Lactobacillus vaccinostercus, Lactobacillus vaginalis, Lactobacillus viridescens, Lactobacillus vitulinus, Lactobacillus xylosus, Lactobacillus yamanashiensis, Lactobacillus yamanashiensis subsp. mali, Lactobacillus yamanashiensis subsp. Yamanashiensis, Lactobacillus zeae, Bifidobacterium adolescentis, Bifidobacterium angulatum, Bifidobacterium bifidum, Bifidobacterium breve, Bifidobacterium catenulatum, Bifidobacterium longum, and Bifidobacterium infantis. In some examples, the LAB is Lactococcus lactis (LL).

In further examples, the bacterium can be selected from the group consisting of Enterococcus alcedinis, Enterococcus aquimarinus, Enterococcus asini, Enterococcus avium, Enterococcus caccae, Enterococcus camelliae, Enterococcus canintestini, Enterococcus canis, Enterococcus casseliflavus, Enterococcus cecorum, Enterococcus columbae, Enterococcus devriesei, Enterococcus diestrammenae, Enterococcus dispar, Enterococcus durans, Enterococcus eurekensis, Enterococcus faecalis, Enterococcus faecium, Enterococcus gallinarum, Enterococcus gilvus, Enterococcus haemoperoxidus, Enterococcus hermanniensis, Enterococcus hirae, Enterococcus italicus, Enterococcus lactis, Enterococcus lemanii, Enterococcus malodoratus, Enterococcus moraviensis, Enterococcus mundtii, Enterococcus olivae, Enterococcus pallens, Enterococcus phoeniculicola, Enterococcus plantarum, Enterococcus pseudoavium, Enterococcus quebecensis, Enterococcus raffinosus, Enterococcus ratti, Enterococcus rivorum, Enterococcus rotai, Enterococcus saccharolyticus, Enterococcus silesiacus, Enterococcus solitarius, Enterococcus sulfureus, Enterococcus termitis, Enterococcus thailandicus, Enterococcus ureasiticus, Enterococcus ureilyticus, Enterococcus viikkiensis, Enterococcus villorum, and Enterococcus xiangfangensis,

In further examples, the bacterium can be selected from the group consisting of Streptococcus agalactiae, Streptococcus anginosus, Streptococcus bovis, Streptococcus canis, Streptococcus constellatus, Streptococcus dysgalactiae, Streptococcus equinus, Streptococcus iniae, Streptococcus intermedius, Streptococcus milleri, Streptococcus mitis, Streptococcus mutans, Streptococcus oralis, Streptococcus parasanguinis, Streptococcus peroris, Streptococcus pneumoniae, Streptococcus pseudopneumoniae, Streptococcus pyogenes, Streptococcus ratti, Streptococcus salivarius, Streptococcus tigurinus, Streptococcus thennophilus, Streptococcus sanguinis, Streptococcus sobrinus, Streptococcus suis, Streptococcus uberis, Streptococcus vestibularis, Streptococcus viridans, and Streptococcus zooepidemicus.

In a particular aspect of the present invention, the Gram-positive food grade bacterial strain is Lactococcus lactis or any of its subspecies, including Lactococcus lactis subsp. Cremoris, Lactococcus lactis subsp. Hordniae, and Lactococcus lactis subsp. Lucas. Exemplary recombinant Gram-positive bacterial strains can be a biologically contained system, such as the plasmid free Lactococcus lactis strain MG1363, that lost the ability of normal growth and acid production in milk (Gasson, M. J. (1983) J. Bacteriol. 154: 1-9); or the threonine- and pyrimidine-auxotroph derivative L. lactis strains (Sorensen et al. (2000) Appl. Environ. Microbiol. 66: 1253-1258; Glenting et al. (2002) 68: 5051-5056).

In one embodiment of the present invention, the recombinant bacterial host-vector system is a biologically contained system. Biological containment is known to the person skilled in the art and can be realized by the introduction of an auxotrophic mutation, for example a suicidal auxotrophic mutation such as the ThyA mutation, or its equivalents, debilitating DNA synthesis. Other examples of auxotrophic mutations can debilitate RNA, cell wall or protein synthesis. Alternatively, wherein one or both of the IL-10 polypeptide and CeD-specific antigen are expressed from a plasmid, the biological containment can be realized at the level of the plasmid carrying the gene encoding the IL-10 polypeptide or CeD-specific antigen, such as, for example, by using an unstable episomal construct, which is lost after a few generations. Several levels of containment, such as plasmid instability and auxotrophy, can be combined to ensure a high level of containment, if desired.

### Constructs

In the present invention, the microorganism (e.g., the non-pathogenic gram-positive bacterium) can deliver the IL-10 polypeptide and the CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) at the intended site, i.e., the mucosa. For example, the microorganism (e.g., LAB) expresses the IL-10 polypeptide, after which the IL-10 polypeptide is secreted (if a secreted form of IL-10 is used). Hence, the microorganism (e.g., LAB), such as L. lactis, expresses IL-10 and expresses at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope at the site of an intended mucosa, e.g., in the gastrointestinal tract. In embodiments, the microorganism delivers only two therapeutic proteins, e.g., IL-10 polypeptide and the CeD-specific antigen, to the intended site. In other embodiments, the microorganism delivers at least three therapeutic proteins, including IL-10 polypeptide and the CeD-specific antigen, to the intended site.

Alternatively, two separate microorganisms, each expressing a therapeutic protein, can deliver the therapeutic proteins at the intended site. For instance, a first microorganism (e.g., the non-pathogenic gram-positive bacterium) can deliver the IL-10 polypeptide and a second microorganism (e.g., the non-pathogenic gram-positive bacterium) can deliver the CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) at the intended site, i.e., the mucosa. One or both of the first and second microorganisms can deliver one or more further therapeutic proteins to the intended site.

Use of an operon enables expression of the IL-10 polypeptide and CeD-specific antigen polypeptide (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) to be coordinated. Polycistronic expression systems in bacterial host cells are described, e.g., in U.S. Pat. No. 9,920,324 and WO 2012/164083, each of which is incorporated herein by reference in its entirety.

Stably transfected microorganisms are also disclosed, i.e., microorganisms in which the gene coding for the IL-10 polypeptide and the CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) gene has been integrated into the host cell's genome. Techniques for establishing stably transfected microorganisms are known in the art. For instance, the IL-10 polypeptide and the CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) gene may be cloned into the host's genome, e.g. in the chromosome, via homologous recombination. In some microorganisms, an essential gene in the microorganism is disrupted by the homologous recombination event, such as deletion of the gene, one or more amino acid substitutions leading to an inactive form of the protein encoded by the essential gene, or to a frameshift mutation resulting in a truncated form of the protein encoded by the essential gene. The essential gene can be a thyA gene. A preferred technique is described, e.g., in WO 02/090551, which is incorporated herein by reference in its entirety. The plasmid may be a self-replicating, for example carrying one or more genes of interest and one or more resistance markers. Then, the transforming plasmid can be any plasmid, as long as it cannot complement the disrupted essential gene, e.g., thyA gene. Alternatively, the plasmid is an integrative plasmid. In the latter case, the integrative plasmid itself may be used to disrupt the essential gene, by causing integration at the locus of the essential gene, e.g., thyA site, because of which the function of the essential gene, e.g., the thyA gene, is disrupted. In some cases, the essential gene, such as the thyA gene, is replaced by double homologous recombination by a cassette comprising the gene or genes of interest, flanked by targeting sequences that target the insertion to the essential gene, such as the thyA target site. It will be appreciated that that these targeting sequences are sufficiently long and sufficiently homologous to enable integration of the gene of interest into the target site. In some examples, an IL-10 expression cassette of the present disclosure is integrated at the thyA locus.

The genetic construct encoding the IL-10 polypeptide and the CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) may be integrated into the microbial genomic DNA, e.g., bacterial or yeast chromosome, e.g., Lactococcus chromosome. In the latter case, a single or multiple copies of the nucleic acid may be integrated; the integration may occur at a random site of the chromosome or, as described above, at a predetermined site thereof, for example at a predetermined site, such as, in a non-limiting example, in the eno locus or the thyA locus of Lactococcus, e.g., Lactococcus lactis.

Hence, the genetic construct encoding the IL-10 polypeptide and the CD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) may further comprise sequences configured to effect insertion of the genetic construct into the genome, e.g., a chromosome, of a host cell.

In some examples, insertion of the genetic construct into particular sites within a genome, e.g., chromosome, of a host cell may be facilitated by homologous recombination. For instance, the genetic construct the invention may comprise one or more regions of homology to the said site of integration within the genome e.g., a chromosome, of the host cell. The sequence at the said genome, e.g., chromosome, site may be natural, i.e., as occurring in nature, or may be an exogenous sequence introduced by previous genetic engineering. For instance, the region(s) of homology may be at least 50 bp, 100 bp, 200 bp, 300 bp, 400 bp, 500 bp, 600 bp 700 bp, 800 bp, 900 bp, 1000 bp, or more.

In one example, two regions of homology may be included, one flanking each side of the relevant expression units present in the genetic construct of the invention. Such configuration may advantageously insert the relevant sequences, i.e., at least the ones encoding and effecting the expression of the antigen of interest, in host cells. Ways of performing homologous recombination, especially in bacterial hosts, and selecting for recombinants, are generally known in the art.

Transformation methods of microorganisms are known to the person skilled in the art, such as for instance protoplast transformation and electroporation.

A high degree of expression can be achieved by using homologous expression and/or secretion signals on the expression vectors present in the microorganism, e.g., L. lactis. Expression signals will be apparent to the person skilled in the art. The expression vector can be optimized for expression depending on the microorganism, e.g., L. lactis, it is incorporated in. For instance, specific expression vectors that give sufficient levels of expression in Lactococcus, Lactobacillus lactis, L. casei and L. plantarum are known. Moreover, systems are known which have been developed for the expression of heterologous antigens in the non-pathogenic, non-colonizing, non-invasive food-grade bacterium Lactococcus lactis (see U.S. Pat. No. 6,221,648, which is incorporated herein by reference). An exemplary construct comprising a multi-copy expression vector is described in PCT/NL95/00135 (WO-A-96/32487). Such a construct is particularly suitable for expression of a desired antigen in a lactic acid bacterium, in particular in a Lactobacillus, at a high level of expression, and also can be used advantageously to direct the expressed product to the surface of the bacterial cell. Such constructs (e.g., as described in Application No. PCT/NL95/00135) comprising sequences encoding the IL-10 polypeptide and/or CeD-specific antigen may be characterized in that the nucleic acid sequence encoding the IL-10 polypeptide and/or CeD-specific antigen (e.g., an HLA-DQ2 -specific epitope and/or an HLA-DQ8-specific epitope) is preceded by a 5′ non-translated nucleic acid sequence comprising at least the minimal sequence required for ribosome recognition and RNA stabilization. This can be followed by a translation initiation codon which may be (immediately) followed by a fragment of at least 5 codons of the 5′ terminal part of the translated nucleic acid sequence of a gene of a lactic acid bacterium or a structural or functional equivalent of the fragment. The fragment may also be controlled by the promoter. The contents of PCT/NL95/00135, including the differing embodiments disclosed therein, and all other documents mentioned in this specification, are incorporated herein by reference. A method is also provided which permits the high level regulated expression of heterologous genes in the host and the coupling of expression to secretion. In another embodiment, the T7 bacteriophage RNA polymerase and its cognate promoter are used to develop a powerful expression system according to WO 93/17117, which is incorporated herein by reference. In one embodiment, the expression plasmid is derived from pT1NX (GenBank: HM585371.1).

A promoter employed in accordance with the present invention is in some cases expressed constitutively in the bacterium. The use of a constitutive promoter avoids the need to supply an inducer or other regulatory signal for expression to take place. In some cases, the promoter directs expression at a level at which the bacterial host cell remains viable, i.e., retains some metabolic activity, even if growth is not maintained. Advantageously then, such expression may be at a low level. For example, where the expression product accumulates intracellularly, the level of expression may lead to accumulation of the expression product at less than about 10% of cellular protein, about or less than about 5%, for example about 1-3%. The promoter may be homologous to the bacterium employed, i.e., one found in that bacterium in nature. For example, a Lactococcal promoter may be used in a Lactococcus. A preferred promoter for use in Lactococcus lactis (or other Lactococci) is “Pl” (SEQ ID NO:--) derived from the chromosome of Lactococcus lactis (Waterfield, N R, Lepage, R W F, Wilson, P W, et al. (1995). “The isolation of lactococcal promoters and their use in investigating bacterial luciferase synthesis in Lactococcus lactis,” Gene 165(1): 9-15). Another promoter is the thyA promoter (Steidler, et al (2003). “Biological containment of genetically modified Lactococcus lactis for intestinal delivery of human interleukin 10,” Nature Biotechnology 21:785-789). Other examples of promoters include, the usp45 promoter, the gapB promoter, the hllA promoter, and the eno promoter. Additional exemplary promoters are described in U.S. Pat. No. 8,759,088 and in U.S. Pat. No. 9,920,324, the disclosures of each which are incorporated herein by reference in their entirety. Further exemplary promoter disclosure is found in WO 2008/084115, WO 2001/039137, U.S. Pat. No. 8, 769,088, and U.S. Publication No. 2012/0183503, each of which is incorporated herein by reference in its entirety.

A promoter employed in accordance with the present invention is in some cases inducibly expressed in the bacterium. Inducible expression can be directly inducible or can be indirectly inducible. A “directly inducible promoter” refers to a regulatory region, wherein the regulatory region is operably linked to a gene encoding IL-10 polypeptide and/or the CeD-specific antigen (e.g., an HLA-DQ2-specific epitope and/or an HLA-DQ8-specific epitope); in the presence of an inducer of said regulatory region, the phenylalanine-metabolizing enzyme is expressed. An “indirectly inducible promoter” refers to a regulatory system comprising two or more regulatory regions, for example, a first regulatory region that is operably linked to a gene encoding a first molecule, e.g., a transcriptional regulator, which is capable of regulating a second regulatory region that is operably linked to a gene encoding IL-10 polypeptide and/or the CeD-specific antigen (e.g., an HLA-DQ2-specific epitope and/or an HLA-DQ8-specific epitope). In the presence of an inducer of the first regulatory region, the second regulatory region may be activated or repressed, thereby activating or repressing expression of the IL-10 polypeptide and/or the CeD-specific antigen (e.g., an HLA-DQ2-specific epitope and/or an HLA-DQ8-specific epitope). Both a directly inducible promoter and an indirectly inducible promoter are encompassed by “inducible promoter.”

“Exogenous environmental conditions” refer to settings or circumstances under which the promoter described above is directly or indirectly induced. In some embodiments, the exogenous environmental conditions are specific to the gut of a mammal. In some embodiments, the exogenous environmental conditions are specific to the upper gastrointestinal tract of a mammal In some embodiments, the exogenous environmental conditions are specific to the lower gastrointestinal tract of a mammal In some embodiments, the exogenous environmental conditions are specific to the small intestine of a mammal In some embodiments, exogenous environmental conditions refer to the presence of molecules or metabolites that are specific to the mammalian gut in a healthy or disease state, e.g., propionate. In some embodiments, the exogenous environmental conditions are low-oxygen, microaerobic, or anaerobic conditions, such as the environment of the mammalian gut.

“Exogenous environmental condition(s)” refer to setting(s) or circumstance(s) under which the promoter described herein is induced. The phrase “exogenous environmental conditions” is meant to refer to the environmental conditions external to the engineered microorganism, but endogenous or native to the host subject environment. Thus, in this context, “exogenous” and “endogenous” may be used interchangeably to refer to environmental conditions in which the environmental conditions are endogenous to a mammalian body, but external or exogenous to an intact microorganism cell. In some embodiments, the exogenous environmental conditions are specific to the gut of a mammal In some embodiments, the exogenous environmental conditions are specific to the upper gastrointestinal tract of a mammal. In some embodiments, the exogenous environmental conditions are specific to the lower gastrointestinal tract of a mammal In some embodiments, the exogenous environmental conditions are specific to the small intestine of a mammal. In some embodiments, the exogenous environmental conditions are low-oxygen, microaerobic, or anaerobic conditions, such as the environment of the mammalian gut. In some embodiments, exogenous environmental conditions are molecules or metabolites that are specific to the mammalian gut, e.g., propionate. In some embodiments, the exogenous environmental condition is a tissue-specific or disease-specific metabolite or molecule(s). Alternatively, the exogenous environmental condition is a low-pH environment. The genetically engineered microorganism may comprise a pH-dependent promoter. In some embodiments, the genetically engineered microorganism of the disclosure comprises an oxygen level-dependent promoter. In some aspects, bacteria have evolved transcription factors that are capable of sensing oxygen levels. Different signaling pathways may be triggered by different oxygen levels and occur with different kinetics.

An “oxygen level-dependent promoter” or “oxygen level-dependent regulatory region” refers to a nucleic acid sequence to which one or more oxygen level-sensing transcription factors is capable of binding, wherein the binding and/or activation of the corresponding transcription factor activates downstream gene expression.

Examples of oxygen level-dependent transcription factors include, but are not limited to, FNR, ANR, and DNR. Corresponding FNR-responsive promoters, ANR-responsive promoters, and DNR-responsive promoters are known in the art (see, e.g., Castiglione et al., 2009, “The transcription factor DNR from Pseudomonas aeruginosa specifically requires nitric oxide and haem for the activation of a target promoter in Escherichia coli,” Microbiology, 155 (Pt 9): 2838-2844; Eiglmeier et al., 1989, “Molecular genetic analysis of FNR-dependent promoters,” Mol. Microbiol., 3(7):869-878; Galimand et al., 1991, (Mar. 1991) “Positive FNR-like control of anaerobic arginine degradation and nitrate respiration in Pseudomonas aeruginosa,” J. Bacteriol., 173(5): 1598-1606; Hasegawa et al., 1998, “Activation of a consensus FNR-dependent promoter by DNR of Pseudomonas aeruginosa in response to nitrite,” FEMS Microbiol. Lett., 166(2): 213-217; Hoeren et al., 1993, “Sequence and expression of the gene encoding the respiratory nitrous-oxide reductase from Paracocous denitrificans,” Eur. J. Biochem., 218(1): 49-57; Salmon et al., 2003, “Global gene expression profiling in Escherichia coli K12—The effects of oxygen availability and FNR,” J. Biol. Chem. 278(32): 29837-29855). Exemplary transcription factors and responsive genes and regulatory regions are disclosed for instance in U.S. Pat. No. 10,195,234 B2.

The nucleic acid construct or constructs may comprise a nucleic acid encoding a secretory signal sequence. Thus, in some embodiments the nucleic acid encoding IL-10 and/or the CeD-specific antigen (e.g., an HLA-DQ2-specific epitope and/or an HLA-DQ8-specific epitope) may provide for secretion of the polypeptides, e.g., by appropriately coupling a nucleic acid sequence encoding a signal sequence to the nucleic acid sequence encoding the polypeptide). Ability of a bacterium harboring the nucleic acid to secrete the antigen may be tested in vitro in culture conditions that maintain viability of the organism. Preferred secretory signal sequences include any of those with activity in Gram positive organisms, such as Bacillus, Clostridium, and Lactobacillus. Such sequences may include the α-amylase secretion leader of Bacillus amyloliquetaciens or the secretion leader of the Staphylokinase enzyme secreted by some strains of Staphylococcus, which is known to function in both Gram-positive and Gram-negative hosts (see “Gene Expression Using Bacillus,” Rapoport (1990) Curr. Opin. Biotechnology 1: 21-27), or leader sequences from numerous other Bacillus enzymes or S-layer proteins (see pp. 341-344 of Harwood and Cutting, MOLECULAR BIOLOGICAL METHODS FOR BACILLUS, John Wiley & Co. 1990). In one embodiment, the secretion signal can be derived from usp45 (Van Asseldonk et al. (1993) Mol. Gen. Genet. 240: 428-434). Such secretion leader is referred to herein, e.g., as SSusp45. In some embodiments, the IL-10 polypeptide is constitutively secreted using SSusp45 (SEQ ID NO: for SL #34). In other examples, the HLA-DQ2-specific epitope and/or HLA-DQ8-specific epitope polypeptide is constitutively secreted using SSusp45 (SEQ ID NO: for SL #34). In yet other examples, both the IL-10 polypeptide and an HLA-DQ2-specific epitope and/or an HLA-DQ8-specific epitope polypeptide are secreted constitutively using SSusp45 (SEQ ID NO: for SL #34). Each and all embodiments are operable without SL #34 as the secretion sequence.

In other examples, the HLA-DQ2-specific epitope and/or HLA-DQ8-specific epitope polypeptide is constitutively secreted using a secretion leader having adequate or improved secretion. “Improved secretion” can encompass one or both quantity and quality of secretion. A non-limiting example of improved secretion quality is a reduction of incomplete protein banding, also referred to as “laddering,” relative to the laddering for a reference secretion leader, such as SSusp45. A secretion leader with adequate or improved secretion can be selected from the group consisting of: SL #1, SL #6, SL #8, SL #9, SL #13, SL #15, SL #17, SL #20, SL #21, SL #22, SL #23, SL #24, SL #25, SL #32, SL #34, SL #35, and SL #36.

TABLE 1

| | | | SEQ | SEQ |
|---|---|---|---|---|
| | | | ID | ID |
| | | Predicted Secretion Leader | NO: | NO: |
| SL# | UniProt | Amino Acid Sequence | (PRT) | (DNA) |
| 1 | A2RHI3 | MKKRVQRNKKRIRWASVLTVFVLLIGII | 34 | 86 |
| | | AIAFA | | |
| 6 | A2RIL8 | MKQKHKLALGASIVALASLGGIKAQA | 35 | 91 |
| 8 | A2RKE6 | MNLAKNWKSFALVAAGAIAVVSLAAC | 36 | 93 |
| | | GKSA | | |
| 9 | A2RLK0 | MLKKIIISAALMASLSAAMIANPAKA | 37 | 94 |
| 13 | P22865 | MKKKIISAILMSTVILSAAAPLSGVYA | 38 | 98 |
| 15 | A2RIG7 | MKKIIYGVGLISLLNVGTIAYG | 39 | 100 |
| 17 | A2RI74 | MKQAKIIGLSTVIALSGIILVACGSKT | 40 | 102 |
| 20 | A2RIV4 | MKKFLLLGATALSLFSLAACSSSN | 41 | 104 |
| 21 | A2RJJ4 | MKKVIKKAAIGMVAFFVVAASGPVFA | 42 | 105 |
| 22 | A2RJL9 | MSKKSIKKITMTVGVGLLTAIMSPSVIN | 43 | 106 |
| | | Q | | |
| 23 | A2RJP5 | MRHKKIYLLLAMIGATSAWTVANENQ | 44 | 107 |
| | | VKA | | |
| 24 | A2RJQ9 | MKKFVLIILLLFSSSILLADKSSA | 45 | 108 |
| 25 | A2RK78 | MKIKYILWVICALLLLNTGPSFA | 46 | 109 |
| 32 | G0WJN9 | MNKLKVTLLASSVVLAATLLSACGSNQ | 47 | 116 |
| | | SSS | | |
| 34 | P22865 | MKKKIISAILMSTVILSAAAPLSGVYA | 38 | 98 |
| | (SSusp45) | | | |
| 35 | P22865* | MKKKIISAILMSTVILSAAAPLSGVYAG | 48 | 119 |
| 36 | P22865** | MKKNIISAILMSTVILSAAAPLSGVYA | 49 | 120 |

In some embodiments, the HLA-DQ2-specific epitope polypeptide is constitutively secreted using a secretion leader selected from the leaders shown in Table 2.

TABLE 2

| | | | SEQ | SEQ |
|---|---|---|---|---|
| | | | ID | ID |
| | | Predicted Secretion Leader | NO: | NO: |
| SL# | UniProt | Amino Acid Sequence | (PRT) | (DNA) |
| 1 | A2RHI3 | MKKRVQRNKKRIRWASVLTVFVLLIGI | 34 | 86 |
| | | IAIAFA | | |
| 6 | A2RIL8 | MKQKHKLALGASIVALASLGGIKAQA | 35 | 91 |
| 8 | A2RKE6 | MNLAKNWKSFALVAAGAIAVVSLAACG | 36 | 93 |
| | | KSA | | |
| 9 | A2RLK0 | MLKKIIISAALMASLSAAMIANPAKA | 37 | 94 |
| 13 | P22865 | MKKKIISAILMSTVILSAAAPLSGVYA | 38 | 98 |
| 15 | A2RIG7 | MKKIIYGVGLISLLNVGTIAYG | 39 | 100 |
| 17 | A2RI74 | MKQAKIIGLSTVIALSGIILVACGSKT | 40 | 102 |
| 20 | A2RIV4 | MKKFLLLGATALSLFSLAACSSSN | 41 | 104 |
| 21 | A2RJJ4 | MKKVIKKAAIGMVAFFVVAASGPVFA | 42 | 105 |
| 22 | A2RJL9 | MSKKSIKKITMTVGVGLLTAIMSPSVI | 43 | 106 |
| | | NQ | | |
| 23 | A2RJP5 | MRHKKIYLLLAMIGATSAWTVANENQV | 44 | 107 |
| | | KA | | |
| 24 | A2RJQ9 | MKKFVLIILLLFSSSILLADKSSA | 45 | 108 |
| 25 | A2RK78 | MKIKYILWVICALLLLNTGPSFA | 46 | 109 |
| 34 | P22865 | MKKKIISAILMSTVILSAAAPLSGVYA | 38 | 98 |
| | (SSusp45) | | | |
| 36 | P22865** | MKKNIISAILMSTVILSAAAPLSGVYA | 49 | 120 |

In some embodiments, the HLA-DQ2-specific epitope polypeptide is constitutively secreted using a secretion leader selected from the leaders shown in Table 3.

TABLE 3

| | | Predicted Secretion Leader | SEQ |
|---|---|---|---|
| SL# | UniProt | Amino Acid Sequence | ID NO: |
| 8 | A2RKE6 | MNLAKNWKSFALVAAGAIAVVSLAACGKSA | 36 |
| 17 | A2RI74 | MKQAKIIGLSTVIALSGIILVACGSKT | 40 |
| 20 | A2RIV4 | MKKFLLLGATALSLFSLAACSSSN | 41 |
| 21 | A2RJJ4 | MKKVIKKAAIGMVAFFVVAASGPVFA | 42 |
| 22 | A2RJL9 | MSKKSIKKITMTVGVGLLTAIMSPSVINQ | 43 |
| 23 | A2RJP5 | MRHKKIYLLLAMIGATSAWTVANENQVKA | 44 |
| 34 | P22865 | MKKKIISAILMSTVILSAAAPLSGVYA | 38 |
| | (SSusp45) | | |

In some embodiments, the HLA-DQ2-specific epitope polypeptide is constitutively secreted using secretion leader #21 (A2RJJ4):

(SEQ ID NO: 42)
MKKVIKKAAIGMVAFFVVAASGPVFA.

In some embodiments, the HLA-DQ2-specific epitope polypeptide is a deamidated HLA-DQ2-specific epitope (dDQ2) and is constitutively secreted using a secretion leader selected from the leaders shown in Table 4.

TABLE 4

| | | Predicted Secretion | SEQ |
|---|---|---|---|
| SL# | UniProt | Leader Sequences | ID NO: |
| 15 | A2RIG7 | MKKIIYGVGLISLLNVGTIAYG | 39 |
| 17 | A2RI74 | MKQAKIIGLSTVIALSGIILVACGSKT | 40 |
| 21 | A2RJJ4 | MKKVIKKAAIGMVAFFVVAASGPVFA | 42 |
| 22 | A2RJL9 | MSKKSIKKITMTVGVGLLTAIMSPSVINQ | 43 |
| 23 | A2RJP5 | MRHKKIYLLLAMIGATSAWTVANENQVKA | 44 |
| 32 | G0WJN9 | MNKLKVTLLASSVVLAATLLSACGSNQSSS | 47 |
| 34 | P22865 | MKKKIISAILMSTVILSAAAPLSGVYA | 38 |
| | (SSusp45) | | |
| 35 | P22865* | MKKKIISAILMSTVILSAAAPLSGVYAG | 48 |
| 36 | P22865** | MKKNIISAILMSTVILSAAAPLSGVYA | 49 |

In some embodiments, the deamidated HLA-DQ2-specific epitope polypeptide is constitutively secreted using a secretion leader selected from the leaders shown in Table 5.

TABLE 5

| | | Predicted Secretion | SEQ |
|---|---|---|---|
| SL# | UniProt | Leader Sequences | ID NO: |
| 17 | A2RI74 | MKQAKIIGLSTVIALSGIILVACGSKT | 40 |
| 21 | A2RJJ4 | MKKVIKKAAIGMVAFFVVAASGPVFA | 42 |
| 22 | A2RJL9 | MSKKSIKKITMTVGVGLLTAIMSPSVINQ | 43 |
| 23 | A2RJP5 | MRHKKIYLLLAMIGATSAWTVANENQVKA | 44 |
| 34 | P22865 | MKKKIISAILMSTVILSAAAPLSGVYA | 38 |
| | (SSusp45) | | |

In some embodiments, the HLA-DQ2-specific epitope polypeptide is constitutively secreted using secretion leader #21 (A2RJJ4; ps356 endolysin):

(SEQ ID NO: 42)
MKKVIKKAAIGMVAFFVVAASGPVFA.

In the alternative, for any of the above described secretion leader embodiments, the epitope polypeptide is inducibly expressed and secreted.

In some embodiments, the secretion leader is a variant having 1, 2, or 3 variant amino acids positions of any of the above-disclosed secretion leaders, having 1, 2, or 3 variant amino acids positions. Starting with any of the disclosed secretion leader sequences, the person of skill in the art can generate mutations in the secretion leader sequence and screen each variant for secretion potency, relative to the original unmutated secretion leader. For instance, the coding sequences of any secretion leader can be mutagenized by any known synthetic biology approach: random point mutation, error prone PCR, site saturation mutagenesis, computer aided design or other. A DQ2 or dDQ2 coding sequence can be linked in-frame (i.e., operably linked) to the 3′ end of the pool of mutagenized secretion leader sequences. Fusion of a secretion leader sequence and a DQ2 epitope or deamidated DQ2 epitope forms the configurations SL::DQ2 or SL::dDQ2. The SL::DQ2 and SL::dDQ2 coding sequences are positioned at an appropriate distance downstream of an L. lactis promoter (P) to obtain P>>SL::DQ2 and P>>SL::dDQ2, thus creating modules for the expression and secretion of DQ2 and dDQ2. L. lactis promoters useful in screening include L. lactis hllA gene promoter (PhllA) and P1. These modules can be cloned into erythromycin selectable L. lactis plasmids and transformed to L. lactis to obtain LL[P>>SL::DQ2] and LL[P>>SL::dDQ2] (d)DQ2 expressing strains. Clones with appropriate secretion levels, e.g., at least about the same as the corresponding non-mutagenized version and/or at least about 3×, about 5×, or about 10× greater than background in the secretion screening method are identified. Selected clones with appropriate secretion levels can then be sequenced, as well, as further characterized by regarding protein expression and secretion analysis by conventional methods, such as filter blotting and quantification, mass spectrometry, and the like.

Specified amino acid changes can also be made to a secretion leader sequence. For example, conservative amino acid changes may be made, which, although they alter the primary sequence of the protein or peptide, do not normally alter its function. Conservative amino acid substitutions typically include substitutions within the following groups:

glycine, alanine
valine, isoleucine, leucine
aspartic acid, glutamic acid
asparagine, glutamine
serine, threonine
lysine, arginine
phenylalanine, tyrosine

Accordingly, variants having 1, 2, or 3 variant amino acids positions of the amino acid sequences of any of the above-disclosed secretion leaders, and having at least about the same secretion potency, are encompassed by this disclosure.

A person of ordinary skill in the art will appreciate that the optimal amount of IL-10 and a gliadin peptide comprising at least one human leukocyte antigen (HLA)-DQ2-specific at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope to be delivered to the subject using the methods of the present disclosure varies, e.g., with the microorganism expressing the IL-10 polypeptide and the a gliadin peptide comprising at least one human leukocyte antigen (HLA)-DQ2-specific at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope polypeptide, and the genetic constructs, e.g., the strength of the promoter used in the genetic constructs. Typically, the microorganism will be administered in an amount equivalent to a particular amount of expressed IL-10 polypeptide and a gliadin peptide comprising at least one human leukocyte antigen (HLA)-DQ2-specific at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope, or in an amount which generates a desired PK profile for the respective IL-10 polypeptide or an a gliadin peptide comprising at least one human leukocyte antigen (HLA)-DQ2-specific at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope in the respective subject. Exemplary daily IL-10 polypeptide or an a gliadin peptide comprising at least one human leukocyte antigen (HLA)-DQ2-specific at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope doses are from about 10 fg to about 100 μg of active polypeptide per day. Other exemplary dose ranges are from about 1 pg to about 100 μg per day; or from about 1 ng to about 100 μg per day.

The above doses may be realized by administering to the subject effective amounts of the microorganism per day, wherein the microorganism is adapted to express a sufficient amount of IL-10 and a CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope polypeptide) to realize the desired dose, such as those above. The microorganism secreting the IL-10 polypeptide and the CeD-specific antigen (e.g., an HLA-DQ2-specific epitope and/or an HLA-DQ8-specific epitope polypeptide) may be delivered in a dose of from about 10⁴ colony forming units (cfu) to about 10¹² cfu per day, in particular from about 10⁶ cfu to about 10¹² cfu per day, more in particular from about 10⁹ cfu to about 10¹² cfu per day. The amount of secreted IL-10 and CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope polypeptide) can be determined based on cfu, for example in accordance with the methods described in Steidler et al., Science 2000; 289(5483): 1352-1355, or by using ELISA. For example, a particular microorganism may secrete at least about 1 ng to about 1μg IL-10 per 10⁹ cfu. Based thereon, the skilled person can calculate the range of IL-10 polypeptide secreted at other cfu doses.

Each of the above doses/dose ranges may be administered in connection with any dosing regimen as described herein. The daily dose may be administered in 1, 2, 3, 4, 5, or 6 portions throughout the day. Further, the daily doses may be administered for any number of days, with any number of rest periods between administration periods. For example, the subject may be administered the microorganism at a dose equivalent to about 0.01 to about 3 M IU of IL-10/day or every other day, for a period of at least about 1 week, at least about 2 weeks, at least about 3 weeks, at least about 4 weeks, at least about 5 weeks, or at least about 6 weeks. In some examples, the subject is administered the microorganism at a dose equivalent to about 0.1 to about 5 MIU/day, or about 0.3 to about 3 MIU, e.g., for about 5 days, about 7 days, or about 14 days. Exemplary doses are described, e.g., in Hartemann et al., Lancet Diabetes Endocrinol. 2013, 1(4): 295-305, the disclosure of which is incorporated herein by reference in its entirety

### Formulations and Regimens

In some methods of the present disclosure, the IL-10 polypeptide and the CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope polypeptide) are administered (delivered) to a subject (e.g., a human CeD-patient) using a microorganism (e.g., LAB) producing both the IL-10 polypeptide and the CeD-specific antigen (e.g., at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope) polypeptide.

In some embodiments, the microorganism (e.g., LAB such as sAGX0868), optionally contained in a composition (e.g., a pharmaceutical composition) of the present disclosure or a unit dosage form of the present disclosure, will be administered, once, twice, three, four, five, or six times daily, e.g., using an oral formulation. In some embodiments, the microorganism is administered every day, every other day, once per week, twice per week, three times per week, or four times per week. In other embodiments, treatment occurs once every two weeks. In other embodiments, treatment occurs once every three weeks. In other embodiments, treatment occurs once per month.

The duration of a treatment cycle is, for example, 7 days to the subject's lifetime, as needed to treat or reverse CeD, or prevent relapse. In some embodiments, a treatment cycle lasts for 21 days to about 2 years. In some embodiments, a treatment cycle lasts for 21 days, 30 days or 42 days to 1.5 years. In other embodiments, the subject will have a treatment cycle that lasts from 21 days, 30 days or 42 days to 1 year. In other embodiments, the subject will have a treatment cycle that lasts from 21 days, 30 days or 42 days to 11 months. In other embodiments, the subject will have a treatment cycle that lasts from 21 days, 30 days or 42 days to 10 months. In other embodiments, the subject will have a treatment cycle that lasts from 21 days, 30 days or 42 days to 9 months. In other embodiments, the subject will have a treatment cycle that lasts from 21 days, 30 days or 42 days to 8 months. In other embodiments, the subject will have a treatment cycle that lasts from 21 days, 30 days or 42 days to 7 months. In other embodiments, the subject will have a treatment cycle that lasts from 21 days, 30 days or 42 days to 6 months. In other embodiments, the subject will have a treatment cycle that lasts from 21 days, 30 days or 42 days to 5 months. In other embodiments, the subject will have a treatment cycle that lasts from 21 days, 30 days or 42 days to 4 months. In other embodiments, the subject will have a treatment cycle that lasts from 21 days, 30 days or 42 days to 3 months. In other embodiments, the subject will have a treatment cycle that lasts from 21 days, 30 days or 42 days to 2 months.

In further embodiments, the treatment cycle will be based on the level of markers that track the progress of disease, including patient reported CeD symptoms, villous atrophy, villous height to crypt depth ratio, IgA anti-tissue transglutaminase (TGA), IgG anti-deamidated gluten peptide (DGP) and other markers disclosed elsewhere herein. The patient may be treated for an additional period to ensure a population of Treg cells that suppress and reverse disease. A subject may also be monitored and treated at the first appearance of any indicia of re-emergent disease.

Daily maintenance doses can be given for a period clinically desirable in the subject, for example from 1 day up to several years (e.g. for the subject's entire remaining life); for example from about (2, 3 or 5 days, 1. 2, or 3 weeks, or 1 month) upwards and/or for example up to about (5 years, 1 year, 6 months, 1 month, 1 week, or 3 or 5 days). Administration of the daily maintenance dose for about 3 to about 5 days or for about 1 week to about 1 year is typical. Nevertheless, unit doses should for example be administered from twice daily to once every two weeks until a therapeutic effect is observed.

The microorganisms producing the IL-10 polypeptide and the CeD-specific antigen (e.g., a gliadin peptide comprising at least one HLA-DQ2-specific epitope, at least one deamidated HLA-DQ2 specific epitope, at least one HLA-DQ8-specific epitope, at least one deamidated HLA-DQ8 specific epitope, or a combination of (i) at least one HLA-DQ2-specific epitope and/or at least one HLA-DQ2 specific epitope, and(ii) at least one HLA-DQ8 -specific epitope and/or at least one HLA-DQ8 specific epitope) polypeptide may be administered to the subject in mono- or combination therapy (e.g., using a co-therapeutic regimen) for the treatment of CeD. “The term “co-therapy,” “co-therapeutic” or variation thereof refers to a treatment regimen, in which the subject adheres to a grain-free diet (GFD) and/or is administered at least one additional therapeutically active agent, such as an additional immuno-modulating compound. Thus, in some embodiments, the compositions of the present disclosure include additional therapeutically active agents. In some embodiments, the compositions of the present disclosure contain at least one additional immuno-modulating substance, such as antibodies (e.g., anti-CD3 antibodies). In some examples, the methods of the present disclosure further include administering to the subject (e.g., a human patient) an additional immuno-modulating substance, such as antibodies (e.g., anti-CD3 antibodies). In some examples, the additional therapeutically active agent excludes anti-CD3 antibodies

### Pharmaceutical Compositions and Carriers

The microorganism (e.g., bacteria, such as LAB described herein) may be administered in pure form, combined with other active ingredients, and/or combined with pharmaceutically acceptable (i.e., nontoxic) excipients or carriers. The term “pharmaceutically acceptable” is used herein in accordance with its art-recognized meaning and refers to carriers that are compatible with the other ingredients of a pharmaceutical composition, and are not deleterious to the recipient thereof.

The compositions of the present disclosure can be prepared in any known or otherwise effective dosage or product form suitable for delivery of the microorganism (e.g., bacteria) to the mucosa, which would include pharmaceutical compositions and dosage forms as well as nutritional product forms.

In some embodiments, the pharmaceutical composition (i.e., formulation) is an oral pharmaceutical composition. In some examples according to this embodiment, the formulation or pharmaceutical composition comprises the non-pathogenic microorganism in a dried form (e.g., dry-powder form; e.g., freeze-dried form) or in compacted form thereof, optionally in combination with other dry carriers. Oral formulations will generally include an inert diluent carrier or an edible carrier.

In some examples, the oral formulation comprises a coating or utilizes an encapsulation strategy, which facilitates the delivery of the formulation into the intestinal tract, and/or allows the microorganism be released and hydrated in the intestinal tract (e.g., the ileum, small intestine, or the colon). Once the microorganism is released from the formulation and sufficiently hydrated, it begins expressing the bioactive polypeptides, which are subsequently released into the surroundings, or expressed on the surface of the microorganism. Such coating and encapsulation strategies (i.e., delayed-release strategies) are known to those of skill in the art. See, e.g., U.S. Pat. No. 5,972,685; WO 2000/18377; and WO 2000/22909, the disclosures of which are incorporated herein by reference in their entirety.

In some embodiments, the disclosure provides a pharmaceutical composition comprising the microorganism (e.g., the non-pathogenic bacteria) in a lyophilized or freeze-dried form, optionally in conjunction with other components, such as dextrans, sodium glutamate, and polyols. Exemplary freeze-dried compositions are described, e.g., in U.S. Patent Application No. 2012/0039853 to Corveleyn et al., the disclosure of which is incorporated herein by reference in its entirety. Exemplary formulations comprise freeze-dried bacteria (e.g., a therapeutically effective amount of the bacteria) and a pharmaceutically acceptable carrier. Freeze-dried bacteria may be prepared in the form of capsules, tablets, granulates and powders, each of which may be administered orally. Alternatively, freeze-dried bacteria may be prepared as aqueous suspensions in suitable media, or lyophilized bacteria may be suspended in a suitable medium, such as a drink, just prior to use. Such composition may additionally contain a stabilizing agent useful to maintain a stable suspension, e.g., without precipitation, aggregation, or floating of the bacterial biomass.

For oral administration, the formulation may be a gastro-resistant oral dosage form. For example, the oral dosage form (e.g., capsules, tablets, pellets, micro-pellets, granulates, and the like) may be coated with a thin layer of excipient (usually polymers, cellulosic derivatives and/or lipophilic materials) that resists dissolution or disruption in the stomach, but not in the intestine, thereby allowing transit through the stomach in favor of disintegration, dissolution and absorption in the intestine (e.g., the small intestine, or the colon).

In some examples, oral formulations may include compounds providing controlled release, sustained release, or prolonged release of the microorganism, and thereby provide controlled release of the desired protein encoded therein. These dosage forms (e.g., tablets or capsules) typically contain conventional and well known excipients, such as lipophilic, polymeric, cellulosic, insoluble, and/or swellable excipients. Controlled release formulations may also be used for any other delivery sites including intestinal, colon, bioadhesion or sublingual delivery (i.e., dental mucosal delivery) and bronchial delivery. When the compositions of the invention are to be administered rectally or vaginally, pharmaceutical formulations may include suppositories and creams. In this instance, the host cells are suspended in a mixture of common excipients also including lipids. Each of the aforementioned formulations are well known in the art and are described, for example, in the following references: Hansel et al. (1990, Pharmaceutical dosage forms and drug delivery systems, 5th edition, William and Wilkins); Chien 1992, Novel drug delivery system, 2nd edition, M. Dekker); Prescott et al. (1989, Novel drug delivery, J. Wiley & Sons); Gazzaniga et al., (1994, Oral delayed release system for colonic specific delivery, Int. J. Pharm. 108: 77-83).

In some embodiments, the oral formulation includes compounds that can enhance mucosal delivery and/or mucosal uptake of the bioactive polypeptides expressed by the microorganism. In other examples, the formulation includes compounds, which enhance the viability of the microorganism within the formulation, and/or once released.

The bacteria of the invention can be suspended in a pharmaceutical formulation for administration to the human or animal having the disease to be treated. Such pharmaceutical formulations include but are not limited to live gram-positive bacteria and a medium suitable for administration. The bacteria may be lyophilized in the presence of common excipients such as lactose, other sugars, alkaline and/or alkali earth stearate, carbonate and/or sulphate (e.g., magnesium stearate, sodium carbonate and sodium sulphate), kaolin, silica, flavorants and aromas. Bacteria so-lyophilized may be prepared in the form of capsules, tablets, granulates and powders (e.g., a mouth rinse powder), each of which may be administered by the oral route. Alternatively, some gram-positive bacteria may be prepared as aqueous suspensions in suitable media, or lyophilized bacteria may be suspended in a suitable medium just prior to use, such medium including the excipients referred to herein and other excipients such as glucose, glycine and sodium saccharinate.

In some examples, the microorganism is locally delivered to the gastrointestinal tract of the subject using any suitable method. For example, microsphere delivery systems could be employed to enhance delivery to the gut. Microsphere delivery systems include microparticles having a coating that provides localized release into the gastrointestinal tract of the subject (e.g., controlled release formulations such as enteric-coated formulations and colonic formulations).

For oral administration, gastroresistant oral dosage forms may be formulated, which dosage forms may also include compounds providing controlled release of the gram-positive bacteria and thereby provide controlled release of the desired protein encoded therein (e.g., IL-10 and an HLA-DQ2-specific epitope and/or an HLA-DQ8-specific epitope). For example, the oral dosage form (including capsules, tablets, pellets, granulates, powders) may be coated with a thin layer of excipient (e.g., polymers, cellulosic derivatives and/or lipophilic materials) that resists dissolution or disruption in the stomach, but not in the intestine, thereby allowing transit through the stomach in favor of disintegration, dissolution and absorption in the intestine.

The oral dosage form may be designed to allow slow release of the gram-positive bacteria and of the produced exogenous proteins, for instance as controlled release, sustained release, prolonged release, sustained action tablets or capsules. These dosage forms usually contain conventional and well-known excipients, such as lipophilic, polymeric, cellulosic, insoluble, and/or swellable excipients. Such formulations are well-known in the art and are described, for example, in the following references: Hansel et al., Pharmaceutical dosage forms and drug delivery systems, 5th edition, William and Wilkins, 1990; Chien 1992, Novel drug delivery system, 2nd edition, M. Dekker; Prescott et al., Novel drug delivery, J.Wiley & Sons, 1989; and Gazzaniga et al., Int. J. Pharm. 108: 77-83 (1994).

The pharmaceutical dosage form (e.g. capsule) may be coated with pH-dependent Eudragit polymers to obtain gastric juice resistance and for the intended delivery at the terminal ileum and colon, where the polymers dissolve at pH 6.5. By using other Eudragit polymers or a different ratio between the polymers, the delayed release profile could be adjusted, to release the bacteria for example in the duodenum or jejenum.

Pharmaceutical compositions contain at least one pharmaceutically acceptable carrier. Non-limiting examples of suitable excipients, diluents, and carriers include preservatives, inorganic salts, acids, bases, buffers, nutrients, vitamins, fillers and extenders such as starch, sugars, mannitol, and silicic derivatives; binding agents such as carboxymethyl cellulose and other cellulose derivatives, alginates, gelatin, and polyvinyl pyrolidone; moisturizing agents such as glycerol/disintegrating agents such as calcium carbonate and sodium bicarbonate; agents for retarding dissolution such as paraffin; resorption accelerators such as quaternary ammonium compounds; surface active agents such as acetyl alcohol, glycerol monostearate; adsorptive carriers such as kaolin and bentonite ; carriers such as propylene glycol and ethyl alcohol, and lubricants such as talc, calcium and magnesium stearate, and solid polyethyl glycols.

Pharmaceutically compatible binding agents, and/or adjuvant materials can be included as part of the composition. Tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials that modify the physical form of the dosage unit, for example, coatings of sugar, shellac, or enteric agents. Further, a syrup may contain, in addition to the active compounds, sucrose as a sweetening agent and certain preservatives, dyes, colorings, and flavorings. It will be appreciated that the form and character of the pharmaceutically acceptable carrier is dictated by the amount of active ingredient with which it is to be combined, the route of administration and other well-known variables. The carrier(s) must be “acceptable” in the sense of being compatible with the other ingredients of the formulation and not deleterious to the recipient thereof.

Alternative preparations for administration include sterile aqueous or nonaqueous solutions, suspensions, and emulsions. Examples of non-aqueous solvents are dimethylsulfoxide, alcohols, propylene glycol, polyethylene glycol, vegetable oils such as olive oil and injectable organic esters such as ethyl oleate. Aqueous carriers include mixtures of alcohols and water, buffered media, and saline. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like. Various liquid formulations are possible for these delivery methods, including saline, alcohol, DMSO, and water-based solutions.

Oral aqueous formulations include excipients, such as pharmaceutical grades of mannitol, lactose, starch, magnesium stearate, sodium saccharine, cellulose, magnesium carbonate and/or the like. These compositions take the form of solutions such as mouthwashes and mouth rinses, further comprising an aqueous carrier such as for example water, alcoholic/aqueous solutions, saline solutions, parenteral vehicles such as sodium chloride, Ringer's dextrose, and the like.

Aqueous mouthwash formulations are well-known to those skilled in the art. Formulations pertaining to mouthwashes and oral rinses are discussed in detail, for example, in U.S. Pat. Nos. 6,387,352, 6,348,187, 6,171,611, 6,165,494, 6,117,417, 5,993,785, 5,695,746, 5,470,561, 4,919,918, U.S. Patent Appl. Pub. No. 2004/0076590, U.S. Patent Appl. Pub. No. 2003/0152530, and U.S. Patent Appl. Pub. No. 2002/0044910, each of which is herein specifically incorporated by reference.

Other additives may be present in the formulations of the present disclosure, such as flavoring, sweetening or coloring agents, or preservatives. Mint, such as from peppermint or spearmint, cinnamon, eucalyptus, citrus, cassia, anise and menthol are examples of suitable flavoring agents. Flavoring agents are for example present in the oral compositions in an amount in the range of from 0 to 3%; up to 2%, such as up to 0.5%, e.g., around 0.2%, in the case of liquid compositions.

Sweeteners include artificial or natural sweetening agents, such as sodium saccharin, sucrose, glucose, saccharin, dextrose, levulose, lactose, mannitol, sorbitol, fructose, maltose, xylitol, thaumatin, aspartame, D-tryptophan, dihydrochalcones, acesulfame, and any combination thereof, which may be present in an amount in the range of from 0 to 2%, for example, up to 1% w/w, such as 0.05 to 0.3% w/w of the oral composition.

Coloring agents are suitable natural or synthetic colors, such as titanium dioxide or CI 42090, or mixtures thereof. Coloring agents are preferably present in the compositions in an amount in the range of from 0 to 3%; for example, up to 0.1%, such as up to 0.05%, e.g., around 0.005-0.0005%, in the case of liquid compositions. Of the usual preservatives, sodium benzoate is preferred in concentrations insufficient substantially to alter the pH of the composition, otherwise the amount of buffering agent may need to be adjusted to arrive at the desired pH.

Other optional ingredients include humectants, surfactants (non-ionic, cationic or amphoteric), thickeners, gums and binding agents. A humectant adds body to the formulation and retains moisture in a dentifrice composition. In addition, a humectant helps to prevent microbial deterioration during storage of the formulation. It also assists in maintaining phase stability and provides a way to formulate a transparent or translucent dentifrice.

Suitable humectants include glycerine, xylitol, glycerol and glycols such as propylene glycol, which may be present, for example, in an amount of up to 50% w/w each, but total humectant is in some cases not more than about 60-80% w/w of the composition. For example, liquid compositions may comprise up to about 30% glycerine plus up to about 5%, for example, about 2% w/w xylitol. Surfactants are preferably not anionic and may include polysorbate 20 or cocoamidobetaine or the like in an amount up to about 6%, for example, about 1.5 to 3%, w/w of the composition.

When the oral compositions of the invention are in a liquid form, it is preferred to include a film-forming agent up to about 3% w/w of the oral composition, such as in the range of from 0 to 0.1%, for example, about 0.001 to 0.01%, such as about 0.005% w/w of the oral composition. Suitable film-formers include (in addition to sodium hyaluronate) those sold under the tradename Gantrez.

Liquid nutritional formulations for oral or enteral administration may comprise one or more nutrients such as fats, carbohydrates, proteins, vitamins, and minerals. Many different sources and types of carbohydrates, lipids, proteins, minerals and vitamins are known and can be used in the nutritional liquid embodiments of the present invention, provided that such nutrients are compatible with the added ingredients in the selected formulation, are safe and effective for their intended use, and do not otherwise unduly impair product performance.

These nutritional liquids are, for example, formulated with sufficient viscosity, flow, or other physical or chemical characteristics to provide a more effective and soothing coating of the mucosa while drinking or administering the nutritional liquid. These nutritional embodiments also in some cases represent a balanced nutritional source suitable for meeting the sole, primary, or supplemental nutrition needs of the individual.

Non-limiting examples of suitable nutritional liquids are described in U.S. Pat. No. 5,700,782 (Hwang et al.); U.S. Pat. No. 5,869,118 (Morris et al.); and U.S. Pat. No. 5,223,285 (DeMichele et al.), which descriptions are incorporated herein by reference in their entireties.

Nutritional proteins suitable for use herein can be hydrolyzed, partially hydrolyzed or non-hydrolyzed, and can be derived from any known or otherwise suitable source such as milk (e g , casein, whey), animal (e g , meat, fish), cereal (e.g., rice, corn), vegetable (e.g., soy), or any combination thereof.

Fats or lipids suitable for use in the nutritional liquids include, but are not limited to, coconut oil, soy oil, corn oil, olive oil, safflower oil, high oleic safflower oil, MCT oil (medium chain triglycerides), sunflower oil, high oleic sunflower oil, structured triglycerides, palm and palm kernel oils, palm olein, canola oil, marine oils, cottonseed oils, and any combination thereof. Carbohydrates suitable for use in the nutritional liquids may be simple or complex, lactose-containing or lactose-free, or any combination thereof. Non-limiting examples of suitable carbohydrates include hydrolyzed cornstarch, maltodextrin, glucose polymers, sucrose, corn syrup, corn syrup solids, rice-derived carbohydrate, glucose, fructose, lactose, high fructose corn syrup and indigestible oligosaccharides such as fructo-oligosaccharides (FOS), and any combination thereof.

The nutritional liquids may further comprise any of a variety of vitamins, non-limiting examples of which include vitamin A, vitamin D, vitamin E, vitamin K, thiamine, riboflavin, pyridoxine, vitamin B12, niacin, folic acid, pantothenic acid, biotin, vitamin C, choline, inositol, salts and derivatives thereof, and any combination thereof.

The nutritional liquids may further comprise any of a variety of minerals known or otherwise suitable for use in patients at risk of or suffering from CeD, non-limiting examples of which include calcium, phosphorus, magnesium iron, selenium, manganese, copper, iodine, sodium, potassium, chloride, and any combination thereof.

The microorganisms and in particular the yeast and bacteria of the present invention can also be formulated as elixirs or solutions for convenient oral or rectal administration or as solutions appropriate for parenteral administration, for instance by intramuscular, subcutaneous or intravenous routes. Additionally, the nucleoside derivatives are also well suited for formulation as a sustained or prolonged release dosage forms, including dosage forms that release active ingredient only or in some cases in a particular part of the intestinal tract, for example over an extended or prolonged period of time to further enhance effectiveness. The coatings, envelopes, and protective matrices in such dosage forms may be made, for example, from polymeric substances or waxes well known in the pharmaceutical arts.

The compositions of the present invention include pharmaceutical dosage forms such as lozenges, troches or pastilles. These are typically discoid-shaped solids containing the active ingredient in a suitably flavored base. The base may be a hard sugar candy, glycerinated gelatin, or the combination of sugar with sufficient mucilage to give it form. Troches are placed in the mouth where they slowly dissolve, liberating the active ingredient for direct contact with the mucosa.

The troche embodiments of the present invention can be prepared, for example, by adding water slowly to a mixture of the powdered active, powdered sugar, and a gum until a pliable mass is formed. A 7% acacia powder can be used to provide sufficient adhesiveness to the mass. The mass is rolled out and the troche pieces cut from the flattened mass, or the mass can be rolled into a cylinder and divided. Each cut or divided piece is shaped and allowed to dry, to thus form the troche dosage form.

If the active ingredient is heat labile, it may be made into a lozenge preparation by compression. For example, the granulation step in the preparation is performed in a manner similar to that used for any compressed tablet. The lozenge is made using heavy compression equipment to give a tablet that is harder than usual as it is desirable for the dosage form to dissolve or disintegrate slowly in the mouth. Ingredients are in some cases selected to promote slow-dissolving characteristics.

In a particular formulation of the present invention, the microorganisms will be incorporated in a bioadhesive carrier containing pre-gelatinized starch and cross-linked poly (acrylic acid) to form a bioadhesive tablet and a bioadhesive gel suitable for buccal application (i.e., having prolonged bioadhesion and sustained drug delivery.

In an alternative embodiment, a powder mixture of non-pathogenic and non-invasive bacterium according to the invention, bioadhesive polymers (pregelatinized starch and cross-linked poly (acrylic acid) coprocessed via spray drying), sodium stearyl fumarate (lubricant), and silicium dioxide (glidant) is processed into tablets (weight: 100 mg; diameter: 7 mm). The methods for the production of these tablets are well known to the person skilled in the art and has been described before for the successful development of bioadhesive tablets containing various drugs (miconazol, testosterone, fluoride, ciprofloxacin) (Bruschi M. L. and de Freitas O., Drug Development and Industrial Pharmacy, 2005 31:293-310). All excipient materials are commercially available in pharmaceutical grades.

To optimize a formulation, the drug load in the tablets and the ratio between starch and poly (acrylic acid) will be varied. Based on previous research, the maximum drug load in the coprocessed bioadhesive carrier is about 60% (w/w) and the starch/poly (acrylic acid) ratio can be varied between 75/25 and 95/5 (w/w). During the optimization study, the bioadhesive properties of the tablets and the drug release from the tablets are the main evaluation parameters, with the standard tablet properties (hardness, friability) as secondary evaluation criteria.

The bacteria are incorporated into an aqueous dispersion of pregelatinized starch and cross-linked poly (acrylic acid). This polymer dispersion is prepared via a standard procedure using a high shear mixer.

Similar to the tablet, the drug load of the gel and the starch/poly (acrylic acid) ratio need to be optimized in order to obtain a gel having optimal adherence to the esophageal mucosa. For a gel, the concentration of the polymers in the dispersion is an additional variable as it determines the viscosity of the gel, hence its muco-adhesive properties.

The model to screen the bioadhesive properties of polymer dispersions to the mucosa of esophagus has been described in detail by Batchelor et al. (Int. J. Pharm., 238: 123-132, 2002).

Other routes and forms of administration include food preparations containing the live microorganisms. In some examples, the bioactive polypeptide-expressing microorganism can be included into a dairy product.

The pharmaceutical compositions of the present invention can be prepared by any known or otherwise effective method for formulating or manufacturing the selected dosage form. For example, the microorganisms can be formulated along with common, e.g., pharmaceutically acceptable carriers, such as excipients and diluents, formed into oral tablets, capsules, sprays, lozenges, treated substrates (e.g., oral or topical swabs, pads, or disposable, non-digestible substrate treated with the compositions of the present invention); oral liquids (e.g., suspensions, solutions, emulsions), powders, suppositories, or any other suitable dosage form. In some embodiments, the present disclosure provides a method for the manufacture of a pharmaceutical composition. Exemplary methods include: contacting the microorganism (e.g., the non-pathogenic bacterium) containing the IL-10 gene and the CeD-specific antigen gene (or which is capable of expressing the IL-10 and the CeD-specific antigen) with a pharmaceutically acceptable carrier, thereby forming the pharmaceutical composition. In some examples, the method further includes: growing the microorganism in a medium. The method may further include freeze-drying a liquid containing the microorganism, wherein the liquid optionally includes the pharmaceutically acceptable carrier.

### Unit Dosage Forms

The current disclosure further provides unit dosage forms comprising a certain amount of a non-pathogenic microorganism optionally in combination with a food-grade or pharmaceutically acceptable carrier, wherein said non-pathogenic microorganism (e.g., the non-pathogenic gram-positive bacterium) comprises: an exogenous nucleic acid encoding an IL-10 polypeptide; and an exogenous nucleic acid encoding a CeD-specific antigen (e.g., a gliadin peptide comprising at least one human leukocyte antigen (HLA)-DQ2-specific, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope). Exemplary unit dosage forms contain from about 1×10³ to about 1×10¹⁴ colony-forming units (cfu) of the non-pathogenic microorganism (e.g., a non-pathogenic gram-positive bacterium). Other exemplary unit dosage forms contain from about 1×10⁴ to about 1×10¹³ cfu of a non-pathogenic microorganism (e.g., a non-pathogenic gram-positive bacterium), or from about 1×10⁴ to about 1×10¹² cfu of a non-pathogenic microorganism (e.g., a non-pathogenic gram-positive bacterium). In other embodiments, the unit dosage form comprises from about 1×10⁵ to about 1×10¹² cfu, or from about 1×10⁶ to about 1×10¹² cfu of the non-pathogenic microorganism (e.g., the non-pathogenic gram-positive bacterium). In other embodiments, the unit dosage form comprises from about 1×10⁸ to about 1×10¹² cfu, or from about 1×10⁹ to about 1×10¹² cfu of the non-pathogenic microorganism (e.g., the non-pathogenic gram-positive bacterium). In yet other embodiments, the unit dosage form comprises from about 1×10⁹ to about 1×10¹¹ cfu, or from about 1×10⁹ to about 1×10¹⁰ cfu of the non-pathogenic microorganism (e.g., the non-pathogenic gram-positive bacterium). In yet other embodiments, the unit dosage form comprises from about 1×10′ to about 1×10¹¹ cfu, or from about 1×10⁸ to about 1×10¹⁰ cfu of the non-pathogenic microorganism (e.g., the non-pathogenic gram-positive bacterium). In some examples, the unit dosage contains about 1×10⁴ to about 1×10¹² colony-forming units (cfu) of sAGX0868. In some examples, the unit dosage form contains from about 1×10⁸ to about 1×10¹¹ cfu, or about 1×10¹⁰ to about 1×10¹¹ cfu, or about 1×10¹¹ cfu sAGX0868.

In yet other embodiments, the unit dosage form comprises from about 1×10⁹ to about 1×10¹⁰ cfu, or from about 1×10⁹ to about 100×10⁹ cfu of the non-pathogenic microorganism (e.g., the non-pathogenic gram-positive bacterium).

The unit dosage form can have any physical form or shape. In some embodiments, the unit dosage form is adapted for oral administration. In some examples according to these embodiments, the unit dosage form is in the form of a capsule, a tablet, or a granule. Exemplary capsules include capsules filled with micro-granules. In some embodiments, the non-pathogenic microorganism (e.g., the non-pathogenic gram-positive bacterium) contained in the dosage form is in a dry-powder form. For example, the microorganism is in a freeze-dried powder form, which is optionally compacted and coated.

The compositions and methods can be better understood by reference to the Examples that follow, but those skilled in the art will appreciate that these are only illustrative of the invention as described more fully in the numbered embodiments and embodiments that follow. Additionally, throughout this application, various publications are cited. The disclosures of these publications are hereby incorporated by reference in their entirety.

### EXAMPLES

### Example 1: Treatment of Celiac Disease in Mice

This experiment describes an in vivo interventional study, i.e., starting L. lactis treatment after initiation of the disease using gluten treatment. This experiment evaluates efficacy of deamidated HLA-DQ8-specific-epitope (“dDQ8”) expressing L. lactis strains to restore oral tolerance towards gluten in a mouse model for CeD. Human CeD patients predominantly express the HLA-DQ2.5 allele. However, HLA-DQ2.5 is not expressed in mice, and because no humanized DQ2 model demonstrating typical CeD features is available, proof-of-concept was pursued using a surrogate dDQ8-secreting L. lactis strain in an HLA DQ8-restricted mouse model. The surrogate dDQ8-secreting L. lactis strain has identical genetic traits as the proposed dDQ2-secreting L. lactis clinical strain, except for the secreted epitope.

### Materials And Methods

A non-exhaustive list of abbreviations used in the following description is provided in Table 6.

TABLE 6

| | |
|---|---|
| APC | Antigen presenting cells |
| CeD | Celiac disease |
| CT | Chemo-trypsin |
| DGP | Deamidated gluten peptides |
| FACS | Fluorescence-activated cell sorting |
| hIL-10 | Human interleukin-10 |
| HLA | Human leukocyte antigen |
| IEC | Intestinal epithelial cell |
| IEL | Intraepithelial cell |
| L. lactis or LL | Lactococcus lactis |
| LP | Lamina propria |
| LPL | Lamina propria lymphocyte |
| NK | Natural killer |
| PBS | Phosphate-buffered saline |
| RPMI | Roswell Park Memorial Institute 1640 Medium |
| RT | Room temperature |
| TG2 | Tissue transglutaminase 2 |
| VA | Villous atrophy |

Overview of Experiment

DQ8-IL15LPxIEC mice were exposed (by diet and gastric gavage) to gluten for 30 days, recovered 30 days on a gluten-free diet (GFD), and then were administered one of 4 strains of L. lactis every day, while the mice were maintained on a GFD for 21 days. Mice were then re-challenged with a gluten-containing diet for 21 days, while continuing the daily L. lactis treatment. At the end of each experiment, mice were euthanized and small intestines were processed for histology (Hematoxylin and Eosin (H&E) staining for pathology, CD3 immunostaining for intraepithelial lymphocytes (IELs) counts), lamina propria (LP) and epithelium isolation for fluorescence-activated cell sorting (FACS) analysis for markers of activation of IELs. The levels of gene expression for epithelial stress markers and cytotoxic molecules were evaluated by quantitative polymerase chain reaction (qPCR)

### Mice

The mouse model of CeD used is a HLA-DQ8 humanized mouse overexpressing the proinflammatory cytokine IL-15 in all tissues, and in particular, in both the intestinal epithelium and lamina propria as IL-15 expression is driven by the Dd and villin promoters. The mice are referred to as DQ8-IL15^{LPxIEC}. DQ8-IL15^{LPxIECL} mice are on a C57BL/6 background and were generated by crossing DQ8-IL15^{LP} mice to DQ8-IL15^{IEC} mice (Kim et al, manuscript in preparation).

DQ8-IL15^{LPxIEC} mice, when exposed to dietary gluten, develop T cell infiltration and intestinal tissue destruction as seen in the human situation. Therefore, this mouse model provides an excellent opportunity to test the therapeutic potential of the AG017 surrogate L. lactis strains.

DQ8-IL15^{LPxIEC} mice were 9 weeks of age at the start of experiment, and both male and female mice were used. Mice were kept on a GFD (Research Diets, AIN-76A) until the start of experiment, when gluten was introduced to the diet to induce CeD. In addition to a GFD, mice were administered approximately 20 mg gliadin (Sigma, G3375) by gastric gavage every other day during this diet.

All animal procedures have been reviewed by the local ethical committee of the University of Chicago, ACUP 71966.

Lactococcus lactis Strains and Culture

The efficacy of L. lactis strains secreting deamidated HLA-DQ8 peptide (Table 7) is examined, with or without co-secreted hIL-2 or hIL-10.

TABLE 7

| L. lactis strain | Common name | Description |
|---|---|---|
| L. lactis-pT1NX | LL-empty vector | L. lactis strain with |
| | | plasmid backbone |
| MG1363[pAGX2263] | LL-[dDQ8] | L. lactis strain with |
| | | plasmid-encoded |
| | | deamidated |
| | | HLA-DQ8-peptide |
| sAGX0487[pAGX2263] | LL-[dDQ8] + IL-10 | L. lactis strain with |
| | | human IL-10 gene |
| | | integrated in the |
| | | genome and plasmid- |
| | | encoded deamidated |
| | | HLA-DQ8-peptide |
| sAGX0526[pAGX2263] | LL-[dDQ8] + IL-2 | L. lactis strain with |
| | | human IL-2 gene |
| | | integrated in the |
| | | genome and plasmid- |
| | | encoded deamidated |
| | | HLA-DQ8-peptide |

L. lactis-pT1NX is an MG1363 strain containing the empty vector pT1NX (GenBank: HM585371.1), and served as control. The plasmid-driven L. lactis strain MG1363[pAGX2263] contains plasmid pAGX2263. In pAGX2263, the hllA promoter (PhllA) drives the expression of a gene encoding a fusion of ps356 endolysin gene secretion leader (SSps356,SL #21) with a fragment encoding deamidated HLA-DQ8-peptide, to allow expression and secretion of deamidated HLA-DQ8-peptide. Plasmid pAGX2263 was electroporated into wild type L. lactis subsp. cremoris strain MG1363.

The plasmid-driven L. lactis strain sAGX0487 [pAGX2263] contains plasmid pAGX2263, described above. Plasmid pAGX2263 was electroporated into sAGX0487. In sAGX0487 (L. lactis subsp. cremoris MG1363: ΔthyA; eno>>SSusp45-hil-10; usp45>>otsB; ΔtrePP; PhllA>>trePTS; ptcC-):
Thymidylate synthase gene (thyA; Gene ID: 4798358) is absent, to warrant environmental containment.
Trehalose-6-phosphate phosphorylase gene (trePP; Gene ID: 4797140) is absent, to allow accumulation of exogenously added trehalose.
Trehalose-6-phosphate phosphatase gene (otsB; Gene ID: 1036914) is positioned downstream of usp45 (Gene ID: 4797218) to facilitate conversion of trehalose-6-phosphate to trehalose. The otsB expression unit was transcriptionally and translationally coupled to usp45 by use of the intergenic region (IR) preceding the highly expressed L. lactis MG1363 50S ribosomal protein L30 gene (rpmD; Gene ID: 4797873).
The constitutive promoter of the HU-like DNA-binding protein gene (PhllA; Gene ID: 4797353) is preceding the putative phosphotransferase genes in the trehalose operon (trePTS; LLMG_RS02300 LLMG_RS02305, Gene ID: 4797778 and Gene ID: 4797093 respectively) to potentiate trehalose uptake.
The gene encoding cellobiose-specific PTS system IIC component (ptcC; GeneID: 4796893) is disrupted (tga at codon position 30 of 446; tga30). This mutation ascertains trehalose retention after accumulation.
A gene encoding a fusion of usp45 secretion leader (SSusp45) with the hil-10 gene, encoding human interleukin-10 (hIL-10; UniProt: P22301, aa 19-178, variant P2A is positioned downstream of the phosphopyruvate hydratase gene (eno; Gene ID: 4797432), to allow expression and secretion of hIL-10. The hil-10 expression unit was transcriptionally and translationally coupled to eno by use of IRrpmD.
   When grown in the presence of trehalose, sAGX0487 accumulates and retains trehalose, which provides protection from bile acid toxicity. Furthermore, sAGX0487 constitutively expresses and secretes hIL-10.

The plasmid-driven L. lactis strain sAGX0526 [pAGX2263] contains plasmid pAGX2263 (described above). Plasmid pAGX2263 was electroporated into sAGX0526. In sAGX0526 (L. lactis subsp. cremoris MG1363: ΔthyA; eno>>SSusp45-hil-2; usp45>>otsB; ΔtrePP; PhllA>>trePTS; ΔptcC):
Thymidylate synthase gene (thyA; Gene ID: 4798358) is absent, to ascertain environmental containment.
Trehalose-6-phosphate phosphorylase gene (trePP; Gene ID: 4797140) is absent, to allow accumulation of exogenous trehalose.
Trehalose-6-phosphate phosphatase (otsB; Gene ID: 1036914) is positioned downstream of unidentified secreted 45-kDa protein gene (usp45; Gene ID: 4797218) to facilitate conversion of trehalose-6-phosphate to trehalose.
The constitutive promoter of the HU-like DNA-binding protein gene (PhllA; Gene ID: 4797353) is preceding the putative phosphotransferase genes in the trehalose operon (trePTS; ptsI and ptsII; LLMG_RS02300 3 and LLMG_RS02305, Gene ID: 4797778 and Gene ID: 4797093 respectively) to potentiate trehalose uptake.
The gene encoding cellobiose-specific PTS system IIC component (Gene ID: 4796893), ptcC, is deleted to increase trehalose retention.
A gene encoding a fusion of usp45 secretion leader (SSusp45) with the hil-2 gene, encoding human interleukin-2 (hIL-2; UniProt: P60568, aa 21-153) is positioned downstream of the phosphopyruvate hydratase gene (eno; Gene ID: 4797432), to allow expression and secretion of hIL-2. The hil-2 expression unit was transcriptionally and translationally coupled to eno by use of IRrpmD.
   When grown in the presence of trehalose, sAGX0526 accumulates and retains trehalose, which provides protection from bile acid toxicity. Furthermore, sAGX0526 constitutively expresses and secretes hIL-2.

Overnight cultures (12-16 hours at 30° C., standing culture) were prepared by inoculating GM17TE broth (39.1 gram per liter (g/l) M17 broth, 0.5% (w/v) glucose, 200 micromolar (μM) thymidine, and 5 microgram per milliliter (μg/ml) erythromycin) with 10 microliter (μl) of the bacterial stocks. These cultures were spun down at 4,000 g for 10 minutes at 4° C., after which the pellet was re-suspended in 2 ml BM9T medium (1× M9 salts, 0.5% casiton, 0.5% glucose, 30 mM NaHCO₃, 20 mM Na₂CO₃, 2 mM MgSO₄, 100 μM CaCl₂, and 200 μM thymidine) and mixed well. Mice received 100 μl dosing solution per oral gavage daily (10⁹ colony-forming units (CFU)). Quality controls were performed by determining the CFU per milliliter (CFU/ml), 1-2× a week by plating 5 dilutions made in M9 buffer.

### Mouse Dissection and Cell Isolation

Mice were sacrificed by cervical dislocation. Mesenteric lymph nodes were extracted, and Peyer's patches were removed from the small intestine before processing. Five millimeters (mm) were taken from the beginning of the duodenum, and jejunum, and the last 5 mm of the ileum and placed in 10% formalin for histology. The small intestine was used for cell extraction as follows: IELs were separated by shaking fragmented small intestines twice in 15 ml Roswell Park Memorial Institute (RPMI) 1640 complemented with 1% dialyzed fetal bovum serum (FBS), 2 mM EDTA and 1.5 mM MgCl₂, for 20 minutes at 37° C. and 220 revolutions per minute (RPM). Epithelial cells were recovered in the medium and filtered through a 100 μm strainer, spun down at 1600 RPM at 4° C. and resuspended in cold FACS buffer (PBS with 2% FBS).

Lamina propria lymphocytes (LPLs) were isolated by two incubations in RPMI 1640 Medium complemented with 20% FBS and 100 U/ml collagenase VIII (Sigma, C2139) for 20 minutes. This was followed by centrifugation of the IEL and LP cells in 40% Percoll (GE Healthcare, 17-0891-01) for 12 minutes at 20° C. at 3,000 RPM with acceleration/break at low (1/1). Pellet was resuspended in FACS buffer, and IEL and LPL cells were counted.

### Pathology

Ileum sections of 5 μM thickness were cut, hematoxylin and eosin (H&E) stained, and scored in a blind fashion. The simple atrophy score was 0 (no or mild atrophy) or 2 (severe or partial villus atrophy). The villous height/crypt depth ratios were obtained from morphometric measurements of six well-orientated villi. The villous height to crypt depth ratio was calculated by dividing the villous height by the corresponding crypt depth. The measurement of the villous height was made from the top to the shoulder of the villous or up to the top of the crypt of Lieberkühn. The crypt depth was measured as the distance from the top of the crypt of Lieberkühn to the deepest level of the crypt. Villous atrophy was demonstrated by a villous height to crypt depth ratio ≤2.

The amount of intraepithelial lymphocytes (IELs) was determined by counting the amount of CD3+ IELs among at least 100 intestinal epithelial cells on ileal sections stained as follows: Tissue sections were deparaffinized and rehydrated through xylenes and serial dilutions of ethanol to distilled water. They were incubated in antigen retrieval buffer (S1699, DAKO) and heated in a steamer over 97° C. for 20 minutes. Anti-CD3 (1:60, Abcam, ab16669, rabbit IgG) was applied on tissue sections for one-hour incubation at room temperature in a humidity chamber. Following TBS wash, the tissue sections were incubated with biotinylated anti-rabbit IgG (1:200, Cat. No. BA-1000, Vector Laboratories) for 30 minutes at room temperature. The antigen-antibody binding was detected by Elite® ABC HRP kit (Cat. No. PK-6100, Vector Laboratories, Burlingame, Calif.) and DAB (Agilent DAKO, K3468) system. Tissue sections were briefly immersed in hematoxylin for counterstaining and were covered with cover glasses.

### Antibodies and Flow Cytometry

One million IELs were stained with a Live/Dead cell marker and the following conjugated antibodies: CD45, TCRαβ, TCRγβ, CD8α, CD8β, CD4, NKG2D, NKG2A.B6, and CD94.

One million LPLs were stained with a Live/Dead cell marker and the following conjugated antibodies: CD45, TCRαβ, CD8α, CD8β, CD4, Tbet, Foxp3, Rorγt. The antibodies used are specified in Table 8.

TABLE 8

| Marker | Fluorophore | Manufacturer | Clone ID | Cat # |
|---|---|---|---|---|
| Live/Dead | AmCyan | Life | — | L34966 |
| | | Technologies | | |
| CD45 | Pacific Blue | Biolegend | 30-F11 | 103126 |
| TCRαβ | BUV 737 | BD Bioscience | H4H57-597 | 564799 |
| TCRγδ | FITC | eBioscience | eBioGL3 | 11-5711-82 |
| | | invitrogen | | |
| CD8α | Percp-Cy5.5 | BD Pharmigen | 53-6.7 | 551162 |
| CD8β | BUV 395 | BD Bioscience | H35-17.2 | 740278 |
| CD4 | BV 786 | BD Bioscience | GK1.5 | 563331 |
| NKG2D | BV 711 | BD Bioscience | LX5 | 563694 |
| NKG2A.B6 | APC | Biolegend | 16A11 | 142807 |
| CD94 | PE-Cy7 | Biolegend | 18d3 | 105509 |
| Granzyme B | PE | Invitrogen | GB12 | MHGB04 |
| Tbet | APC | Biolegend | 4B10 | 644814 |
| Foxp3 | FITC | Invitrogen | FJK-16s | 11-5773-82 |
| | | eBioscience | | |
| Rorγt | PE | Invitrogen | B2D | 12-6981-82 |
| | | eBioscience | | |
| — | FcBlock | Biolegend | 93 | 101302 |

Cells were first incubated for 10 minutes with Fc block (1:300) in FACS buffer, followed by washing and spin down in FACS buffer (200 μl, 5 minutes at 1,600 RPM, 4° C.). Live/dead staining was performed in PBS (1:50), 10 minutes, 4° C., followed by a wash step. Surface stainings were performed in 50 μl FACS buffer for 25 minutes at 4° C., followed by washing. For intracellular stainings, cells were first fixed in 200 μl permeabilization/fixation solution (Invitrogen) for 20 minutes at 4° C., followed by 2 wash steps in permeabilization/wash buffer. Cells were then incubated with antibodies (in permeabilization/wash buffer) for 30 minutes at 4° C. and washed after. Cells were resuspended in 200-400 μl FACS buffer. Flow cytometry was performed with a BD (Becton, Dicking & Company) analyzer and data were analyzed using FlowJo software (Tree Star Inc.). Analysis was done by gating as follows: lymphocytes, live cells, CD45⁺ cells, TCRαβ⁺ cells, CD8αβ⁺CD4⁻, NKG2D⁺NKG2A⁻. The absolute numbers of cells were calculated by multiplying the fraction of CD8αα⁺CD8αβTCRαβ⁺TCRγδ⁺NKG2D⁺NKG2A⁻ by the number of CD3⁺ cells found on histology.

RNA Isolation and qPCR

Two million cells isolated from the epithelial fractions (cell fraction before Percoll separation) were used for RNA extractions using the Qiagen mini kit according to manufacturer's instruction. Expression of cytotoxic molecule perforin by IELs was evaluated in the cell fraction after Percoll separation. 200 ng RNA was transcribed into cDNA using Promega GoScript™ (Promega, Madison, Wis.). qPCR was performed on a LightCycler® 480 (Roche, Indianapolis, Ind.), using SYBR green (TaKaRa Clontech). Expression levels were normalized to Gapdh. Primers are listed in Table 9.

TABLE 9

| Gene | Forward primer | Reverse primer |
|---|---|---|
| Gapdh | AGGTCGGTGTGAACGGATTTG | TGTAGACCATGTAGTTGAGGTCA |
| | (SEQ ID NO: 50) | (SEQ ID NO: 55) |
| Qa-1 | GACCCAGAGTAGTTCACATT | CCACGTAGCCAACGACTATGA |
| | CG (SEQ ID NO: 51) | (SEQ ID NO: 56) |
| Rae1 | TGGAAAGATGATGGGGACCTT | TGGGGGACCTTGAGGTTGATCT |
| | GTGC (SEQ ID NO: 52) | TGG (SEQ ID NO: 57) |
| Mult1 | GCTTCACATAGTGCAGGAGAC | GTGCTTGTGTCAACACGGAATA |
| | (SEQ ID NO: 53) | (SEQ ID NO: 58) |
| Prf1 | GAGAAGACCTATCAGGACCA | AGCCTGTGGTAAGCATG |
| | (SEQ ID NO: 54) | (SEQ ID NO: 59) |

### ELISAs

High-binding ELISA 96-well plates (Corning) were coated with 50 μl of 100 μg/ml chemo-trypsin (CT) digested gliadin or deamidated gluten peptides (DGP) in 100 mM Na₂HPO₄ overnight at 4° C. Plates were washed three times with PBS 0.05% Tween 20 and blocked with 200 μl of 2% BSA in PBS 0.05% Tween 20 for 2 hours at room temperature. Unlabeled IgG2c or IgG (SouthernBiotech) were used as positive control with 7 concentrations (50 ng/ml highest concentration, 2-fold dilutions). Serum was assessed in duplicate at a 1:100 dilution. Sera were incubated overnight at 4° C., and plates were washed three times with PBS 0.05% Tween 20. Anti-mouse IgG2c, or IgG-horseradish peroxidase (HRP) (SouthernBiotech) in blocking buffer (50 μl at 1/500 dilution) was added to plates and incubated for 1 hour at room temperature. Plates were washed five times with PBS containing 0.05% Tween 20. HRP substrate TMB (50 μl) was added, and the reaction stopped by the addition of 50 μl 2N H₂SO₄. Absorbance was read at 450 nm. Levels of anti-gliadin and anti-DGP antibodies were expressed in OD values.

### Statistical Analysis

Data were first analyzed for normal distribution using D′Agostino and Pearson omnibus normality tests. Normally distributed data was analyzed using unpaired two-tailed Student's t-test for single comparisons, and one-way ANOVA for multiple comparisons. ANOVA analysis was followed by a Tukey's post-hoc test. Not normally distributed data was analyzed using unpaired two-tailed Mann-Whitney U-test for single comparisons, or Kruskal-Wallis test with Dunn's multiple comparison test for comparing more than 2 groups. The statistical test used and P-values are indicated in each figure legend. P-values of <0.05 were considered to be statistically significant. *P<0.05. All tests were performed in GraphPad Prism version 7.04 (GraphPad Software, La Jolla Calif. USA, www.graphpad.com).

### Results

To evaluate if the different strains of L. lactis (expressing dDQ8 alone, or together with IL-2 or IL-10) can induce oral tolerance towards gluten, DQ8-IL15^{LPxIEC} mice were fed a gluten-containing diet and gavage every other day with gliadin for 30 days to induce CeD. Mice were then switched back to a gluten-free diet (GFD) for 30 days to recover, before starting a 21-day daily L. lactis administration on a GFD. Mice were then re-challenged with a gluten containing diet for 21 days, without L. lactis treatment.

Mice were genotyped and distributed equally among the groups based on the DQ8 levels. The number of mice per batch and groups is shown in Table 10. During the L. lactis treatment, there was no treatment-related morbidity or mortality observed in the animals.

TABLE 10

| | LL-empty | | | |
|---|---|---|---|---|
| Batch | vector | LL-[dDQ8] | LL-[dDQ8] + IL2 | LL-[dDQ8] + IL10 |
| 1 | 6 | 4 | 5 | 4 |
| 2 | 3 | 3 | 4 | 4 |
| 3 | 2 | 2 | 2 | 4 |
| Total | 11 | 9 | 11 | 12 |

### Pathology

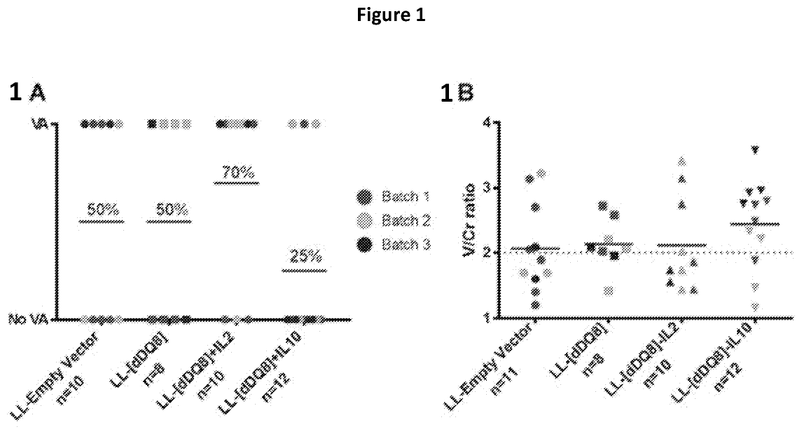

One important determination of CeD in humans is histopathology assessment of small intestinal biopsies (Rubio-Tapia et al., 2013, Am. J. Gastroenterol. 108: 656-676). Therefore, gross pathology was assessed on H&E stained sections to assess the presence (VA) or absence (No VA) of villous atrophy represented by the villous atrophy simple score. Score 0 is no or mild atrophy, while score 2 is severe or partial villous atrophy. This scoring was performed in a blinded manner FIG. 1A shows that 50% of mice treated with the empty vector L. lactis or L. lactis expressing dDQ8, and 70% of the mice in the LL-[dDQ8]+IL-2 group had villous atrophy. Notably, the incidence of villous atrophy was down to 25% in mice treated with LL-[dDQ8]+IL-10.

The villous height to crypt depth ratio (Vh/Cd; “V/Cr” in FIG. 1B) was determined by measuring crypts and villi lengths of up to 6 well-orientated villi per section (FIG. 1B). The mice that received LL-Empty Vector or LL-[dDQ8] had comparable levels of villous atrophy and V/Cr. The V/Cr was higher in the mice treated with LL-[dDQ8]+IL10, while the group administered with LL-[dDQ8]+IL2 had the lowest overall values (not statistically significant). Results obtained from the observation of the ileal sections were in line with the results obtained from the morphometric assessment of the villous height to the crypt depth where a cut-off≤2.0 was used as an indicator of villous atrophy (FIG. 1B): LL-Empty Vector 55%, LL-[dDQ8] 25%, LL-[dDQ8]+IL2 60%, and LL-[dDQ8]+IL10 25% respectively.

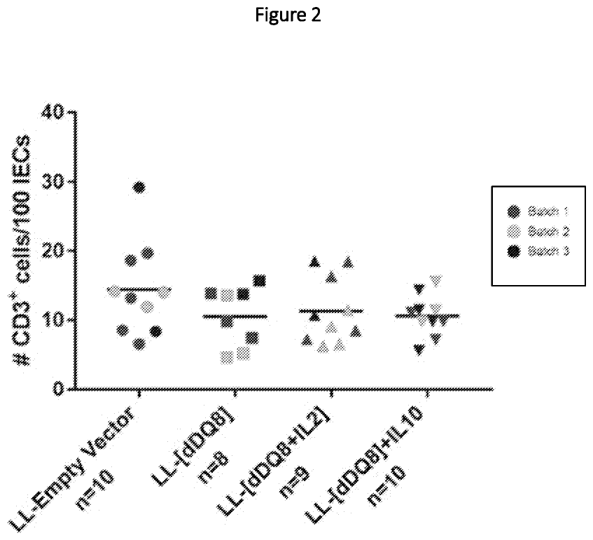

Another hallmark of CeD is intraepithelial lymphocytosis. As shown in FIG. 2, there was an overall, albeit non-significant, decrease in CD3+ IELs in the mice treated with the different strains of LL as compared to the control group.

### Flow Cytometry

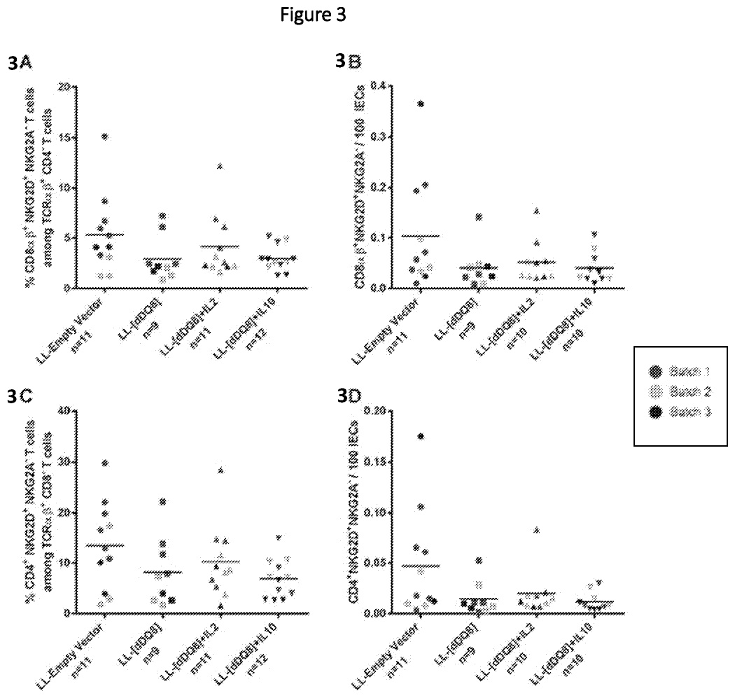

The tissue destruction in CeD is thought to be mediated by cytotoxic CD8+ IELs expressing activating NK receptors such as NKG2D that recognize non classical MHC class I molecules on the surface of epithelial cells (see, e.g., Hüe, et al. 2004, “A Direct Role for NKG2D/MICA Interaction in Villous Atrophy during Celiac Disease,” Immunity 21: 367-377; Meresse, et al, 2006, Reprogramming of CTLs into natural killer—like cells in celiac disease. J. Exp. Med. 203: 1343-1355). As shown in FIG. 3, the frequency of activating NKG2D on CD8αβ⁺ cells was decreased in all LL-[dDQ8] treatment groups compared to empty vector controls. This difference was most clear in the LL-[dDQ8] and LL-[dDQ8]+IL-10 treated groups by both the percentage and absolute number of cells (FIGS. 3A and 3B). These data suggest a trend indicating a decrease in cytolytic T cells in the LL-[dDQ8]+IL-10 treated group. However, differences between the groups were not statistically significant. Similar trends were observed in the CD4 cell compartments (FIGS. 3C and 3D), where differences were also not significant.

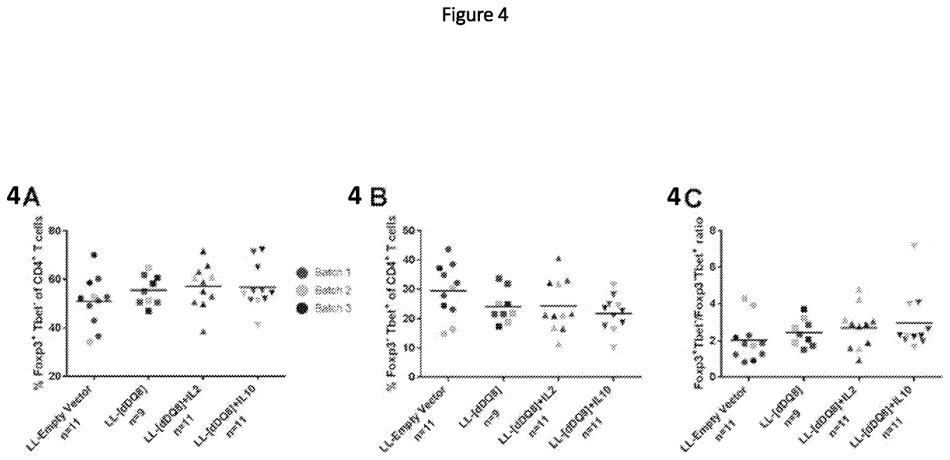

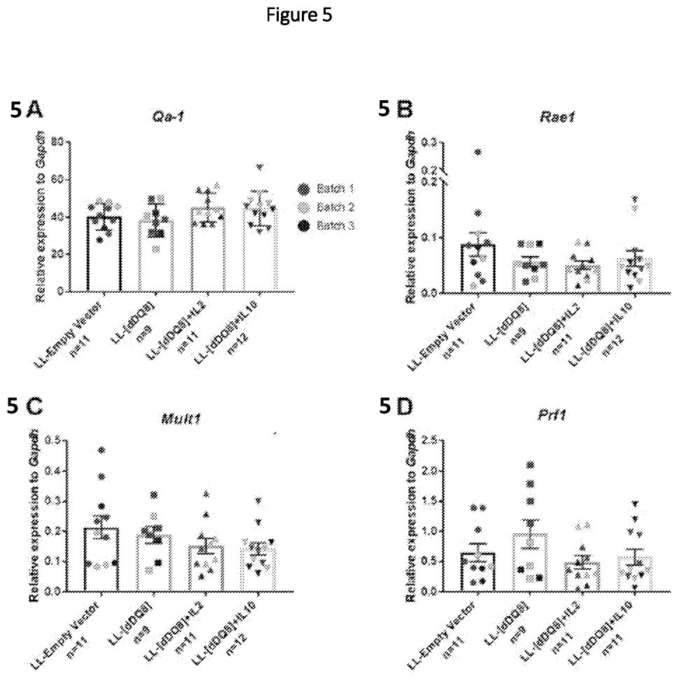

The expression of the regulatory T cell marker Foxp3, as well as the T_{H}1 cell marker Tbet, were also evaluated by flow cytometry. All the LL-[dDQ8]-treated groups had increased frequencies of Foxp3β⁺Tbet⁻ cells (FIG. 4A), and lower levels of Foxp3⁻Tbetβ⁺ cells (FIG. 4B). The ratio of Foxp3β⁺Tbet to Foxp3⁻Tberβ⁺ cells (FIG. 4C) was increased in all LL-[dDQ8]-treated groups, in particular for the LL-[dDQ8]+IL-10 treated group. Thus, a trend for an increase of tolerogenic T cells over pro-inflammatory T cells was observed. However, no statistically significant differences between groups was found.

qPCR

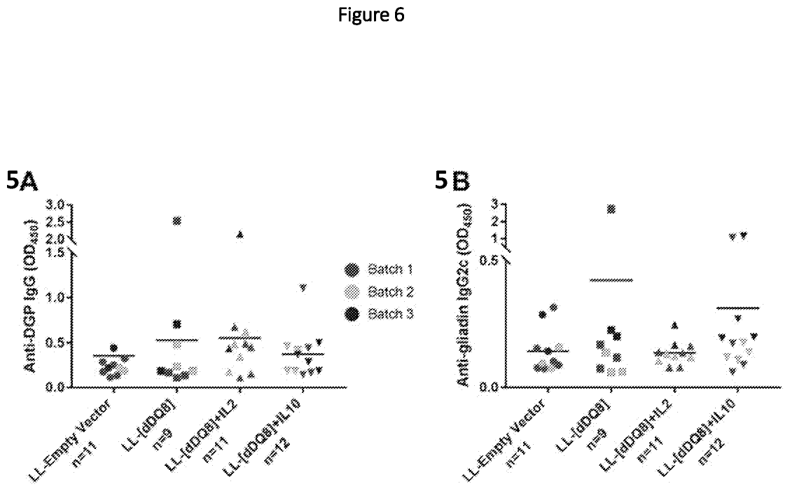

RNA was extracted from isolated epithelial cells and qPCR was performed to evaluate the expression levels of genes encoding Qa-1, and Rae-1 and Mult1, which are epithelial stress markers and ligands for activating NK receptors expressed by IELs, as well as the cytotoxic molecule perforin (Prf1). FIG. 6A shows that the expression levels of Qa-1 were higher for the groups administered with LL-[dDQ8]+IL-2 and LL-[dDQ8]+IL-10, whereas it seemed unaltered for LL-[dDQ8] and the control group. On the other hand, the levels of expression of Rae-1 and Multi were decreased in all the LL-[dDQ8]-treated mice, and in particular in the LL-[dDQ8]+IL-10-treated group (FIGS. 6B and 6C, respectively). Perforin was found to be down-regulated in the LL-[dDQ8]+IL-10 treated group compared to the other groups (FIG. 6D).

### ELISA

The presence of anti-deamidated gluten peptides (DGP) IgG, as well as anti-gliadin IgG2c antibodies was evaluated in the serum by ELISA. No marked differences in the levels of antibodies between groups were observed (FIGS. 6A and 6B, respectively).

### Discussion

The aim of this study was to test if genetically modified L. lactis strains expressing HLA-dDQ8 are capable of restoring oral tolerance towards gluten in a mouse model of CeD. Different strains of L. lactis, expressing either dDQ8 alone, or together with hIL-2 or hIL-10, were used and compared to a control L. lactis containing an empty vector.

At the start of the protocol, mice received a gluten containing diet for 30 days, followed by 30 days recovery on a GFD. Mice were then treated once daily with one of four L. lactis strains (Table 7) for a total of 42 days; first 21 days combined with a GFD, and then 21 days combined with gluten through diet and gliadin gavage. This treatment led to atrophy in the control group (treated with LL-Empty Vector) in 50% of the mice, compared to 50% when treated with LL-[dDQ8], 70% with LL-[dDQ8]+IL-2, and 25% with LL-[dDQ8]+IL-10 (FIG. 2A). These data indicate that the latter treatment was most successful in preventing the onset of gluten-induced atrophy. Villi lengths and crypt depths were quantified and expressed as the Vh:Cd ratio (labeled as “V/Cr” in FIG. 1B). This ratio was in concordance with the simplified atrophy score, as indicated by the higher Vh:Cd ratio in the LL-[dDQ8]+IL-10 treated mice. Overall, the histology data shows a decrease in villous atrophy in the LL-[dDQ8]+IL-10 treated animals.

CeD is also characterized by an increase in intraepithelial lymphocytes. Consistent with a decreased incidence of villous atrophy observed in LL-[dDQ8]+IL-10 treated mice, a reduced infiltration of CD3⁺ intraepithelial lymphocytes was also observed in LL-[dDQ8]+IL-10 treated mice.

Separation of the epithelial and lamina propria compartments allowed for staining and analyzing cell types present in respective compartments. When analyzing the presence of the activating and inhibitory NK receptors NKG2D and NKG2A, respectively, lower percentages and amounts of CD8αβ⁺ and CD4⁺ cells expressing NKG2D (FIGS. 3A and 3B, and FIGS. 3C and 3D, respectively) were observed in all 3 LL-[dDQ8] treatment groups compared to the LL control group. In addition, an overall increased in the frequencies of regulatory T cells was observed in all LL-[dDQ8]-treated groups (FIG. 4A and 4C), while inflammatory CD4⁺Tbet⁺ cells frequencies were decreased (FIG. 4B). The shift from T_{H}1 cells towards Treg cells was more obvious for LL-[dDQ8]+IL-10 treated group (FIG. 4C).

Together, these data show that cytotoxic T cells expressing activating NKG2D are decreased in mice treated with LL-[dDQ8]+IL-2 and LL-[dDQ8]+IL-10. At the same time, inflammatory T cells are less abundant and replaced by regulatory T cells, indicating an environment more prone to tolerance than activation. Interestingly, mice treated with LL-[dDQ8]+IL-2 had overall the least favorable histological outcome as determined by atrophy.

Qa-1 is the murine homologue of HLA-E MHC class I molecule, which preferentially binds CD94/NKG2A, targeting activated lymphocytes (Yu et al., 2018, Recent advances in CD8+ regulatory T cell research. Oncol. Lett. 15: 8187-8194). Therefore, mRNA expression of Qa-1 was assessed. Qa-1 expression is increased in the LL-[dDQ8]+IL-2 and LL-[dDQ8]+IL-10 treated groups compared to controls (FIG. 4A).

Rae1 and Mult1 are NKG2D ligands (Vivier, et al, 2002, Lymphocyte activation via NKG2D: towards a new paradigm in immune recognition? Curr. Opin. Immunol. 14: 306-311; Samarakoon et al., 208 09, Murine NKG2D ligands: “Double, double toil and trouble”, Mol. Immunol. 46: 1011-1019). The levels of expression of Rae1 and Mult1 were down regulated in all LL-[dDQ8] treatment groups compared to LL-Empty vector controls (FIG. 4B and 4C).

Perforin is a cytotoxic molecule (Golstein, et al. 2018, An early history of T cell-mediated cytotoxicity, Nat. Rev. Immunol. 18: 527-535; Voskoboinik, et al., 2015, Perforin and granzymes: function, dysfunction and human pathology, Nat. Rev. Immunol. 15: 388-400). Therefore, expression of Prf1 was also assessed. Prf1 was down-regulated in mice treated with LL-[dDQ8]+IL-10, while the other groups are largely comparable to control values (FIG. 4D).

On whole, and consistent with a decrease in the amount of cytotoxic lymphocytes, these biomarker expression results point out a beneficial effect of the administration of LL-[dDQ8] that lower the threshold of activation at the epithelial level.

In conclusion, these data indicate that LL-[dDQ8]+IL-10 was capable of reducing disease burden in treated DQ8-IL15^{LPxIEC} animals compared to the other groups, with the most supportive factor being reduced villous atrophy. Decreased cells with activating NKG2D in the CD4⁺ and CD8αβ⁺ populations, with increased percentages of Foxp3⁺ Tregs, were observed. On the transcriptional level, increased expression of NKG2D inhibitory factor Qa-1, and decreased levels of NKG2D activating factors (Rael, Multi), as well as decreased Prfl levels, was observed, and which was most apparent in the LL-[dDQ8]+IL-10 treated group. Even though the results did not achieve statistical significance, most parameters analyzed suggest that the LL-[dDQ8]+IL-10 treatment may be beneficial and has the potential to prevent villous atrophy in a therapeutic scheme mimicking CeD patients on a gluten-free diet (GFD) that are challenged for a prolonged period with high amounts of gluten (similar ones found in a normal diet). Although the LL-[dDQ8] treatment was able to some extent to prevent villous trophy after gluten challenge, this effect was less marked than the LL-[dDQ8]+IL-10 treatment. In addition, there was no obvious effect on any of the inflammatory markers tested.

### Example 2: Treatment of Celiac Disease in Mice

This experiment describes a further in vivo interventional study, i.e., starting L. lactis treatment after initiation of the disease using gluten treatment. The previous study in this mouse model showed that LL-[dDQ8]+IL10 was most efficacious in preventing villus atrophy. In this study, it was further investigated whether a 21 day treatment was enough and sufficient to achieve similar results to the previous study, in which LL was administered for 42 days. In addition, LL expressing only IL10 was included to evaluate the necessity of dDQ8 in the restoration of oral tolerance towards gluten, thereby preventing the recurrence of CeD-like pathology in DQ8-IL15^{LPxIEC} mice.

### Materials and Methods

Abbreviations used in this example are presented in Table 6 in Example 1.

### Overview of Experiment

DQ8-IL15^{LPxIEC} mice were exposed to gluten for 30 days, recovered 30 days on a gluten free diet (GFD), and then were administered one of 3 strains of L. lactis every day, while the mice were maintained on a GFD for 21 days. Mice were then re-challenged with a gluten-containing diet for 21 days, without LL treatment.

As in Example 1, at the end of each experiment, mice were euthanized and small intestines were processed for histology (Hematoxylin and Eosin (H&E) staining for pathology, CD3 immunostaining for intraepithelial lymphocytes (IELs) counts), lamina propria (LP) and epithelium isolation for fluorescence-activated cell sorting (FACS) analysis for markers of activation of IELs. The levels of gene expression for epithelial stress markers and cytotoxic molecules were evaluated by quantitative polymerase chain reaction (qPCR).

### Mice

DQ8-IL15^{LPxIEC} mice used in this experiment are described in the Example 1.

DQ8-IL15^{LPxIEC} mice were 9 weeks of age at the start of experiment, and both male and female mice were used. Mice were kept on a gluten free diet (Research Diets, AIN-76A) until the start of the experiments, when gluten was introduced to the diet to induce CeD. In addition to a gluten-containing diet, mice were administered approximately 20 mg gliadin (Sigma, G3375) by gastric gavage every other day during this diet.

All animal procedures were reviewed by the local ethical committee of the University of Chicago, ACUP 71966.

Lactococcus lactis Strains and Culture

The efficacy of L. lactis strains secreting IL-10, with or without co-secreted deamidated HLA-DQ8 peptide (Table 11) was examined

TABLE 11

| L. lactis strain | Common name | Description |
|---|---|---|
| L. lactis-pT1NX | LL-empty vector | L. lactis strain with |
| | | plasmid backbone |
| sAGX0487 | LL-IL10 | L. lactis strain with |
| | | with human IL-10 |
| | | gene integrated |
| | | in the genome |
| sAGX0487[pAGX2263] | LL-[dDQ8] + IL-10 | L. lactis strain with |
| | | human IL-10 gene |
| | | integrated in the |
| | | genome and plasmid- |
| | | encoded deamidated |
| | | HLA-DQ8-peptide |

L. lactis-pT1NX is the same one used in Example 1; it is an MG1363 strain containing the empty vector pT1NX, and served as control.

In sAGX0487 (L. lactis subsp. cremoris MG1363: ΔthyA; eno>>SSusp45-hil-10; usp45>>otsB; ΔtrePP; PhllA>>trePTS; ΔptcC-):
Thymidylate synthase gene (thyA; Gene ID: 4798358) is absent, to warrant environmental containment.
Trehalose-6-phosphate phosphorylase gene (trePP; Gene ID: 4797140) is absent, to allow accumulation of exogenously added trehalose.
Trehalose-6-phosphate phosphatase gene (otsB; Gene ID: 1036914) is positioned downstream of usp45 (Gene ID: 4797218) to facilitate conversion of trehalose-6-phosphate to trehalose. The otsB expression unit was transcriptionally and translationally coupled to usp45 by use of the intergenic region (IR) preceding the highly expressed L. lactis MG1363 50S ribosomal protein L30 gene (rpmD; Gene ID: 4797873).
The constitutive promoter of the HU-like DNA-binding protein gene (PhllA; Gene ID: 4797353; Locus tag LLMG_RS02525) is preceding the putative phosphotransferase genes in the trehalose operon (trePTS; llmg_0453 and llmg_0454; Gene ID: 4797778 and Gene ID: 4797093 respectively) to potentiate trehalose uptake.
The gene encoding cellobiose-specific PTS system IIC component (ptcC; GeneID: 4796893) is disrupted (tga at codon position 30 of 446; tga30). This mutation ascertains trehalose retention after accumulation.
A gene encoding a fusion of usp45 secretion leader (SSusp45) with the hil-10 gene, encoding human interleukin-10 (hIL-10; UniProt: P22301, aa 19-178, variant P2A) is positioned downstream of the phosphopyruvate hydratase gene (eno; Gene ID: 4797432), to allow expression and secretion of hIL-10.
   When grown in the presence of trehalose, sAGX0487 accumulates and retains trehalose, which provides protection from bile acid toxicity. Furthermore, sAGX0487 constitutively expresses and secretes hIL-10.

The plasmid-driven L. lactis strain sAGX0487[pAGX2263] is the same one used in Example 1.

The strain culture conditions are the same as described in Example 1.

Mouse dissection and cell isolation, Pathology, Antibodies and flow cytometry, RNA isolation and qPCR methods used in this Example are the same as described in Example 1.

### Statistical Analysis

Data were first analyzed for normal distribution using D'Agostino and Pearson omnibus normality tests. Normally distributed data was analyzed using unpaired two-tailed Student's t-test for single comparisons, and one-way ANOVA for multiple comparisons. ANOVA analysis was followed by a Tukey's post-hoc test. The statistical test used and P-values are indicated in each figure legend. P-values of <0.05 were considered to be statistically significant. *P<0.05. **P,0.01. All tests were performed in GraphPad Prism version 7.04 (GraphPad Software, La Jolla California USA, www.graphpad.com).

### Results

To evaluate if the different strains of L. lactis (expressing IL-10 alone, or together with dDQ8) can induce oral tolerance towards gluten, DQ8-IL15^{LPxIEL} mice were fed a gluten-containing diet and gavage every other day with gliadin for 30 days to induce CeD (Days 0-30). Mice were then switched back to a GFD for 30 days to recover (Days 31-59), before starting a 21-day daily L. lactis administration on a GFD (Days 60-81). Mice were then re-challenged with a gluten containing diet for 21 days, without L. lactis treatment (Days 82-102).

Mice were genotyped and distributed equally among the groups based on the DQ8 levels. The number of mice per batch and groups is shown in Table 12. During the L. lactis treatment, there was no treatment-related morbidity or mortality observed in the animals.

TABLE 12

| | | LL- | | | LL- | LL- | |
|---|---|---|---|---|---|---|---|
| | | [dDQ8] + | | | empty | [dDQ8] + | LL- |
| Batch | Vehicle | IL10 | GFD | Vehicle | vector | IL10 | IL10 |
| 5 | | | | 5 | | 4 | |
| 6 | | | | 6 | | 6 | |
| 7 | | | 3 | 4 | 4 | 3 | 3 |
| 9 | 4 | 3 | 2 | | 1 | 2 | 3 |
| Total | 4 | 3 | 5 | 15 | 5 | 15 | 6 |

| | | |
|---|---|---|
| Never gluten | Gluten | Gluten initiation + challenge |
| | initiation | |

### Pathology

One important determination of CeD in humans is histopathology assessment of small intestinal biopsies (Rubio-Tapia et al., 2013, Am. J. Gastroenterol. 108:656-676). Therefore, gross pathology was assessed on H&E stained sections to assess the presence (VA) or absence (No VA) represented by the villous atrophy simple score. Score 0 is no or mild atrophy, while score 2 is severe or partial villous atrophy. This scoring was performed in a blinded manner

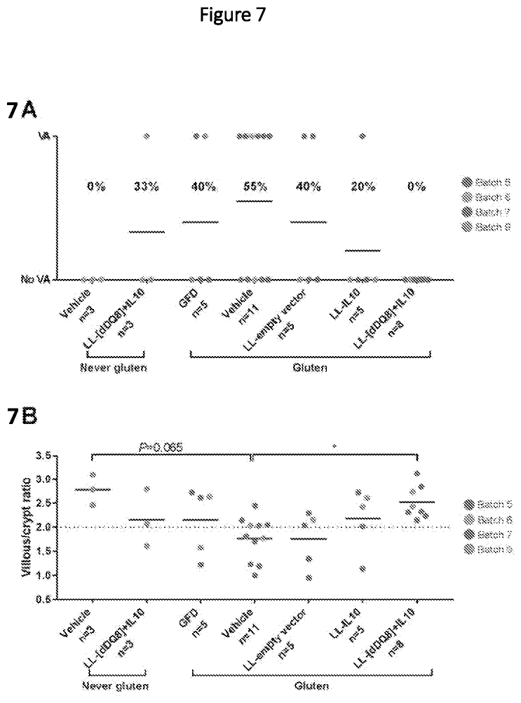

The villous atrophy data are depicted in FIG. 7A. In the two groups that never received gluten, atrophy was present in 0% and 33% of mice treated with vehicle or LL-[dDQ8]+IL10 respectively. Of the mice that received the gluten-initiation, but not the final gluten-challenge, 40% developed atrophy. Of the mice that received the gluten initiation and the final gluten challenge, atrophy was present in 55% and 40% in the vehicle-treated and LL-empty vector treated control groups, respectively. Of the mice that received the gluten initiation, treatment with LL-IL10 and the final gluten challenge, atrophy was present in 20%. Notably, of the mice that received the gluten initiation, treatment with LL-[dDQ8]+IL10 and the final gluten challenge, no atrophy was present.

The villous height to crypt depth ratio (V/Cr) was determined by measuring crypts and villi lengths of up to 6 well-orientated villi per section. The data are shown in FIG. 7B. Results obtained from the observation of the ileal sections were in line with the results obtained from the morphometric assessment of the villous height to the crypt depth where a cut-off ≤2.0 was used as an indicator of villous atrophy. This resulted in the following atrophy levels per group in the mice never having received gluten: vehicle 0%, LL-[dDQ8]+IL10 33%. In the mice that received gluten this was: GFD 40%, vehicle 55%, LL-Empty Vector 40%, LL-IL10 20%, and LL-[dDQ8]+IL10 0% respectively.

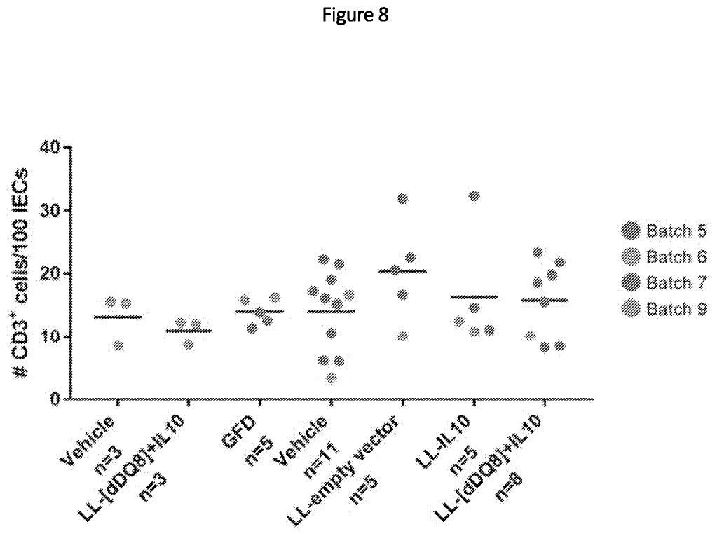

Another hallmark of CeD is intraepithelial lymphocytosis. As shown in FIG. 8, the data of CD3 counts on histological sections does not show any clear differences among the groups.

### Flow Cytometry

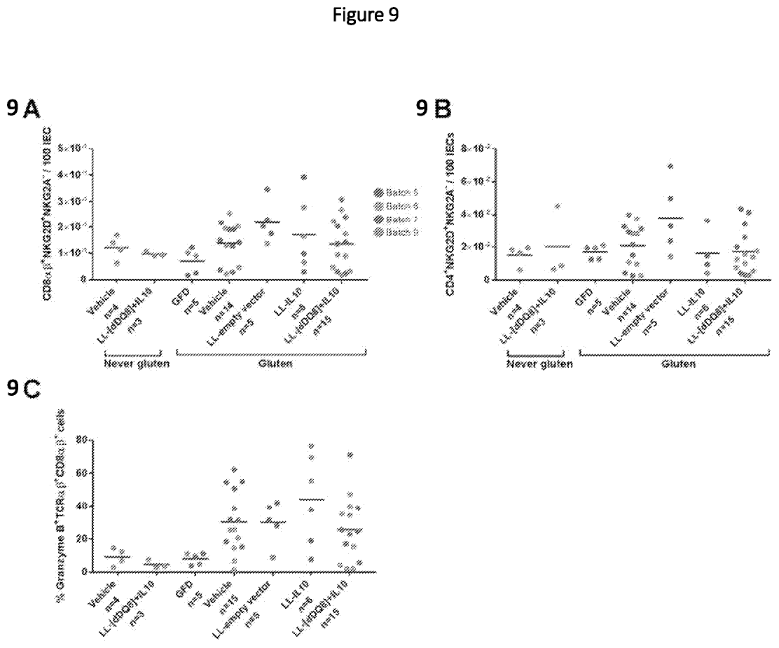

The tissue destruction in CeD is thought to be mediated by cytotoxic CD8+ IELs expressing activating NK receptors such as NKG2D that recognize non classical MHC class I molecules on the surface of epithelial cells (see, e.g., Hüe, et al. 2004, Immunity 21: 367-377; Meresse, et al, 2006 J. Exp. Med. 203:1343-1355). As shown in FIG. 9A, the absolute numbers of CD8+NKG2D+ cells (determined by the number of CD3+ cells by histology and the frequencies on FACS) were higher in mice that received the final gluten challenge than those who did not, but there was no clear trend for lower numbers in LL-IL10 or LL-[dDQ8]+IL10 treated mice and the differences were not statistically significant. The NKG2D+ population of CD4+ was unaltered between the never gluten and gluten receiving mice, and there were no clear differences between the control groups and LL-treated mice (FIG. 9B). Finally, the expression of granzyme B by CD8+ cells was low in the mice that never received gluten, and higher in comparison in the mice that received gluten, both only at start and final gluten challenge (FIG. 9C). No clear differences were observed between controls and LL-IL10 or LL-[dDQ8]+IL10 treated mice.

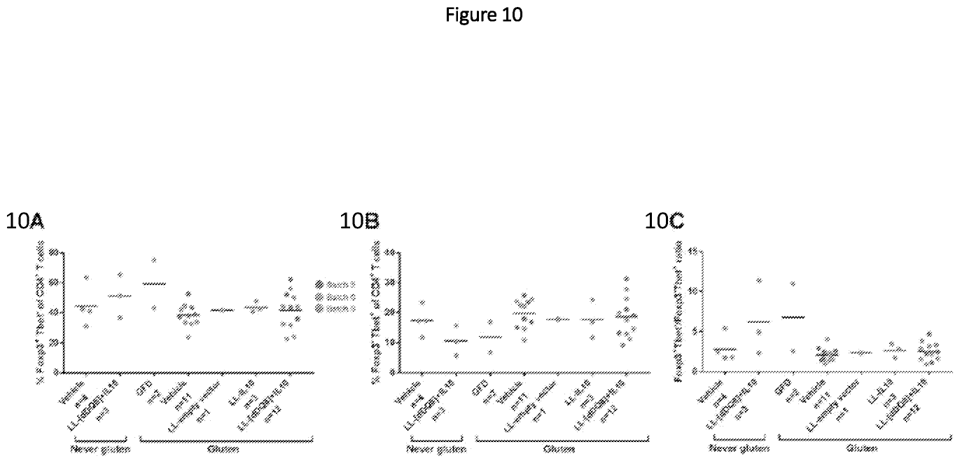

The expression of the regulatory T cell marker Foxp3, as well as the T_{H}1 cell marker Tbet, were also evaluated by flow cytometry. No clear trends are detectable in the Foxp3⁺Tbet⁻ T cells (FIG. 10A) or in the Foxp3⁻Tbet⁺CD4⁺ population (FIG. 10B) or the ratio of Foxp3⁺Tbet⁻ ove Foxp3⁻Tbet⁺ CD4⁺ cells (FIG. 10C). There were no statistically significant differences between the groups.

qPCR

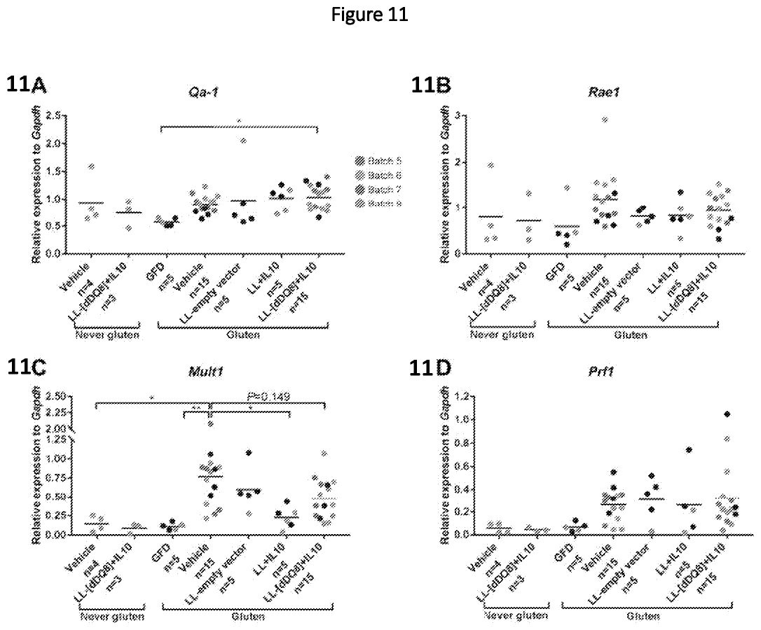

RNA was extracted from isolated epithelial cells (cell fraction before Percoll separation) and qPCR was performed to evaluate the expression levels of genes encoding Qa-1, and Rae-1 and Mult1, which are epithelial stress and ligands for activating NK receptors expressed by IELs. Expression of cytotoxic molecule perforin by IELs was evaluated in the cell fraction after Percoll separation. The data are shown in FIG. 11.

FIG. 11A shows that the expression levels of Qa-1 were slightly elevated in the groups administered with LL compared to the vehicle treated group, and also in the LL-IL10 and LL-[dDQ8]+IL10 groups vs. LL-empty vector. The only significant up-regulation detected was between the GFD group and LL-[dDQ8]+IL10 treated animals The expression levels of Rae-1 were decreased in all the LL-treated mice, but no significant differences were detected when comparing LL-IL10 and LL-[dDQ8]+IL10 groups versus LL-empty vector (FIG. 11B). The LL-[dDQ8]+IL10 group's expression was slightly higher compared to LL-empty vector treated mice. Mult1 expression was increased in the gluten-treated mice compared to all groups not subjected to the final gluten challenge (GFD group) or that never received gluten (FIG. 11C). The Mult1 expression was decreased in the LL-IL10 and LL-[dDQ8]+IL10 treated mice, when compared to vehicle treated mice, as well as to LL-empty vector; the decrease was most striking in the LL-IL10 treated group, which was significant, when compared to the vehicle treated group (FIG. 11C). Overall, none of the groups reached the low levels of Multi expression that were seen in mice that never received gluten or did not receive the final gluten challenge. FIG. 11D depicts the expression of Prf1 data. Expression of Prf1 was low in the mice that never received gluten, or the mice that did not receive the gluten free challenge (GFD group). Gluten-treatment increased Prf1 expression in all groups, and the level of expression was not significantly altered between the vehicle control group and any of the three LL-receiving groups of mice, nor between the LL-empty vector group and either of the LL-IL10 or LL-[dDQ8]+IL10 groups.

### Discussion

The aim of this study was to test whether genetically modified L. lactis strains expressing hIL10 alone, or together with HLA-dDQ8, are capable of restoring oral tolerance towards gluten in a mouse model of CeD. In this second PD study, the focus was on the efficacy of a 21 days treatment with LL, whereas in PD-1 (Example 1) a 42-day treatment was evaluated. Besides the control L. lactis containing an empty vector, vehicle treated mice were included.

At the start of the protocol, mice received a gluten containing diet for 30 days, followed by 30 days recovery on a GFD. Mice were then treated once daily with L. lactis strains (Table 11) for a total of 21 days on a GFD. Mice were then re-challenged with gluten for 21 days, through diet and gliadin gavage. For further experimentation, one can perform RNAseq analysis on mice that never received gluten (sham fed), and a group that never received gluten and was administered with LL-[dDQ8]+IL10.

Results show presence of villous atrophy in the vehicle treated control group at 55%, compared to 40% when treated with the LL-empty vector control strain. Treatment with LL-IL10 reduced atrophy in mice to 20%, and atrophy was not present in mice treated with LL-[dDQ8]+IL10 (FIG. 7A), indicating that the latter treatment was most successful in preventing the onset of gluten-induced villous atrophy. In the mice that had never received gluten, the vehicle treated mice did not develop atrophy, while in the LL-[dDQ8]+IL10 treated mice 1 out of 3 developed villous atrophy; the latter result may occur given the genetic background of the mice overexpressing IL-15. In the GFD group, which did not receive the final gluten challenge, 2 of 5 mice still had atrophy.

Villi lengths and crypt depths were quantified and expressed as the V:Cr ratio, and this ratio was in concordance with the simplified atrophy score, as indicated by the higher V:Cr ratio in the LL-[dDQ8]+IL10 treated mice (FIG. 7B). Overall, the histology data shows a decrease in villous atrophy in the LL-IL10 and LL-[dDQ8]+IL10 treated animals, with highest efficacy in the latter group.

CeD is also characterized by an increase in intraepithelial lymphocytes, though in this study no clear differences in CD3+ IELs on histology were found.

Separation of the epithelial and lamina propria compartments allowed for staining and analyzing cell types present in respective compartments. When analyzing the presence of the activating and inhibitory NK receptors NKG2D and NKG2A, respectively, no differences in the numbers of CD8αβ⁺ and CD4⁺ cells expressing NKG2D (FIGS. 9A and 9B) in LL-IL10 or LL-[dDQ8]+IL10 treatment groups compared to the LL control groups were found. Granzyme B was also analyzed and showed only a clear difference between the mice that did not receive gluten, and those with a gluten challenge. Granzyme B+ CD8 cells in the treatment groups (LL-IL10 or LL-[dDQ8]+IL10) were not different in numbers from vehicle and LL-empty vector control groups (FIG. 9C).

In the lamina propria lymphocytes, a very minor increase in frequencies of regulatory T cells was observed in the LL-IL10 and LL-[dDQ8]+IL10 treated groups (FIG. 10A and 10C), while inflammatory CD4⁺Tbet³⁰ cells frequencies were somewhat decreased (FIG. 10B), which represent desirable outcomes for treatment. In Example 1, where the LL treatment duration was 42 days, a somewhat stronger reduction in NKG2D³⁰ cells, and increases in Foxp3³⁰ cells (FIGS. 4A-4C), was observed, but similar to this Example, no statistically significant results were detected.

mRNA expression of Qa-1, the murine homologue of HLA-E MHC class I molecule, which preferentially binds CD94/NKG2A, targeting activated lymphocytes was assessed. Qa-1 expression is slightly increased in the LL-IL10 and LL-[dDQ8]+IL10 treated groups compared to vehicle treatment and also versus LL-empty vector treated mice (not significant; FIG. 11A). The levels of expression of Rae1, a NKG2D ligand, was down regulated in all LL-treatment groups compared to vehicle control, but there was no difference in expression between the control LL strain (LL-empty vector) and LL-IL10 or LL-[dDQ8]+IL10 (FIG. 11B). Mult1, another NKG2D ligand, is also decreased in LL-treated groups versus vehicle-treated mice. This decrease is most evident in the LL-IL10 treated group and also versus. LL-empty vector; this decrease is also present in the LL-[dDQ8]+IL10 treated mice, but less pronounced (FIG. 11C). Lastly, the expression of the cytotoxic molecule perforin was assessed in the IEL fraction after Percoll purification. Prf1 is low in mice that had never received gluten, or did not receive the gluten challenge (FIG. 11D). Between the mice that had received gluten, there were no clear or statistically significant differences (FIG. 11D). Overall, the effect of LL-[dDQ8]+IL10 on expression of these biomarkers was less strong than the results observed in Example 1, and in particular versus the LL-empty vector control group. Even though there were no statistically significant differences detected in Example 1, the trends in Qa-1, and Rae1 and Mult1 expression were slightly clearer.

In conclusion, these data indicate that LL-[dDQ8]+IL10 was capable of reducing disease burden in treated DQ8-IL15^{LPxIEC} animals compared to the other groups, with the most supportive factor being reduced villous atrophy. While a decrease of activating NKG2D on CD8⁺ and CD4⁺ T cells was not detected as after a longer treatment period of 42 days (Example 1), at the transcriptional level an increased expression of NKG2D inhibitory factor Qa-1, and decreased levels of NKG2D activating factors (Rae1, Mult1) was found, which was most apparent in the LL-[dDQ8]+IL10 treated group. The LL-IL10 treatment reduced villous trophy after gluten challenge, however this effect was less marked than the LL-[dDQ8]+IL10 treatment, indicating a possible requirement for further tolerization or immune inactivation by the co-presentation of dDQ8.

Even though the results did not achieve statistical significance, most parameters analyzed suggest that the LL-[dDQ8]+IL10 treatment is beneficial and has the potential to prevent villous atrophy in a therapeutic scheme mimicking CeD patients on a GFD that are challenged with gluten.

### Example 3: Secretion Leaders for DQ2 and dDQ2 Epitopes in L. lactis Strains

Identification of appropriate combinations of secretion leaders for HLA-DQ2 epitopes is an important factor for the design and development of clinical strains, in order to efficiently express the HLA-restricted epitopes, DQ2 and dDQ2. This experiment is directed to identifying secretion leaders with suitable adequate secretion, and also, to see if secretion leaders having improved secretion relative to the secretion leader (SSusp45) from unidentified secreted 45 kDa protein precursor (Usp45; UniProt P22865) could be identified.

This experiment is designed to identify secretion leaders for HLA-DQ2 epitopes. The immunodominant site for DQ2.5 is on α2-gliadin (Alpha-gliadin; UniProt Q9M4L6_wheat). The site is a protease-resistant 33-mer (LQLQPFPQPQLPYPQPQLPYPQPQLPYPQPQPF (SEQ ID NO: 3); DQ2) that has 6 overlapping DQ2.5 restricted epitopes. The majority of the HLA-DQ2 restricted T-cell responses are against the deamidated 33-mer (LQLQPFPQPELPYPQPQLPYPQPELPYPQPQPF (SEQ ID NO: 7); dDQ2). Both DQ2 and dDQ2 peptide sequence was reverse translated to obtain DQ2 and dDQ2 coding sequences, using preferred L. lactis codon usage, which are shown in Table 13. The sequences were produced as synthetic DNA.

TABLE 13

| | | SEQ ID |
|---|---|---|
| Target | Synthetic DNA Coding Sequence | NO: |
| DQ2 | CTTCAACTTCAACCATTTCCACAACCACAACTTCCATACCCACAA | 60 |
| | CCACAACTTCCATACCCACAACCACAACTTCCATACCCACAACCA | |
| | CAACCATTTTAA | |
| dDQ2 | CTTCAACTTCAACCATTTCCACAACCAGAACTTCCATACCCACAA | 61 |
| | CCACAACTTCCATACCCACAACCAGAACTTCCATACCCACAACCA | |
| | CAACCAT TTTAA | |

Candidate secretion leader sequences for testing were obtained by identifying L. lactis MG1363 proteins in public databases predicted to be extracellular; the public databases were: PSORTdb (db.psort.org/browse/genome?id=8347) (Peabody et al, 2016, PSORTdb: expanding the bacteria and archaea protein subcellular localization database to better reflect diversity in cell envelope structures, Nucleic Acids Res. 44(D1):D663-8; Yu et al., 2011, PSORTdb—an expanded, auto-updated, user-friendly protein subcellular localization database for Bacteria and Archaea, Nucleic Acids Res. 39:D241-244 (Database issue); Rey et al, 2005, PSORTdb: A Database of Subcellular Localizations for Bacteria, Nucleic Acids Res. 33:D164-168 (Database issue) and UniProt database, searching for sequences in L. lactis MG1363 predicted to have signal peptide sequences. Thirty-six (36) predicted secretion leader (SL) sequences were identified. The sequences and UniProt number and name of the parent protein are provided in Table 14. A mutant version of A2RHV3 and two mutants of P22865 were also identified. Without being limited by theory, it is believed that an error in the synthetic DNA probably gave a selective advantage to the recombinant molecule so it could be isolated. A2RIG7 (numbers 15 and 18) was inadvertently included in the list twice and was tested in duplicate. Two mutants of P22865 were also identified.

TABLE 14

| | | | | SEQ ID | SEQ ID |
|---|---|---|---|---|---|
| | | | Predicted Secretion | NO: | NO: |
| SL# | UniProt | Name | Leader Sequence | (PRT) | (DNA) |
| 1 | A2RHI3 | putative | MKKRVQRNKKRIRWASV | 34 | 86 |
| | | xylanase/chitin | LTVFVLLIGIIAIAFA | | |
| | | deacetylase | | | |
| 2 | A2RHV3 | putative secreted | MSITATIAAGATALTLLGA | 62 | 87 |
| | | protein | GGAAA | | |
| 3 | | | MSITATIAAGATALTLLGA | 63 | 88 |
| | | | GGAAAVNA | | |
| 4 | A2RHZ5 | N- | MPVSRVKVKNRHLKKKT | | |
| | | acetylglucosaminidase | KKPLAFYKPATKFAGAVL | | |
| | | | IAGTLTTTHELLLQQTSPM | 64 | 89 |
| | | | VQA | | |
| 5 | A2R107 | endo-1,4-beta- | MSQKRSARSKSSKK | 65 | 90 |
| | | xylanase D | | | |
| 6 | A2RIL8 | N- | MKQKHKLALGASIVALAS | 35 | 91 |
| | | acetylglucosaminidase | LGGIKAQA | | |
| 7 | A2RK75 | putative secreted | MTPKTKAAVLTGTIDSTG | 66 | 92 |
| | | protein | AVTGVTG | | |
| 8 | A2RKE6 | sugar ABC | MNLAKNWKSFALVAAGA | 36 | 93 |
| | | transporter | IAVVSLAACGKSA | | |
| | | substrate-binding | | | |
| | | protein | | | |
| 9 | A2RLK0 | gamma- | MLKKIIISAALMASLSAAM | 37 | 94 |
| | | glutamyl- | IANPAKA | | |
| | | diamino acid- | | | |
| | | endopeptidase | | | |
| 10 | Q8KKF9 | N- | MVNTQVKRVKKQKFIAG | 67 | 95 |
| | | acetylmuramoyl- | TALLLGMATFGMVGKA | | |
| | | L-alanine | | | |
| | | amidase | | | |
| 11 | A2RN73 | hypothetical | MLLSVLPVNLLGVMKVD | 68 | 96 |
| | | protein | A | | |
| | | llmg_2194 | | | |
| 12 | A2RN78 | acidic | MISVKKRKNIKVFLITASI | 69 | 97 |
| | | endochitinase | GIVALGGQRVLADA | | |
| | | precursor | | | |
| 13 | P22865 | secreted 45 kDa | MKKKIISAILMSTVILSAA | 38 | 98 |
| | | protein precursor | APLSGVYA | | |
| 14 | A2RHU8 | hypothetical | MIKLKKSHIISLILFSGLLLV | 70 | 99 |
| | | protein | EPVLA | | |
| | | llmg_0229 | | | |
| 15 | A2RIG7 | hypothetical | MKKIIYGVGLISLLNVGTI | 39 | 100 |
| | | protein | AYG | | |
| | | llmg_0458 | | | |
| 16 | A2RL19 | hypothetical | MKIKNLLMAATTVATLG | 71 | 101 |
| | | protein | AIGTVSAQASA | | |
| | | llmg_1399 | | | |
| 17 | A2RI74 | dipeptide- | MKQAKIIGLSTVIALSGIIL | 40 | 102 |
| | | binding protein | VACGSKT | | |
| | | precursor | | | |
| 18 | A2RIG7 | hypothetical | MKKIIYGVGLISLLNVGTI | 39 | 100 |
| | | protein | AYG | | |
| | | llmg_0458 | | | |
| 19 | A2RIL6 | peptide binding | MNKSKIIAFSAVSLSAALL | 72 | 103 |
| | | protein | LTACGNSSS | | |
| 20 | A2RIV4 | hypothetical | MKKFLLLGATALSLFSLA | 41 | 104 |
| | | protein | ACSSSN | | |
| | | llmg_0601 | | | |
| 21 | A2RJJ4 | ps356 endolysin | MKKVIKKAAIGMVAFFVV | 42 | 105 |
| | | | AASGPVFA | | |
| 22 | A2RJL9 | hypothetical | MSKKSIKKITMTVGVGLL | 43 | 106 |
| | | protein | TAIMSPSVINQ | | |
| | | llmg_0877 | | | |
| 23 | A2RJP5 | hypothetical | MRHKKIYLLLAMIGATSA | 44 | 107 |
| | | protein | WTVANENQVKA | | |
| | | llmg_0904 | | | |
| 24 | A2RJQ9 | hypothetical | MKKFVLIILLLFSSSILLAD | 45 | 108 |
| | | protein | KSSA | | |
| | | llmg_0918 | | | |
| 25 | A2RK78 | hypothetical | MKIKYILWVICALLLLNTG | 46 | 109 |
| | | protein | PSFA | | |
| | | llmg_1094 | | | |
| 26 | A2RKB1 | cell wall surface | MEMQKKKAPRKKGKVIT | | |
| | | anchor family | KRKVLSATMSGTLLMTSV | 73 | 110 |
| | | protein | IIPTAYSLLSNQITAKA | | |
| 27 | A2RKT3 | hypothetical | MKFNKKRVAIATFIALIFV | | |
| | | protein | SFFTISSIQDNQTNA | 74 | 111 |
| | | llmg_1306 | | | |
| 28 | A2RL18 | cell surface | MKKTLRDQLLGVSKAHL | | |
| | | antigen I/II | NWKNKTKVFIYGTAILLM | 75 | 112 |
| | | precursor | VAPNLASSVSRASA | | |
| 29 | A2RLU8 | hypothetical | MKSPSKFWLLSTGILLSLL | | |
| | | protein | VTSLPLAVKA | 76 | 113 |
| | | llmg_1698 | | | |
| 30 | A2RM44 | hypothetical | MSILAFALVLIFGFVSQNA | | |
| | | protein | FA | 77 | 114 |
| | | llmg_1800 | | | |
| 31 | A2RM46 | hypothetical | MKLNSLNKKFALASVSLL | | |
| | | protein | TISTLAGFGGLVNVNA | 78 | 115 |
| | | llmg_1802 | | | |
| 32 | G0WJN9 | oligopeptide- | MNKLKVTLLASSVVLAAT | | |
| | | binding protein | LLSACGSNQSSS | 47 | 116 |
| | | oppA | | | |
| 33 | A2RME7 | foldase prsA | MKFKKLGLVMATVFAGA | 79 | 117 |
| | | | ALVTLSGCSSSDS | | |
| 34 | P22865 | secreted 45 kDa | MKKKIISAILMSTVILSAA | | |
| | | protein precursor | APLSGVYA | 38 | 118 |
| 35 | P22865* | secreted 45 kDa | MKKKIISAILMSTVILSAA | | |
| | | protein precursor + | APLSGVYAG | 48 | 119 |
| | | Glycine | | | |
| 36 | P22865** | secreted 45 kDa | MKKNIISAILMSTVILSAA | | |
| | | protein precursor | APLSGVYA | 49 | 120 |
| | | K4N mutation | | | |

The DQ2 and dDQ2 coding sequences were linked in-frame (i.e., operably linked) to coding sequences of the 3′ end of a collection of 36 selected L. lactis secretion leaders, to form the configuration SL::DQ2 and SL::dDQ2. The SL::DQ2 and SL::dDQ2 coding sequences were positioned at appropriate distance downstream of the L. lactis hllA gene promoter (PhllA) to obtain PhllA>>SL::DQ2 and PhllA>>SL::dDQ2, thus creating modules for the expression and secretion of DQ2 and dDQ2. These modules were cloned into erythromycin selectable L. lactis plasmids and transformed to L. lactis to obtain LL[PhllA>>SL::DQ2] and LL[PhllA>>SL::dDQ2], designated as pAGX2211 and pAGX2212, respectively. MG1363[pAGX0043], which is an L. lactis strain comprising a plasmid expressing SL::DQ2 wherein the SL is SSusp45 and expression is under control of promoter P1 (P1>>SSusp45-DQ2), was used as a positive control.

Approximately 6000 colonies were obtained after bulk transformation of L. lactis, and approximately 600 clones were tested using six 96 well plates each for DQ2 or dDQ2 secretion by ELISA. Hypothetically, each 96-well plate contains a pool of six different secretion leaders (see Tables Ex. J and K). In brief, Nunc MaxiSorp™ F96 plates (Thermo Fisher Scientific, Waltham, Mass.; #442404) were coated with crude supernatant and incubated overnight. After blocking with 0.1% casein in PNS, rabbit DQ2 antiserum (Thermo #OR245368_2), anti-rabbit HRP (Southern Biotech #4030-05) and TMB Chromogen solution (Thermo Fisher Scientific #002023) were used for detection. The reaction was stopped by adding 1M hydrochloric acid (HCl), and the absorbance was read at 450 nm for measuring and 595 nm as reference. MG1363[pAGX0043] was used as positive control (A1 on each 96 well plate), and MG1363[pT1NX] (which is L. lactis strain with plasmid backbone, i.e., L. lactis with an empty vector) was used as the negative control (A2 on each 96-well plate). High secreting clones in this assay were selected for sequencing. In these experiments, the rabbit DQ2 antiserum appeared to have a lower specificity, therefore “high secreters” were identified as clones wherein secretion was about 3× higher than background (well A2 served as background for each plate). A total of 228 colonies were identified as high secreters for DQ2, and 225 colonies were identified as high secreters for dDQ2.

A summary of the secretion leaders potentially present in each of the six 96-well plates in the ELISA test, and the number of clones with 100% correct sequence is provided in Table 15 for the DQ2 clones and in Table 16 for the dDQ2 clones. In both tables, P22865* and P22865** (corresponding to SL candidate numbers 35 and 36 respectively in Table 14) indicate variants of the secretion leader of usp45 (SSusp45), which is a well-known state-of-the-art secretion leader. As noted previously, A2RIG7 was duplicated (see Plate_3).

TABLE 15
Sequencing data of DQ2 clones sorted by plate number

| | | # clones with 100% | # clones |
|---|---|---|---|
| Plate_number | SL | correct sequence | validated on WB |
| Plate_1 | A2RHI3 | 2 | 1 |
| | A2RHV3 | 0 | 0 |
| | A2RHV3 | 0 | 0 |
| | A2RHZ5 | 0 | 0 |
| | A2RI07 | 0 | 0 |
| | A2RIL8 | 2 | 1 |
| Plate_2 | A2RK75 | 0 | 0 |
| | A2RKE6 | 2 | 1 |
| | A2RLK0 | 3 | 1 |
| | Q8KKF9 | 0 | 0 |
| | A2RN73 | 0 | 0 |
| | A2RN78 | 0 | 0 |
| Plate_3 | P22865 | 1 | 1 |
| | A2RHU8 | 1 | 1 |
| | A2RIG7 | 30 | 2 |
| | A2RL19 | 0 | 0 |
| | A2RI74 | 4 | 2 |
| | (A2RIG7) | | |
| Plate_4 | A2RIL6 | 0 | 0 |
| | A2RIV4 | 1 | 1 |
| | A2RJJ4 | 12 | 3 |
| | A2RJL9 | 1 | 1 |
| | A2RJP5 | 6 | 2 |
| | A2RJQ9 | 3 | 2 |
| Plate_5 | A2RK78 | 4 | 2 |
| | A2RKB1 | 0 | 0 |
| | A2RKT3 | 0 | 0 |
| | A2RL18 | 0 | 0 |
| | A2RLU8 | 0 | 0 |
| | A2RM44 | 0 | 0 |
| Plate_6 | A2RM46 | 0 | 0 |
| | G0WJN9 | 0 | 0 |
| | A2RME7 | 0 | 0 |
| | P22865 | 1 | 1 |
| | P22865* | 0 | 0 |
| | P22865** | 1 | 1 |

TABLE 16
Sequencing data of dDQ2 clones sorted by plate number

| | | # clones with 100% | # clones |
|---|---|---|---|
| Plate_number | SL | correct sequence | validated on WB |
| Plate_1 | A2RHI3 | 0 | 0 |
| | A2RHV3 | 0 | 0 |
| | A2RHV3 | 0 | 0 |
| | A2RHZ5 | 0 | 0 |
| | A2RI07 | 0 | 0 |
| | A2RIL8 | 0 | 0 |
| Plate_2 | A2RK75 | 0 | 0 |
| | A2RKE6 | 0 | 0 |
| | A2RLK0 | 0 | 0 |
| | Q8KKF9 | 0 | 0 |
| | A2RN73 | 0 | 0 |
| | A2RN78 | 0 | 0 |
| Plate_3 | P22865 | 0 | 0 |
| | A2RHU8 | 0 | 0 |
| | A2RIG7 | 14 | 2 |
| | A2RL19 | 0 | 0 |
| | A2RI74 | 2 | 1 |
| | (A2RIG7) | | |
| Plate_4 | A2RIL6 | 0 | 0 |
| | A2RIV4 | 0 | 0 |
| | A2RJJ4 | 8 | 2 |
| | A2RJL9 | 3 | 2 |
| | A2RJP5 | 2 | 1 |
| | A2RJQ9 | 1 | 1 |
| Plate_5 | A2RK78 | 0 | 0 |
| | A2RKB1 | 0 | 0 |
| | A2RKT3 | 0 | 0 |
| | A2RL18 | 0 | 0 |
| | A2RLU8 | 0 | 0 |
| | A2RM44 | 0 | 0 |
| Plate_6 | A2RM46 | 0 | 0 |
| | G0WJN9 | 3 | 2 |
| | A2RME7 | 0 | 0 |
| | P22865 | 1 | 1 |
| | P22865* | 3 | 2 |
| | P22865** | 1 | 1 |

In Tables 15 and 16, the number of clones of each secretion leader further validated by western blot analysis is also indicated. From this, a presentative number of clones were selected for further validation on western blot. Specifically, 23 individual clones, representing 16 different secretion leaders, were tested for DQ2, and 15 individual clones, representing 10 different secretion leaders, were tested for dDQ2.

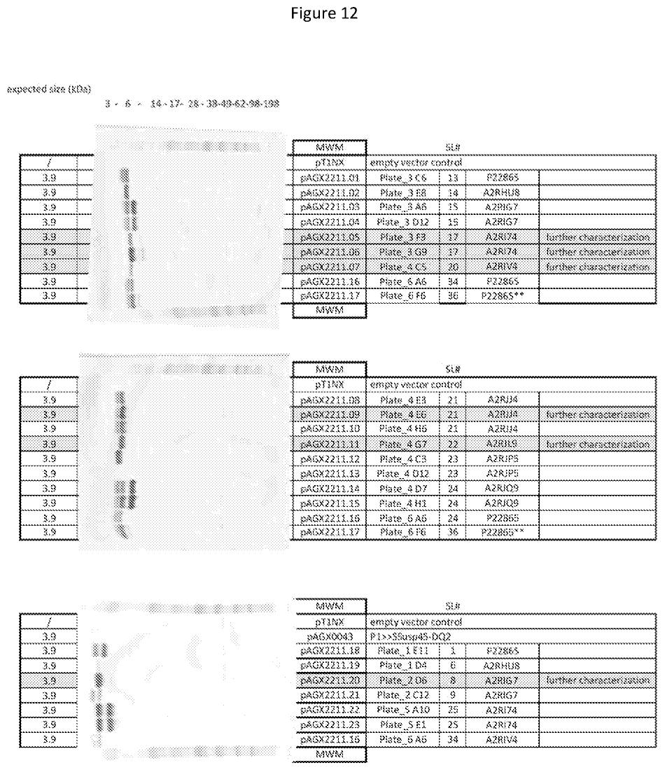

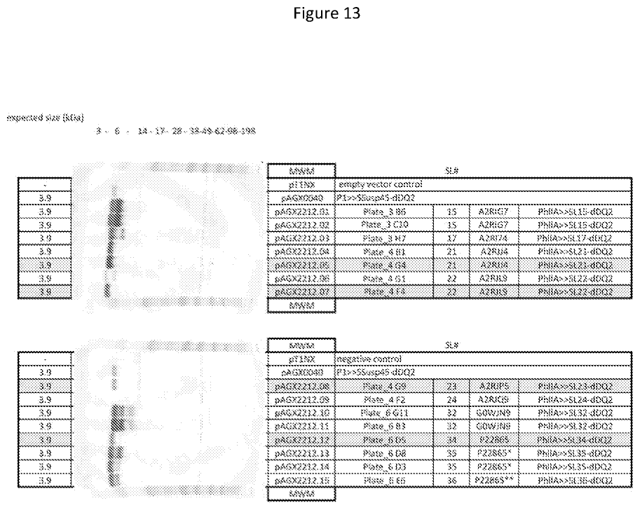

Western blots were prepared using conventional methods. In the western blots, equivalents of 1 ml of culture supernatant were used Immunoblots were revealed with DQ2 antibody OR245368_2 (Thermo) that reacts with both DQ2 and dDQ2. Results for DQ2 candidate secretion leaders is shown in FIG. 12 and for dDQ2 candidate secretion leaders is shown in FIG. 13.

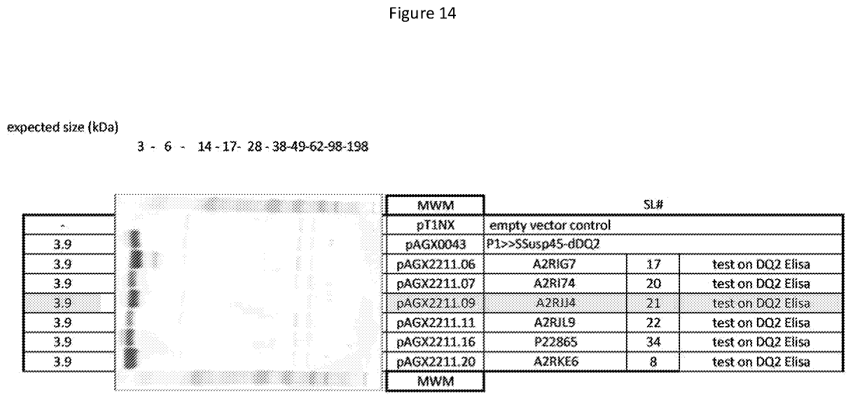

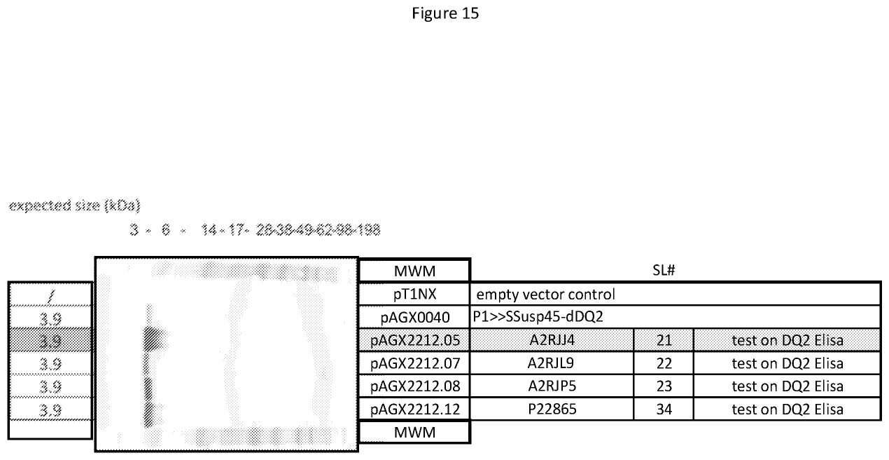

Based in the western blot results, selected secretion leaders were chosen for DQ2 and dDQ2. Western blots of the selected secretion leaders for DQ2 and dDQ2 are shown in FIG. 14 and FIG. 15, respectively. For both DQ2 and dDQ2, secretion leader #21 (A2RJJ4) was identified as the main secretion leader for use in the construction of clinical grade strains.

### Example 4: Construction of a Clinical-Grade L. lactis Secreting a dDQ2 Epitope and hIL10

A Lactococcus lactis strain (sAGX0868) secreting both a deamidated DQ2 epitope (dDQ2) from wheat gliadin and human IL-10 was generated in an MG1363 parental strain by introduction of an expression cassette for dDQ2 and human IL-10 using methods previously described. See, e.g., Steidler L. et al., Nat. Biotechnol. 2003; 21:785-789; and Steidler L, Rottiers P; Annals of the New York Academy of Sciences 2006; 1072:176-186. Methods to introduce changes into the L. lactis chromosome make use of double homologous recombination. A conditionally replicative carrier plasmid derived from pORI19 and containing an erythromycin selection marker, was constructed in the repA+ L. lactis strain LL108. Carrier plasmids were designed in such way that the cargo of interest was cloned in between up to 1 kb cross over (XO) areas, identical to the ones flanking the wild type sequence on the bacterial chromosome. This plasmid was introduced into MG1363 or any of its derivatives (repA−). Resistant colonies were selected on agar plates containing erythromycin and a first homologous recombination either at the 5′ or 3′ target sites was verified by PCR screening. Release of erythromycin selection enabled the excision of the carrier plasmid from the bacterial chromosome by a second homologous recombination, at either the 5′ or 3′ target site. The final genetic structure of the clinical-grade strain was extensively documented by both Sanger and Illumina full genome sequencing. There are no plasmids or residual erythromycin resistance in the final clinical strain. See, e.g., Steidler, L., et al., Nat. Biotechnol. 2003, 21(7): 785-789.

sAGX0868 is a derivative of Lactococcus lactis (L. lactis) MG1363. In sAGX0868:
Thymidylate synthase gene (thyA; Gene ID: 4798358) is absent, to warrant environmental containment (Steidler, L., et al., Nat. Biotechnol. 2003, 21(7): 785-789).
Trehalose-6-phosphate phosphorylase gene (trePP; Gene ID: 4797140) is absent, to allow accumulation of exogenously added trehalose.
Trehalose-6-phosphate phosphatase gene (otsB; Gene ID: 1036914) is positioned downstream of usp45 (Gene ID: 4797218) to facilitate conversion of trehalose-6-phosphate to trehalose. The otsB expression unit was transcriptionally and translationally coupled to usp45 by use of the intergenic region (IR) preceding the highly expressed L. lactis MG1363 505 ribosomal protein L30 gene (rpmD; Gene ID: 4797873).
The constitutive promoter of the HU-like DNA-binding protein gene (PhllA; Gene ID: 4797353) is preceding the putative phosphotransferase genes in the trehalose operon (trePTS; LLMG_RS02300 and LLMG_RS02305, Gene ID: 4797778 and Gene ID: 4797093 respectively) to potentiate trehalose uptake.
The gene encoding cellobiose-specific PTS system IIC component (Gene ID: 4796893), ptcC, is deleted (ΔptcC). This mutation ascertains trehalose retention after accumulation.
Insertion of a fragment encoding a fusion usp45 secretion leader (SSusp45) with the hil-10 gene, encoding human interleukin-10 (hIL-10; UniProt: P22301, aa 19-178, variant P2A [1]), downstream of the phosphopyruvate hydratase gene (eno; Gene ID: 4797432). To allow expression and secretion of hIL-10, the hil-10 expression unit was transcriptionally and translationally coupled to eno by use of IRrpmD.
Insertion, downstream of the hil-10 gene, of a fragment encoding a fusion of ps356 endolysin gene (ps356; Gene ID: 4798697) secretion leader (SSps356) with a fragment encoding deamidated DQ2 (ddq2), a protease-resistant 33-mer based on 6 overlapping α1- and α2-gliadin epitopes (UniProt: Q9M4L6_wheat, amino acids 57-89, glutamine deamidation at positions 66 and 80). To allow expression and secretion of dDQ2, the ddq2 expression unit was transcriptionally and translationally coupled to hil-10 by use of IR preceding the highly expressed L. lactis MG1363 50S ribosomal protein L14 gene (rplN; Gene ID: 4799034).

All genetic traits of sAGX0868 reside on the bacterial chromosome. The genetic background of sAGX0868 warrants:
Constitutive secretion of hIL-10.
Constitutive secretion of dDQ2.
Strict dependence on exogenously added thymidine for growth and survival.
The capacity to accumulate and retain trehalose and so acquire the capacity to resist bile acid toxicity.

FIG. 16 shows a schematic overview of relevant genetic loci of sAGX0868 as described: eno>>hil-10>>ddq2, ΔthyA, otsB, trePTS, ΔtrePP, ΔptcC, (/truncated/) genetic characters, intergenic regions (IR), PCR amplification product sizes (bp).

trePTS, ΔtrePP

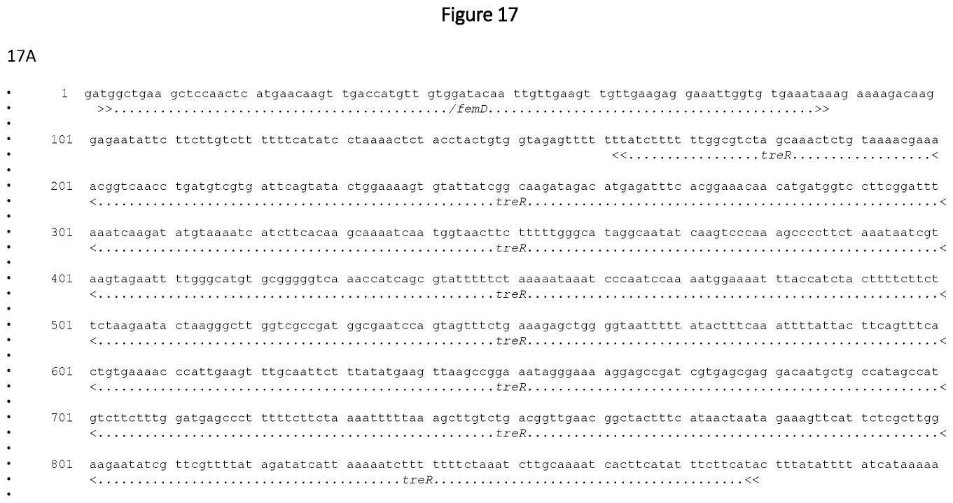

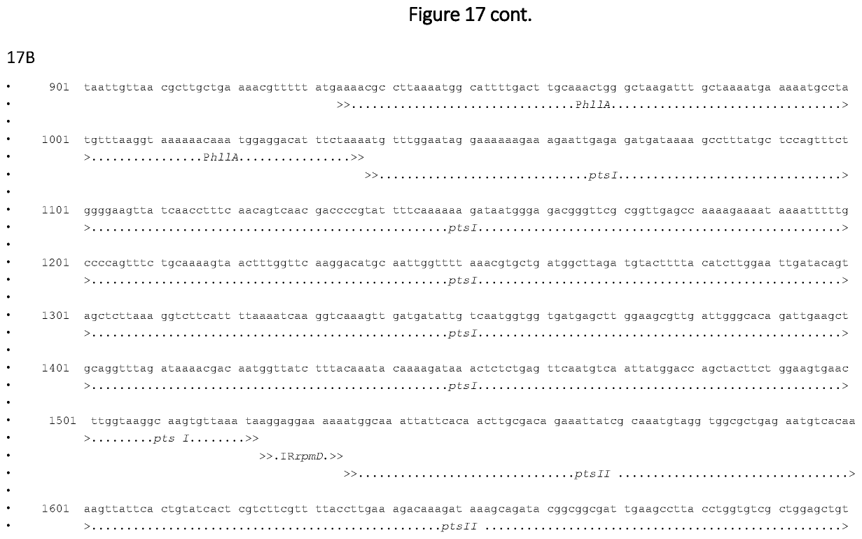

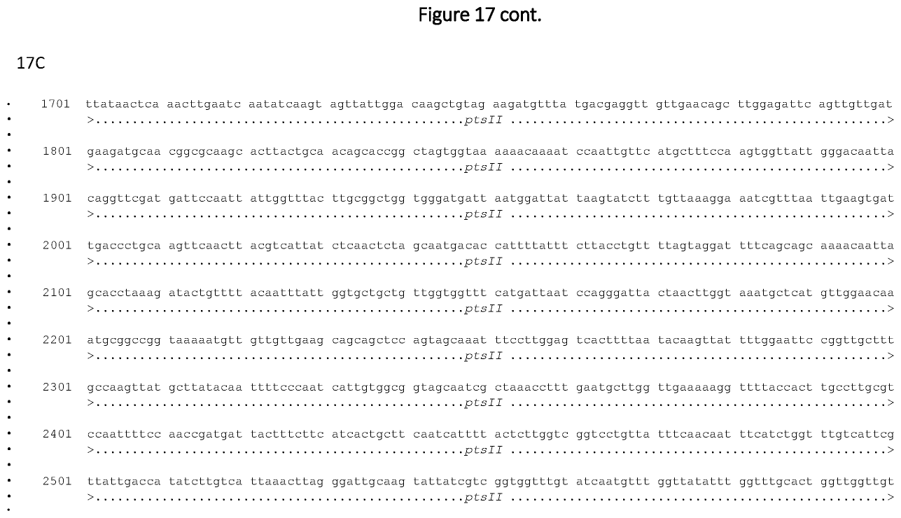

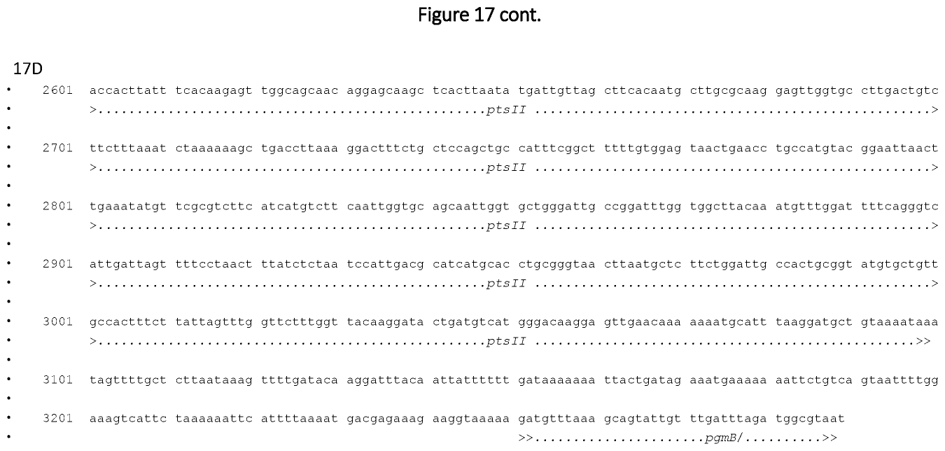

Deletion of trehalose-6-phosphate phosphorylase gene (trePP; Gene ID: 4797140). Insertion of the constitutive promoter of the HU-like DNA-binding protein gene (PhllA; Gene ID: 4797353) to precede the putative phosphotransferase genes in the trehalose operon (trePTS; LLMG_RS02300 and LLMG_RS02305; ptsI and ptsII; Gene ID: 4797778 and Gene ID: 4797093 respectively). Insertion of the intergenic region preceding the highly expressed L. lactis MG1363 50S ribosomal protein L30 gene (rpmD; Gene ID: 4797873) in-between ptsl and ptsII. (FIG. 17).

otsB

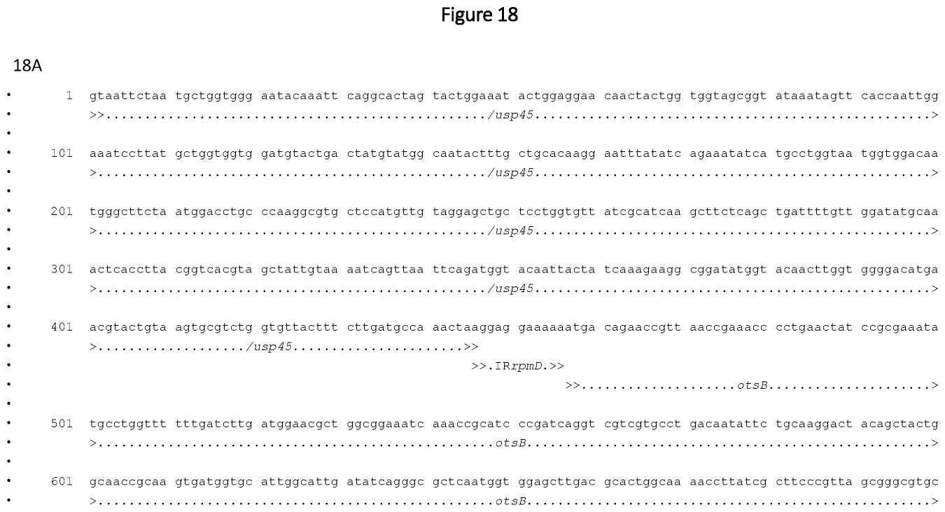

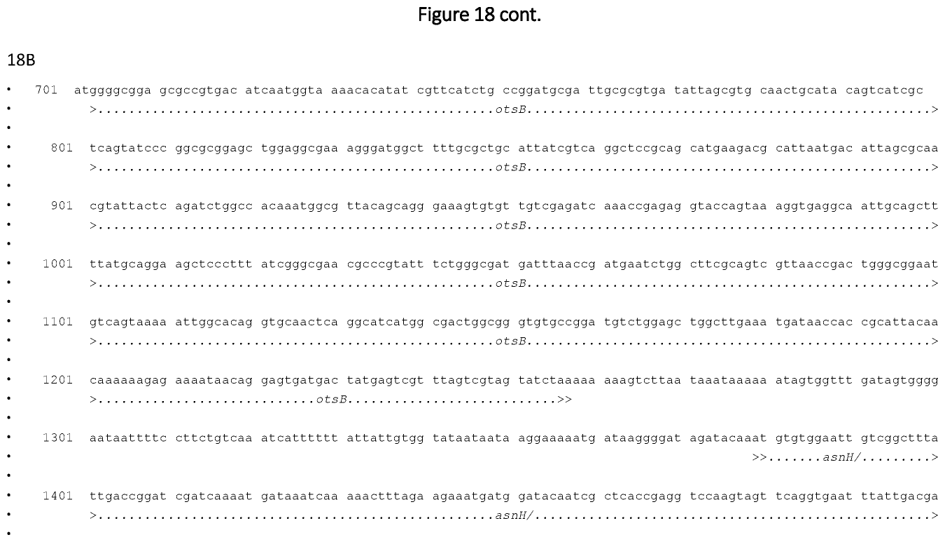

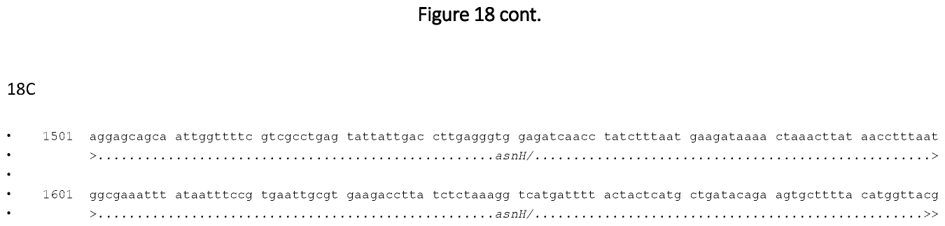

Insertion of trehalose-6-phosphate phosphatase gene (otsB; Gene ID: 1036914) downstream of unidentified secreted 45-kDa protein gene (usp45; Gene ID: 4797218). Insertion of the intergenic region preceding the highly expressed L. lactis MG1363 50S ribosomal protein L30 gene (rpmD; Gene ID: 4797873) between usp45 and otsB. (FIG. 18).

ΔptcC

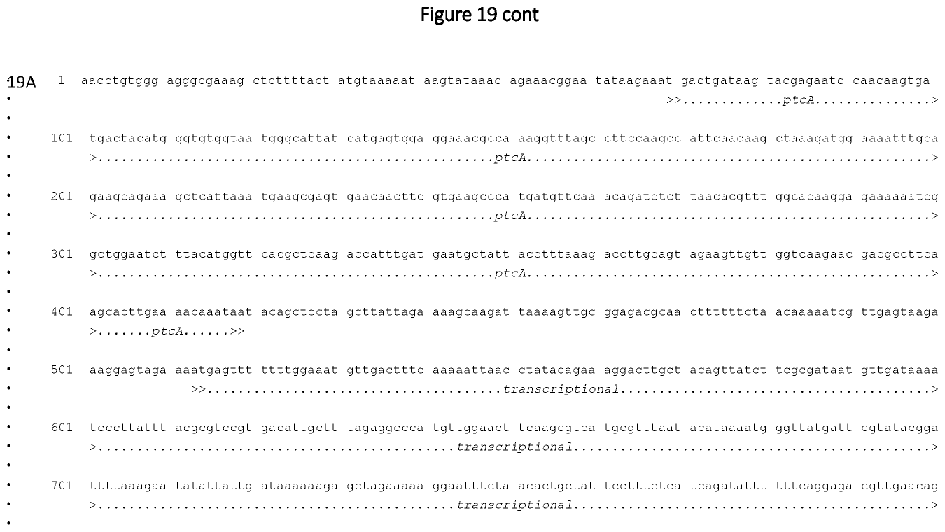

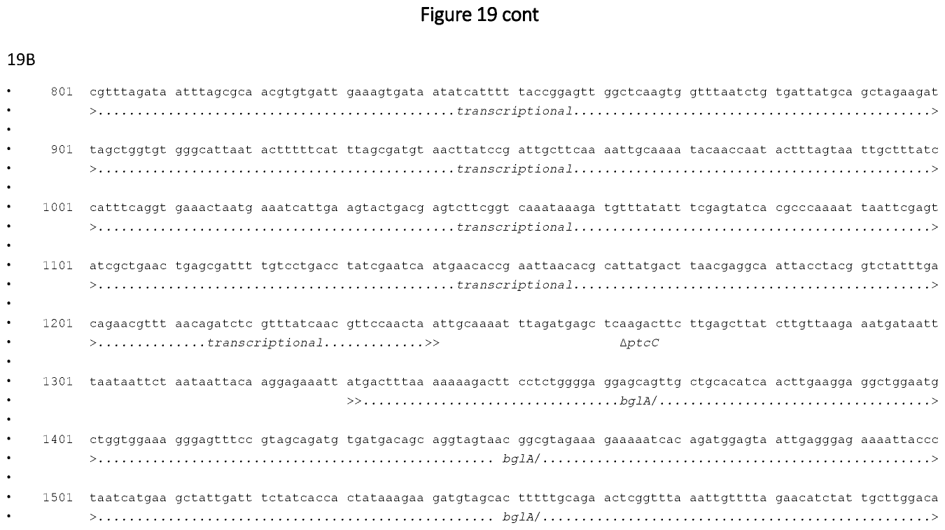

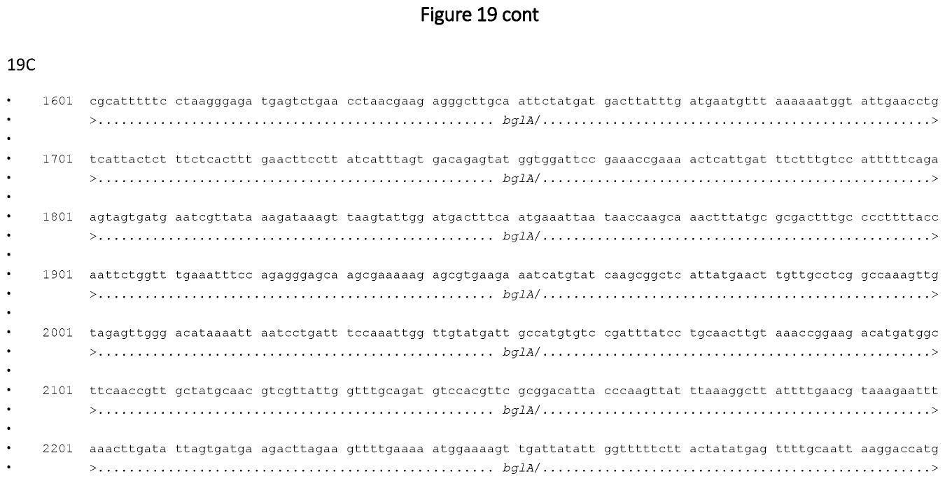

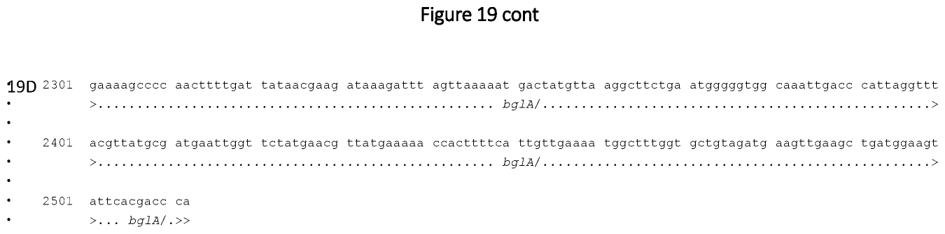

Deletion of the gene encoding cellobiose-specific PTS system IIC component (ptcC; Gene ID: 4796893). (FIG. 19).

ΔthyA

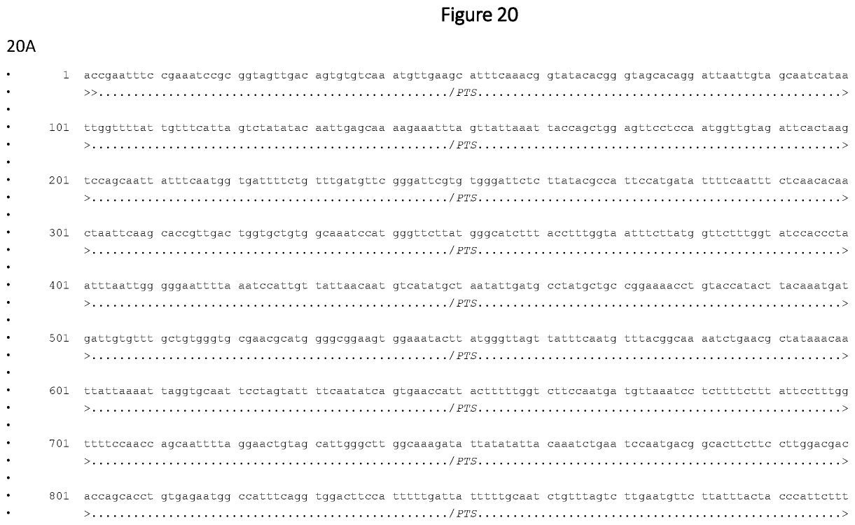

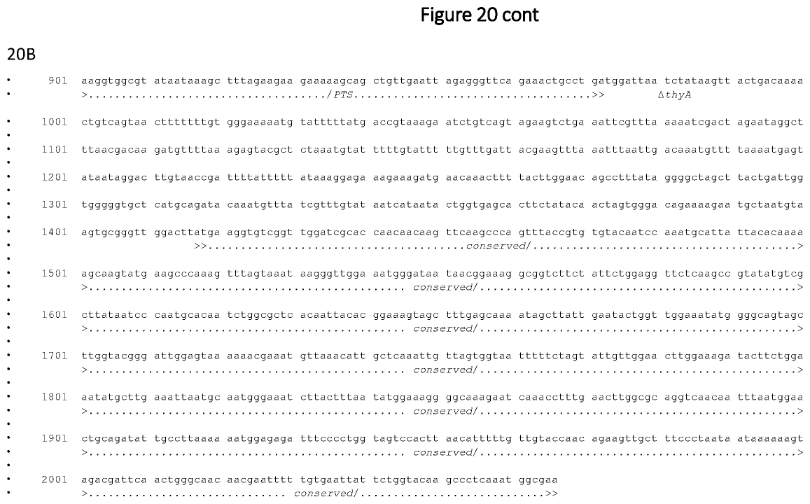

Deletion of thymidylate synthase gene (thyA; Gene ID: 4798358). (FIG. 20).

eno>>hil-10>>ddq2

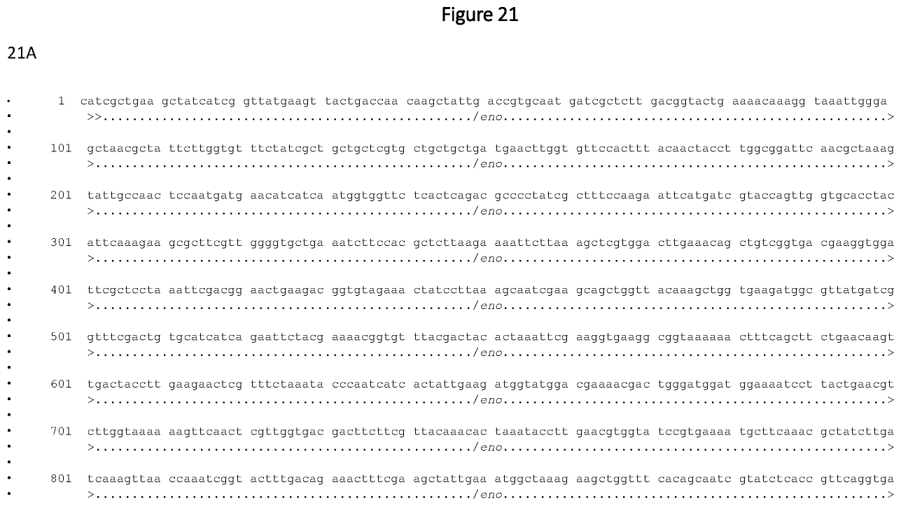

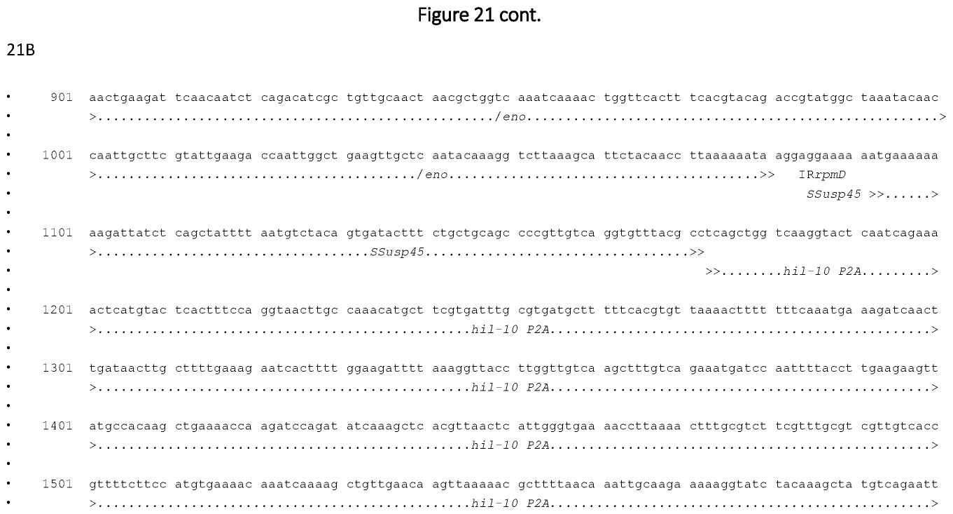

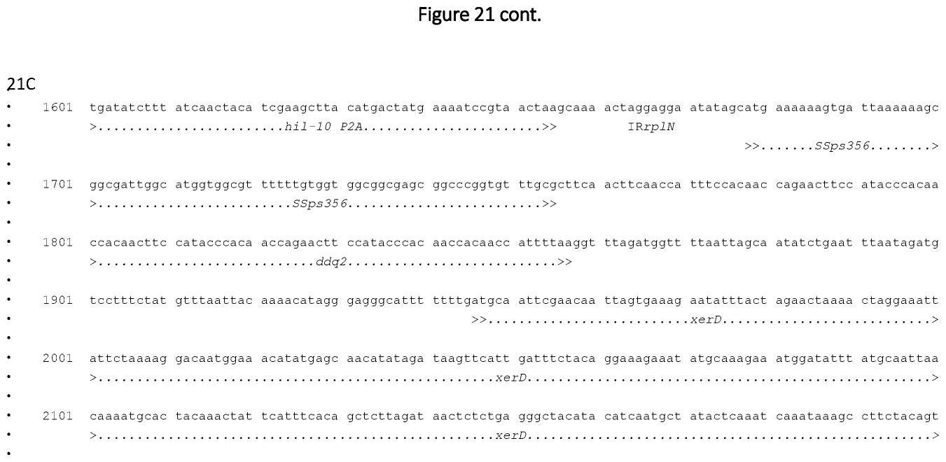

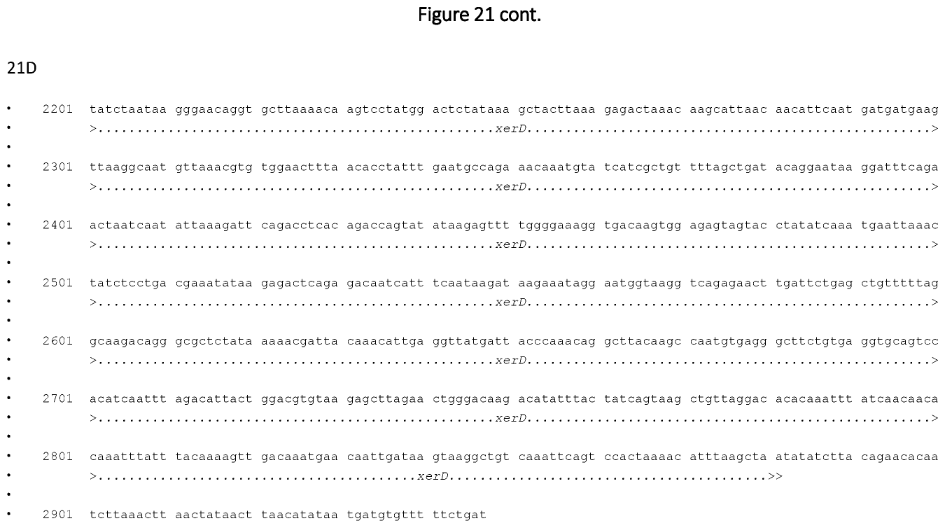

Insertion of a gene encoding a fusion of usp45 secretion leader (SSusp45) with the hil-10 gene, encoding human interleukin-10 (hIL-10; UniProt: P22301, aa 19-178, variant P2A; Steidler et al., Nat. Biotechnol. 2003, 21(7): 785-789) downstream of the phosphopyruvate hydratase gene (eno; Gene ID: 4797432), to allow expression and secretion of hIL-10. The hil-10 expression unit is transcriptionally and translationally coupled to eno by use of IRrpmD. (FIGS. 21A-21C).

A gene encoding a fusion of ps356 secretion leader (SSps356) with a fragment encoding deamidated DQ2 (ddq2), a protease-resistant 33-mer based on 6 overlapping α1- and α2-gliadin epitopes (UniProt: Q9M4L6_wheat, amino acids 57-89, glutamine deamidation at positions 66 and 80), is positioned downstream of this hil-10 gene, to allow expression and secretion of dDQ2. The ddq2 expression unit is transcriptionally and translationally coupled to hil-10 by use of IR preceding the highly expressed L. lactis MG1363 50S ribosomal protein L14 gene (rplN; Gene ID: 4799034). (FIGS. 21C and 21D).

### Example 5: Contemplated embodiments for L. lactis Secreting a dDQ2 Epitope and hIL10

A variety of further embodiments are contemplated for CeD-specific antigen and IL-10 expression units for alternative strain construction. The expression units preferably comprise integration of the expression unit(s) downstream of a highly expressing endogenous gene. Highly expressed endogenous genes can be identified, for instance, by proteomic and/or RNAseq analysis and subsequent validation by use of a reporter gene construct, e.g. PgeneX>>geneX>>GUS. As used throughout the specification “>>” represents a suitable expression link such as direct fusion of a promoter to a gene: PgeneX>>geneX or coupling of 2 genes through an intergenic region: geneX>>geneY.

The cassettes depicted optionally further comprise components described herein. For instance, the cassettes can further comprise at least one intergenic region transcriptionally coupling, e.g., the CeD-specific antigen to the endogenous gene. The cassettes can further comprise a secretion leader 5′ to each of the CeD-specific antigen and IL-10, wherein the secretion leader is transcriptionally and translationally coupled to the polypeptide, i.e., the CeD-specific antigen. Thus, in the cassettes depicted, “IL-10” can represent a coding sequence of a fusion polypeptide comprising a secretion leader fused to IL-10, and “ddq2” can represent a fusion polypeptide comprising a secretion leader fused to ddq2.

The CeD-specific antigen used in these examples is ddq2, however, the embodiments are not restricted to ddq2.

Exemplary embodiments for combined expression units integrated into the bacterial chromosome, downstream of (i.e., 3′ to) a highly expressed endogenous gene are shown in Table 17.

The following genes are referenced below:

| | | Discontinued GeneID; |
|---|---|---|
| Gene | Description | Locus tag NEW; OLD |
| tufA | elongation factor Tu | Gene ID 4798092; Locus tag |
| | | LLMG_RS10245; llmg_2050 |
| sodA | superoxide dismutase | Gene ID: 4796682; Locus tag |
| | | LLMG_RS02190; llmg_0429 |
| pdhD | dihydrolipoyl | Gene ID 4798159; Locus tag |
| | dehydrogenase | LLMG_RS00390; llmg_0071 |

TABLE 17
Combined-Polycistronic Expression
Cassettes including endogenous gene

| Cassette no. | |
|---|---|
| 5.1 | PgapB >> gapB >> IL-10 >> ddq2 |
| 5.2 | PgapB >> gapB >> ddq2 >> IL-10 |
| 5.3 | PpdhD >> pdhD >> IL-10 >> ddq2 |
| 5.4 | PpdhD >> pdhD >> ddq2 >> IL-10 |
| 5.5 | PsodA >> sodA >> IL-10 >> ddq2 |
| 5.6 | PsodA >> sodA >> ddq2 >> IL-10 |
| 5.7 | PtufA >> tufA >> IL-10 >> ddq2 |
| 5.8 | PtufA >> tufA >> ddq2 >> IL-10 |

Exemplary embodiments for two separate expression units, each integrated into the bacterial chromosome, downstream of (i.e., 3′ to) a highly expressed endogenous gene are shown in Table 18.

TABLE 18
Separated Expression Cassettes including endogenous gene

| Cassette no. | |
|---|---|
| | CeD-specific antigen cassette |
| 5.9 | Peno >> eno >> ddq2 |
| 5.9 | |
| 5.9 | |
| 5.9 | |
| 5.10 | PgapB >> gapB >> ddq2 |
| 5.10 | |
| 5.10 | |
| 5.10 | |
| 5.11 | PpdhD >> pdhD >> ddq2 |
| 5.11 | |
| 5.11 | |
| 5.11 | |
| 5.12 | PsodA >> sodA >> ddq2 |
| 5.12 | |
| 5.12 | |
| 5.12 | |
| 5.13 | PtufA >> tufA >> ddq2 |
| 5.13 | |
| 5.13 | |
| 5.13 | |
| | IL-10 cassette |
| 5.14 | PgapB >> gapB >> IL-10 |
| 5.15 | PpdhD >> pdhD >> IL-10 |
| 5.16 | PsodA >> sodA >> IL-10 |
| 5.17 | PtufA >> tufA >> IL-10 |
| 5.18 | Peno >> eno >> IL-10 |
| 5.15 | PpdhD >> pdhD >> IL-10 |
| 5.16 | PsodA >> sodA >> IL-10 |
| 5.17 | PtufA >> tufA >> IL-10 |
| 5.18 | Peno >> eno >> IL-10 |
| 5.14 | PgapB >> gapB >> IL-10 |
| 5.16 | PsodA >> sodA >> IL-10 |
| 5.17 | PtufA >> tufA >> IL-10 |
| 5.18 | Peno >> eno >> IL-10 |
| 5.14 | PgapB >> gapB >> IL-10 |
| 5.15 | PpdhD >> pdhD >> IL-10 |
| 5.17 | PtufA >> tufA >> IL-10 |
| 5.18 | Peno >> eno >> IL-10 |
| 5.14 | PgapB >> gapB >> IL-10 |
| 5.15 | PpdhD >> pdhD >> IL-10 |
| 5.16 | PsodA >> sodA >> IL-10 |

Embodiments comprising an endogenous promoter without its associated endogenous gene are also contemplated. Such embodiments may be difficult to construct due to instability arising from robust expression from promoters in multi-copy plasmids. These problems may affect the creation and propagation of the intermediate components used in strain construction.

Exemplary embodiments for combined expression units integrated into the bacterial chromosome, downstream of (i.e., 3′ to) a highly expressed endogenous promoter are shown in Table 19.

TABLE 19
Combined-Polycistronic Expression Cassettes
including endogenous promoter

| Cassette no. | |
|---|---|
| 5.19 | Peno >> IL-10 >> ddq2 |
| 5.20 | Peno >> ddq2 >> IL-10 |
| 5.21 | PgapB >> IL-10 >> ddq2 |
| 5.22 | PgapB >> ddq2 >> IL-10 |
| 5.23 | PpdhD >> IL-10 >> ddq2 |
| 5.24 | PpdhD >> ddq2 >> IL-10 |
| 5.25 | PsodA >> IL-10 >> ddq2 |
| 5.26 | PsodA >> ddq2 >> IL-10 |
| 5.27 | PtufA >> IL-10 >> ddq2 |
| 5.28 | PtufA >> ddq2 >> IL-10 |
| 5.29 | PhllA >> IL-10 >> ddq2 |
| 5.30 | PhllA >> ddq2 >> IL-10 |
| 5.31 | Pdps >> IL-10 >> ddq2 |
| 5.32 | Pdps >> ddq2 >> IL-10 |
| 5.33 | PthyA >> IL-10 >> ddq2 |
| 5.34 | PthyA >> ddq2 >> IL-10 |
| 5.35 | PpepV >> IL-10 >> ddq2 |
| 5.36 | PpepV >> ddq2 >> IL-10 |
| 5.37 | PpepQ >> IL-10 >> ddq2 |
| 5.38 | PpepQ >> ddq2 >> IL-10 |

Exemplary embodiments for two separate expression units, each integrated into the bacterial chromosome, downstream of (i.e., 3′ to) a highly expressed endogenous promoter are shown in Table 20.

TABLE 20
Separated Expression Cassettes

| Cassette no. | |
|---|---|
| | CeD-specific antigen cassette |
| 5.39 | Peno >> ddq2 |
| 5.39 | |
| 5.39 | |
| 5.39 | |
| 5.39 | |
| 5.39 | |
| 5.39 | |
| 5.39 | |
| 5.40 | PgapB >> ddq2 |
| 5.40 | |
| 5.40 | |
| 5.40 | |
| 5.40 | |
| 5.40 | |
| 5.40 | |
| 5.40 | |
| 5.41 | PsodA >> ddq2 |
| 5.41 | |
| 5.41 | |
| 5.41 | |
| 5.41 | |
| 5.41 | |
| 5.41 | |
| 5.41 | |
| 5.42 | PtufA >> ddq2 |
| 5.42 | |
| 5.42 | |
| 5.42 | |
| 5.42 | |
| 5.42 | |
| 5.42 | |
| 5.42 | |
| 5.43 | PhllA >> ddq2 |
| 5.43 | |
| 5.43 | |
| 5.43 | |
| 5.43 | |
| 5.43 | |
| 5.43 | |
| 5.43 | |
| 5.44 | Pdps >> ddq2 |
| 5.44 | |
| 5.44 | |
| 5.44 | |
| 5.44 | |
| 5.44 | |
| 5.44 | |
| 5.44 | |
| 5.45 | PthyA >> ddq2 |
| 5.45 | |
| 5.45 | |
| 5.45 | |
| 5.45 | |
| 5.45 | |
| 5.45 | |
| 5.45 | |
| 5.46 | PpepV >> ddq2 |
| 5.46 | |
| 5.46 | |
| 5.46 | |
| 5.46 | |
| 5.46 | |
| 5.46 | |
| 5.46 | |
| 5.47 | PpepQ >> ddq2 |
| 5.47 | |
| 5.47 | |
| 5.47 | |
| 5.47 | |
| 5.47 | |
| 5.47 | |
| 5.47 | |
| | IL-10 cassette |
| 5.48 | PgapB >> IL-10 |
| 5.49 | PsodA >> IL-10 |
| 5.50 | PtufA >> IL-10 |
| 5.51 | PhllA >> IL-10 |
| 5.52 | Pdps >> IL-10 |
| 5.53 | PthyA >> IL-10 |
| 5.54 | PpepV >> IL-10 |
| 5.55 | PpepQ >> IL-10 |
| 5.56 | Peno >> IL-10 |
| 5.49 | PsodA >> IL-10 |
| 5.50 | PtufA >> IL-10 |
| 5.51 | PhllA >> IL-10 |
| 5.52 | Pdps >> IL-10 |
| 5.53 | PthyA >> IL-10 |
| 5.54 | PpepV >> IL-10 |
| 5.55 | PpepQ >> IL-10 |
| 5.56 | Peno >> IL-10 |
| 5.57 | PgapB >> IL-10 |
| 5.50 | PtufA >> IL-10 |
| 5.51 | PhllA >> IL-10 |
| 5.52 | Pdps >> IL-10 |
| 5.53 | PthyA >> IL-10 |
| 5.54 | PpepV >> IL-10 |
| 5.55 | PpepQ >> IL-10 |
| 5.56 | Peno >> IL-10 |
| 5.57 | PgapB >> IL-10 |
| 5.58 | PsodA >> IL-10 |
| 5.51 | PhllA >> IL-10 |
| 5.52 | Pdps >> IL-10 |
| 5.53 | PthyA >> IL-10 |
| 5.54 | PpepV >> IL-10 |
| 5.55 | PpepQ >> IL-10 |
| 5.56 | Peno >> IL-10 |
| 5.57 | PgapB >> IL-10 |
| 5.58 | PsodA >> IL-10 |
| 5.59 | PtufA >> IL-10 |
| 5.52 | Pdps >> IL-10 |
| 5.53 | PthyA >> IL-10 |
| 5.54 | PpepV >> IL-10 |
| 5.55 | PpepQ >> IL-10 |
| 5.56 | Peno >> IL-10 |
| 5.57 | PgapB >> IL-10 |
| 5.58 | PsodA >> IL-10 |
| 5.59 | PtufA >> IL-10 |
| 5.51 | PhllA >> IL-10 |
| 5.53 | PthyA >> IL-10 |
| 5.54 | PpepV >> IL-10 |
| 5.55 | PpepQ >> IL-10 |
| 5.56 | Peno >> IL-10 |
| 5.57 | PgapB >> IL-10 |
| 5.58 | PsodA >> IL-10 |
| 5.59 | PtufA >> IL-10 |
| 5.51 | PhllA >> IL-10 |
| 5.52 | Pdps >> IL-10 |
| 5.54 | PpepV >> IL-10 |
| 5.55 | PpepQ >> IL-10 |
| 5.56 | Peno >> IL-10 |
| 5.57 | PgapB >> IL-10 |
| 5.58 | PsodA >> IL-10 |
| 5.59 | PtufA >> IL-10 |
| 5.51 | PhllA >> IL-10 |
| 5.52 | Pdps >> IL-10 |
| 5.53 | PthyA >> IL-10 |
| 5.55 | PpepQ >> IL-10 |
| 5.56 | Peno >> IL-10 |
| 5.57 | PgapB >> IL-10 |
| 5.58 | PsodA >> IL-10 |
| 5.59 | PtufA >> IL-10 |
| 5.51 | PhllA >> IL-10 |
| 5.52 | Pdps >> IL-10 |
| 5.53 | PthyA >> IL-10 |
| 5.54 | PpepV >> IL-10 |

Further contemplated are combinations of an expression unit with both endogenous promoter and the endogenous gene, such as those in Table 18, with an expression unit with only an endogenous promoter, such as those in Table 20.

Non-limiting examples include:

| Cassette no. | |
|---|---|
| | CeD-specific antigen cassette |
| 5.9 | Peno >> eno >> ddq2 |
| 5.39 | Peno >> ddq2 |
| | IL-10 cassette |
| 5.57 | PgapB >> IL-10 |
| 5.14 | PgapB >> gapB >> IL-10 |

Further contemplated embodiments are expression units as described above, with one expression unit (i.e., IL-10) on the chromosome and another expression unit (i.e., ddq2) on an episome, as well as embodiments with both expression units on an episome, wherein the episome is stabilized by food grade, non-antibiotic selection through auxotrophy.

A summary of some of the genes referred to in this disclosure is provided in Table 21.

TABLE 21

| Gene | | NCBI Gene | old locus | current locus |
|---|---|---|---|---|
| name | Descriptive Name | ID | tag | tag |
| hllA | L. lactis MG1363 HU-like | 4797353 | | LLMG_RS02525 |
| | DNA-binding protein gene | | | |
| eno | L. lactis MG1363 | 4797432 | llmg_0617 | LLMG_RS03215 |
| | phosphopyruvate hydratase gene | | | |
| rpIN | L. lactis MG1363 50S | 4799034 | | LLMG_RS11895 |
| | ribosomal protein L14 gene | | | |
| ptsI | L. lactis MG1363 sucrose- | 4797778 | llmg_0453 | LLMG_RS02300 |
| | specific PTS enzyme IIABC | | | |
| | (also referred to herein as | | | |
| | L. lactis trehalose transporter | | | |
| | (putative phosphotransferase | | | |
| | genes in the trehalose operon | | | |
| | trePTS) | | | |
| ptsII | L. lactis MG1363 beta- | 4797093 | llmg_0454 | LLMG_RS02305 |
| | glucoside-specific PTS system | | | |
| | IIABC component | | | |
| | (also referred to herein as | | | |
| | L. lactis trehalose transporter | | | |
| | (putative phosphotransferase | | | |
| | genes in the trehalose operon | | | |
| | trePTS) | | | |
| otsB | Escherichia coli CFT073 | 1036914 | | c2311 |
| | trehalose-6-phosphate | | | |
| | phosphatase gene | | | |
| usp45 | L. lactis MG1363 secreted 45 | 4797218 | | LLMG_RS12595 |
| | kDa protein precursor | | | |
| | (as so referred to herein as | | | |
| | L. lactis unidentified secreted | | | |
| | 45-kDa protein gene) | | | |
| ptcC | L. lactis MG1363 cellobiose- | 4796893 | llmg_0440 | LLMG_RS02240 |
| | specific PTS system IIC | | | |
| | component | | | |
| trePP | L. lactis MG1363 | 4797140 | llmg_0455 | LLMG_RS02310 |
| | trehalose/maltose hydrolase | | | |
| | (also referred to herein as | | | |
| | L. lactis MG1363 trehalose-6- | | | |
| | phosphate phosphorylase gene) | | | |
| thyA | L. lactis MG1363 thymidylate | 4798358 | llmg_0964 | LLMG_RS04905 |
| | synthase gene | | | |
| rpmD | L. lactis MG1363 50S ribosomal | 4797873 | llmg_2363 | LLMG_RS11850 |
| | protein L30 gene | | | |
| tufA | L. lactis MG1363 elongation | 4798092 | llmg_2050 | LLMG_RS10245 |
| | factor Tu | | | |
| sodA | L. lactis MG1363 SodA protein | 4796682 | llmg_0429 | LLMG_RS02190 |
| | (also referred to herein as | | | |
| | L. lactis MG1363 superoxide | | | |
| | dismutase) | | | |
| pdhD | L. lactis MG1363 dihydrolipoyl | 4798159 | llmg_0071 | LLMG_RS00390 |
| | dehydrogenase | | | |
| gapB | L. lactis MG1363 | 4797877 | llmg_2539 | LLMG_RS12755 |
| | glyceraldehyde 3-phosphate | | | |
| | dehydrogenase | | | |
| IL-10 | Human interleukin-10 | UniProtKB: | | |
| | | P22301 | | |
| IL-2 | Human interleukin-2 | UniProtKB: | | |
| | | P60568 | | |

### Exemplary Embodiments

Embodiment 1. A lactic acid bacterium (LAB) comprising:

(i) an exogenous nucleic acid encoding human interleukin-10 (hIL-10) and

(ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,

wherein said exogenous nucleic acid encoding hIL-10 and said exogenous nucleic acid encoding a gliadin polypeptide are chromosomally integrated in the LAB.

Embodiment 2. A lactic acid bacterium (LAB) comprising an exogenous nucleic acid encoding a secretion leader sequence fused in frame to a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope, wherein said exogenous nucleic acid is chromosomally integrated in the LAB.

Embodiment 3. The LAB of Embodiment 1, wherein said exogenous nucleic acid encoding the gliadin polypeptide further encodes a secretion leader sequence fused to said gliadin polypeptide coding sequence.

Embodiment 4. The LAB of Embodiment 1 or 3, comprising a polycistronic expression unit comprising said exogenous nucleic acid encoding hIL-10 and said exogenous nucleic acid encoding the gliadin polypeptide.

Embodiment 5. The LAB of Embodiment 1,3, or 4, wherein said LAB constitutively expresses and secretes said hIL-10 and said gliadin polypeptide.

Embodiment 6. The LAB of any one of Embodiments 1 to 5, wherein said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36, and variants thereof having 1, 2, or 3 variant amino acid positions.

Embodiment 7. The LAB of any one of Embodiments 1 to 6, wherein said gliadin polypeptide comprises:

(a) an HLA-DQ2 specific epitope and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, and SL #36; or

(b) a deamidated HLA-DQ2 specific epitope, and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #25, and SL #36.

Embodiment 8. The LAB of any one of Embodiments 1 to 7, wherein said exogenous nucleic acid encoding a gliadin polypeptide encodes a gliadin polypeptide comprising or consisting of:

(DQ2)
(SEQ ID NO: 3)
LQLQPFPQPQLPYPQPQLPYPQPQLPYPQPQPF,
(dDQ2)
(SEQ ID NO: 7)
LQLQPFPQPELPYPQPQLPYPQPELPYPQPQPF,
or
(SEQ ID NO: 33)
LQLQPFPQPELPYPQPELPYPQPELPYPQPQPF.

Embodiment 9. The LAB of any one of Embodiment 1 to 8, wherein said exogenous nucleic acid encoding a gliadin polypeptide encodes a gliadin polypeptide comprising or consisting of: LQLQPFPQPELPYPQPQLPYPQPELPYPQPQPF (SEQ ID NO: 7) (dDQ2), and a secretion leader selected from the secretion leader group consisting of SL #17, SL #21, SL #22, and SL #23.

Embodiment 10. The LAB of Embodiments 1, 3, 4 or 5, comprising the following chromosomally integrated polycistronic expression cassettes:
a. a first polycistronic expression cassette comprising an eno promoter positioned 5′ of an eno gene, a first intergenic region, an hIL-10 secretion leader sequence, said exogenous nucleic acid encoding hIL-10; a second intergenic region, a gliadin polypeptide secretion leader sequence, and said exogenous nucleic acid encoding said gliadin polypeptide;
b. a second polycistronic expression cassette comprising a usp45 promoter, usp45, and an exogenous nucleic acid encoding a trehalose-6-phosphate phosphatase and optionally an intergenic region, such as rpmD, between said usp45 and said exogenous nucleic acid encoding said trehalose-6-phosphate phosphatase; and
c. a third polycistronic expression cassette comprising nucleic acid encoding one or more trehalose transporters positioned 3′ of an hllA promoter (PhllA);
   and genetically modified to include:
d. inactivation or deletion of a trehalose-6-phosphate phosphorylase gene (trePP);
e. inactivation or deletion of a gene encoding a cellobiose-specific PTS system IIC component (ptcC); and
f. deletion of a thymidylate synthase gene (thyA).

Embodiment 11. The LAB of Embodiment 10, wherein said gliadin polypeptide comprises:

(a) an HLA-DQ2 specific epitope and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, and SL #36; or

(b) a deamidated HLA-DQ2 specific epitope, and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #25, and SL #36.

Embodiment 12. The LAB of Embodiment 1, which is sAGX0868.

Embodiment 13 A composition comprising:
(a) a lactic acid bacterium (LAB) of any one of Embodiments 1 to 12;
   or
(b) a first LAB containing an exogenous nucleic acid encoding an interleukin-10 (IL-10) polypeptide and expresses the IL-10 polypeptide; and
   a second LAB containing an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope,

wherein said exogenous nucleic acid encoding hIL-10 and said exogenous nucleic acid encoding a gliadin polypeptide are chromosomally integrated in the LAB.

Embodiment 14. Use of the LAB of any one of Embodiments 1 to 12 or the composition of Embodiment 13 in the treatment of celiac disease.

Embodiment 15. Use of the LAB of any one of Embodiments 1 to 12 or the composition of Embodiment 13 for the preparation of a medicament for the treatment of celiac disease.

Embodiment 16. A polynucleotide sequence comprising:
(a) a polycistronic expression unit comprising:

(i) a nucleic acid encoding hIL-10, and

(ii) a nucleic acid encoding a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope,

wherein said nucleic acid encoding hIL-10 further encodes a secretion leader sequence fused to said hIL-10, and wherein said nucleic acid encoding said gliadin polypeptide further encodes a secretion leader sequence fused to said gliadin polypeptide; or
(b) a polycistronic integration vector comprising

(i) a first intergenic region,

(ii) a first open reading frame encoding a first therapeutic protein,

(iii) a second intergenic region, and

(iv) a second open reading frame encoding a second therapeutic protein,

wherein the first intergenic region is transcriptionally coupled at its 3′ end to the first open reading frame, the second intergenic region is transcriptionally coupled to the 3′ end of the first open reading frame, and the second intergenic region is transcriptionally coupled at its 3′ end to the second open reading frame.

Embodiment 17. A method of inducing oral tolerance to gluten in a subject at risk of celiac disease, comprising administering to a subject at risk of celiac disease a therapeutically effective amount of a lactic acid bacterium (LAB) engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,

wherein said exogenous nucleic acid encoding IL-10 and said exogenous nucleic acid encoding a gliadin polypeptide are chromosomally integrated in the LAB, thereby inducing oral tolerance.

Embodiment 18. The method of Embodiment 17, wherein said interleukin-10 is human interleukin-10 (hIL-10).

Embodiment 19. The method of Embodiment 17 or 18, wherein said subject at risk of celiac disease exhibits a risk factor, wherein the risk factor is a genetic predisposition.

Embodiment 20. The method of any one of Embodiments 17 tol9 wherein administering the therapeutically effective amount of said LAB in said subject increases tolerance-inducing lymphocytes in a sample of lamina propria cells of said subject.

Embodiment 21. The method of any one of Embodiments 17 to20, wherein administering the therapeutically effective amount of said LAB in said subject increases CD4⁺ Foxp3⁺ regulatory T cells in a sample of lamina propria cells of said subject.

Embodiment 22. The method of any one of Embodiments 17 to2l, wherein administering the therapeutically effective amount of said LAB in said subject increases a ratio of CD4⁺ Foxp3⁺ regulatory T cells over T_{H}1 cells expressing Tbet in a sample of lamina propria cell of said subject.

Embodiment 23. The method of any one of Embodiments 17 to 22, wherein the development of villous atrophy upon exposure to gluten is prevented, inhibited or minimized in said subject.

Embodiment 24. A method of reducing villous atrophy in a subject diagnosed with celiac disease, comprising administering to said subject having villous atrophy a therapeutically effective amount of a LAB engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2 specific epitope and/or at least one deamidated HLA-DQ2 specific epitope, and (b) at least one HLA-DQ8 specific epitope and/or at least one deamidated HLA-DQ8 specific epitope,

wherein LAB produces at least a 55% reduction of the villous atrophy relative to a reference LAB that does not express IL-10 and the gliadin polypeptide in a mouse model of celiac disease.

Embodiment 25. The method of Embodiment 24, wherein said interleukin-10 is human interleukin-10 (hIL-10).

Embodiment 26. The method of Embodiment 24 or 25, where the villous atrophy is present due to intestinal gluten exposure.

Embodiment 27. The method of any one of Embodiments 24 to 26, wherein said LAB produces at least a 60%, 65%, 70%, 75%, 80%, 85%, 90%, 91%, 92%, 93%, 94%, 95%, 96%, 97% 98%, 99% or 100% reduction of the villous atrophy relative to the reference LAB that does not express IL-10 and the gliadin polypeptide in a mouse model of celiac disease.

Embodiment 28. The method of any one of Embodiments 24 to 27, wherein said administering:

a. reduces intraepithelial lymphocytosis in said subject as compared to intraepithelial lymphocytosis prior to administration to said subject and/or reduces the level of CD3⁺ intraepithelial lymphocytes (IELs) in a sample obtained from said subject as compared to CD3⁺ IELs present in a sample obtained from said subject prior to the administering step;

b. reduces the number of cytotoxic CD8⁺ IELs in said subject as compared to said cytotoxic CD8⁺ IELs present in a sample of said subject prior to administration;

c. reduces the level of Foxp3⁻Tbet⁺CD4⁺ T cells of said subject as compared to said Foxp3⁻Tbet⁺CD4⁺ T cells present in a sample of said subject prior to administration and/or increases the level of Foxp3⁺ Tbet⁻CD4⁺ T cells in a sample of lamina propria lymphocytes of said subject compared to said Foxp3⁻Tbet⁺CD4⁺ T cells present in a sample of said subject prior to administration;

d. prevents, inhibits or minimizes villous atrophy recurrence in said subject upon exposure to gluten; or

e. improves villous height (Vh)-to-crypt depth (Cd) ratio in said subject and/or restores the Vh/Cd ratio to a normal range in said subject.

Embodiment 29. The method of any one of Embodiments 17 to 28, wherein said LAB is said LAB of any one of Embodiments 1 to 12.

Embodiments 30. The method of any one of Embodiments 17 to 29, wherein said LAB is administered in a unit dosage form comprising from about 10⁴ colony forming units (cfu) to about 10¹² cfu per day, from about 10⁶ cfu to about 10¹² cfu per day, or from about 10⁹ cfu to about 10¹² cfu per day.

Embodiment 31. The method of any one of Embodiments 17 to 30, wherein said LAB is sAGX0868.

### Further Exemplary Embodiments

Embodiment 101. A lactic acid bacterium (LAB) comprising:
(i) an exogenous nucleic acid encoding human interleukin-10 (hIL-10) and
(ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,
wherein said exogenous nucleic acid encoding hIL-10 and said exogenous nucleic acid encoding a gliadin polypeptide are chromosomally integrated in the LAB.

Embodiment 102. The LAB of Embodiment 101, wherein said exogenous nucleic acid encoding the hIL-10 further encodes a secretion leader sequence fused to said hIL-10 coding sequence.

Embodiment 103. The LAB of Embodiment 102, wherein said hIL-10 is secreted as a mature hIL-10 without said secretion leader.

Embodiment 104. The LAB of Embodiment 103, wherein said hIL-10 comprises alanine (Ala) instead of proline (Pro) at position 2 of the mature sequence.

Embodiment 105. The LAB of Embodiment 101, wherein said exogenous nucleic acid encoding the gliadin polypeptide further encodes a secretion leader sequence fused to said gliadin polypeptide coding sequence.

Embodiment 106. The LAB of Embodiment 105, wherein said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36, and variants thereof having 1, 2, or 3 variant amino acid positions.

Embodiment 107. The LAB of Embodiment 105, wherein said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36.

Embodiment 108. The LAB of Embodiment 105, wherein said gliadin polypeptide comprises an HLA-DQ2 specific epitope and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, and SL #36.

Embodiment 109. The LAB of Embodiment 105, wherein said gliadin polypeptide comprises a deamidated HLA-DQ2 specific epitope, and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #25, and SL #36

Embodiment 110. The LAB of Embodiment 102, wherein said gliadin polypeptide comprises an α1- and/or an α2-gliadin epitope.

Embodiment 111. The LAB of Embodiment 102, wherein said exogenous nucleic acid encoding a gliadin polypeptide encodes a gliadin polypeptide comprising or consisting of:

(DQ2)
(SEQ ID NO: 3)
LQLQPFPQPQLPYPQPQLPYPQPQLPYPQPQPF,
(dDQ2)
(SEQ ID NO: 7)
LQLQPFPQPELPYPQPQLPYPQPELPYPQPQPF,
or
(SEQ ID NO: 33)
LQLQPFPQPELPYPQPELPYPQPELPYPQPQPF.

Embodiment 112. The LAB of Embodiment 102, wherein said exogenous nucleic acid encoding a gliadin polypeptide encodes a gliadin polypeptide comprising or consisting of: LQLQPFPQPELPYPQPQLPYPQPELPYPQPQPF (SEQ ID NO: 7) (dDQ2), and further encodes a secretion leader selected from the secretion leader group consisting of SL #17, SL #21, SL #22, and SL #23.

Embodiment 113. The LAB of Embodiment 101, comprising a polycistronic expression unit comprising said exogenous nucleic acid encoding hIL-10 and said exogenous nucleic acid encoding the gliadin polypeptide.

Embodiment 114. The LAB of Embodiment 113, wherein said polycistronic expression unit comprises
(i) an endogenous gene promoter of an endogenous gene,
(ii) the endogenous gene positioned 3′ of the endogenous gene promoter,
(iii) an intergenic region, and
(iv) said exogenous nucleic acid encoding hIL-10,
wherein said exogenous nucleic acid encoding hIL-10 further encodes a secretion leader sequence fused in frame to said hIL-10 coding sequence, and wherein said endogenous gene and said exogenous nucleic acid encoding hIL-10 are transcriptionally and translationally coupled by said intergenic region.

Embodiment 115. The LAB of Embodiment 114, where said polycistronic expression unit further comprises
(i) a second intergenic region positioned 3′ of said exogenous nucleic acid encoding hIL-10, and
(ii) said exogenous nucleic acid encoding the gliadin polypeptide,
wherein said exogenous nucleic acid encoding said gliadin polypeptide further encodes a secretion leader sequence fused in frame to said gliadin polypeptide, and wherein said exogenous nucleic acid encoding said gliadin polypeptide and said exogenous nucleic acid encoding hIL-10 are transcriptionally and translationally coupled by the second intergenic region.

Embodiment 116. The LAB of Embodiment 113, wherein said polycistronic expression unit comprises:
(i) an endogenous gene promoter of an endogenous gene,
(ii) the endogenous gene positioned 3′ of the endogenous gene promoter,
(iii) an intergenic region, and
(iv) said exogenous nucleic acid encoding the gliadin polypeptide,
wherein said exogenous nucleic acid encoding said gliadin polypeptide further encodes a secretion leader sequence fused to said gliadin polypeptide, and wherein said endogenous gene and said exogenous nucleic acid encoding said gliadin polypeptide are transcriptionally and translationally coupled by said intergenic region.

Embodiment 117. The LAB of Embodiment 116, where said polycistronic expression unit further comprises:
(i) a second intergenic region positioned 3′ of said exogenous nucleic acid encoding said gliadin polypeptide, and
(ii) said exogenous nucleic acid encoding hIL-10,
wherein said exogenous nucleic acid encoding hIL-10 further encodes a secretion leader sequence fused to said hIL-10 coding sequence, and wherein said exogenous nucleic acid encoding hIL-10 and said exogenous nucleic acid encoding said gliadin polypeptide are transcriptionally and translationally coupled by the second intergenic region.

Embodiment 118. The LAB of Embodiment 101, wherein said LAB constitutively expresses and secretes said hIL-10 and said gliadin polypeptide.

Embodiment 119. The LAB of Embodiment 101, comprising the following chromosomally integrated polycistronic expression cassettes:
f. a first polycistronic expression cassette comprising an eno promoter positioned 5′ of an eno gene, a first intergenic region, an hIL-10 secretion leader sequence, said exogenous nucleic acid encoding hIL-10; a second intergenic region, a gliadin polypeptide secretion leader sequence, and said exogenous nucleic acid encoding said gliadin polypeptide;
g. a second polycistronic expression cassette comprising a usp45 promoter, usp45, and an exogenous nucleic acid encoding a trehalose-6-phosphate phosphatase and optionally an intergenic region, such as rpmD, between said usp45 and said exogenous nucleic acid encoding said trehalose-6-phosphate phosphatase; and
h. a third polycistronic expression cassette comprising nucleic acid encoding one or more trehalose transporters positioned 3′ of an hllA promoter (PhllA);
   and genetically modified to include:
i. inactivation or deletion of a trehalose-6-phosphate phosphorylase gene (trePP);
j. inactivation or deletion of a gene encoding a cellobiose-specific PTS system IIC component (ptcC); and
k. deletion of a thymidylate synthase gene (thyA).

Embodiment 120. The LAB of Embodiment 119, wherein said trehalose-6-phosphate phosphatase is Escherichia coli otsB.

Embodiment 121. The LAB of Embodiment 119 or 120, wherein the third polycistronic expression cassette comprises trehalose transporters genes LLMG_RS02300 and LLMG_RS02305.

Embodiment 122. A lactic acid bacterium (LAB) comprising an exogenous nucleic acid encoding a secretion leader sequence fused in frame to a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope, wherein said exogenous nucleic acid is chromosomally integrated in the LAB.

Embodiment 123. The LAB of Embodiment 122, wherein said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36, and variants thereof having 1, 2, or 3 variant amino acid positions.

Embodiment 124. The LAB of Embodiment 122 or 123, wherein said exogenous nucleic acid encoding a gliadin polypeptide encodes a gliadin polypeptide comprising or consisting of:

(DQ2)
(SEQ ID NO: 3)
LQLQPFPQPQLPYPQPQLPYPQPQLPYPQPQPF,
(dDQ2)
(SEQ ID NO: 7)
LQLQPFPQPELPYPQPQLPYPQPELPYPQPQPF,
or
(SEQ ID NO: 33)
LQLQPFPQPELPYPQPELPYPQPELPYPQPQPF.

Embodiment 125. The LAB of Embodiment 122, wherein said exogenous nucleic acid encoding a gliadin polypeptide encodes a gliadin polypeptide comprising or consisting of: LQLQPFPQPELPYPQPQLPYPQPELPYPQPQPF (SEQ ID NO: 7) (dDQ2), and a secretion leader selected from the secretion leader group consisting of SL #17, SL #21, SL #22, and SL #23.

Embodiment 126. A composition comprising the LAB of any one of embodiments 101 to 125.

Embodiment 127. A composition comprising:

a first LAB containing an exogenous nucleic acid encoding an interleukin-10 (IL-10) polypeptide and expresses the IL-10 polypeptide; and

a second LAB containing an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope.

Embodiment 128. A composition comprising:
(a) a lactic acid bacterium (LAB) comprising:

(i) an exogenous nucleic acid encoding human interleukin-10 (hIL-10) and

(ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,

wherein said exogenous nucleic acid encoding hIL-10 and said exogenous nucleic acid encoding a gliadin polypeptide are chromosomally integrated in the LAB;

or
(b) a first LAB containing an exogenous nucleic acid encoding an interleukin-10 (IL-10) polypeptide and expresses the IL-10 polypeptide; and

a second LAB containing an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope,

wherein said exogenous nucleic acid encoding hIL-10 and said exogenous nucleic acid encoding a gliadin polypeptide are chromosomally integrated in the LAB;

or
(c) a lactic acid bacterium (LAB) comprising an exogenous nucleic acid encoding a secretion leader sequence fused in frame to a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope, wherein said exogenous nucleic acid is chromosomally integrated in the LAB;

Embodiment 129. Use of the LAB of any one of embodiments 1 to 121 or the composition of Embodiment C or Embodiment CC in the treatment of celiac disease.

Embodiment 130. Use of the LAB of any one of embodiments 1 to 121 or the composition of Embodiment C or Embodiment CC for the preparation of a medicament for the treatment of celiac disease.

Embodiment 131. A polynucleotide sequence comprising a polycistronic expression unit comprising

(i) a nucleic acid encoding hIL-10, and

(ii) a nucleic acid encoding a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope,

wherein said nucleic acid encoding hIL-10 further encodes a secretion leader sequence fused to said hIL-10, and wherein said nucleic acid encoding said gliadin polypeptide further encodes a secretion leader sequence fused to said gliadin polypeptide.

Embodiment 132. The polynucleotide sequence of Embodiment 131, wherein said nucleic acid encoding said gliadin polypeptide and said nucleic acid encoding hIL-10 are transcriptionally and translationally coupled by an intergenic region.

Embodiment 133. The polynucleotide sequence of Embodiment 132, further comprising an L. lactis promoter positioned 5′ to said exogenous nucleic acid encoding hIL-10, wherein said exogenous nucleic acid encoding hIL-10 is transcriptionally controlled by said L. lactis promoter.

Embodiment 134. The polynucleotide sequence of Embodiment 133, wherein said L. lactis promoter is selected from the group comprising eno promoter, P1 promoter, usp45 promoter, gapB promoter, thyA promoter, and hllA promoter.

Embodiment 135. A polynucleotide sequence comprising a polycistronic integration vector comprising
(i) a first intergenic region,
(ii) a first open reading frame encoding a first therapeutic protein,
(iii) a second intergenic region, and
(iv) a second open reading frame encoding a second therapeutic protein,
wherein the first intergenic region is transcriptionally coupled at its 3′ end to the first open reading frame, the second intergenic region is transcriptionally coupled to the 3′ end of the first open reading frame, and the second intergenic region is transcriptionally coupled at its 3′ end to the second open reading frame.

Embodiment 136. The polynucleotide sequence of Embodiment 135, wherein one of either the first open reading frame and second open reading frame encodes hIL-10, and the other of the first open reading frame and second open reading frame encodes a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope.

Embodiment 137: The polynucleotide sequence of Embodiment 136, wherein the first open reading frame further encodes a secretion leader sequence fused to the first therapeutic protein and the second open reading frame further encodes a secretion leader sequence fused to the second therapeutic protein.

Embodiment 138: The polynucleotide sequence of any of Embodiements 135 to 137, further comprising nucleic acid sequences flanking the 5′ and 3′ ends of the at least one intergenic region transcriptionally coupled to at least one open reading frame or coding region, wherein the 5′ flanking nucleic acid comprises a nucleic acid sequence that is identical to coding sequence at the 3′ end of an integration target gene.

Embodiment 139: A polynucleotide sequence comprising:
(a) a polycistronic expression unit comprising:

(i) a nucleic acid encoding hIL-10, and

(ii) a nucleic acid encoding a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope,

wherein said nucleic acid encoding hIL-10 further encodes a secretion leader sequence fused to said hIL-10, and wherein said nucleic acid encoding said gliadin polypeptide further encodes a secretion leader sequence fused to said gliadin polypeptide; or
(b) a polycistronic integration vector comprising

(i) a first intergenic region,

(ii) a first open reading frame encoding a first therapeutic protein,

(iii) a second intergenic region, and

(iv) a second open reading frame encoding a second therapeutic protein,

wherein the first intergenic region is transcriptionally coupled at its 3′ end to the first open reading frame, the second intergenic region is transcriptionally coupled to the 3′ end of the first open reading frame, and the second intergenic region is transcriptionally coupled at its 3′ end to the second open reading frame.

Embodiment 140. A method of inducing oral tolerance to gluten in a subject at risk of celiac disease, comprising administering to a subject at risk of celiac disease a therapeutically effective amount of a lactic acid bacterium (LAB) engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,

wherein said exogenous nucleic acid encoding IL-10 and said exogenous nucleic acid encoding a gliadin polypeptide are chromosomally integrated in the LAB, thereby inducing oral tolerance.

Embodiment 141. The method of Embodiment 140, wherein said interleukin-10 is human interleukin-10 (hIL-10).

Embodiment 142. The method of Embodiment 140, wherein said subject at risk of celiac disease exhibits a risk factor, wherein the risk factor is a genetic predisposition.

Embodiment 143. The method of Embodiment 140, wherein administering the therapeutically effective amount of said LAB in said subject increases tolerance-inducing lymphocytes in a sample of lamina propria cells of said subject.

Embodiment 144. The method of Embodiment 140, wherein administering the therapeutically effective amount of said LAB in said subject increases CD4+ Foxp3⁺ regulatory T cells in a sample of lamina propria cells of said subject.

Embodiment 145. The method of Embodiment 140, wherein administering the therapeutically effective amount of said LAB in said subject increases a ratio of CD4⁺ Foxp3⁺ regulatory T cells over T_{H}1 cells expressing Tbet in a sample of lamina propria cell of said subject.

Embodiment 146. The method of Embodiment 140, wherein the development of villous atrophy upon exposure to gluten is prevented, inhibited or minimized in said subject.

Embodiment 147. The method of any one of embodiments 140 to 146, wherein the LAB is said LAB of any one of embodiments 101 to 121.

Embodiment 148. A method of reducing villous atrophy in a subject diagnosed with celiac disease, comprising administering to said subject having villous atrophy a therapeutically effective amount of a LAB engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2 specific epitope and/or at least one deamidated HLA-DQ2 specific epitope, and (b) at least one HLA-DQ8 specific epitope and/or at least one deamidated HLA-DQ8 specific epitope,

wherein LAB produces at least a 55% reduction of the villous atrophy relative to a reference LAB that does not express IL-10 and the gliadin polypeptide in a mouse model of celiac disease.

Embodiment 149. The method of Embodiment 148, wherein said interleukin-10 is human interleukin-10 (hIL-10).

Embodiment 150. The method of Embodiment 148, where the villous atrophy is present due to intestinal gluten exposure.

Embodiment 151. The method of Embodiment 148, wherein said LAB produces at least a 60%, 65%, 70%, 75%, 80%, 85%, 90%, 91%, 92%, 93%, 94%, 95%, 96%, 97% 98%, 99% or 100% reduction of the villous atrophy relative to the reference LAB that does not express IL-10 and the gliadin polypeptide in a mouse model of celiac disease.

Embodiment 152. The method of any one of embodiments 148 to 151, wherein said LAB is said LAB of any one of embodiments 101 to 121.

Embodiment 153. The method of any one of embodiments 148 to 151, wherein said administering:

a. reduces intraepithelial lymphocytosis in said subject as compared to intraepithelial lymphocytosis prior to administration to said subject and/or reduces the level of CD3⁺ intraepithelial lymphocytes (IELs) in a sample obtained from said subject as compared to CD3⁺ IELs present in a sample obtained from said subject prior to the administering step;

b. reduces the number of cytotoxic CD8+ IELs in said subject as compared to said cytotoxic CD8+ IELs present in a sample of said subject prior to administration;

c. reduces the level of Foxp3⁻Tbet⁺CD4⁺ T cells of said subject as compared to said Foxp3⁻Tbet⁺CD4⁺ T cells present in a sample of said subject prior to administration and/or increases the level of Foxp3⁺TberCD4⁺ T cells in a sample of lamina propria lymphocytes of said subject compared to said Foxp3⁻Tbet⁺CD4⁺ T cells present in a sample of said subject prior to administration;

d. prevents, inhibits or minimizes villous atrophy recurrence in said subject upon exposure to gluten; or e. improves villous height (Vh)-to-crypt depth (Cd) ratio in said subject and/or restores the Vh/Cd ratio to a normal range in said subject.

1. A lactic acid bacterium (LAB) comprising:
(i) an exogenous nucleic acid encoding human interleukin-10 (hIL-10) and
(ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,
wherein said exogenous nucleic acid encoding hIL-10 and said exogenous nucleic acid encoding a gliadin polypeptide are chromosomally integrated in the LAB.

2. A lactic acid bacterium (LAB) comprising an exogenous nucleic acid encoding a secretion leader sequence fused in frame to a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2 specific epitope and/or at least one deamidated HLA-DQ2 specific epitope, and (ii) at least one HLA-DQ8 specific epitope and/or at least one deamidated HLA-DQ8 specific epitope, wherein said exogenous nucleic acid is chromosomally integrated in the LAB.

3. (canceled)

4. The LAB of claim 1, comprising a polycistronic expression unit comprising said exogenous nucleic acid encoding hIL-10 and said exogenous nucleic acid encoding the gliadin polypeptide.

5. The LAB of claim 4, wherein said LAB constitutively expresses and secretes said hIL-10 and said gliadin polypeptide.

6. The LAB of claim 3, wherein a secretion leader is fused to said gliadin polypeptide, and wherein said secretion leader is selected from the secretion leader group consisting of SL #1, SL #6, SL #8, SL #9, SL #13, SL #15, SL #17, SL #20, SL #21, SL #22, SL #23, SL #24, SL #25, SL #32, SL #35, and SL #36, and variants thereof having 1, 2, or 3 variant amino acid positions.

7. The LAB of claim 6, wherein said gliadin polypeptide comprises:
(a) an HLA-DQ2 specific epitope and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, and SL #36; or
(b) a deamidated HLA-DQ2 specific epitope, and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #25, and SL #36.

8. The LAB of claim 7, wherein said exogenous nucleic acid encoding a gliadin polypeptide encodes a gliadin polypeptide comprising or consisting of:
(DQ2)
(SEQ ID NO: 3)
LQLQPFPQPQLPYPQPQLPYPQPQLPYPQPQPF,
(dDQ2)
(SEQ ID NO: 7)
LQLQPFPQPELPYPQPQLPYPQPELPYPQPQPF,
or
(SEQ ID NO: 33)
LQLQPFPQPELPYPQPELPYPQPELPYPQPQPF.

9. (canceled)

10. The LAB of claim 5, comprising the following chromosomally integrated polycistronic expression cassettes:
a. a first polycistronic expression cassette comprising an eno promoter positioned 5′ of an eno gene, a first intergenic region, an hIL-10 secretion leader sequence, said exogenous nucleic acid encoding hIL-10; a second intergenic region, a gliadin polypeptide secretion leader sequence, and said exogenous nucleic acid encoding said gliadin polypeptide;
b. a second polycistronic expression cassette comprising a usp45 promoter, usp45, and an exogenous nucleic acid encoding a trehalose-6-phosphate phosphatase and optionally an intergenic region, such as rpmD, between said usp45 and said exogenous nucleic acid encoding said trehalose-6-phosphate phosphatase; and
c. a third polycistronic expression cassette comprising nucleic acid encoding one or more trehalose transporters positioned 3′ of an hllA promoter (PhllA);
and genetically modified to include:
d. inactivation or deletion of a trehalose-6-phosphate phosphorylase gene (trePP);
e. inactivation or deletion of a gene encoding a cellobiose-specific PTS system IIC component (ptcC); and
f. deletion of a thymidylate synthase gene (thyA).

11. The LAB of claim 10, wherein said gliadin polypeptide comprises:
(a) an HLA-DQ2 specific epitope and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #24, SL #25, and SL #36; or
(b) a deamidated HLA-DQ2 specific epitope, and said secretion leader fused to said gliadin polypeptide is selected from the secretion leader group consisting of SL #1, SL #6, SL #8, SL #9, SL #13, SL #15, SL #17, SL #20, SL #21, SL #22, SL #23, SL #25, and SL #36.

12. The LAB of claim 1, which is sAGX0868.

13. A composition comprising:
(a) a lactic acid bacterium (LAB) of claim 1;
or
(b) a first LAB containing an exogenous nucleic acid encoding an interleukin-10 (IL-10) polypeptide and expresses the IL-10 polypeptide; and
a second LAB containing an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope,
wherein said exogenous nucleic acid encoding hIL-10 and said exogenous nucleic acid encoding a gliadin polypeptide are chromosomally integrated in the LAB.

14-15. (canceled)

16. A polynucleotide sequence comprising:
(a) a polycistronic expression unit comprising:
(i) a nucleic acid encoding hIL-10, and
(ii) a nucleic acid encoding a gliadin polypeptide comprising at least one HLA-DQ2-specific epitope, at least one deamidated HLA-DQ2 specific epitope, at least one HLA-DQ8 specific epitope, at least one deamidated HLA-DQ8 specific epitope, or a combination of (i) at least one HLA-DQ2-specific epitope and/or at least one deamidated HLA-DQ2 specific epitope, and (ii) at least one HLA-DQ8 specific epitope and/or at least one deamidated HLA-DQ8 specific epitope,
wherein said nucleic acid encoding hIL-10 further encodes a secretion leader sequence fused to said hIL-10, and wherein said nucleic acid encoding said gliadin polypeptide further encodes a secretion leader sequence fused to said gliadin polypeptide; or
(b) a polycistronic integration vector comprising
(i) a first intergenic region,
(ii) a first open reading frame encoding a first therapeutic protein,
(iii) a second intergenic region, and
(iv) a second open reading frame encoding a second therapeutic protein,
wherein the first intergenic region is transcriptionally coupled at its 3′ end to the first open reading frame, the second intergenic region is transcriptionally coupled to the 3′ end of the first open reading frame, and the second intergenic region is transcriptionally coupled at its 3′ end to the second open reading frame.

17. A method of inducing oral tolerance to gluten in a subject at risk of celiac disease, comprising administering to a subject at risk of celiac disease a therapeutically effective amount of a lactic acid bacterium (LAB) engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,
wherein said exogenous nucleic acid encoding IL-10 and said exogenous nucleic acid encoding a gliadin polypeptide are chromosomally integrated in the LAB, thereby inducing oral tolerance.

18-19. (canceled)

20. The method of claim 17 wherein administering the therapeutically effective amount of said LAB in said subject (i) increases tolerance-inducing lymphocytes in a sample of lamina propria cells of said subject; (ii) increases CD4+ Foxp3+ regulatory T cells in a sample of lamina propria cells of said subject; (iii) increases a ratio of CD4⁺ Foxp3⁺ regulatory T cells over T_{H}1 cells expressing Tbet in a sample of lamina propria cell of said subject; or (iv) prevents, inhibits, or minimizes the development of villous atrophy upon exposure to gluten in said subject.

21-23. (canceled)

24. A method of reducing villous atrophy in a subject diagnosed with celiac disease, comprising administering to said subject having villous atrophy a therapeutically effective amount of a LAB engineered to express (i) interleukin-10 (IL-10) and (ii) a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2 specific epitope and/or at least one deamidated HLA-DQ2 specific epitope, and (b) at least one HLA-DQ8 specific epitope and/or at least one deamidated HLA-DQ8 specific epitope,
wherein LAB produces at least a 55% reduction of the villous atrophy relative to a reference LAB that does not express IL-10 and the gliadin polypeptide in a mouse model of celiac disease.

25. (canceled)

26. The method of claim 24, where the villous atrophy is present due to intestinal gluten exposure.

27. The method of claim 24, wherein said LAB produces at least a 60%, 65%, 70%, 75%, 80%, 85%, 90%, 91%, 92%, 93%, 94%, 95%, 96%, 97% 98%, 99% or 100% reduction of the villous atrophy relative to the reference LAB that does not express IL-10 and the gliadin polypeptide in a mouse model of celiac disease.

28. The method of claim 24, wherein said administering:
a. reduces intraepithelial lymphocytosis in said subject as compared to intraepithelial lymphocytosis prior to administration to said subject and/or reduces the level of CD3⁺ intraepithelial lymphocytes (IELs) in a sample obtained from said subject as compared to CD3⁺ IELs present in a sample obtained from said subject prior to the administering step;
b. reduces the number of cytotoxic CD8⁺ IELs in said subject as compared to said cytotoxic CD8⁺ IELs present in a sample of said subject prior to administration;
c. reduces the level of Foxp3⁻Tbet⁺CD4⁺ T cells of said subject as compared to said Foxp3⁻Tbet⁺CD4⁺ T cells present in a sample of said subject prior to administration and/or increases the level of Foxp3⁺Tbet⁻CD4⁺ T cells in a sample of lamina propria lymphocytes of said subject compared to said Foxp3⁻Tbet⁺CD4⁺ T cells present in a sample of said subject prior to administration;
d. prevents, inhibits or minimizes villous atrophy recurrence in said subject upon exposure to gluten; or
e. improves villous height (Vh)-to-crypt depth (Cd) ratio in said subject and/or restores the Vh/Cd ratio to a normal range in said subject.

29. The method of claim 17, wherein said LAB comprises:
(i) an exogenous nucleic acid encoding human interleukin-10 (hIL-10) and
(ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,
and wherein said exogenous nucleic acid encoding hIL-10 and said exogenous nucleic acid encoding a gliadin polypeptide are chromosomally integrated in the LAB.

30. The method of claim 17, wherein said LAB is administered in a unit dosage form comprising from about 10⁴ colony forming units (cfu) to about 10¹² cfu per day, from about 10⁶ cfu to about 10¹² cfu per day, or from about 10⁹ cfu to about 10¹² cfu per day.

31. The method of claim 17, wherein said LAB is sAGX0868.

32. The method of claim 24, wherein said LAB comprises:
(i) an exogenous nucleic acid encoding human interleukin-10 (hIL-10) and
(ii) an exogenous nucleic acid encoding a gliadin polypeptide comprising at least one HLA-DQ2 specific epitope, at least one deamidated HLA-DQ2 specific epitope, at least one HLA-DQ8 specific epitope, at least one deamidated HLA-DQ8 specific epitope, or a combination of (a) at least one HLA-DQ2-specific epitope and/or at least one deamidated HLA-DQ2 specific epitope, and (b) at least one HLA-DQ8 specific epitope and/or at least one deamidated HLA-DQ8 specific epitope,
and wherein said exogenous nucleic acid encoding hIL-10 and said exogenous nucleic acid encoding a gliadin polypeptide are chromosomally integrated in the LAB.

33. The method of claim 24, wherein said LAB is administered in a unit dosage form comprising from about 10⁴ colony forming units (cfu) to about 10¹² cfu per day, from about 10⁶ cfu to about 10¹² cfu per day, or from about 10⁹ cfu to about 10¹² cfu per day.

34. The method of claim 24, wherein said LAB is sAGX0868.


SEQUENCE LISTING (121 sequences)

SEQ ID NO: 1 - protein, 290 aa, Triticum aestivum
MVRVPVPQLQPQNPSQQQPQEQVPLVQQQQFPGQQQPFPPQQPYPQPQPFPSQQPYLQLQPFPQPQLPYPQPQLPYPQPQLPYPQPQPFRPQQPYPQSQPQYSQPQQPISQQQQQQQQQQQQKQQQQQQQQILQQILQQQLIPCRDVVLQQHSIAYGSSQVLQQSTYQLVQQLCCQQLWQIPEQSRCQAIHNVVHAIILHQQQQQQQQQQQQPLSQVSFQQPQQQYPSGQGSFQPSQQNPQAQGSVQPQQLPQFEEIRNLALETLPAMCNVYIPPYCTIAPVGIFGTNYR

SEQ ID NO: 2 - DNA, 810 bp, Triticum aestivum
atggttagagttccagtgccacaattgcagccacaaaatccatctcagcaacagccacaagagcaagttccattggtacaacaacaacaatttctagggcagcaacaaccatttccaccacaacaaccatatccacagccgcaaccatttccatcacaactaccatatctgcagctgcaaccatttccgcagccgcaactaccatattcacagccacaaccatttcgaccacaacaaccatatccacaaccgcaaccacagtattcgcaaccacaacaaccaatttcacagcagcagcagcagcagcagcagcagcaacaacaacaacaacaacaacaacaaatccttcaacaaattttgcaacaacaactgattccatgcatggatgttgtattgcagcaacacaacatagcgcatggaagatcacaagttttgcaacaaagtacttaccagctgttgcaagaattgtgttgtcaacacctatggcagatccctgagcagtcgcagtgccaggccatccacaatgttgttcatgctattattctgcatcaacaacaaaaacaacaacaacaaccatcgagccaggtctccttccaacagcctctgcaacaatatccattaggccagggctccttccggccatctcagcaaaacccacaggcccagggctctgtccagcctcaacaactgccccagttcgaggaaataaggaacctagcgctacagacgctacctgcaatgtgcaatgtctacatccctccatattgcaccatcgcgccatttggcatcttcggtactaactatcgatga

SEQ ID NO: 3 - protein, 33 aa, Triticum aestivum
LQLQPFPQPQLPYPQPQLPYPQPQLPYPQPQPF

SEQ ID NO: 4 - DNA, 99 bp, Triticum aestivum
cttcaacttcaaccatttccacaaccacaacttccatacccacaaccacaacttccatacccacaaccacaacttccatacccacaaccacaaccattt

SEQ ID NO: 5 - protein, 18 aa, Triticum aestivum
QYPSGQGSFQPSQQNPQA

SEQ ID NO: 6 - DNA, 54 bp, Triticum aestivum
caatacccatcaggtcaaggttcatttcaaccatcacaacaaaacccacaagct

SEQ ID NO: 7 - protein, 33 aa, Artificial Sequence
  source: /note=“Description of Artificial Sequence Synthetic polypeptide”
LQLQPFPQPELPYPQPQLPYPQPELPYPQPQPF

SEQ ID NO: 8 - DNA, 99 bp, Artificial Sequence
  source: /note=“Description of Artificial Sequence Synthetic oligonucleotide”
cttcaacttcaaccatttccacaaccagaacttccatacccacaaccacaacttccatacccacaaccagaacttccatacccacaaccacaaccattt

SEQ ID NO: 9 - protein, 18 aa, Artificial Sequence
  source: /note=“Description of Artificial Sequence Synthetic peptide”
QYPSGEGSFQPSQENPQA

SEQ ID NO: 10 - DNA, 54 bp, Artificial Sequence
  source: /note=“Description of Artificial Sequence Synthetic oligonucleotide”
caatacccatcaggtgaaggttcattccaaccatcacaagaaaacccacaagct

SEQ ID NO: 11 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 12 - protein, 160 aa, Artificial Sequence
  source: /note=“Description of Artificial Sequence Synthetic polypeptide”
SAGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 13 - DNA, 480 bp, Artificial Sequence
  source: /note=“Description of Artificial Sequence Synthetic polynucleotide”
tcagctggtcaaggtactcaatcagaaaactcatgtactcactttccaggtaacttgccaaacatgcttcgtgatttgcgtgatgctttttcacgtgttaaaactttttttcaaatgaaagatcaacttgataacttgcttttgaaagaatcacttttggaagattttaaaggttaccttggttgtcaagctttgtcagaaatgatccaattttaccttgaagaagttatgccacaagctgaaaaccaagatccagatatcaaagctcacgttaactcattgggtgaaaaccttaaaactttgcgtcttcgtttgcgtcgttgtcaccgttttcttccatgtgaaaacaaatcaaaagctgttgaacaagttaaaaacgcttttaacaaattgcaagaaaaaggtatctacaaagctatgtcagaatttgatatctttatcaactacatcgaagcttacatgactatgaaaatccgtaac

SEQ ID NO: 14 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 15 - DNA, 402 bp, Homo sapiens
gctccaacttcatcatcaactaaaaaaactcaattgcaacttgaacacttgcttttggatcttcaaatgatcttgaacggtatcaacaactacaaaaacccaaaacttactcgtatgttgacttttaaattttacatgccaaaaaaagctactgaacttaaacacttgcaatgtcttgaagaagaattgaaaccacttgaagaagttttgaaccttgctcaatcaaaaaactttcacttgcgtccacgtgatcttatctcaaacatcaacgttatcgttttggaacttaaaggttcagaaactacttttatgtgtgaatacgctgatgaaactgctactatcgttgaatttttgaaccgttggatcactttttgtcaatcaatcatctcaactttgacttaa

SEQ ID NO: 16 - protein, 100 aa, Homo sapiens
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSE

SEQ ID NO: 17 - DNA, 16 bp, Lactococcus lactis
taaggaggaaaaaatg

SEQ ID NO: 18 - DNA, 10 bp, Lactococcus lactis
ggaggaaaaa

SEQ ID NO: 19 - protein, 27 aa, Unknown
  source: /note=“Description of Unknown secretion sequence”
MKKKIISAILMSTVILSAAAPLSGVYA

SEQ ID NO: 20 - DNA, 81 bp, Unknown
  source: /note=“Description of Unknown secretion sequence”
atgaaaaaaaagattatctcagctattttaatgtctacagtgatactttctgctgcagccccgttgtcaggtgtttacgcc

SEQ ID NO: 21 - DNA, 81 bp, Unknown
  source: /note=“Description of Unknown secretion sequence”
atgaagaagaaaatcattagtgccatcttaatgtctacagtgattctttcagctgcagctcctttatcaggcgtttatgca

SEQ ID NO: 22 - DNA, 564 bp, Artificial Sequence
  source: /note=“Description of Artificial Sequence Synthetic polynucleotide”
atgaaaaaaaagattatctcagctattttaatgtctacagtgatactttctgctgcagccccgttgtcaggtgtttacgcctcagctggtcaaggtactcaatcagaaaactcatgtactcactttccaggtaacttgccaaacatgcttcgtgatttgcgtgatgctttttcacgtgttaaaactttttttcaaatgaaagatcaacttgataacttgcttttgaaagaatcacttttggaagattttaaaggttaccttggttgtcaagctttgtcagaaatgatccaattttaccttgaagaagttatgccacaagctgaaaaccaagatccagatatcaaagctcacgttaactcattgggtgaaaaccttaaaactttgcgtcttcgtttgcgtcgttgtcaccgttttcttccatgtgaaaacaaatcaaaagctgttgaacaagttaaaaacgcttttaacaaattgcaagaaaaaggtatctacaaagctatgtcagaatttgatatctttatcaactacatcgaagcttacatgactatgaaaatccgtaactaa

SEQ ID NO: 23 - DNA, 107 bp, Lactococcus lactis
aaaacgccttaaaatggcattttgacttgcaaactgggctaagatttgctaaaatgaaaaatgcctatgtttaaggtaaaaaacaaatggaggacatttctaaaatg

SEQ ID NO: 24 - DNA, 22 bp, Lactococcus lactis
gcaaaactaggaggaatatagc

SEQ ID NO: 25 - DNA, 180 bp, Artificial Sequence
  source: /note=“Description of Artificial Sequence Synthetic polynucleotide”
atgaaaaaagtgattaaaaaagcggcgattggcatggtggcgttttttgtggtggcggcgagcggcccggtgtttgcgcttcaacttcaaccatttccacaaccagaacttccatacccacaaccacaacttccatacccacaaccagaacttccatacccacaaccacaaccattttaa

SEQ ID NO: 26 - DNA, 3289 bp, Artificial Sequence
  source: /note=“Description of Artificial Sequence Synthetic polynucleotide”
gatggctgaagctccaactcatgaacaagttgaccatgttgtggatacaattgttgaagttgttgaagaggaaattggtgtgaaataaagaaaagacaaggagaatattcttcttgtcttttttcatatcctaaaactctacctactgtggtagagtttttttatcttttttggcgtctagcaaactctgtaaaacgaaaacggtcaacctgatgtcgtgattcagtatactggaaaagtgtattatcggcaagatagacatgagatttcacggaaacaacatgatggtccttcggatttaaatcaagatatgtaaaatcatcttcacaagcaaaatcaatggtaacttctttttgggcataggcaatatcaagtcccaaagccccttctaaataatcgtaagtagaattttgggcatgtgcgggggtcaaaccatcagcgtatttttctaaaaataaatcccaatccaaaatggaaaatttaccatctacttttcttcttctaagaatactaagggcttggtcgccgatggcgaatccagtagtttctgaaagagctggggtaatttttatactttcaaattttattacttcagtttcactgtgaaaacccattgaagtttgcaattctttatatgaagttaagccggaaatagggaaaaggagccgatcgtgagcgaggacaatgctgccatagccatgtcttctttggatgagccctttttcttctaaaatttttaaagcttgtctgacggttgaacggctactttcataactaatagaaagttcattctcgcttggaagaatatcgttcgttttatagatatcattaaaaatctttttttctaaatcttgcaaaatcacttcatatttcttcatactttatattttatcataaaaataattgttaacgcttgctgaaaacgtttttatgaaaacgccttaaaatggcattttgacttgcaaactgggctaagatttgctaaaatgaaaaatgcctatgtttaaggtaaaaaacaaatggaggacatttctaaaatgtttggaataggaaaaaagaaagaattgagagatgataaaagcctttatgctccagtttctggggaagttatcaacctttcaacagtcaacgaccccgtattttcaaaaaagataatgggagacgggttcgcggttgagccaaaagaaaataaaatttttgccccagtttctgcaaaagtaactttggttcaaggacatgcaattggttttaaacgtgctgatggcttagatgtacttttacatcttggaattgatacagtagctcttaaaggtcttcattttaaaatcaaggtcaaagttgatgatattgtcaatggtggtgatgagcttggaagcgttgattgggcacagattgaagctgcaggtttagataaaacgacaatggttatctttacaaatacaaaagataaactctctgagttcaatgtcaattatggaccagctacttctggaagtgaacttggtaaggcaagtgttaaataaggaggaaaaaatggcaaattattcacaacttgcgacagaaattatcgcaaatgtaggtggcgctgagaatgtcacaaaagttattcactgtatcactcgtcttcgttttaccttgaaagacaaagataaagcagatacggcggcgattgaagccttacctggtgtcgctggagctgtttataactcaaacttgaatcaatatcaagtagttattggacaagctgtagaagatgtttatgacgaggttgttgaacagcttggagattcagttgttgatgaagatgcaacggcgcaagcacttactgcaacagcaccggctagtggtaaaaaacaaaatccaattgttcatgctttccaagtggttattgggacaattacaggttcgatgattccaattattggtttacttgcggctggtgggatgattaatggattattaagtatctttgttaaaggaaatcgtttaattgaagtgattgaccctgcaagttcaacttacgtcattatctcaactctagcaatgacaccattttatttcttacctgttttagtaggattttcagcagcaaaacaattagcacctaaagatactgttttacaatttattggtgctgctgttggtggtttcatgattaatccagggattactaacttggtaaatgctcatgttggaacaaatgcggccggtaaaaatgttgttgttgaagcagcagctccagtagcaaatttccttggagtcacttttaatacaagttattttggaattccggttgctttgccaagttatgcttatacaattttcccaatcattgtggcggtagcaatcgctaaacctttgaatgcttggttgaaaaaggttttaccacttgccttgcgtccaattttccaaccgatgattactttcttcatcactgcttcaatcattttactcttggtcggtcctgttatttcaacaatttcatctggtttgtcattcgttattgaccatatcttgtcattaaacttagggattgcaagtattatcgtcggtggtttgtatcaatgtttggttatatttggtttgcactggttggttgtaccacttatttcacaagagttggcagcaacaggagcaagctcacttaatatgattgttagcttcacaatgcttgcgcaaggagttggtgccttgactgtcttctttaaatctaaaaaagctgaccttaaaggactttctgctccagctgccatttcggctttttgtggagtaactgaacctgccatgtacggaattaacttgaaatatgttcgcgtcttcatcatgtcttcaattggtgcagcaattggtgctgggattgccggatttggtggcttacaaatgtttggattttcagggtcattgattagttttcctaactttatctctaatccattgacgcatcatgcacctgcgggtaacttaatgctcttctggattgccactgcggtatgtgctgttgccactttcttattagtttggttctttggttacaaggatactgatgtcatgggacaaggagttgaacaaaaaaatgcatttaaggatgctgtaaaataaatagttttgctcttaataaagttttgatacaaggatttacaattattttttgataaaaaaattactgatagaaatgaaaaaaattctgtcagtaattttggaaagtcattctaaaaaattcattttaaaatgacgagaaagaaggtaaaaagatgtttaaagcagtattgtttgatttagatggcgtaat

SEQ ID NO: 27 - DNA, 1700 bp, Artificial Sequence
  source: /note=“Description of Artificial Sequence Synthetic polynucleotide”
gtaattctaatgctggtgggaatacaaattcaggcactagtactggaaatactggaggaacaactactggtggtagcggtataaatagttcaccaattggaaatccttatgctggtggtggatgtactgactatgtatggcaatactttgctgcacaaggaatttatatcagaaatatcatgcctggtaatggtggacaatgggcttctaatggacctgcccaaggcgtgctccatgttgtaggagctgctcctggtgttatcgcatcaagcttctcagctgattttgttggatatgcaaactcaccttacggtcacgtagctattgtaaaatcagttaattcagatggtacaattactatcaaagaaggcggatatggtacaacttggtggggacatgaacgtactgtaagtgcgtctggtgttactttcttgatgccaaactaaggaggaaaaaatgacagaaccgttaaccgaaacccctgaactatccgcgaaatatgcctggttttttgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaaaaaaagtcttaataaataaaaaatagtggtttgatagtggggaataattttccttctgtcaaatcattttttattattgtggtataataataaggaaaaatgataaggggatagatacaaatgtgtggaattgtcggctttattgaccggatcgatcaaaatgataaatcaaaaactttagaagaaatgatggatacaatcgctcaccgaggtccaagtagttcaggtgaatttattgacgaaggagcagcaattggttttcgtcgcctgagtattattgaccttgagggtggagatcaacctatctttaatgaagataaaactaaacttataacctttaatggcgaaatttataatttccgtgaattgcgtgaagaccttatctctaaaggtcatgattttactactcatgctgatacagaagtgcttttacatggttacg

SEQ ID NO: 28 - DNA, 2512 bp, Artificial Sequence
  source: /note=“Description of Artificial Sequence Synthetic polynucleotide”
aacctgtgggagggcgaaagctcttttactatgtaaaaataagtataaacagaaacggaatataagaaatgactgataagtacgagaatccaacaagtgatgactacatgggtgtggtaatgggcattatcatgagtggaggaaacgccaaaggtttagccttccaagccattcaacaagctaaagatggaaaatttgcagaagcagaaagctcattaaatgaagcgagtgaacaacttcgtgaagcccatgatgttcaaacagatctcttaacacgtttggcacaaggagaaaaaatcggctggaatctttacatggttcacgctcaagaccatttgatgaatgctattacctttaaagaccttgcagtagaagttgttggtcaagaacgacgccttcaagcacttgaaaacaaataatacagctcctagcttattagaaaagcaagattaaaagttgcggagacgcaacttttttctaacaaaaatcgttgagtaagaaaggagtagaaaatgagtttttttggaaatgttgactttcaaaaattaacctatacagaaaggacttgctacagttatcttcgcgataatgttgataaaatcccttatttacgcgtccgtgacattgctttagaggcccatgttggaacttcaagcgtcatgcgtttaatacataaaatgggttatgattcgtatacggattttaaagaatatattattgataaaaaagagctagaaaaaggaatttctaacactgctattcctttctcatcagatattttttcaggagacgttgaacagcgtttagataatttagcgcaacgtgtgattgaaagtgataatatcatttttaccggagttggctcaagtggtttaatctgtgattatgcagctagaagattagctggtgtgggcattaatactttttcatttagcgatgtaacttatccgattgcttcaaaattgcaaaatacaaccaatactttagtaattgctttatccatttcaggtgaaactaatgaaatcattgaagtactgacgagtcttcggtcaaataaagatgtttatatttcgagtatcacgcccaaaattaattcgagtatcgctgaactgagcgattttgtcctgacctatcgaatcaatgaacaccgaattaacacgcattatgacttaacgaggcaattacctacggtctatttgacagaacgtttaacagatctcgtttatcaacgttccaactaattgcaaaatttagatgagctcaagacttcttgagcttatcttgttaagaaatgataatttaataattctaataattacaaggagaaattatgactttaaaaaaagacttcctctggggaggagcagttgctgcacatcaacttgaaggaggctggaatgctggtggaaagggagtttccgtagcagatgtgatgacagcaggtagtaacggcgtagaaagaaaaatcacagatggagtaattgagggagaaaattaccctaatcatgaagctattgatttctatcaccactataaagaagatgtagcactttttgcagaactcggtttaaattgttttagaacatctattgcttggacacgcatttttcctaagggagatgagtctgaacctaacgaagagggcttgcaattctatgatgacttatttgatgaatgtttaaaaaatggtattgaacctgtcattactctttctcactttgaacttccttatcatttagtgacagagtatggtggattccgaaaccgaaaactcattgatttctttgtccatttttcagaagtagtgatgaatcgttataaagataaagttaagtattggatgactttcaatgaaattaataaccaagcaaactttatgcgcgactttgccccttttaccaattctggtttgaaatttccagagggagcaagcgaaaaagagcgtgaagaaatcatgtatcaagcggctcattatgaacttgttgcctcggccaaagttgtagagttgggacataaaattaatcctgatttccaaattggttgtatgattgccatgtgtccgatttatcctgcaacttgtaaaccggaagacatgatggcttcaaccgttgctatgcaacgtcgttattggtttgcagatgtccacgttcgcggacattacccaagttatttaaaggcttattttgaacgtaaagaatttaaacttgatattagtgatgaagacttagaagttttgaaaaatggaaaagttgattatattggtttttcttactatatgagttttgcaattaaggaccatggaaaagccccaacttttgattataacgaagataaagatttagttaaaaatgactatgttaaggcttctgaatgggggtggcaaattgacccattaggtttacgttatgcgatgaattggttctatgaacgttatgaaaaaccacttttcattgttgaaaatggctttggtgctgtagatgaagttgaagctgatggaagtattcacgaccca

SEQ ID NO: 29 - DNA, 2066 bp, Artificial Sequence
  source: /note=“Description of Artificial Sequence Synthetic polynucleotide”
accgaatttccgaaatccgcggtagttgacagtgtgtcaaatgttgaagcatttcaaacggtatacacgggtagcacaggattaattgtagcaatcataattggttttattgtttcattagtctatatacaattgagcaaaagaaatttagttattaaattaccagctggagttcctccaatggttgtagattcactaagtccagcaattatttcaatggtgattttctgtttgatgttcgggattcgtgtgggattctcttatacgccattccatgatattttcaatttctcaacacaactaattcaagcaccgttgactggtgctgtggcaaatccatgggttcttatgggcatctttacctttggtaatttcttatggttctttggtatccaccctaatttaattgggggaattttaaatccattgttattaacaatgtcatatgctaatattgatgcctatgctgccggaaaacctgtaccatacttacaaatgatgattgtgtttgctgtgggtgcgaacgcatggggcggaagtggaaatacttatgggttagttatttcaatgtttacggcaaaatctgaacgctataaacaattattaaaattaggtgcaattcctagtattttcaatatcagtgaaccattactttttggtcttccaatgatgttaaatcctcttttctttattcctttggttttccaaccagcaattttaggaactgtagcattgggcttggcaaagatattatatattacaaatctgaatccaatgacggcacttcttccttggacgacaccagcacctgtgagaatggccatttcaggtggacttccatttttgattatttttgcaatctgtttagtcttgaatgttcttatttactacccattctttaaggtggcgtataataaagctttagaagaagaaaaagcagctgttgaattagagggttcagaaactgcctgatggattaatctataagttactgacaaaactgtcagtaactttttttgtgggaaaaatgtatttttatgaccgtaaagaatctgtcagtagaagtctgaaattcgtttaaaaatcgactagaataggctttaacgacaagatgttttaaagagtacgctctaaatgtatttttgtatttttgtttgattacgaagtttaaatttaattgacaaatgttttaaaatgagtataataggacttgtaaccgattttatttttataaaggagaaagaaagatgaacaaacttttacttggaacagcctttataggggctagcttactgattggtgggggtgctcatgcagatacaaatgtttatcgtttgtataatcataatactggtgagcacttctatacaactagtgggacagaaaagaatgctaatgtaagtgcgggttggacttatgaaggtgtcggttggatcgcaccaacaacaagttcaagcccagtttaccgtgtgtacaatccaaatgcattattacacaaaaagcaagtatgaagcccaaagtttagtaaataagggttggaaatgggataataacggaaaggcggtcttctattctggaggttctcaagccgtatatgtcgcttataatcccaatgcacaatctggcgctcacaattacacggaaagtagctttgagcaaaatagcttattgaatactggttggaaatatggggcagtagcttggtacgggattggagtaaaaaacgaaatgttaaacattgctcaaattgttagtggtaatttttctagtattgttggaacttggaaagatacttctggaaatatgcttgaaattaatgcaatgggaaatcttactttaatatggaaaggggcaaagaatcaaacctttgaacttggcgcaggtcaacaatttaatggaactgcagatattgccttaaaaaatggagagatttcccctggtagtccacttaacatttttgttgtaccaacagaagttgctttccctaataataaaaaagtagacgattcaactgggcaacaacgaatttttgtgaattattctggtacaagccctcaaatggcgaa

SEQ ID NO: 30 - DNA, 2947 bp, Artificial Sequence
  source: /note=“Description of Artificial Sequence Synthetic polynucleotide”
catcgctgaagctatcatcggttatgaagttactgaccaacaagctattgaccgtgcaatgatcgctcttgacggtactgaaaacaaaggtaaattgggagctaacgctattcttggtgtttctatcgctgctgctcgtgctgctgctgatgaacttggtgttccactttacaactaccttggcggattcaacgctaaagtattgccaactccaatgatgaacatcatcaatggtggttctcactcagacgcccctatcgctttccaagaattcatgatcgtaccagttggtgcacctacattcaaagaagcgcttcgttggggtgctgaaatcttccacgctcttaagaaaattcttaaagctcgtggacttgaaacagctgtcggtgacgaaggtggattcgctcctaaattcgacggaactgaagacggtgtagaaactatccttaaagcaatcgaagcagctggttacaaagctggtgaagatggcgttatgatcggtttcgactgtgcatcatcagaattctacgaaaacggtgtttacgactacactaaattcgaaggtgaaggcggtaaaaaactttcagcttctgaacaagttgactaccttgaagaactcgtttctaaatacccaatcatcactattgaagatggtatggacgaaaacgactgggatggatggaaaatccttactgaacgtcttggtaaaaaagttcaactcgttggtgacgacttcttcgttacaaacactaaataccttgaacgtggtatccgtgaaaatgcttcaaacgctatcttgatcaaagttaaccaaatcggtactttgacagaaactttcgaagctattgaaatggctaaagaagctggtttcacagcaatcgtatctcaccgttcaggtgaaactgaagattcaacaatctcagacatcgctgttgcaactaacgctggtcaaatcaaaactggttcactttcacgtacagaccgtatggctaaatacaaccaattgcttcgtattgaagaccaattggctgaagttgctcaatacaaaggtcttaaagcattctacaaccttaaaaaataaggaggaaaaaatgaaaaaaaagattatctcagctattttaatgtctacagtgatactttctgctgcagccccgttgtcaggtgtttacgcctcagctggtcaaggtactcaatcagaaaactcatgtactcactttccaggtaacttgccaaacatgcttcgtgatttgcgtgatgctttttcacgtgttaaaactttttttcaaatgaaagatcaacttgataacttgcttttgaaagaatcacttttggaagattttaaaggttaccttggttgtcaagctttgtcagaaatgatccaattttaccttgaagaagttatgccacaagctgaaaaccaagatccagatatcaaagctcacgttaactcattgggtgaaaaccttaaaactttgcgtcttcgtttgcgtcgttgtcaccgttttcttccatgtgaaaacaaatcaaaagctgttgaacaagttaaaaacgcttttaacaaattgcaagaaaaaggtatctacaaagctatgtcagaatttgatatctttatcaactacatcgaagcttacatgactatgaaaatccgtaactaagcaaaactaggaggaatatagcatgaaaaaagtgattaaaaaagcggcgattggcatggtggcgttttttgtggtggcggcgagcggcccggtgtttgcgcttcaacttcaaccatttccacaaccagaacttccatacccacaaccacaacttccatacccacaaccagaacttccatacccacaaccacaaccattttaaggtttagatggttttaattagcaatatctgaatttaatagatgtcctttctatgtttaattacaaaacatagggagggcatttttttgatgcaattcgaacaattagtgaaagaatatttactagaactaaaactaggaaattattctaaaaggacaatggaaacatatgagcaacatatagataagttcattgatttctacaggaaagaaatatgcaaagaaatggatatttatgcaattaacaaaatgcactacaaactattcatttcacagctcttagataactctctgagggctacatacatcaatgctatactcaaatcaaataaagccttctacagttatctaataagggaacaggtgcttaaaacaagtcctatggactctataaagctacttaaagagactaaacaagcattaacaacattcaatgatgatgaagttaaggcaatgttaaacgtgtggaactttaacacctatttgaatgccagaaacaaatgtatcatcgctgttttagctgatacaggaataaggatttcagaactaatcaatattaaagattcagacctcacagaccagtatataagagttttggggaaaggtgacaagtggagagtagtacctatatcaaatgaattaaactatctcctgacgaaatataagagactcagagacaatcatttcaataagataagaaataggaatggtaaggtcagagaacttgattctgagctgtttttaggcaagacagggcgctctataaaaacgattacaaacattgaggttatgattacccaaacaggcttacaagccaatgtgagggcttctgtgaggtgcagtccacatcaatttagacattactggacgtgtaagagcttagaactgggacaagacatatttactatcagtaagctgttaggacacacaaatttatcaacaacacaaatttatttacaaaagttgacaaatgaacaattgataagtaaggctgtcaaattcagtccactaaaacatttaagctaatatatcttacagaacacaatcttaaacttaactataacttaacatataatgatgtgtttttctgat

SEQ ID NO: 31 - protein, 295 aa, Secale cereale
MKTFLILSLLAIVATTTTIAVRVPVPQLQPQNPSQQQPQEQVPLVQQQQFPGQQQPFPPRQPYPQPQPFPSQQPYLQLQPFPQPQQPYPQPQLLYPQPQPFRPQQPYPQPQPQYSQPQQPISQQQQQQQQQQQQQILQQILQQQLIPCRDVVLQQHSIAHGSSQVLQQSTYQLVQQLCCQQLWQIPEQSRCQAIHNVVHAIILHQQQQQQQQQQQQQQQPLSQVSFQQPQQQYPSGQGSFQPSQQNPQAQGSVQPQQLPQFEEIRNLALETLPAMCNVYIPPYCTIAPVGIFGTN

SEQ ID NO: 32 - protein, 293 aa, Hordeum vulgare
MKTFLIFALLAIAATSTIAQQQPFPQQPIPQQPQPYPQQPQPYPQQPFPPQQPFPQQPVPQQPQPYPQQPFPPQQPFPQQPPFWQQKPFPQQPPFGLQQPILSQQQPCTPQQTPLPQGQLYQTLLQLQIQYVHPSILQQLNPCKVFLQQQCSPVPVPQRIARSQMLQQSSCHVLQQQCCQQLPQIPEQFRHEAIRAIVYSIFLQEQPQQLVEGVSQPQQQLWPQQVGQCSFQQPQPQQVGQQQQVPQSAFLQPHQIAQLEATTSIALRTLPMMCSVNVPLYRILRGVGPSVGV

SEQ ID NO: 33 - protein, 33 aa, Artificial Sequence
  source: /note=“Description of Artificial Sequence Synthetic polypeptide”
LQLQPFPQPELPYPQPELPYPQPELPYPQPQPF

SEQ ID NO: 34 - protein, 33 aa, Lactococcus lactis
MKKRVQRNKKRIRWASVLTVFVLLIGIIAIAFA

SEQ ID NO: 35 - protein, 26 aa, Lactococcus lactis
MKQKHKLALGASIVALASLGGIKAQA

SEQ ID NO: 36 - protein, 30 aa, Lactococcus lactis
MNLAKNWKSFALVAAGAIAVVSLAACGKSA

SEQ ID NO: 37 - protein, 26 aa, Lactococcus lactis
MLKKIIISAALMASLSAAMIANPAKA

SEQ ID NO: 38 - protein, 27 aa, Lactococcus lactis
MKKKIISAILMSTVILSAAAPLSGVYA

SEQ ID NO: 39 - protein, 22 aa, Lactococcus lactis
MKKIIYGVGLISLLNVGTIAYG

SEQ ID NO: 40 - protein, 27 aa, Lactococcus lactis
MKQAKIIGLSTVIALSGIILVACGSKT

SEQ ID NO: 41 - protein, 24 aa, Lactococcus lactis
MKKFLLLGATALSLFSLAACSSSN

SEQ ID NO: 42 - protein, 26 aa, Lactococcus lactis
MKKVIKKAAIGMVAFFVVAASGPVFA

SEQ ID NO: 43 - protein, 29 aa, Lactococcus lactis
MSKKSIKKITMTVGVGLLTAIMSPSVINQ

SEQ ID NO: 44 - protein, 29 aa, Lactococcus lactis
MRHKKIYLLLAMIGATSAWTVANENQVKA

SEQ ID NO: 45 - protein, 24 aa, Lactococcus lactis
MKKFVLIILLLFSSSILLADKSSA

SEQ ID NO: 46 - protein, 23 aa, Lactococcus lactis
MKIKYILWVICALLLLNTGPSFA

SEQ ID NO: 47 - protein, 30 aa, Lactococcus lactis
MNKLKVTLLASSVVLAATLLSACGSNQSSS

SEQ ID NO: 48 - protein, 28 aa, Lactococcus lactis
MKKKIISAILMSTVILSAAAPLSGVYAG

SEQ ID NO: 49 - protein, 27 aa, Lactococcus lactis
MKKNIISAILMSTVILSAAAPLSGVYA

SEQ ID NO: 50 - DNA, 21 bp, Artificial Sequence
  source: /note=“Description of Artificial Sequence Synthetic primer”
aggtcggtgtgaacggatttg

SEQ ID NO: 51 - DNA, 22 bp, Artificial Sequence
  source: /note=“Description of Artificial Sequence Synthetic primer”
gacccagagtagttcacattcg

SEQ ID NO: 52 - DNA, 25 bp, Artificial Sequence
  source: /note=“Description of Artificial Sequence Synthetic primer”
tggaaagatgatggggaccttgtgc

SEQ ID NO: 53 - DNA, 21 bp, Artificial Sequence
  source: /note=“Description of Artificial Sequence Synthetic primer”
gcttcacatagtgcaggagac

SEQ ID NO: 54 - DNA, 20 bp, Artificial Sequence
  source: /note=“Description of Artificial Sequence Synthetic primer”
gagaagacctatcaggacca

SEQ ID NO: 55 - DNA, 23 bp, Artificial Sequence
  source: /note=“Description of Artificial Sequence Synthetic primer”
tgtagaccatgtagttgaggtca

SEQ ID NO: 56 - DNA, 21 bp, Artificial Sequence
  source: /note=“Description of Artificial Sequence Synthetic primer”
ccacgtagccaacgactatga

SEQ ID NO: 57 - DNA, 25 bp, Artificial Sequence
  source: /note=“Description of Artificial Sequence Synthetic primer”
tgggggaccttgaggttgatcttgg

SEQ ID NO: 58 - DNA, 22 bp, Artificial Sequence
  source: /note=“Description of Artificial Sequence Synthetic primer”
gtgcttgtgtcaacacggaata

SEQ ID NO: 59 - DNA, 17 bp, Artificial Sequence
  source: /note=“Description of Artificial Sequence Synthetic primer”
agcctgtggtaagcatg

SEQ ID NO: 60 - DNA, 102 bp, Artificial Sequence
  source: /note=“Description of Artificial Sequence Synthetic polynucleotide”
cttcaacttcaaccatttccacaaccacaacttccatacccacaaccacaacttccatacccacaaccacaacttccatacccacaaccacaaccattttaa

SEQ ID NO: 61 - DNA, 102 bp, Artificial Sequence
  source: /note=“Description of Artificial Sequence Synthetic polynucleotide”
cttcaacttcaaccatttccacaaccagaacttccatacccacaaccacaacttccatacccacaaccagaacttccatacccacaaccacaaccattttaa

SEQ ID NO: 62 - protein, 24 aa, Lactococcus lactis
MSITATIAAGATALTLLGAGGAAA

SEQ ID NO: 63 - protein, 27 aa, Lactococcus lactis
MSITATIAAGATALTLLGAGGAAAVNA

SEQ ID NO: 64 - protein, 57 aa, Lactococcus lactis
MPVSRVKVKNRHLKKKTKKPLAFYKPATKFAGAVLIAGTLTTTHELLLQQTSPMVQA

SEQ ID NO: 65 - protein, 14 aa, Lactococcus lactis
MSQKRSARSKSSKK

SEQ ID NO: 66 - protein, 25 aa, Lactococcus lactis
MTPKTKAAVLTGTIDSTGAVTGVTG

SEQ ID NO: 67 - protein, 33 aa, Lactococcus lactis
MVNTQVKRVKKQKFIAGTALLLGMATFGMVGKA

SEQ ID NO: 68 - protein, 18 aa, Lactococcus lactis
MLLSVLPVNLLGVMKVDA

SEQ ID NO: 69 - protein, 33 aa, Lactococcus lactis
MISVKKRKNIKVFLITASIGIVALGGQRVLADA

SEQ ID NO: 70 - protein, 25 aa, Lactococcus lactis
MKLKKSHIISLILFSGLLLVEPVLA

SEQ ID NO: 71 - protein, 28 aa, Lactococcus lactis
MKIKNLLMAATTVATLGAIGTVSAQASA

SEQ ID NO: 72 - protein, 28 aa, Lactococcus lactis
MNKSKIIAFSAVSLSAALLLTACGNSSS

SEQ ID NO: 73 - protein, 52 aa, Lactococcus lactis
MEMQKKKAPRKKGKVITKRKVLSATMSGTLLMTSVIIPTAYSLLSNQITAKA

SEQ ID NO: 74 - protein, 34 aa, Lactococcus lactis
MKFNKKRVAIATFIALIFVSFFTISSIQDNQTNA

SEQ ID NO: 75 - protein, 49 aa, Lactococcus lactis
MKKTLRDQLLGVSKAHLNWKNKTKVFIYGTAILLMVAPNLASSVSRASA

SEQ ID NO: 76 - protein, 29 aa, Lactococcus lactis
MKSPSKFWLLSTGILLSLLVTSLPLAVKA

SEQ ID NO: 77 - protein, 21 aa, Lactococcus lactis
MSILAFALVLIFGFVSQNAFA

SEQ ID NO: 78 - protein, 34 aa, Lactococcus lactis
MKLNSLNKKFALASVSLLTISTLAGFGGLVNVNA

SEQ ID NO: 79 - protein, 30 aa, Lactococcus lactis
MKFKKLGLVMATVFAGAALVTLSGCSSSDS

SEQ ID NO: 80 - DNA, 99 bp, Artificial Sequence
  source: /note=“Description of Artificial Sequence Synthetic oligonucleotide”
cttcaacttcaaccatttccacaaccagaacttccatacccacaaccagaacttccatacccacaaccagaacttccatacccacaaccacaaccattt

SEQ ID NO: 81 - DNA, 807 bp, Triticum aestivum
atggttagagttccagtgccacaattgcagccacaaaatccatctcagcaacagccacaagagcaagttccattggtacaacaacaacaatttctagggcagcaacaaccatttccaccacaacaaccatatccacagccgcaaccatttccatcacaactaccatatctgcagctgcaaccatttccgcagccgcaactaccatattcacagccacaaccatttcgaccacaacaaccatatccacaaccgcaaccacagtattcgcaaccacaacaaccaatttcacagcagcagcagcagcagcagcagcagcaacaacaacaacaacaacaacaacaaatccttcaacaaattttgcaacaacaactgattccatgcatggatgttgtattgcagcaacacaacatagcgcatggaagatcacaagttttgcaacaaagtacttaccagctgttgcaagaattgtgttgtcaacacctatggcagatccctgagcagtcgcagtgccaggccatccacaatgttgttcatgctattattctgcatcaacaacaaaaacaacaacaacaaccatcgagccaggtctccttccaacagcctctgcaacaatatccattaggccagggctccttccggccatctcagcaaaacccacaggcccagggctctgtccagcctcaacaactgccccagttcgaggaaataaggaacctagcgctacagacgctacctgcaatgtgcaatgtctacatccctccatattgcaccatcgcgccatttggcatcttcggtactaactatcga

SEQ ID NO: 82 - DNA, 885 bp, Secale cereale
atgaagacctttctcatcctttccctccttgctattgtagcaaccaccaccacaattgcagttagagttccagtgccacaattgcagccacaaaatccatctcagcaacaaccacaagagcaagttccattggtacaacaacaacaatttccagggcagcaacaaccatttccaccacgacagccatatccgcagccgcagccatttccatcacaacaaccatatctgcagctgcaaccatttccgcagccgcaacaaccatatccgcagccgcaactactatatccgcagccgcaaccatttcgaccacaacaaccatatccacaaccgcaaccacagtattcgcaaccacaacaaccaatttcgcagcagcagcagcagcaacaacaacaacaacaacaacagatccttcaacaaattttgcaacaacaactgattccatgcagggatgttgtattgcaacaacacagcatagcgcatggaagctcacaagttttgcaacaaagtacttaccaactggtgcaacaattgtgttgtcagcagctgtggcagatccccgagcagtcgcggtgccaagccattcacaatgttgttcatgctattattctgcatcaacagcaacaacaacaacaacaacaacaacaacaacaacaacaaccgttgagccaggtctccttccaacagcctcaacaacaatatccatcaggccagggctccttccagccatctcagcaaaacccacaggcccagggctctgtccagcctcaacaactgccccagtttgaggaaataaggaacctagcgctagagacgctacctgcaatgtgcaatgtctatatccctccatattgcaccattgctccagttggcatcttcggtactaac

SEQ ID NO: 83 - DNA, 28 bp, Lactococcus lactis
taagcaaaactaggaggaatatagcatg

SEQ ID NO: 84 - DNA, 462 bp, Homo sapiens
atgtacaggatgcaactcctgtcttgcattgcactaagtcttgcacttgtcacaaacagtgctccaacttcatcatcaactaaaaaaactcaattgcaacttgaacacttgcttttggatcttcaaatgatcttgaacggtatcaacaactacaaaaacccaaaacttactcgtatgttgacttttaaattttacatgccaaaaaaagctactgaacttaaacacttgcaatgtcttgaagaagaattgaaaccacttgaagaagttttgaaccttgctcaatcaaaaaactttcacttgcgtccacgtgatcttatctcaaacatcaacgttatcgttttggaacttaaaggttcagaaactacttttatgtgtgaatacgctgatgaaactgctactatcgttgaatttttgaaccgttggatcactttttgtcaatcaatcatctcaactttgacttaa

SEQ ID NO: 85 - DNA, 139 bp, Unknown
  source: /note=“Description of Unknown P1 promoter sequence”
aagtcatcttacctcttttattagttttttcttataatctaatgataacatttttataattaatctataaaccatatccctctttggaatcaaaatttattatctactcctttgtagatatgttataatacaagtatca

SEQ ID NO: 86 - DNA, 99 bp, Lactococcus lactis
atgaaaaaacgcgtgcagcgcaacaaaaaacgcattcgctgggcgagcgtgctgaccgtgtttgtgctgctgattggcattattgcgattgcgtttgcg

SEQ ID NO: 87 - DNA, 72 bp, Lactococcus lactis
atgagcattaccgcgaccattgcggcgggcgcgaccgcgctgaccctgctgggcgcgggcggcgcggcggcg

SEQ ID NO: 88 - DNA, 81 bp, Lactococcus lactis
atgagcattaccgcgaccattgcggcgggcgcgaccgcgctgaccctgctgggcgcgggcggcgcggcggcggtgaacgcg

SEQ ID NO: 89 - DNA, 171 bp, Lactococcus lactis
atgccggtgagccgcgtgaaagtgaaaaaccgccatctgaaaaaaaaaaccaaaaaaccgctggcgttttataaaccggcgaccaaatttgcgggcgcggtgctgattgcgggcaccctgaccaccacccatgaactgctgctgcagcagaccagcccgatggtgcaggcg

SEQ ID NO: 90 - DNA, 42 bp, Lactococcus lactis
atgagccagaaacgcagcgcgcgcagcaaaagcagcaaaaaa

SEQ ID NO: 91 - DNA, 78 bp, Lactococcus lactis
atgaaacagaaacataaactggcgctgggcgcgagcattgtggcgctggcgagcctgggcggcattaaagcgcaggcg

SEQ ID NO: 92 - DNA, 75 bp, Lactococcus lactis
atgaccccgaaaaccaaagcggcggtgctgaccggcaccattgatagcaccggcgcggtgaccggcgtgaccggc

SEQ ID NO: 93 - DNA, 90 bp, Lactococcus lactis
atgaacctggcgaaaaactggaaaagctttgcgctggtggcggcgggcgcgattgcggtggtgagcctggcggcgtgcggcaaaagcgcg

SEQ ID NO: 94 - DNA, 78 bp, Lactococcus lactis
atgctgaaaaaaattattattagcgcggcgctgatggcgagcctgagcgcggcgatgattgcgaacccggcgaaagcg

SEQ ID NO: 95 - DNA, 99 bp, Lactococcus lactis
atggtgaacacccaggtgaaacgcgtgaaaaaacagaaatttattgcgggcaccgcgctgctgctgggcatggcgacctttggcatggtgggcaaagcg

SEQ ID NO: 96 - DNA, 54 bp, Lactococcus lactis
atgctgctgagcgtgctgccggtgaacctgctgggcgtgatgaaagtggatgcg

SEQ ID NO: 97 - DNA, 99 bp, Lactococcus lactis
atgattagcgtgaaaaaacgcaaaaacattaaagtgtttctgattaccgcgagcattggcattgtggcgctgggcggccagcgcgtgctggcggatgcg

SEQ ID NO: 98 - DNA, 81 bp, Lactococcus lactis
atgaaaaaaaaaattattagcgcgattctgatgagcaccgtgattctgagcgcggcggcgccgctgagcggcgtgtatgcg

SEQ ID NO: 99 - DNA, 75 bp, Lactococcus lactis
atgaaactgaaaaaaagccatattattagcctgattctgtttagcggcctgctgctggtggaaccggtgctggcg

SEQ ID NO: 100 - DNA, 66 bp, Lactococcus lactis
atgaaaaaaattatttatggcgtgggcctgattagcctgctgaacgtgggcaccattgcgtatggc

SEQ ID NO: 101 - DNA, 84 bp, Lactococcus lactis
atgaaaattaaaaacctgctgatggcggcgaccaccgtggcgaccctgggcgcgattggcaccgtgagcgcgcaggcgagcgcg

SEQ ID NO: 102 - DNA, 81 bp, Lactococcus lactis
atgaaacaggcgaaaattattggcctgagcaccgtgattgcgctgagcggcattattctggtggcgtgcggcagcaaaacc

SEQ ID NO: 103 - DNA, 84 bp, Lactococcus lactis
atgaacaaaagcaaaattattgcgtttagcgcggtgagcctgagcgcggcgctgctgctgaccgcgtgcggcaacagcagcagc

SEQ ID NO: 104 - DNA, 72 bp, Lactococcus lactis
atgaaaaaatttctgctgctgggcgcgaccgcgctgagcctgtttagcctggcggcgtgcagcagcagcaac

SEQ ID NO: 105 - DNA, 78 bp, Lactococcus lactis
atgaaaaaagtgattaaaaaagcggcgattggcatggtggcgttttttgtggtggcggcgagcggcccggtgtttgcg

SEQ ID NO: 106 - DNA, 87 bp, Lactococcus lactis
atgagcaaaaaaagcattaaaaaaattaccatgaccgtgggcgtgggcctgctgaccgcgattatgagcccgagcgtgattaaccag

SEQ ID NO: 107 - DNA, 87 bp, Lactococcus lactis
atgcgccataaaaaaatttatctgctgctggcgatgattggcgcgaccagcgcgtggaccgtggcgaacgaaaaccaggtgaaagcg

SEQ ID NO: 108 - DNA, 72 bp, Lactococcus lactis
atgaaaaaatttgtgctgattattctgctgctgtttagcagcagcattctgctggcggataaaagcagcgcg

SEQ ID NO: 109 - DNA, 69 bp, Lactococcus lactis
atgaaaattaaatatattctgtgggtgatttgcgcgctgctgctgctgaacaccggcccgagctttgcg

SEQ ID NO: 110 - DNA, 156 bp, Lactococcus lactis
atggaaatgcagaaaaaaaaagcgccgcgcaaaaaaggcaaagtgattaccaaacgcaaagtgctgagcgcgaccatgagcggcaccctgctgatgaccagcgtgattattccgaccgcgtatagcctgctgagcaaccagattaccgcgaaagcg

SEQ ID NO: 111 - DNA, 102 bp, Lactococcus lactis
atgaaatttaacaaaaaacgcgtggcgattgcgacctttattgcgctgatttttgtgagcttttttaccattagcagcattcaggataaccagaccaacgcg

SEQ ID NO: 112 - DNA, 147 bp, Lactococcus lactis
atgaaaaaaaccctgcgcgatcagctgctgggcgtgagcaaagcgcatctgaactggaaaaacaaaaccaaagtgtttatttatggcaccgcgattctgctgatggtggcgccgaacctggcgagcagcgtgagccgcgcgagcgcg

SEQ ID NO: 113 - DNA, 87 bp, Lactococcus lactis
atgaaaagcccgagcaaattttggctgctgagcaccggcattctgctgagcctgctggtgaccagcctgccgctggcggtgaaagcg

SEQ ID NO: 114 - DNA, 63 bp, Lactococcus lactis
atgagcattctggcgtttgcgctggtgctgatttttggctttgtgagccagaacgcgtttgcg

SEQ ID NO: 115 - DNA, 102 bp, Lactococcus lactis
atgaaactgaacagcctgaacaaaaaatttgcgctggcgagcgtgagcctgctgaccattagcaccctggcgggctttggcggcctggtgaacgtgaacgcg

SEQ ID NO: 116 - DNA, 90 bp, Lactococcus lactis
atgaacaaactgaaagtgaccctgctggcgagcagcgtggtgctggcggcgaccctgctgagcgcgtgcggcagcaaccagagcagcagc

SEQ ID NO: 117 - DNA, 90 bp, Lactococcus lactis
atgaaatttaaaaaactgggcctggtgatggcgaccgtgtttgcgggcgcggcgctggtgaccctgagcggctgcagcagcagcgatagc

SEQ ID NO: 118 - DNA, 81 bp, Lactococcus lactis
atgaaaaaaaagattattagtgctatcttgatgagcacagttatcttgagtgcggccgctccactatcaggggtatatgct

SEQ ID NO: 119 - DNA, 84 bp, Lactococcus lactis
atgaaaaaaaaaattattagcgcgattctgatgagcaccgtgattctgagcgcggcggcgccgctgagcggcgtgtatgcgggc

SEQ ID NO: 120 - DNA, 81 bp, Lactococcus lactis
atgaaaaaaaacattatctcagctattttaatgtctacagtgatactttctgctgcagccccgttgtcaggtgtttacgcc

SEQ ID NO: 121 - DNA, 3289 bp, Artificial Sequence
  source: /note=“Description of Artificial Sequence Synthetic polynucleotide”
gatggctgaagctccaactcatgaacaagttgaccatgttgtggatacaattgttgaagttgttgaagaggaaattggtgtgaaataaagaaaagacaaggagaatattcttcttgtcttttttcatatcctaaaactctacctactgtggtagagtttttttatcttttttggcgtctagcaaactctgtaaaacgaaaacggtcaacctgatgtcgtgattcagtatactggaaaagtgtattatcggcaagatagacatgagatttcacggaaacaacatgatggtccttcggatttaaatcaagatatgtaaaatcatcttcacaagcaaaatcaatggtaacttctttttgggcataggcaatatcaagtcccaaagccccttctaaataatcgtaagtagaattttgggcatgtgcgggggtcaaaccatcagcgtatttttctaaaaataaatcccaatccaaaatggaaaatttaccatctacttttcttcttctaagaatactaagggcttggtcgccgatggcgaatccagtagtttctgaaagagctggggtaatttttatactttcaaattttattacttcagtttcactgtgaaaacccattgaagtttgcaattctttatatgaagttaagccggaaatagggaaaaggagccgatcgtgagcgaggacaatgctgccatagccatgtcttctttggatgagccctttttcttctaaaatttttaaagcttgtctgacggttgaacggctactttcataactaatagaaagttcattctcgcttggaagaatatcgttcgttttatagatatcattaaaaatctttttttctaaatcttgcaaaatcacttcatatttcttcatactttatattttatcataaaaataattgttaacgcttgctgaaaacgtttttatgaaaacgccttaaaatggcattttgacttgcaaactgggctaagatttgctaaaatgaaaaatgcctatgtttaaggtaaaaaacaaatggaggacatttctaaaatgtttggaataggaaaaaagaaagaattgagagatgataaaagcctttatgctccagtttctggggaagttatcaacctttcaacagtcaacgaccccgtattttcaaaaaagataatgggagacgggttcgcggttgagccaaaagaaaataaaatttttgccccagtttctgcaaaagtaactttggttcaaggacatgcaattggttttaaacgtgctgatggcttagatgtacttttacatcttggaattgatacagtagctcttaaaggtcttcattttaaaatcaaggtcaaagttgatgatattgtcaatggtggtgatgagcttggaagcgttgattgggcacagattgaagctgcaggtttagataaaacgacaatggttatctttacaaatacaaaagataaactctctgagttcaatgtcaattatggaccagctacttctggaagtgaacttggtaaggcaagtgttaaataaggaggaaaaaatggcaaattattcacaacttgcgacagaaattatcgcaaatgtaggtggcgctgagaatgtcacaaaagttattcactgtatcactcgtcttcgttttaccttgaaagacaaagataaagcagatacggcggcgattgaagccttacctggtgtcgctggagctgtttataactcaaacttgaatcaatatcaagtagttattggacaagctgtagaagatgtttatgacgaggttgttgaacagcttggagattcagttgttgatgaagatgcaacggcgcaagcacttgctgcaacagcaccggctagtggtaaaaaacaaaatccaattgttcatgctttccaagtggttattgggacaattacaggttcgatgattccaattattggtttacttgcggctggtgggatgattaatggattattaagtatctttgttaaaggaaatcgtttaattgaagtgattgaccctgcaagttcaacttacgtcattatctcaactctagcaatgacaccattttatttcttacctgttttagtaggattttcagcagcaaaacaattagcacctaaagatactgttttacaatttattggtgctgctgttggtggtttcatgattaatccagggattactaacttggtaaatgctcatgttggaacaaatgcggccggtaaaaatgttgttgttgaagcagcagctccagtagcaaatttccttggagtcacttttaatacaagttattttggaattccggttgctttgccaagttatgcttatacaattttcccaatcattgtggcggtagcaatcgctaaacctttgaatgcttggttgaaaaaggttttaccacttgccttgcgtccaattttccaaccgatgattactttcttcatcactgcttcaatcattttactcttggtcggtcctgttatttcaacaatttcatctggtttgtcattcgttattgaccatatcttgtcattaaacttagggattgcaagtattatcgtcggtggtttgtatcaatgtttggttatatttggtttgcactggttggttgtaccacttatttcacaagagttggcagcaacaggagcaagctcacttaatatgattgttagcttcacaatgcttgcgcaaggagttggtgccttgactgtcttctttaaatctaaaaaagctgaccttaaaggactttctgctccagctgccatttcggctttttgtggagtaactgaacctgccatgtacggaattaacttgaaatatgttcgcgtcttcatcatgtcttcaattggtgcagcaattggtgctgggattgccggatttggtggcttacaaatgtttggattttcagggtcattgattagttttcctaactttatctctaatccattgacgcatcatgcacctgcgggtaacttaatgctcttctggattgccactgcggtatgtgctgttgccactttcttattagtttggttctttggttacaaggatactgatgtcatgggacaaggagttgaacaaaaaaatgcatttaaggatgctgtaaaataaatagttttgctcttaataaagttttgatacaaggatttacaattattttttgataaaaaaattactgatagaaatgaaaaaaattctgtcagtaattttggaaagtcattctaaaaaattcattttaaaatgacgagaaagaaggtaaaaagatgtttaaagcagtattgtttgatttagatggcgtaat